(19) United States
(12) Patent Application Publication
(10) Pub. No.: US 2023/0406854 A1
(43) Pub. Date: Dec. 21, 2023

(54) COVALENT KRAS-BINDING COMPOUNDS FOR THERAPEUTIC PURPOSES

(71) Applicant: Umbra Therapeutics, Inc., Lexington, MA (US)

(72) Inventors: Guillaume Barbe, Milton, MA (US); Mark George Sauliner, Higganum, CT (US); William John Greenlee, Teaneck, NJ (US); Soumya Ray, Quincy, MA (US)

(73) Assignee: Umbra Therapeutics, Inc., Lexington, MA (US)

(21) Appl. No.: 18/242,469

(22) Filed: Sep. 5, 2023

Related U.S. Application Data
(63) Continuation of application No. PCT/US2022/018998, filed on Mar. 4, 2022.
(60) Provisional application No. 63/157,405, filed on Mar. 5, 2021.

(51) Int. Cl. C07D 471/04

(52) U.S. Cl. CPC C07D 471/04

(57) ABSTRACT

Heteroaryl sulfonyl compounds and compositions that have a KRAS Recognition Moiety bound to an electrophile for the selective covalent modification of KRAS, for example a mutant KRAS, to treat KRAS-mediated disorders are described.

### CROSS-REFERENCE TO RELATED APPLICATIONS

This application is a continuation of International Patent Application No. PCT/US2022/018998, filed in the U.S. Receiving Office on Mar. 4, 2022, which claims the benefit of U.S. Provisional Application No. 63/157,405, filed on Mar. 5, 2021. The entirety of each of these applications is hereby incorporated by reference for all purposes.

### FIELD OF THE INVENTION

This invention provides heteroaryl sulfonyl compounds, compositions, and methods of use thereof, that include a KRAS Recognition Moiety for the selective covalent modification of KRAS, for example a mutant KRAS, to treat KRAS-mediated disorders, typically through tyrosine or lysine.

### BACKGROUND OF THE INVENTION

Most cells in the body are terminally differentiated with protective mechanisms to prevent cellular proliferation. A small subset of cells undergoes cellular proliferation mainly to replenish tissue or blood components, such as hematopoietic cells and their progeny. The body maintains a careful balance between terminal differentiation and cellular proliferation through a complex network of cellular signaling.

For the cell to properly respond to external signals, it uses a carefully balanced relay of signaling proteins. One such relay protein is KRAS, which was first identified as an oncogene in Kirsten RAt Sarcoma virus. KRAS is an essential component of the mitogen-activated protein kinase (“MAPK”) pathway, which regulates cell cycling and proliferation. More specifically, it is involved in signaling pathways that initiate extracellularly and are transferred to the cell nucleus, causing the cell to grow, divide or differentiate. KRAS acts by dephosphorylating guanosine 5′-triphosphate to guanosine 5′-diphosphate and is thus referred to as a GTPase.

If KRAS is mutated or upregulated, abnormal cellular proliferation can occur. For example, a single mutation of G12C or G12D can cause KRAS to continuously activate downstream proteins, regardless of input from extracellular sources. This mutation leads to unregulated cell growth and is a driver mutation in up to 20% of human cancers, including 90% of pancreatic cancers and greater than 40% of colon cancers (Cox et al. Nat. Rev. Drug Discov. 13, 828-851 (2014); Gorfe Cell, 50006-3495 (19) 32753-5 (2020)).

There are several compounds in development that target KRAS for the treatment of cancer. Initial compound library screening efforts were used to find known molecules that phenotypically inhibit KRAS driven cancer such as oncrasin, tolperisone, lanperisone, and DCAI. (Wang et al. J. Med Chem. 56(13), 5219-5230). There has also been limited success in the development of classic reversible allosteric KRAS inhibitors (McCarthy et al. ACS Omega, 4, 2921-2930 (2019)). Most KRAS inhibitors that are currently being developed covalently target the G12C cysteine mutation instead of reversibly binding the allosteric binding pocket. (Goebel et al. RSC Medicinal Chemistry 11, 260 (2020)). Examples of covalent KRAS inhibitors include adagrasib which is being developed by Mirati Therapeutics, sotorasib which is being developed by Amgen, and ARS-3248 and ARS-1620 which are being developed by Johnson and Johnson/Wellspring Bioscience. (Gilson et al. Cancers 12(5), 1341 (2020)). These inhibitors react irreversibly with cysteine in KRAS G12C (Goebel et al. RSC Medicinal Chemistry 11, 260 (2020)). Despite the extensive effort to develop a KRAS inhibitor, there are currently no specific FDA approved drugs that act on KRAS.

The Scripps Research Institute filed three PCT Applications, WO2015/188120, WO2018/102433, and WO2019/139979 describing fluorosulfur (VI) compounds and uses thereof via reactions with phenols. This chemistry, known as SuFEx (Sulfur-Fluoride Exchange), has also been studied by the Sharpless lab at the Scripps Research Institute, which has published a number of papers on the topic (Dong et al. Angew. Chem. Int. Ed Engl. 53(36), 9466-9470 (2014); Qin et al. Angew. Chem. Int. Ed 55(45), 14155-14158 (2016); Gao et al. Angew. Chem. Int. Ed 57(7), 1939-1943 (2018); Guo et al. Angew. Chem. Int. Ed 57(10), 2605-2610 (2017); Gahtory et al. Chemistry 24(41), 10550-10556, (2018); Smedley et al. Angew. Chem. Int. Ed 58(14), 4552-4556 (2019); Liu et al. Angew. Chem. Int. Ed 58(24), 8029-8033 (2019); Dong et al. Angew. Chem. Int. Ed 53(36), 9430-9448 (2014); Li et al. Angew. Chem. Int. Ed 56(11), 2903-2908 (2017); Zheng et al. PNAS 116(38) 18808-18814 (2019); Wang et al. Angew. Chem. Int. Ed 56(37), 11203-11208 (2017); Liu et al. J. Am. Chem. Soc. 140, 2919-2925 (2018); and Chen et al. J. Am. Chem. Soc. 138, 7353-7364 (2016)). Similar sulfonyl fluoride chemistries have been developed for the purpose of biorthogonal protein labelling (Narayanan et al. Chem. Sci. 6(5): 2650-2659 (2015) and Gu et al. J. Chem. Biol. 20(4), 541-548 (2013)) These strategies employ electrophilic sulfonyl compounds with a fluoride leaving group to react with a variety of nucleophiles.

Ku-Lung Hsu, et al., at University of Virginia have described sulfonyl-containing heteroaryl compound which have been named SuTEx compounds (Sulfur-Triazole Exchange)(Hahm et al. Nat. Chem. Bio. 16, 150-159 (2020); Brulet et al. J. Am. Chem. Soc. 142(18), 8270-8280 (2020); Borne et al. Development and biological applications of sulfur-triazole exchange (SuTEx) chemistry RSC Chem. Biol. (2021); and Huang et al. Chemoproteomic profiling of kinases in live cells using electrophilic sulfonyl triazole probes Chem. Sci. (2021)). See also WO 2020/214336 (Sulfur-heterocycle exchange chemistry) and WO 2021/016263 (Cysteine Binding Compositions and Methods of Use Thereof), filed by University of Virginia as assignee, and Hsu, et al. as inventors. Additional publications on the use of SuTEx molecules include Grams et al. Reactive chemistry for covalent probe and therapeutic development Trends in Pharmacological Sciences (2022) and Toroitich et al. Discovery off a cell-active SuTEx ligand of prostaglandin reductase 2 ChemBioChem (2021).

It is an object of the present invention to provide new compositions of matter and their methods of use and manufacture to treat diseases mediated by KRAS.

### SUMMARY OF THE INVENTION

Heteroaryl sulfonyl compounds and their uses and manufacture are provided that covalently modify KRAS to treat a disease mediated by KRAS, for example a mutant KRAS, in a host, typically a human. The heteroaryl sulfonyl compound is first typically selectively non-covalently bound to KRAS by association of KRAS with a KRAS Recognition Moiety in the heteroaryl sulfonyl compound. In a typical second step, a reactive tyrosine residue on KRAS attacks the sulfonyl moiety in the heteroaryl sulfonyl compound of the present invention to form a covalent bond between the tyrosine and the compound and force the elimination of a Leaving Group from the compound. In another aspect a reactive lysine residue on KRAS attacks the heteroaryl sulfonyl compound of the present invention to form a covalent bond between the lysine and the compound and force the elimination of a Leaving Group from the compound.

In a principal aspect of the invention the heteroaryl sulfonyl compound covalently modifies a mutant KRAS. Non-limiting examples of mutant KRAS include codon 12 mutations such as KRAS G12D, KRAS G12C, or KRAS G12V; codon 13 mutations such as KRAS G13C or KRAS G13D; codon 18 mutations such as KRAS A18D; codon 61 mutations such as KRAS Q61H; and codon 117 mutations such as KRAS K117N.

The heteroaryl sulfonyl compounds of the present invention are uniquely designed for KRAS specificity, for example mutant KRAS, to maximize therapeutic effect and minimize off-target toxicity, by inclusion of a specific KRAS Recognition Moiety as described further herein that selectively bind KRAS, for example mutant KRAS, for further covalent linkage. In this way, the heteroaryl sulfonyl compound of the present invention exerts precise control over the targeted silencing, destruction or inactivation of KRAS, for example mutant KRAS, while limiting unacceptable off-target effects.

KRAS Recognition Moiety is a molecule that has a functional group linking it to the heteroaryl sulfonyl compound of the present invention, and is, for example a synthetic or naturally occurring small molecule that binds to KRAS, for example mutant KRAS, as an inhibitor or alternatively has no apparent biological effect on KRAS. In non-limiting embodiments, the KRAS Recognition Moiety is a protein binding domain of a drug or pharmaceutically active compound which modulates KRAS (or the full drug or pharmaceutically active compound). In alternative embodiments, the KRAS Recognition Moiety may be a peptide, RNA, DNA, oligonucleotide, or another biologic compound or fragment thereof which can be suitably stabilized, as necessary.

The heteroaryl sulfonyl compounds described herein can take advantage of the variable electrophilic properties of heteroaryl sulfonyl compounds to covalently modify KRAS, resulting in a decrease or termination of its biological activity.

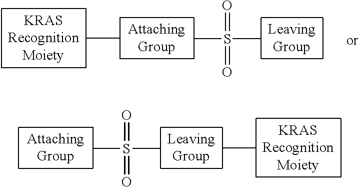

The covalent-binding heteroaryl sulfonyl compounds of the present invention include a KRAS Recognition Moiety, a Leaving Group, and an Attaching Group. The heteroaryl sulfonyl compounds are oriented such that the Leaving Group is on one side of the S(O)₂ electrophile and the Attaching Group is on the other. The KRAS Recognition Moiety is located either on the Leaving Group or the Attaching Group in a manner that allows it to associate with KRAS as described herein.

In some embodiments, the Leaving Group is a monocyclic or bicyclic heteroaryl group bound to the sulfur atom through a S—N bond. For example, as used herein, R¹ and R⁴ are typically Leaving Groups. The Leaving Group is eliminated when the heteroaryl sulfonyl compound undergoes nucleophilic attack by an amino acid, for example a tyrosine or lysine, of KRAS. The Attaching Group, along with the S(O)₂ group to which it is bound, remains on KRAS after covalent modification. For example, as used herein, R², R⁵, and R¹³ are Attaching Groups.

A non-limiting example of the covalent modification of KRAS via a tyrosine that reacts with the heteroaryl sulfonyl compound of the present invention is provided below:

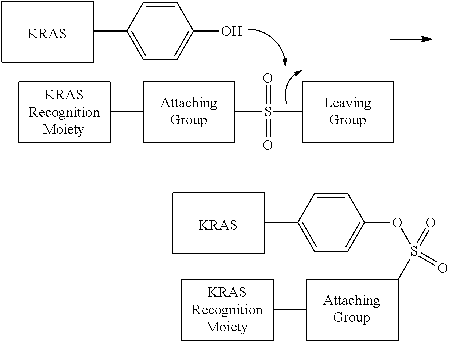

The KRAS Recognition Moiety brings the activated heteroaryl sulfonyl compound of the present invention into close proximity with a reactive amino acid of KRAS, for example mutant KRAS, resulting in covalent modification of KRAS and resultant amelioration or elimination of a disease or KRAS-mediated disorder.

The heteroaryl sulfonyl compound of the present invention is used to modulate KRAS's biological activity by covalently modifying the protein, for example, by covalently modifying a tyrosine, or alternatively, a lysine moiety in or near an allosteric binding pocket, for example the Switch-II Pocket (Ostrem et al. Nature, 503(7477), 548-551, (2013)).

In certain aspects, a heteroaryl sulfonyl compound of Formula I, Formula II, Formula III, Formula IV, Formula V, Formula VI, or Formula VII, is provided:

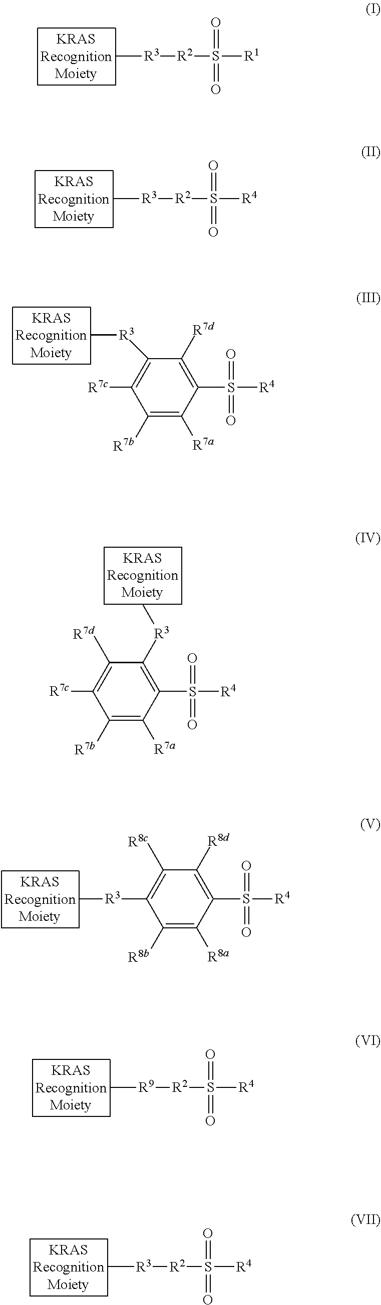

or a pharmaceutically acceptable salt, N-oxide, isotopic derivative, or prodrug thereof, optionally in a pharmaceutically acceptable carrier to form a pharmaceutical composition; wherein:

R¹ is selected from:

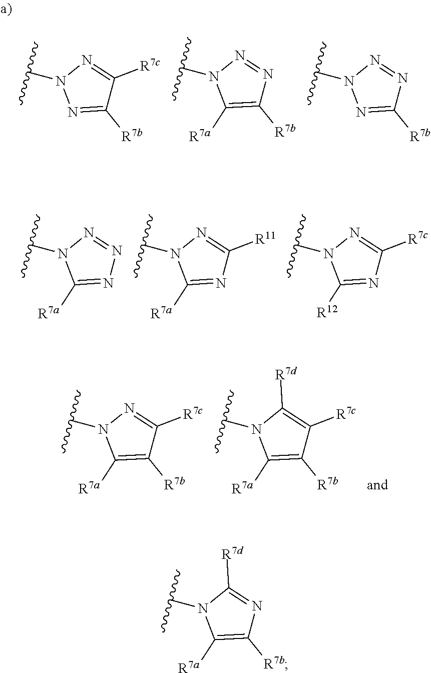

b) a bicyclic heteroaryl or tricyclic heteroaryl, each of which is optionally substituted as allowed by valence with 1, 2, 3, or 4 substituents selected from R⁷;
   R² is a bivalent moiety independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which except bond is optionally substituted as allowed by valence with 1, 2, 3, or 4 substituents selected from R⁷;
   in principal embodiments R² is selected from alkyl, alkenyl, haloalkyl, cycloalkyl, aryl, bicycle, tricycle, and heteroaryl, each of which is optionally substituted as allowed by valence with 1, 2, 3, or 4 substituents selected from R⁷;
   R³ is a bivalent moiety independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which except bond is optionally substituted as allowed by valence with 1, 2, 3, or 4 substituents selected from R⁷;
   or R³ is a bivalent moiety independently selected at each instance from bond, alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents selected from R⁷;
   and wherein R² and R³ are selected such that a suitably stable and suitably nontoxic compound for in vivo administration in a host is achieved, and in certain embodiments, in a way that avoids undesired repetition of atoms or moieties, such as in nonlimiting examples, S, O, or a combination thereof (i.e., that would otherwise form a disulfide or peroxide bond), as well known to skilled artisans;
   p is independently selected from 1, 2, 3, 4, 5, and 6;
   R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;
   R⁵ is a bivalent moiety selected from alkyl, alkenyl, haloalkyl, cycloalkyl, naphthyl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;
   in principal embodiments R⁵ is selected from alkyl, alkenyl, haloalkyl, cycloalkyl, naphthyl, heterocycle, bicycle, tricycle, and heteroaryl, each of which is optionally substituted as allowed by valence with 1, 2, 3, or 4 substituents selected from R⁷;
   and wherein R³ and R⁵ are selected such that a suitably stable and suitably nontoxic compound for in vivo administration in a host is achieved, and in certain embodiments, in a way that avoids undesired repetition of atoms or moieties, such as in nonlimiting examples, S, O, or a combination thereof (i.e., that would otherwise form a disulfide or peroxide bond), as well known to skilled artisans;
   R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;
   R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
   R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶;
   R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;
   R¹⁹ is independently selected at each instance from hydrogen, alkyl, haloalkyl, cycloalkyl, aryl, heterocycle, and heteroaryl;
   R^{8a}, R^{8b}, R^{8c}, and R^{8d} are independently selected at each instance from R⁷ and R¹² wherein at least one of R^{8a}, R^{8b}, R^{8c}, and R^{8d} is R¹²;
   R⁹ is a bivalent moiety selected from alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;
   or R⁹ is a bivalent moiety selected from alkyl, alkenyl, haloalkyl, cycloalkyl, heterocycle, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;
   R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R¹⁷; and
   R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷; and
   KRAS Recognition Moiety is a molecule which can bind to or otherwise anchors too KRAS.

In certain embodiments KRAS has a reactive tyrosine which covalently binds to the heteroaryl sulfonyl compounds of the present invention. In certain embodiments the reactive tyrosine is in an allosteric site, for example the Switch-II Pocket (Ostrem et al. Nature, 503(7477), 548-551, (2013)).

In certain embodiments KRAS has a reactive lysine which covalently binds to the heteroaryl sulfonyl compounds of the present invention. In certain embodiments the reactive lysine is in an allosteric site, for example the Switch-II Pocket.

In certain embodiments KRAS has a reactive cysteine which covalently binds to the heteroaryl sulfonyl compounds of the present invention. In certain embodiments the reactive cysteine is in an allosteric site, for example the Switch-II Pocket.

Assays and/or spectroscopic techniques to confirm covalent binding are described in the paper by Brulet et. al. titled “Liganding Functional Tyrosine Sites on Proteins Using Sulfur-Triazole Exchange Chemistry” JACS 2020, 142, 8270-8280 or the paper by Hahm et. al. titled “Global targeting of functional tyrosines using sulfur triazole exchange chemistry” Nature Chem. Biol. 2020, 16(2), 150-159.

In certain embodiments the heteroaryl sulfonyl compound of the present invention primarily covalently modifies a specific tyrosine or lysine in KRAS. In other embodiments, a selected heteroaryl sulfonyl compound of the present invention reacts with two or more different tyrosines and/or lysines in KRAS. In certain embodiments the heteroaryl sulfonyl compound of the present invention is more than about 5-, 10-, 15-, 20-, 25-, 50-, 75-, or 100-fold more selective for one specific amino acid, for example a specific tyrosine, than other amino acids of KRAS.

In certain embodiments one or more amino acid other than tyrosine or lysine is covalently modified by a heteroaryl sulfonyl compound of the present invention. For example, the amino acid that is covalently modified is cysteine, arginine, histidine, serine, threonine, or tryptophan.

In one aspect a heteroaryl sulfonyl compound of Formula VIII is provided:

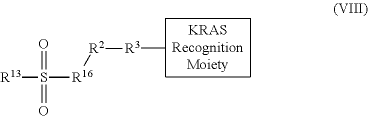

or a pharmaceutically acceptable salt, N-oxide, isotopic derivative, or prodrug thereof, optionally in a pharmaceutically acceptable carrier to form a composition; wherein:
R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;
R¹⁶ is a heteroaryl group, where the bond to the sulfur atom is through one of the nitrogen atoms present in the cycle, and R¹⁶ is optionally substituted as allowed by valence with 1, 2, 3, or 4 substituents selected from R⁷;
   and all other variables are as defined herein.

In a typical embodiment, R² is selected in each instance from bond, alkyl, alkenyl, haloalkyl, cycloalkyl, aryl, heterocycle, —(CH₂)ₚ—C(O)—, —(OCH₂CH₂)ₚ—, —C(O)—, —NR⁶C(O)—, and heteroaryl, each of which except bond is optionally substituted as allowed by valence with 1, 2, 3, or 4 substituents selected from R⁷, wherein if R² is bond, R³ is R³*; wherein
R³* is selected in each instance from bond, alkyl, alkenyl, haloalkyl, cycloalkyl, aryl, heterocycle, —(CH₂)ₚ—C(O)—, —(OCH₂CH₂)ₚ—, —C(O)—, —NR⁶C(O)—, bicycle, and heteroaryl, each of which except bond is optionally substituted as allowed by valence with 1, 2, 3, or 4 substituents selected from R⁷.

In a typical embodiment R⁵ is selected from alkyl, alkenyl, haloalkyl, cycloalkyl, naphthyl, heterocycle, —(OCH₂CH₂)ₚ—, —C(O)—, —NR⁶C(O)—, bicycle, and heteroaryl, each of which is optionally substituted as allowed by valence with 1, 2, 3, or 4 substituents selected from R⁷.

In certain embodiments of the invention, the KRAS Recognition Moiety is a small organic molecule (i.e., a non-biologic) that adequately binds to KRAS in a manner that it is covalently modified. In other embodiments of the invention, the KRAS Recognition Moiety is a peptide or oligonucleotide that adequately binds to the protein in such a manner that it is covalently modified. In certain embodiments the KRAS Recognition Moiety is a residue of a pharmaceutically active compound that binds to KRAS (for example but not limited to a compound of the sort that would be reviewed as a drug by CDER of the FDA, or an approved or clinical stage drug) or a peptide, protein or biologic or a binding fragment thereof that adequately binds to the protein in such a manner that it is covalently modified. A plethora of illustrative nonlimiting examples of KRAS Recognition Moieties for use in the heteroaryl sulfonyl compound of the present invention are provided in the detailed description and additional KRAS Recognition Moieties are readily apparent.

In principle embodiments, the KRAS Recognition Moiety is not a fluorophore, is not a detectable labeling group, and is not a moiety comprising an alkyne. In certain embodiments the KRAS Recognition Moiety is selective for KRAS and is not a non-selective protein binder, for example a promiscuous binder of a range of enzymes or of kinases.

In principle embodiments, the heteroaryl sulfonyl compound of the present invention is also not a chemical probe used to perturb the function of a variety of proteins in a biological sample, but instead a focused KRAS binder.

In certain embodiments the KRAS Recognition Moiety is

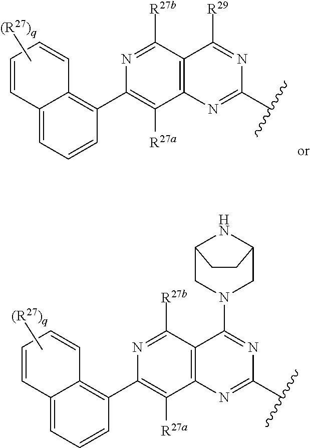

wherein
R^{27a} and R^{27b} are independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R¹⁸;
R²⁹ is independently selected from hydrogen, alkyl, haloalkyl, cycloalkyl, aryl, heterocycle, halogen and heteroaryl each of which is optionally substituted with 1 or 2 substituents independently selected from R¹⁷;
q is 0, 1, 2, 3, 4, or 5;
and wherein all other variables are as defined herein.

In certain embodiments q is 0, 1, 2, or 3. In certain embodiments q is 0. In certain embodiments q is 1. In certain embodiments q is 2.

In certain embodiments R²⁹ is a bicyclic heterocycle;

In certain embodiments the KRAS Recognition Moiety is

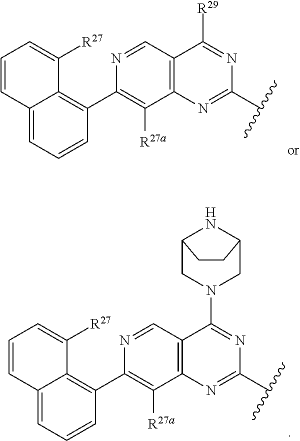

In certain embodiments the KRAS Recognition Moiety is

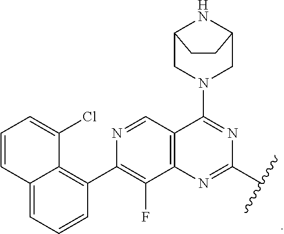 [SMILES: *c1nc(N2CC3CCC(C2)N3)c2cnc(-c3cccc4cccc(Cl)c34)c(F)c2n1]

The present invention focuses on the covalent modification of KRAS to treat diseases, for example, abnormal cellular proliferation such as tumors and cancer. In certain embodiments, a method of treating a disorder mediated by a KRAS is provided comprising administering an effective amount of a heteroaryl sulfonyl compound of Formula I, Formula II, Formula III, Formula IV, Formula V, Formula VI, Formula VII, or Formula VIII, to a patient in need thereof, for example a human, or a pharmaceutically acceptable salt thereof optionally in a pharmaceutically acceptable carrier. For example, in one embodiment, a heteroaryl sulfonyl compound of Formula I, Formula II, Formula III, Formula IV, Formula V, Formula VI, Formula VII, or Formula VIII, is administered to a human to treat a cancer or tumor where the heteroaryl sulfonyl compound has a KRAS Recognition Moiety and the tumor or cancer is mediated by KRAS.

In certain embodiments, the heteroaryl sulfonyl compound described herein does not have to be administered in as high of a dose or as frequently as the classic KRAS inhibitor corresponding to the KRAS Recognition Moiety incorporated into the heteroaryl sulfonyl compound alone for treatment of a disorder. In certain embodiments, a heteroaryl sulfonyl compound of the present invention has fewer or less severe side-effects in the treatment of a disorder mediated by KRAS, than the classic KRAS inhibitor corresponding to the KRAS Recognition Moiety incorporated into the heteroaryl sulfonyl compound alone. In certain embodiments, the heteroaryl sulfonyl compound of the present invention is more efficacious in the treatment of a disorder mediated by KRAS than the classic KRAS inhibitor corresponding to the KRAS Recognition Moiety incorporated into the heteroaryl sulfonyl compound alone.

In principal embodiments, a heteroaryl sulfonyl compound described herein is useful to treat a disorder, for example abnormal cellular proliferation, such as a tumor or cancer, wherein KRAS is mutated. In other embodiments a heteroaryl sulfonyl compound described herein is useful to treat a disorder wherein KRAS is not mutated. In certain embodiments a heteroaryl sulfonyl compound described herein is at least about 2-, 3-, 4-, 5-, 10-, 50-, 100-, 200-, 300-, 400-, 500-, or 1,000-fold more selective for a mutated KRAS than the wild-type KRAS.

In certain embodiments, a heteroaryl sulfonyl compound of the present invention is useful as a therapeutic agent, when administered in an effective amount to a patient, for the treatment of a medical disorder that can be treated with the KRAS Recognition Moiety.

The heteroaryl sulfonyl compounds of the present invention can be administered in any manner that allows the heteroaryl sulfonyl compound to covalently modify KRAS. As such, examples of methods to deliver the heteroaryl sulfonyl compounds of the present invention include, but are not limited to, systemic, parenteral, topical, oral, intravenous, buccal, sublingual, subcutaneous, or transnasal administration.

In certain embodiments, the heteroaryl sulfonyl compound of the present invention has at least one desired isotopic substitution of an atom, at an amount above the natural abundance of the isotope, i.e., enriched.

In certain embodiments, the heteroaryl sulfonyl compound of the present invention includes a deuterium or multiple deuterium atoms. Deuterium is not considered or used herein as a detectable labeling group.

Another aspect of the present invention provides a heteroaryl sulfonyl compound as described herein, or an enantiomer, diastereomer, or stereoisomer thereof, or pharmaceutically acceptable salt, hydrate, or solvate thereof, or a pharmaceutical composition, for use in the manufacture of a medicament for treating or preventing a disease in which KRAS, for example mutant KRAS, plays a role.

In one embodiment, the heteroaryl sulfonyl compound of the present invention is not fluorescent, including but not limited to not a fluorophore.

In other aspects a heteroaryl sulfonyl compound of Formula I′, Formula II′, Formula III′, Formula IV′, Formula V′, Formula VI′, Formula VII′, or Formula VIII′, is provided:

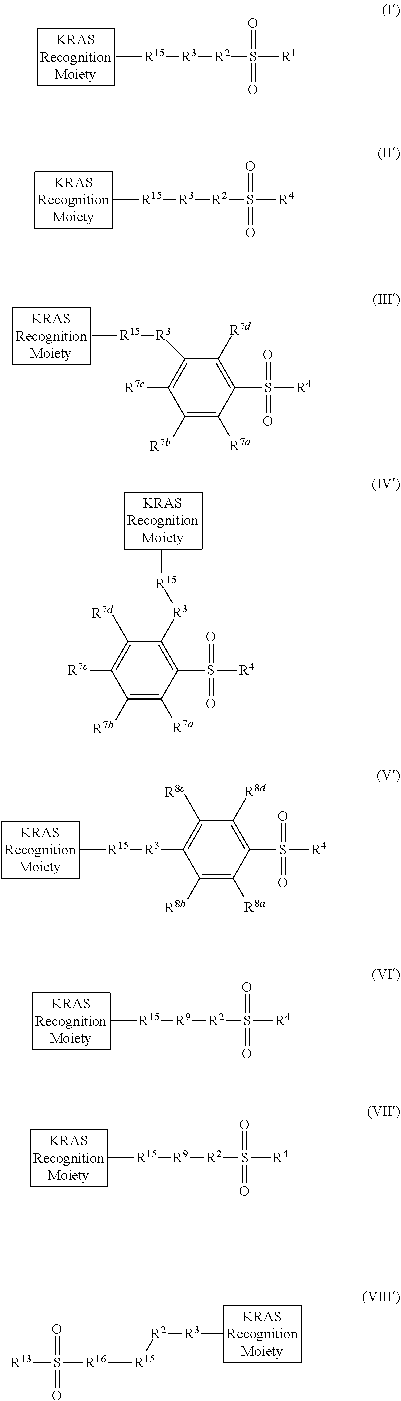

or a pharmaceutically acceptable salt, N-oxide, isotopic derivative, or prodrug thereof, optionally in a pharmaceutically acceptable carrier to form a pharmaceutical composition;
wherein R¹⁵ is a bivalent moiety selected from the group consisting of alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents independently selected from R⁷; and wherein all other variables are as defined herein.

Additional features and advantages of the present application will be apparent from the following detailed description.

The present invention thus includes at least the following features:
(a) A heteroaryl sulfonyl compound of Formula I, Formula II, Formula III, Formula IV, Formula V, Formula VI, Formula VII, or Formula VIII, as described herein, or a pharmaceutically acceptable salt or isotopic derivative (including a deuterated derivative) thereof;
(b) A method for treating a disorder mediated by KRAS, for example mutant KRAS, comprising administering an effective amount of a heteroaryl sulfonyl compound of Formula I, Formula II, Formula III, Formula IV, Formula V, Formula VI, Formula VII, or Formula VIII, or pharmaceutically acceptable salt thereof, as described herein, to a patient in need thereof;
(c) A heteroaryl sulfonyl compound of Formula I, Formula II, Formula III, Formula IV, Formula V, Formula VI, Formula VII, or Formula VIII, or a pharmaceutically acceptable salt thereof for use in the treatment of a disorder that is mediated by KRAS, for example mutant KRAS;
(d) Use of a heteroaryl sulfonyl compound of Formula I, Formula II, Formula III, Formula IV, Formula V, Formula VI, Formula VII, or Formula VIII, or a pharmaceutically acceptable salt thereof, in an effective amount in the treatment of a patient in need thereof, typically a human, with disorder mediated by KRAS, for example mutant KRAS;
(e) Use of a heteroaryl sulfonyl compound of Formula I, Formula II, Formula III, Formula IV, Formula V, Formula VI, Formula VII, or Formula VIII, or a pharmaceutically acceptable salt thereof in the manufacture of a medicament for the treatment of a disorder mediated by KRAS, for example mutant KRAS;
(f) A pharmaceutical composition comprising an effective patient-treating amount of a heteroaryl sulfonyl compound of Formula I, Formula II, Formula III, Formula IV, Formula V, Formula VI, Formula VII, or Formula VIII, or a pharmaceutically acceptable salt thereof; and a pharmaceutically acceptable carrier or diluent;
(g) A heteroaryl sulfonyl compound of Formula I, Formula II, Formula III, Formula IV, Formula V, Formula VI, Formula VII, or Formula VIII, as described herein as a mixture of enantiomers or diastereomers (as relevant), including as a racemate;
(h) A heteroaryl sulfonyl compound of Formula I, Formula II, Formula III, Formula IV, Formula V, Formula VI, Formula VII, or Formula VIII, as described herein in enantiomerically or diastereomerically (as relevant) enriched form, including an isolated enantiomer or diastereomer (i.e., greater than 85, 90, 95, 97, or 99% pure); and
(i) A process for the preparation of therapeutic products that contain an effective amount of a heteroaryl sulfonyl compound of Formula I, Formula II, Formula III, Formula IV, Formula V, Formula VI, Formula VII, or Formula VIII, or a pharmaceutically acceptable salt thereof, as described herein.
(j) A heteroaryl sulfonyl compound of Formula I′, Formula II′, Formula III′, Formula IV′, Formula V′, Formula VI′, Formula VII′, or Formula VIII′, as described herein, or a pharmaceutically acceptable salt or isotopic derivative (including a deuterated derivative) thereof;
(k) A method for treating a disorder mediated by KRAS, for example mutant KRAS, comprising administering an effective amount of a heteroaryl sulfonyl compound of Formula I′, Formula II′, Formula III′, Formula IV′, Formula V′, Formula VI′, Formula VII′, or Formula VIII′, or pharmaceutically acceptable salt thereof, as described herein, to a patient in need thereof,
(l) A heteroaryl sulfonyl compound of Formula I′, Formula II′, Formula III′, Formula IV′, Formula V′, Formula VI′, Formula VII′, or Formula VIII′, or a pharmaceutically acceptable salt thereof for use in the treatment of a disorder that is mediated by KRAS, for example mutant KRAS;
(m) Use of a heteroaryl sulfonyl compound of Formula I′, Formula II′, Formula III′, Formula IV′, Formula V′, Formula VI′, Formula VII′, or Formula VIII′, or a pharmaceutically acceptable salt thereof, in an effective amount in the treatment of a patient in need thereof, typically a human, with disorder mediated by KRAS, for example mutant KRAS;
(n) Use of a heteroaryl sulfonyl compound of Formula I′, Formula II′, Formula III′, Formula IV′, Formula V′, Formula VI′, Formula VII′, or Formula VIII′, or a pharmaceutically acceptable salt thereof in the manufacture of a medicament for the treatment of a disorder mediated by KRAS, for example mutant KRAS;
(o) A pharmaceutical composition comprising an effective patient-treating amount of a heteroaryl sulfonyl compound of Formula I′, Formula II′, Formula III′, Formula IV′, Formula V′, Formula VI′, Formula VII′, or Formula VIII′, or a pharmaceutically acceptable salt thereof; and a pharmaceutically acceptable carrier or diluent;
(p) A heteroaryl sulfonyl compound of Formula I′, Formula II′, Formula III′, Formula IV′, Formula V′, Formula VI′, Formula VII′, or Formula VIII′, as described herein as a mixture of enantiomers or diastereomers (as relevant), including as a racemate;
(q) A heteroaryl sulfonyl compound of Formula I′, Formula II′, Formula III′, Formula IV′, Formula V′, Formula VI′, Formula VII′, or Formula VIII′, as described herein in enantiomerically or diastereomerically (as relevant) enriched form, including an isolated enantiomer or diastereomer (i.e., greater than 85, 90, 95, 97, or 99% pure); and
(r) A process for the preparation of therapeutic products that contain an effective amount of a heteroaryl sulfonyl compound of Formula I′, Formula II′, Formula III′, Formula IV′, Formula V′, Formula VI′, Formula VII′, or Formula VIII′, or a pharmaceutically acceptable salt thereof, as described herein.

### BRIEF DESCRIPTION OF THE FIGURES

As used in the figures R¹⁷ is independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R¹⁸;

As used in the figures n is 0, 1, 2, 3, or 4.

As used in the figures

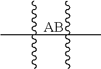 [SMILES: **]

is an Anchor Bond. Anchor Bond is the chemical bond between the KRAS Recognition Moiety and the rest of the molecule for example a bond to R³, R⁹, or R¹⁶, as appropriate.

In the context of crystal structures three letter codes used below refer to specific ligands in the RCSB PDB database which is accessible on https://www.rcsb.org/.

FIG. 1A, 1B, 1C, 1D, 1E, IF, and 1G present non-limiting examples of ligands that bind to Kirsten Rat Sarcoma (KRAS), including the compounds EZZ, F0N, F0K, F0B, GNP, 1M1, 18F, 1M0, 17X, 91D, 91G, K9J, K9M, MZQ, MZN, JJN, MOV, 6ZD, O67, O5Y, O5V, O5S, C8G, 8ZG, D2W, D2Z, R6W, 5UT, 91S, M1X, M1R, QH4, OHY, D1W, and D1Z. For additional non-limiting examples and related ligands, see ligands identified by Zhou et al., “KRASQ61HPreferentially Signals through MAPK in a RAF Dimer-Dependent Manner in Non-Small Cell Lung Cancer”, Cancer Res., 2020, 80: 3719-3731; Poulin et al., “Tissue-Specific Oncogenic Activity of KRASA146T”, Cancer Discov., 2019, 9: 738-755; Kessler et al., “Drugging an undruggable pocket on KRAS”, Proc Natl Acad Sci USA., 2019, 116: 15823-15829; Hobbs et al., “Atypical KRASG12RMutant Is Impaired in PI3K Signaling and Macropinocytosis in Pancreatic Cancer”, Cancer Discov., 2020, 10: 104-123; Zimmermann et al., “Small molecule inhibition of the KRAS PDEd interaction impairs oncogenic KRAS signalling”, Nature, 2013, 497: 638-642; Zeng et al., “Potent and Selective Covalent Quinazoline Inhibitors of KRAS G12C”, Cell Chem Biol., 2017, 24: 1005-1016.e3; Fell et al., “Discovery of Tetrahydropyridopyrimidines as Irreversible Covalent Inhibitors of KRAS-G12C with In Vivo Activity”, ACS Med Chem Lett., 2018, 9: 1230-1234; Fell et al., “Discovery of Tetrahydropyridopyrimidines as Irreversible Covalent Inhibitors of KRAS-G12C with In Vivo Activity”, ACS Med Chem Lett., 2018, 9: 1230-1234; Mortier et al., “Computationally Empowered Workflow Identifies Novel Covalent Allosteric Binders for KRASG12C”, ChemMedChem., 2020, 15: 827-832; Bergner et al., “KRAS Binders Hidden in Nature”, Chemistry, 2019, 25: 12037-12041; Canon et al., “The clinical KRAS(G12C) inhibitor AMG 510 drives anti-tumour immunity”, Nature, 2019, 575: 217-223; Xoing et al., “Covalent Guanosine Mimetic Inhibitors of G12C KRAS”, ACS Med Chem Lett., 2017 8: 61-66; Shin et al., “Discovery of N-(1-Acryloylazetidin-3-yl)-2-(1H-indol-1-yl)acetamides as Covalent Inhibitors of KRASG12C”, ACS Med Chem Lett., 2019, 10: 1302-1308; Hansen et al., “The reactivity-driven biochemical mechanism of covalent KRASG12Cinhibitors”, Nat Struct Mol Biol., 2018, 25: 454-462; Quevedo et al., “Small molecule inhibitors of RAS-effector protein interactions derived using an intracellular antibody fragment”, Nat Commun., 2018, 9: 3169-3169; Kessler et al., “crystal structure of an active KRAS G12D (GPPCP) dimer in complex with BI-5747”, to be published; Patricelli et al., “Selective Inhibition of Oncogenic KRAS Output with Small Molecules Targeting the Inactive State”, Cancer Discov., 2016, 6: 316-329; Janes et al., “Targeting KRAS Mutant Cancers with a Covalent G12C-Specific Inhibitor”, Cell, 2018, 172: 578-589.e17; Fell et al., “Identification of the Clinical Development Candidate MRTX849, a Covalent KRASG12C Inhibitor for the Treatment of Cancer”, J Med Chem., 2020, 63: 6679-6693; Lanman et al., “Discovery of a Covalent Inhibitor of KRASG12C(AMG 510) for the Treatment of Solid Tumors”, J Med Chem., 2020, 63: 52-65.

FIG. 2 is a non-limiting example of a Formula described herein.

### DETAILED DESCRIPTION OF THE INVENTION

Heteroaryl sulfonyl compounds and their use and manufacture are provided that covalently modify KRAS, for example mutant KRAS, to treat a disease that is mediated by KRAS in a host, typically a human. In one aspect of the invention a heteroaryl sulfonyl compound described herein reacts with a tyrosine residue on KRAS, for example mutant KRAS, to form a covalent bond. In another aspect a heteroaryl sulfonyl compound described herein reacts with a lysine residue on KRAS, for example mutant KRAS, to form a covalent bond. The invention provides a heteroaryl sulfonyl compound of Formula I, Formula II, Formula III, Formula IV, Formula V, Formula VI, Formula VII, or Formula VIII, or a pharmaceutically acceptable salt thereof that includes a KRAS Recognition Moiety that provides specificity to the heteroaryl sulfonyl compound, and an electrophilic sulfonyl (SO₂) that reacts with the target tyrosine or lysine to create a covalent bond between KRAS and the presently described inhibitor.

The heteroaryl sulfonyl compound as described herein in principle embodiments has a stable shelf life for at least 2 months, 3 months, 6 months or 1 year or more neat or as part of a pharmaceutically acceptable dosage form, and itself is pharmaceutically acceptable.

In one aspect a heteroaryl sulfonyl compound of Formula I, Formula II, Formula III, Formula IV, Formula V, Formula VI, Formula VII, or Formula VIII, or a pharmaceutically acceptable salt thereof, is provided:

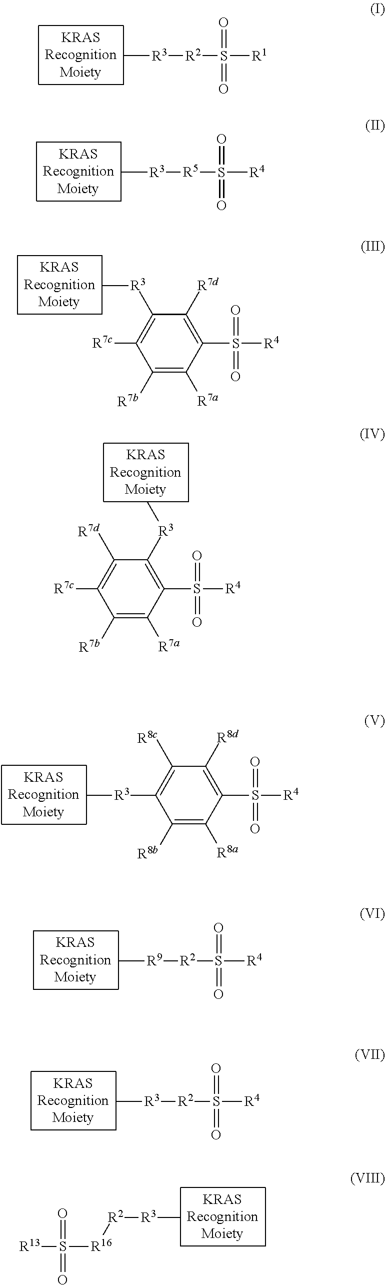

wherein the variables are as defined herein.

### Embodiments of Formula I

In certain embodiments the heteroaryl sulfonyl compound of Formula I is selected from:

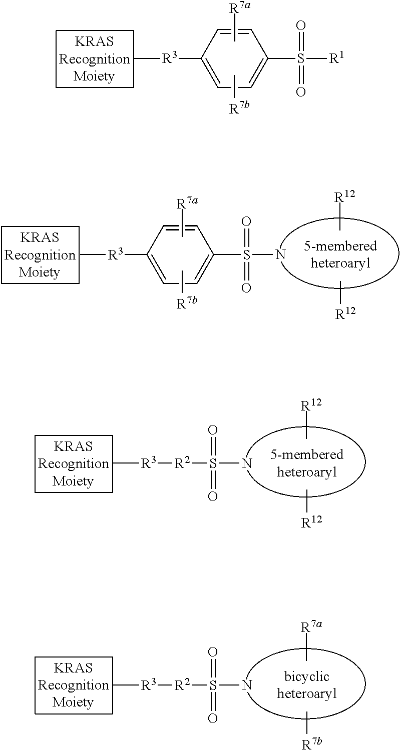

or a pharmaceutically acceptable salt thereof.

### Embodiments of Formula II

In certain embodiments the heteroaryl sulfonyl compound of Formula II is selected from:

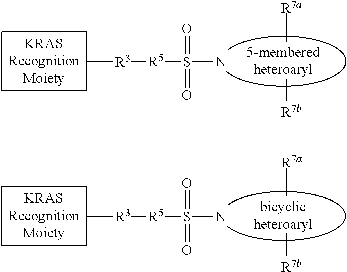

or a pharmaceutically acceptable salt thereof.

### Embodiments of Formula III

In certain embodiments the heteroaryl sulfonyl compound of Formula III is selected from:

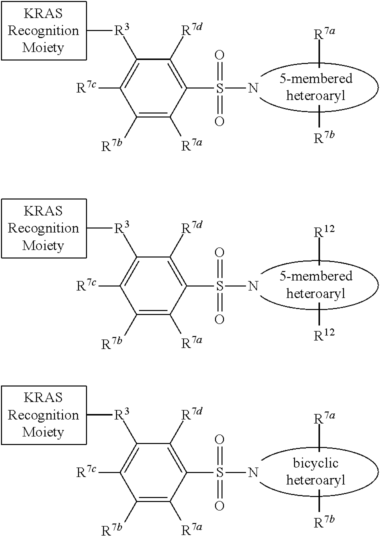

or a pharmaceutically acceptable salt thereof.

### Embodiments of Formula IV

In certain embodiments the heteroaryl sulfonyl compound of Formula IV is selected from:

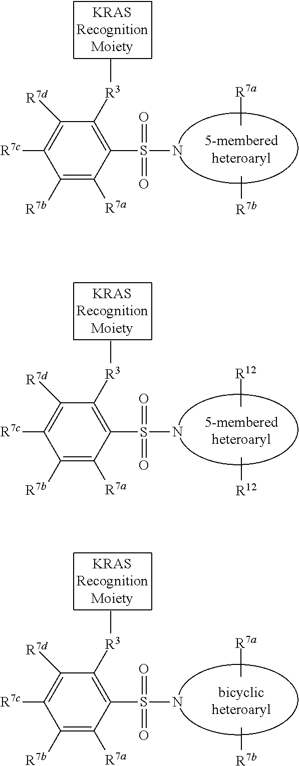

or a pharmaceutically acceptable salt thereof.

### Embodiments of Formula V

In certain embodiments the heteroaryl sulfonyl compound of Formula V is selected from:

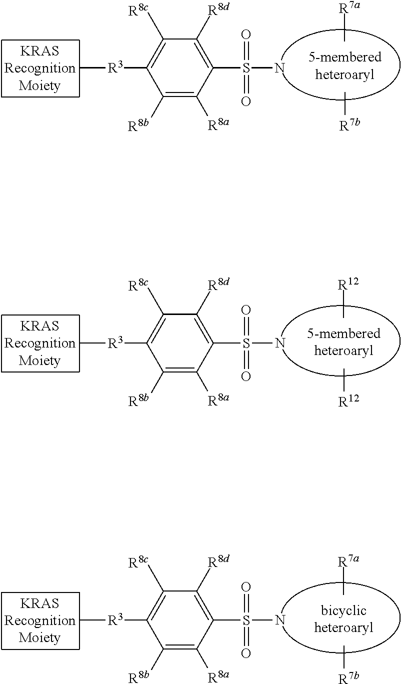

or a pharmaceutically acceptable salt thereof.

### Embodiments of Formula VI

In certain embodiments the heteroaryl sulfonyl compound of Formula VI is selected from:

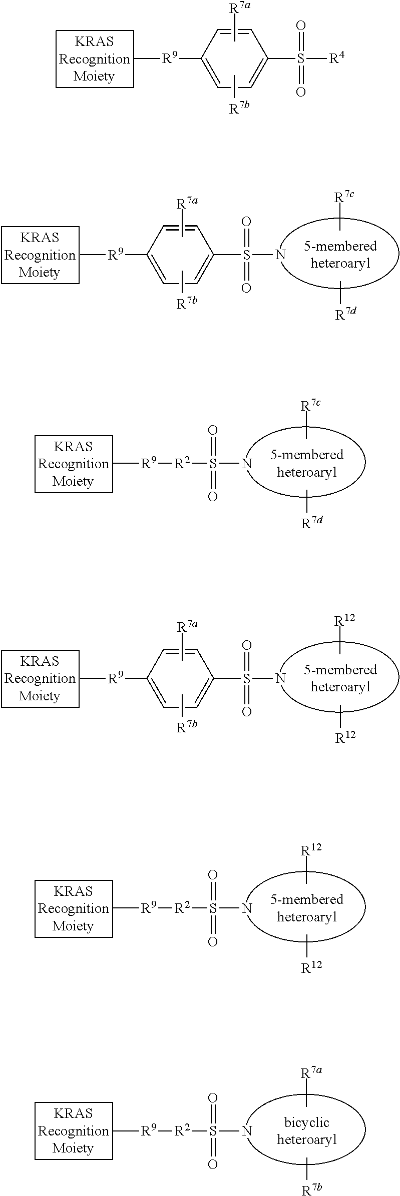

or a pharmaceutically acceptable salt thereof.

### Embodiments of Formula VII

In certain embodiments the heteroaryl sulfonyl compound of Formula VII is selected from:

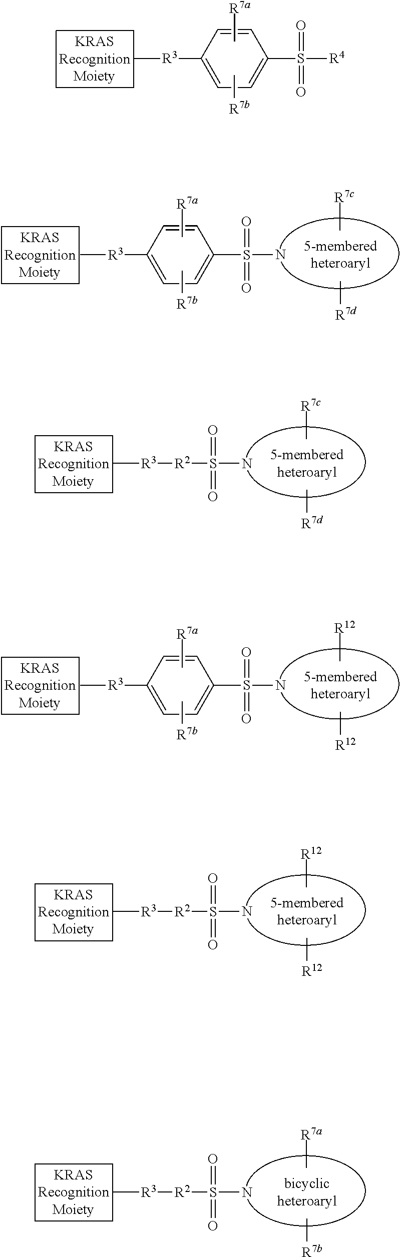

or a pharmaceutically acceptable salt thereof.

### Embodiments of Formula VIII

In certain embodiments the heteroaryl sulfonyl compound of Formula VIII is selected from:

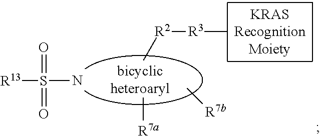

or a pharmaceutically acceptable salt thereof.

In certain embodiments the heteroaryl sulfonyl compound of Formula VIII is selected from:

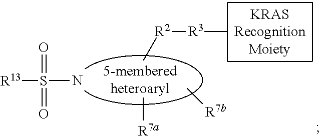

or a pharmaceutically acceptable salt thereof.

### Embodiments of Formula I′

In certain embodiments the heteroaryl sulfonyl compound of Formula I′ is selected from:

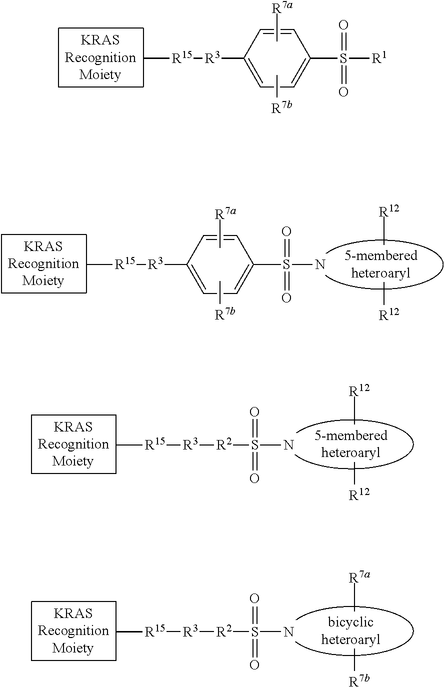

or a pharmaceutically acceptable salt thereof.

### Embodiments of Formula II′

In certain embodiments the heteroaryl sulfonyl compound of Formula II′ is selected from:

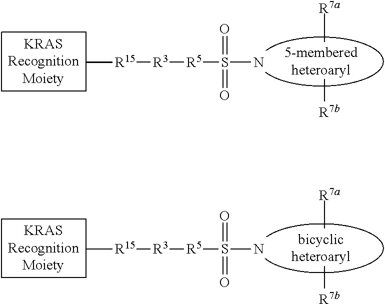

or a pharmaceutically acceptable salt thereof.

### Embodiments of Formula III′

In certain embodiments the heteroaryl sulfonyl compound of Formula III′ is selected from:

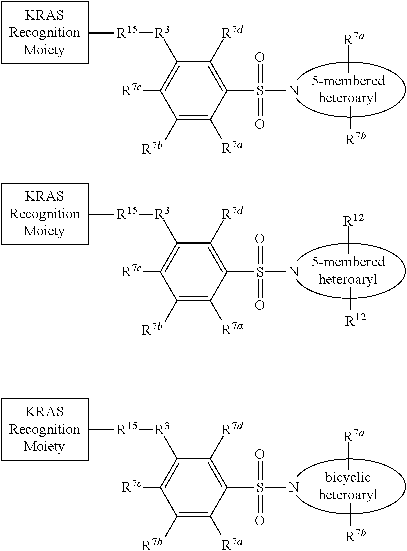

or a pharmaceutically acceptable salt thereof.

### Embodiments of Formula IV′

In certain embodiments the heteroaryl sulfonyl compound of Formula IV′ is selected from:

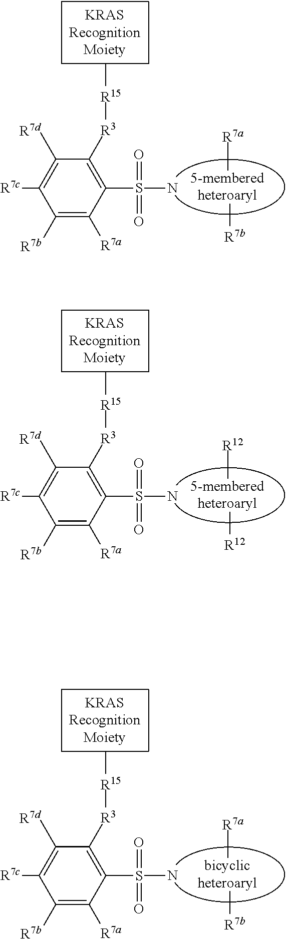

or a pharmaceutically acceptable salt thereof.

### Embodiments of Formula V′

In certain embodiments the heteroaryl sulfonyl compound of Formula V′ is selected from:

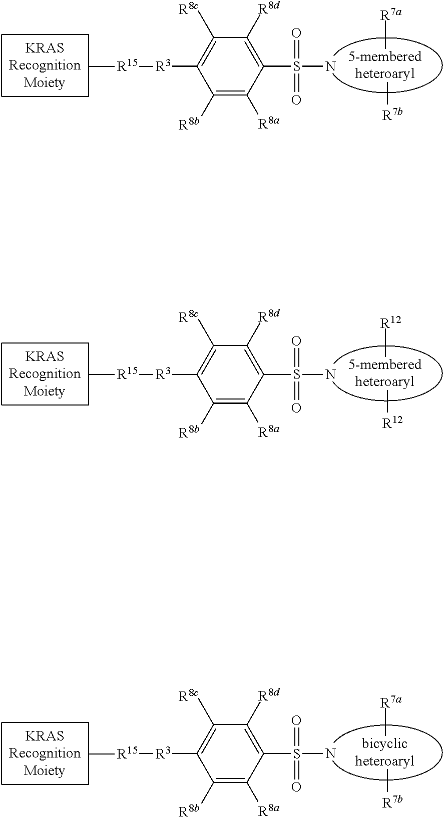

or a pharmaceutically acceptable salt thereof.

### Embodiments of Formula VI′

In certain embodiments the heteroaryl sulfonyl compound of Formula VI′ is selected from:

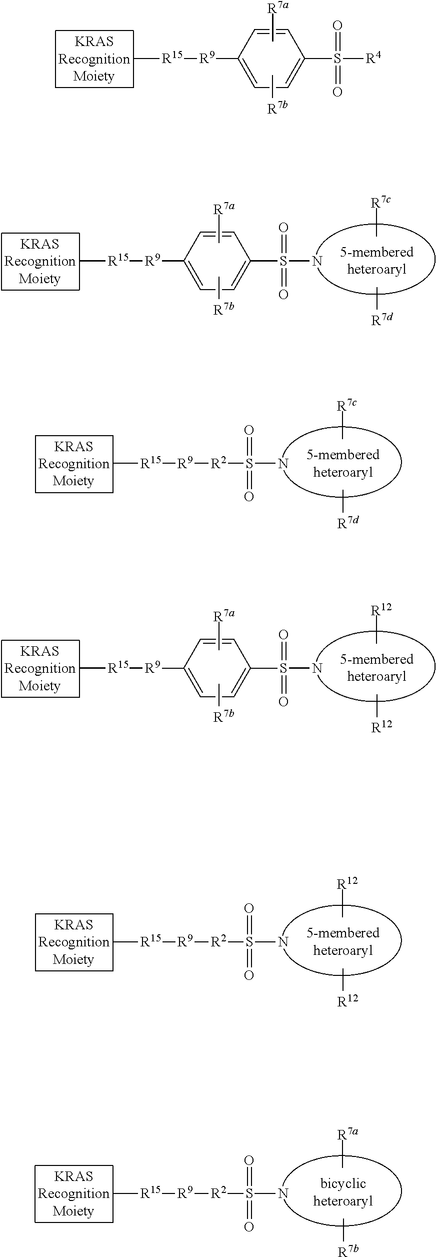

or a pharmaceutically acceptable salt thereof.

### Embodiments of Formula VII′

In certain embodiments the heteroaryl sulfonyl compound of Formula VII′ is selected from:

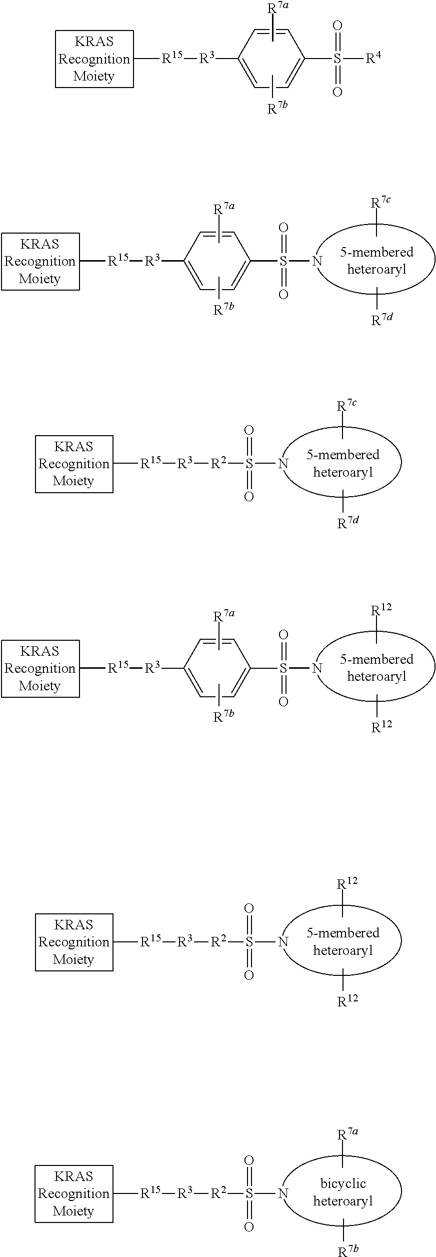

or a pharmaceutically acceptable salt thereof.

### Embodiments of Formula VIII′

In certain embodiments the heteroaryl sulfonyl compound of Formula VIII′ is selected from:

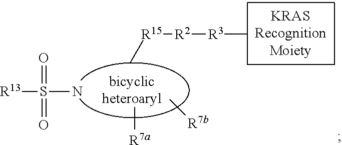

or a pharmaceutically acceptable salt thereof.

In certain embodiments the heteroaryl sulfonyl compound of Formula VIII′ is selected from:

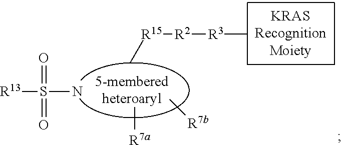

or a pharmaceutically acceptable salt thereof.

### ADDITIONAL EMBODIMENTS

In certain embodiments the heteroaryl sulfonyl compound of the present invention is selected from:

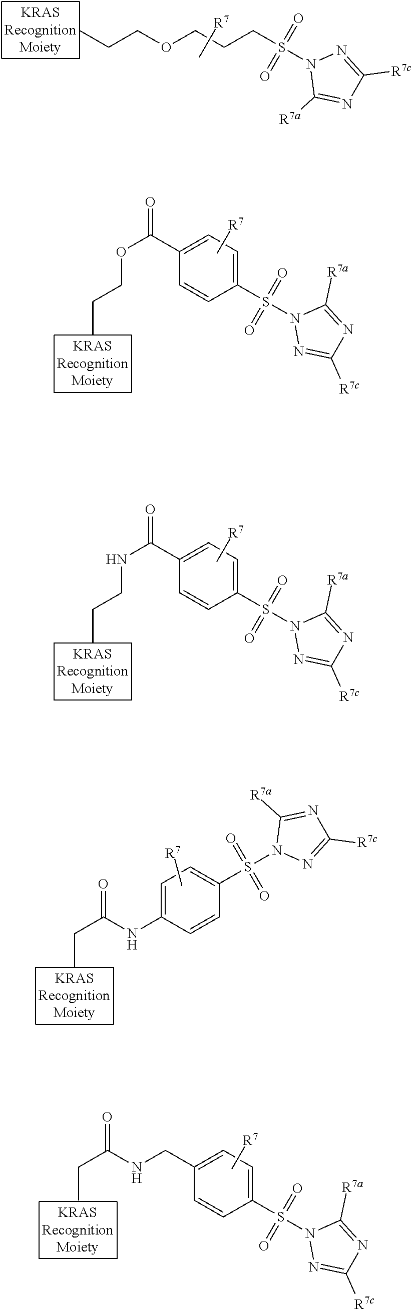

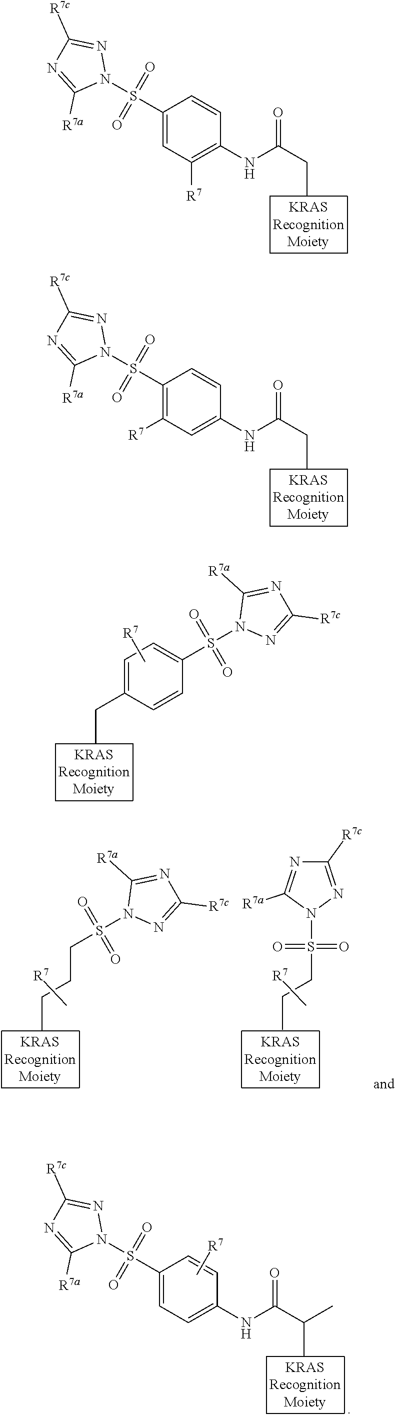

In certain embodiments the heteroaryl sulfonyl compound of the present invention is selected from:

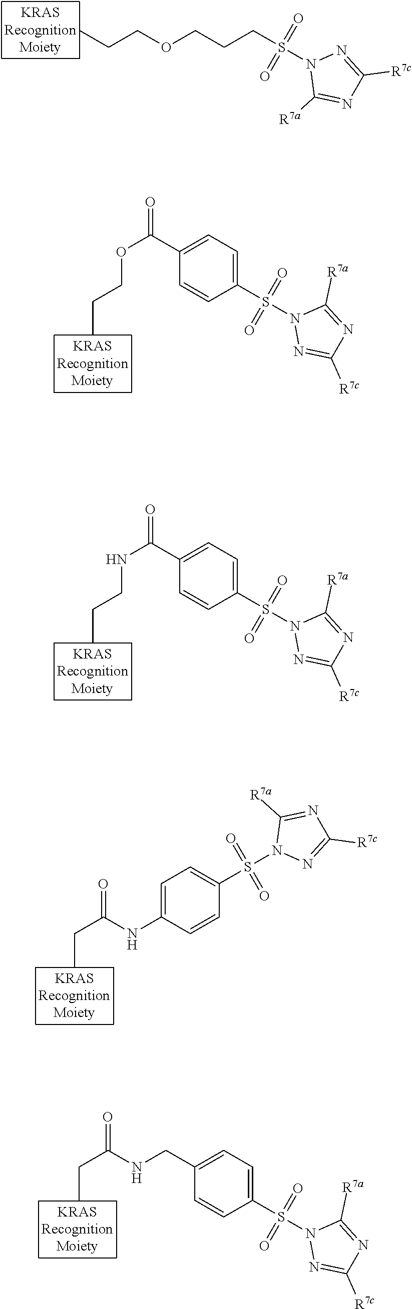

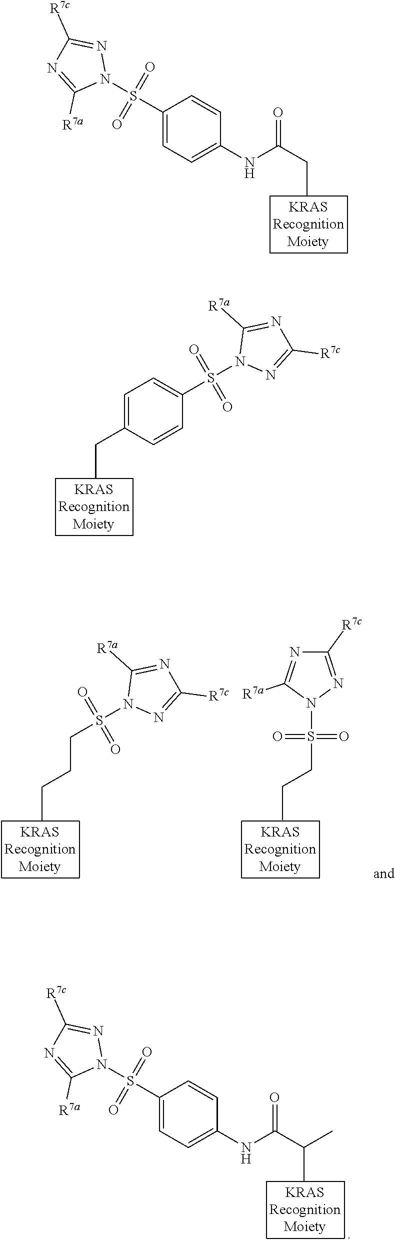

In certain embodiments the heteroaryl sulfonyl compound of the present invention is selected from:

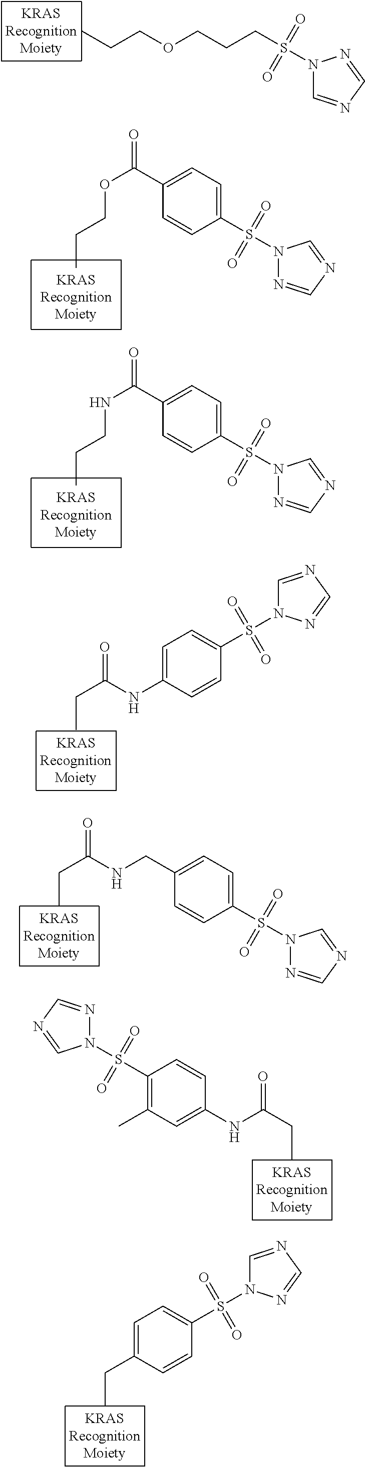

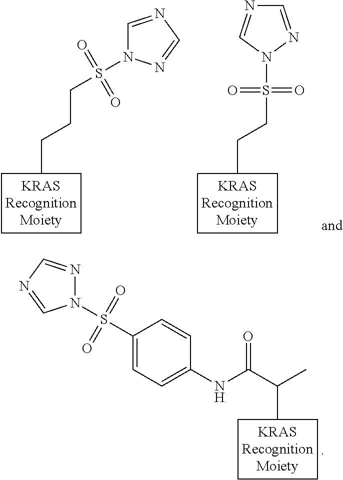 [SMILES: CCCCS(=O)(=O)n1cncn1 ; CCCS(=O)(=O)n1cncn1 ; CC(C)C(=O)Nc1ccc(S(=O)(=O)n2cncn2)cc1]

In certain embodiments the heteroaryl sulfonyl compound of the present invention is selected from:

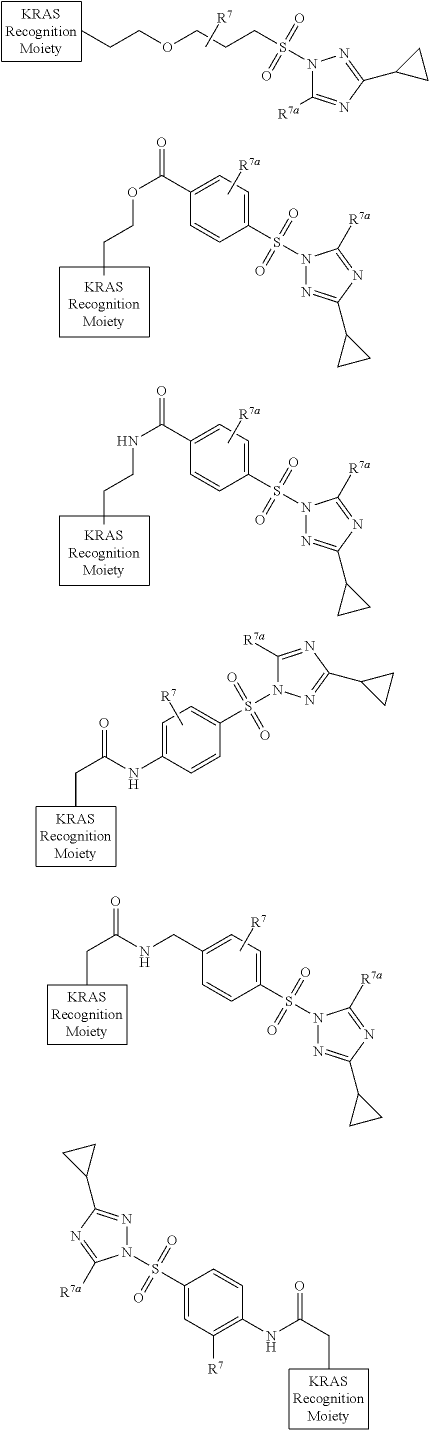

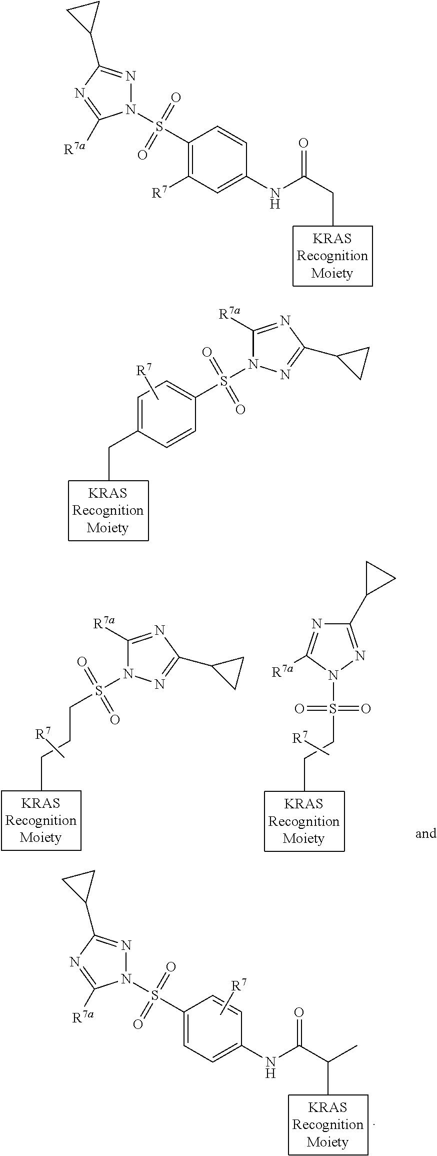

In certain embodiments the heteroaryl sulfonyl compound of the present invention is selected from:

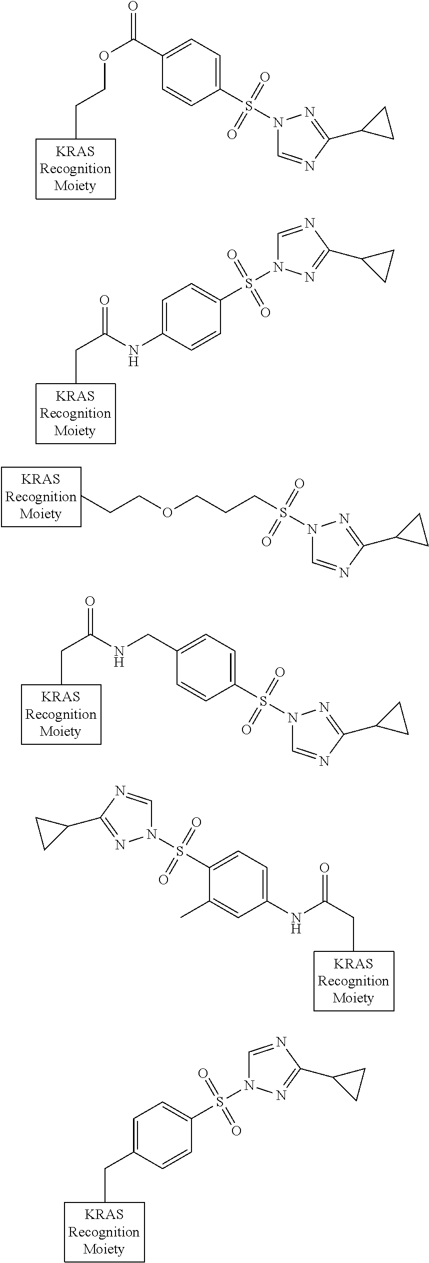 [SMILES: CCCOC(=O)c1ccc(S(=O)(=O)n2cnc(C3CC3)n2)cc1 ; CCC(=O)Nc1ccc(S(=O)(=O)n2cnc(C3CC3)n2)cc1 ; CCCOCCCS(=O)(=O)n1cnc(C2CC2)n1 ; CCC(=O)NCc1ccc(S(=O)(=O)n2cnc(C3CC3)n2)cc1 ; CCC(=O)Nc1ccc(S(=O)(=O)n2cnc(C3CC3)n2)c(C)c1 ; CCc1ccc(S(=O)(=O)n2cnc(C3CC3)n2)cc1]

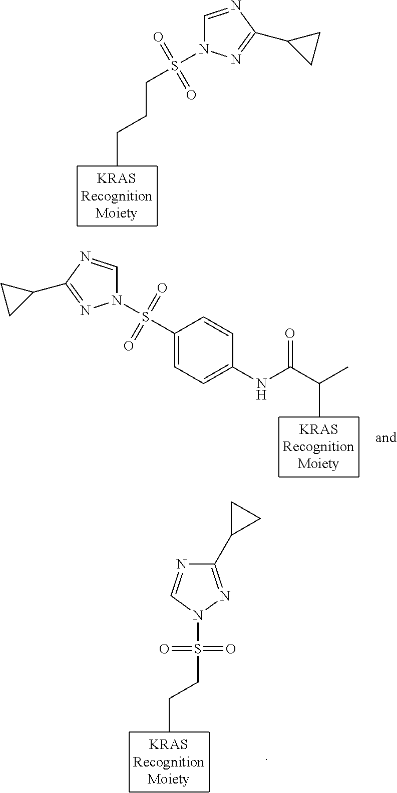 [SMILES: CCCCS(=O)(=O)n1cnc(C2CC2)n1 ; CC(C)C(=O)Nc1ccc(S(=O)(=O)n2cnc(C3CC3)n2)cc1 ; CCCS(=O)(=O)n1cnc(C2CC2)n1]

In certain embodiments the heteroaryl sulfonyl compound of the present invention is

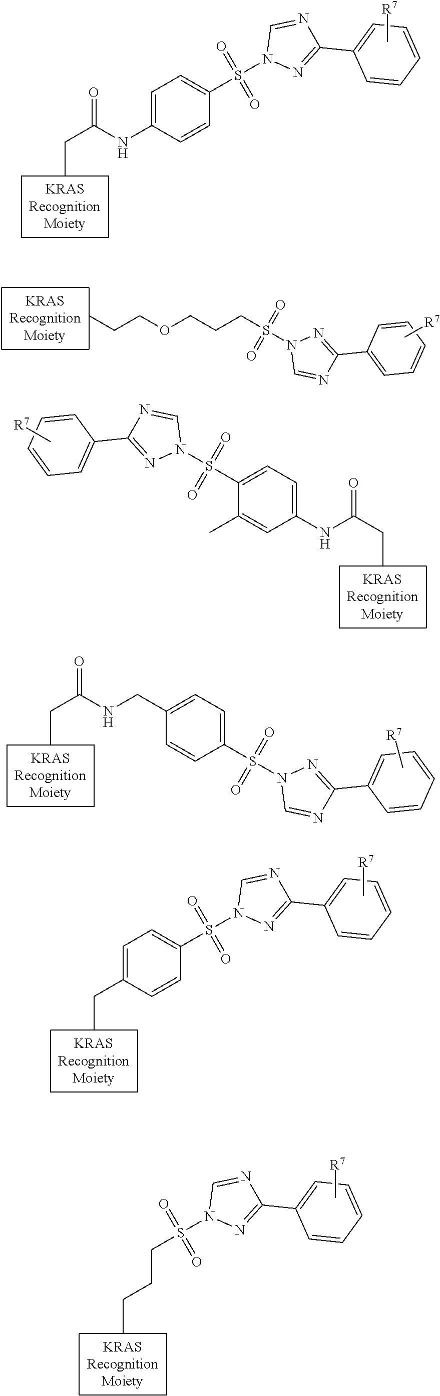

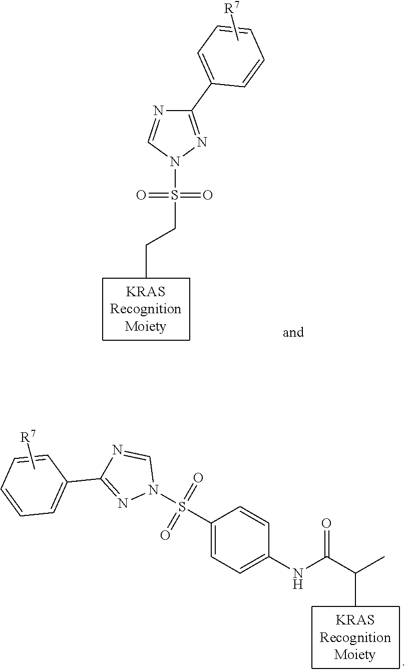

In certain embodiments the heteroaryl sulfonyl compound of the present invention is selected from:

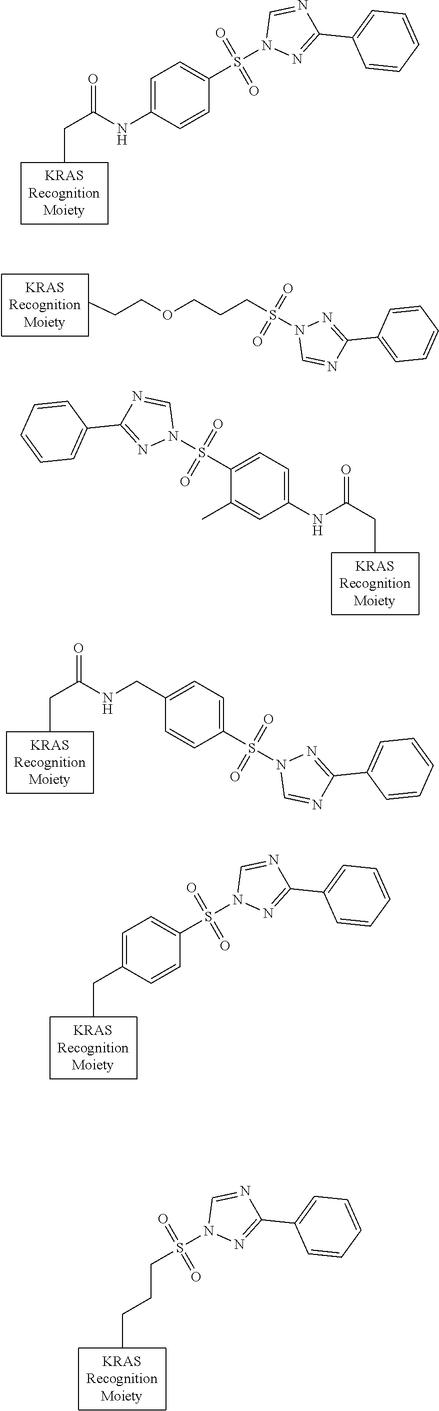 [SMILES: CCC(=O)Nc1ccc(S(=O)(=O)n2cnc(-c3ccccc3)n2)cc1 ; CCCOCCCS(=O)(=O)n1cnc(-c2ccccc2)n1 ; CCC(=O)Nc1ccc(S(=O)(=O)n2cnc(-c3ccccc3)n2)c(C)c1 ; CCC(=O)NCc1ccc(S(=O)(=O)n2cnc(-c3ccccc3)n2)cc1 ; CCc1ccc(S(=O)(=O)n2cnc(-c3ccccc3)n2)cc1 ; CCCCS(=O)(=O)n1cnc(-c2ccccc2)n1]

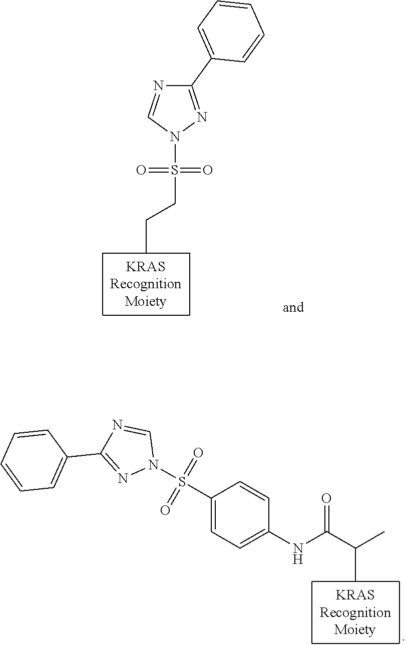 [SMILES: CCCS(=O)(=O)n1cnc(-c2ccccc2)n1 ; CC(C)C(=O)Nc1ccc(S(=O)(=O)n2cnc(-c3ccccc3)n2)cc1]

In certain embodiments the heteroaryl sulfonyl compound of the present invention is selected from:

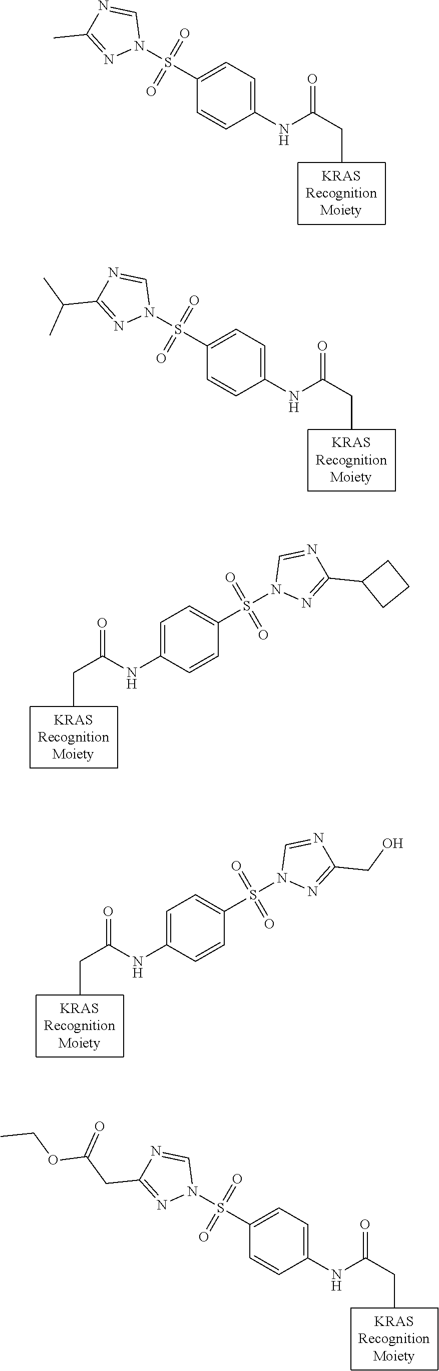 [SMILES: CCC(=O)Nc1ccc(S(=O)(=O)n2cnc(C)n2)cc1 ; CCC(=O)Nc1ccc(S(=O)(=O)n2cnc(C(C)C)n2)cc1 ; CCC(=O)Nc1ccc(S(=O)(=O)n2cnc(C3CCC3)n2)cc1 ; CCC(=O)Nc1ccc(S(=O)(=O)n2cnc(CO)n2)cc1 ; CCOC(=O)Cc1ncn(S(=O)(=O)c2ccc(NC(=O)CC)cc2)n1]

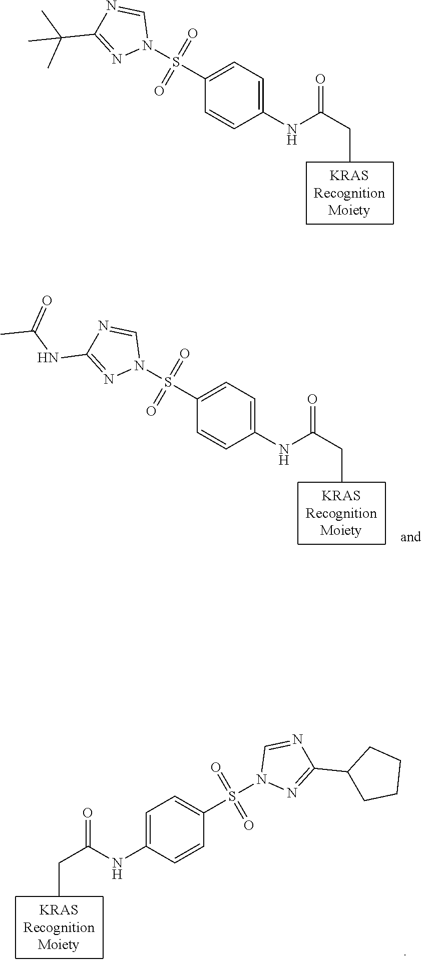 [SMILES: CCC(=O)Nc1ccc(S(=O)(=O)n2cnc(C(C)(C)C)n2)cc1 ; CCC(=O)Nc1ccc(S(=O)(=O)n2cnc(NC(C)=O)n2)cc1 ; CCC(=O)Nc1ccc(S(=O)(=O)n2cnc(C3CCCC3)n2)cc1]

In certain embodiments, the heteroaryl sulfonyl compound of the present invention is selected from:

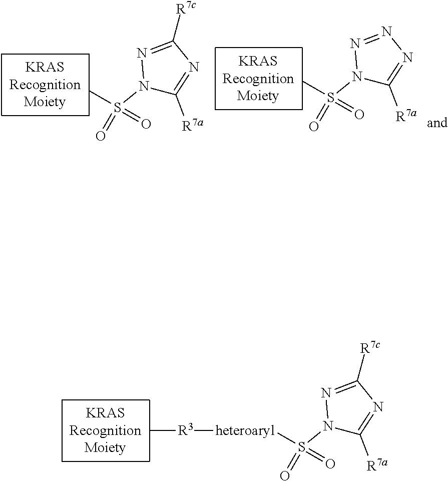

In certain embodiments, the heteroaryl sulfonyl compound of the present invention is selected from:

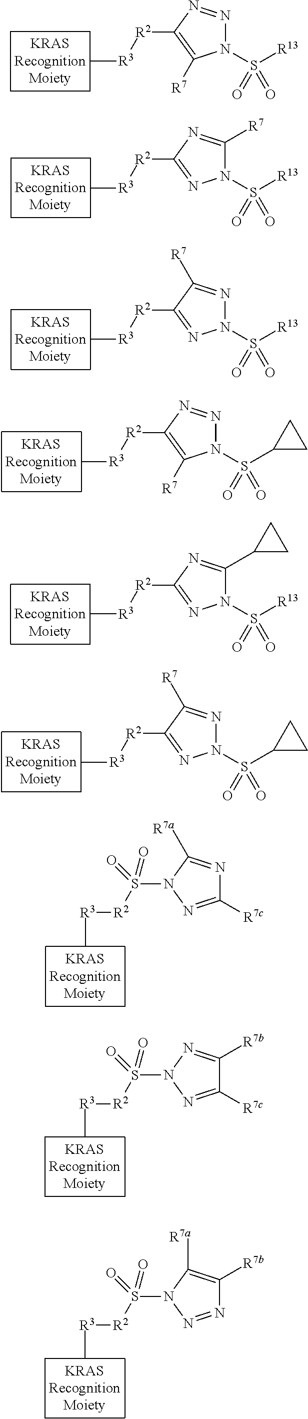

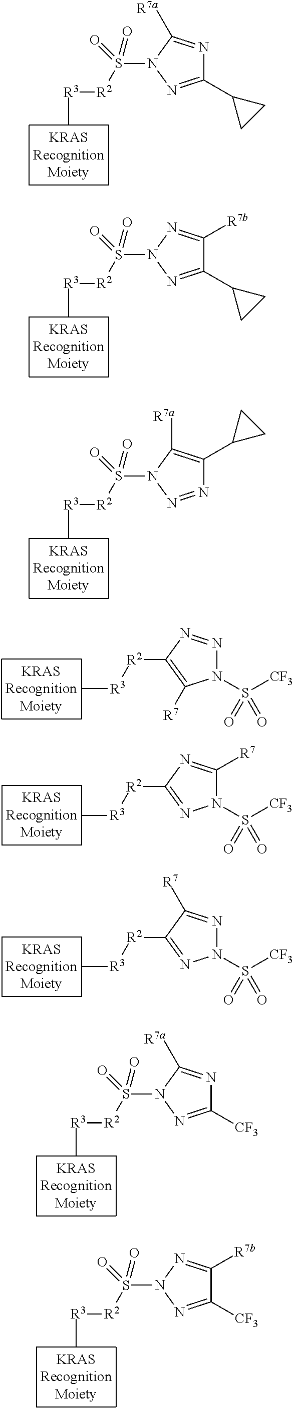

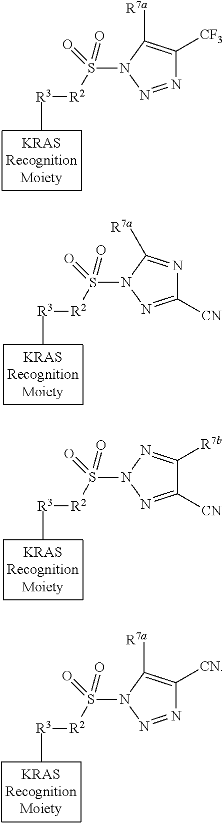

In certain embodiments, the heteroaryl sulfonyl compound of the present invention is selected from:

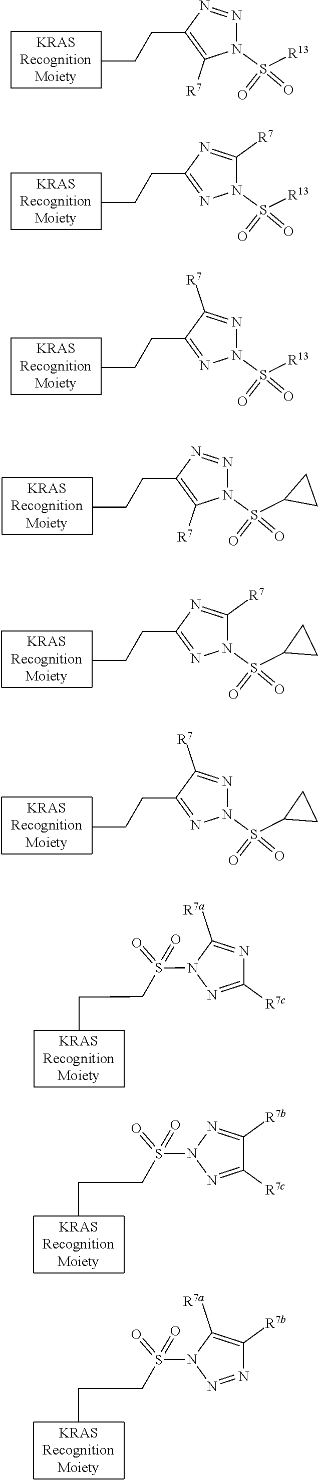

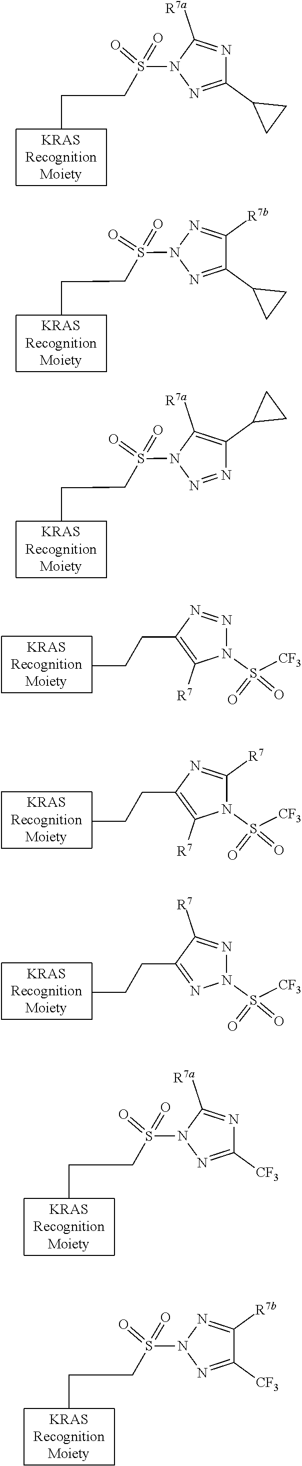

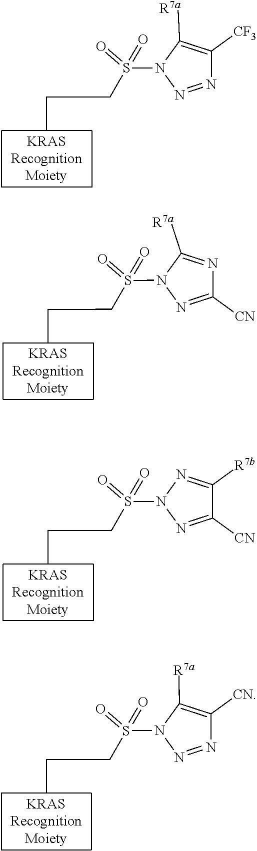

In certain embodiments, the heteroaryl sulfonyl compound of the present invention is selected from:

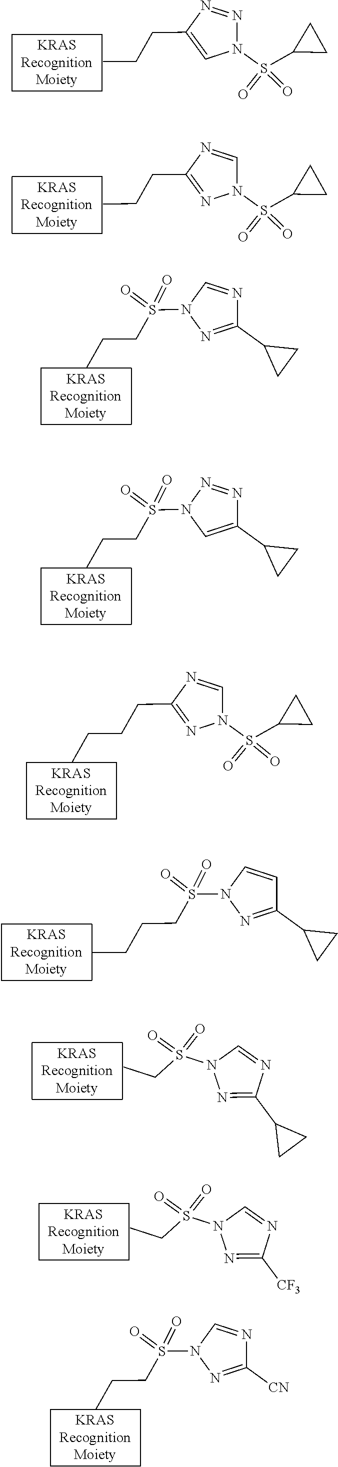

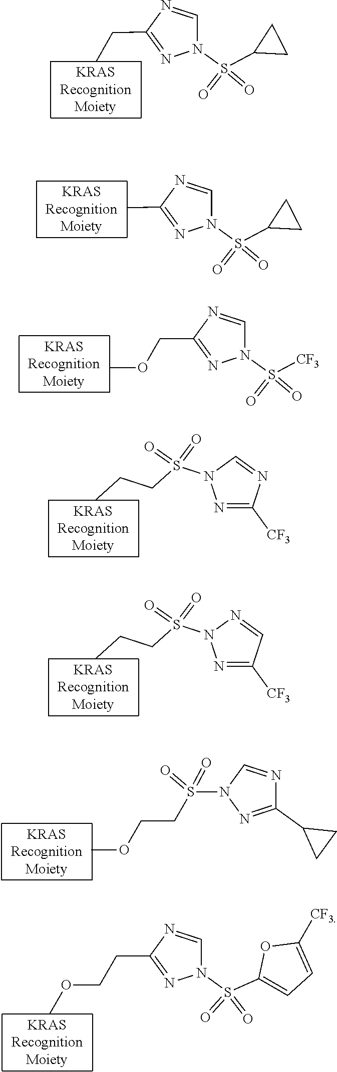

In certain embodiments, the heteroaryl sulfonyl compound of the present invention is selected from:

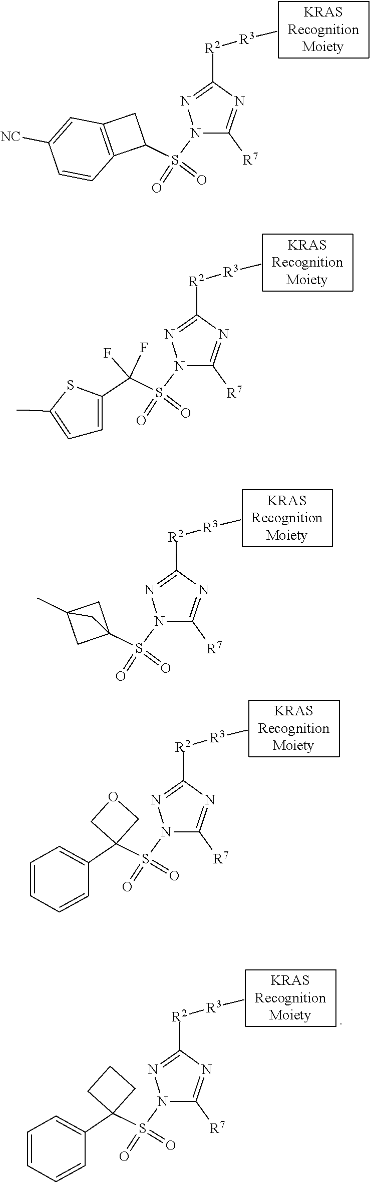

In certain embodiments, the heteroaryl sulfonyl compound of the present invention is selected from:

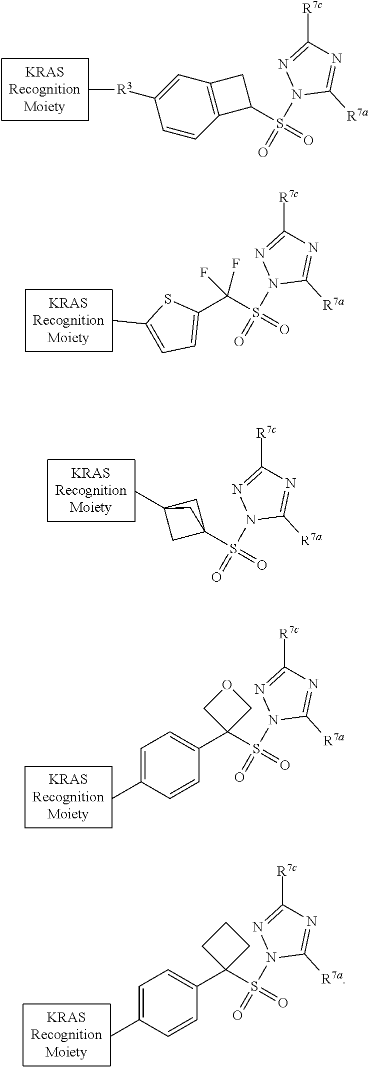

In certain embodiments, the heteroaryl sulfonyl compound of the present invention is selected from:

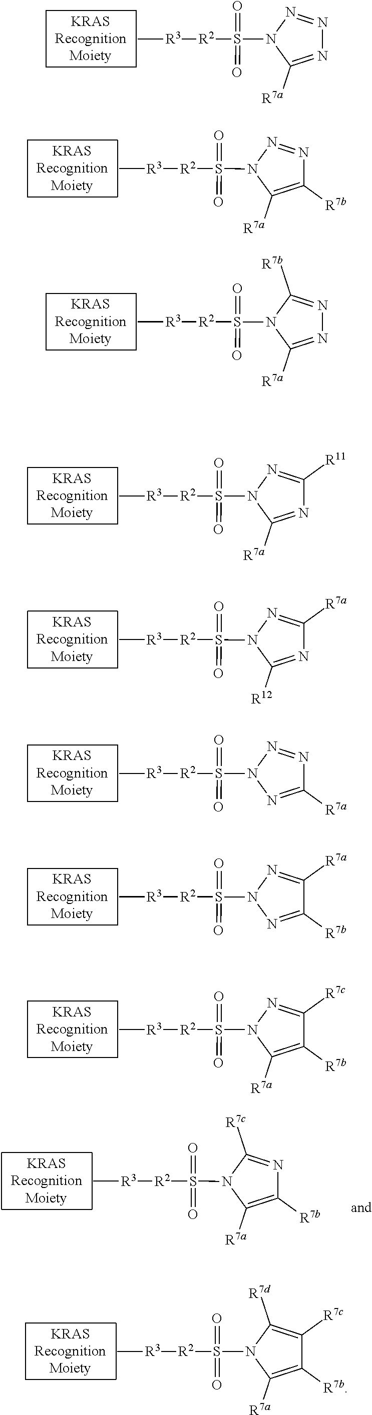

In certain embodiments, the heteroaryl sulfonyl compound of the present invention is selected from:

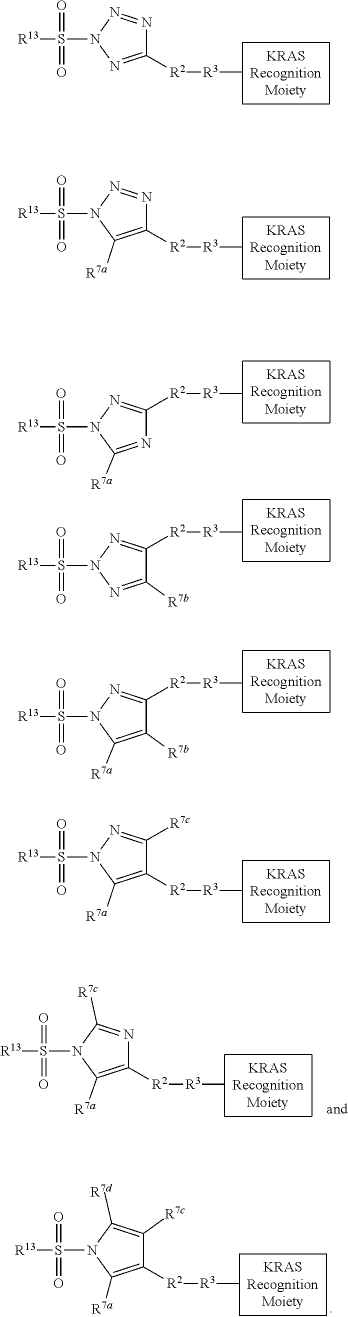

In certain embodiments, the heteroaryl sulfonyl compound of the present invention is selected from:

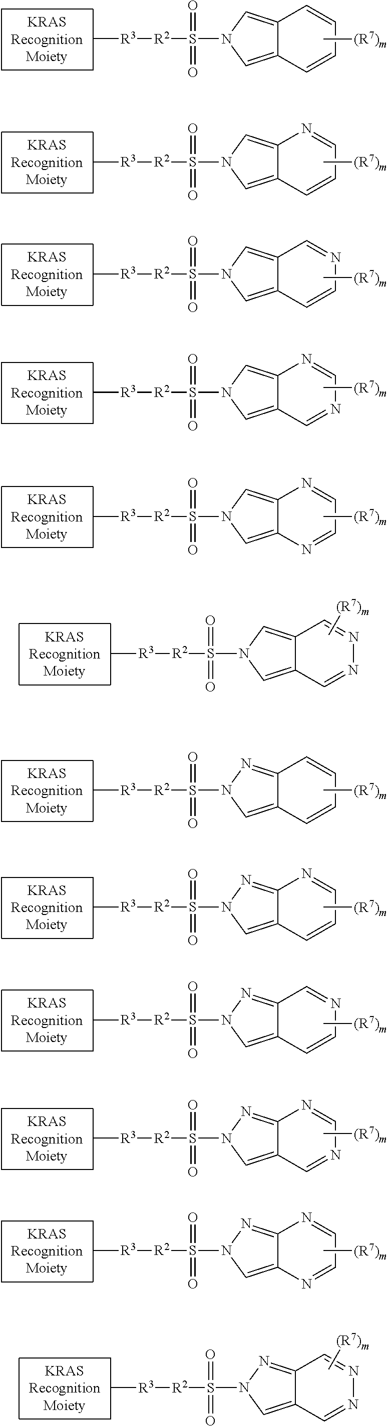

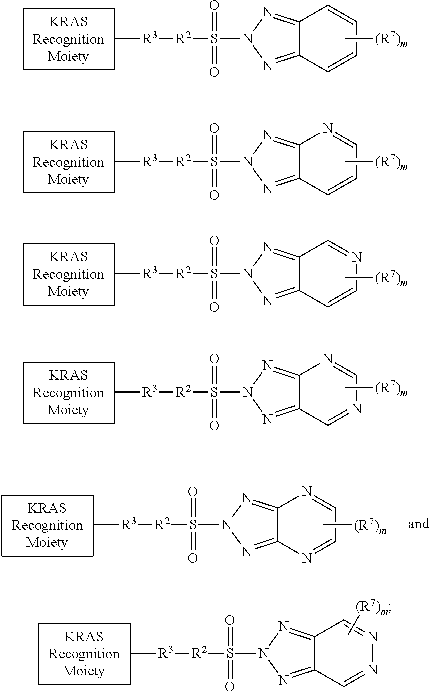

wherein m is independently selected from 1, 2, 3, and 4; and a floating bond on one ring of a bicyclic system means the substituent or substituents are optionally placed on any ring of the system.

For example,

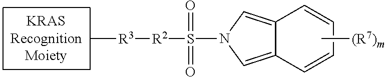

represents, but is not limited to:

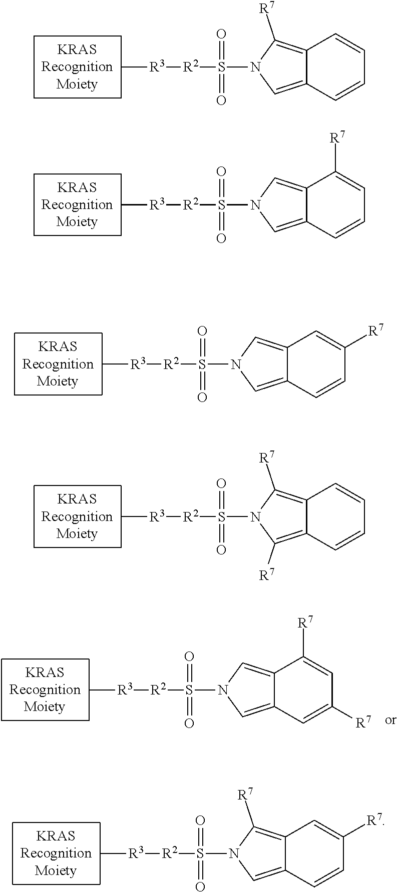

In certain embodiments, the heteroaryl sulfonyl compound of the present invention is selected from:

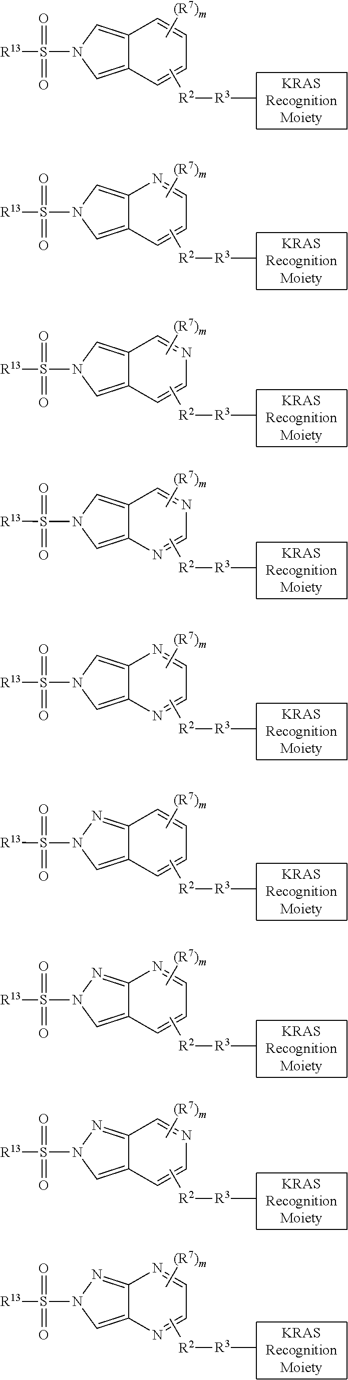

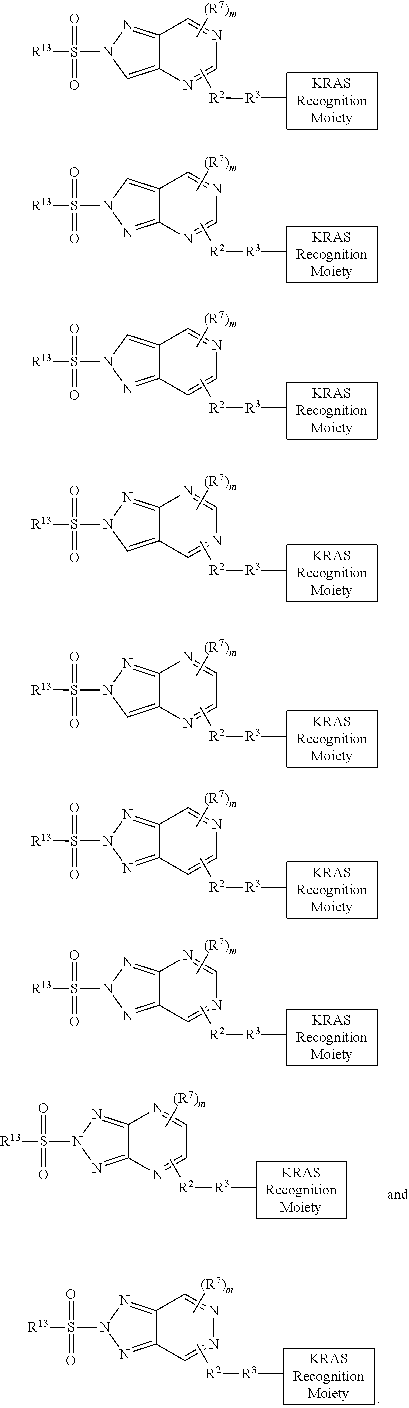

In certain embodiments, the heteroaryl sulfonyl compound of the present invention is selected from:

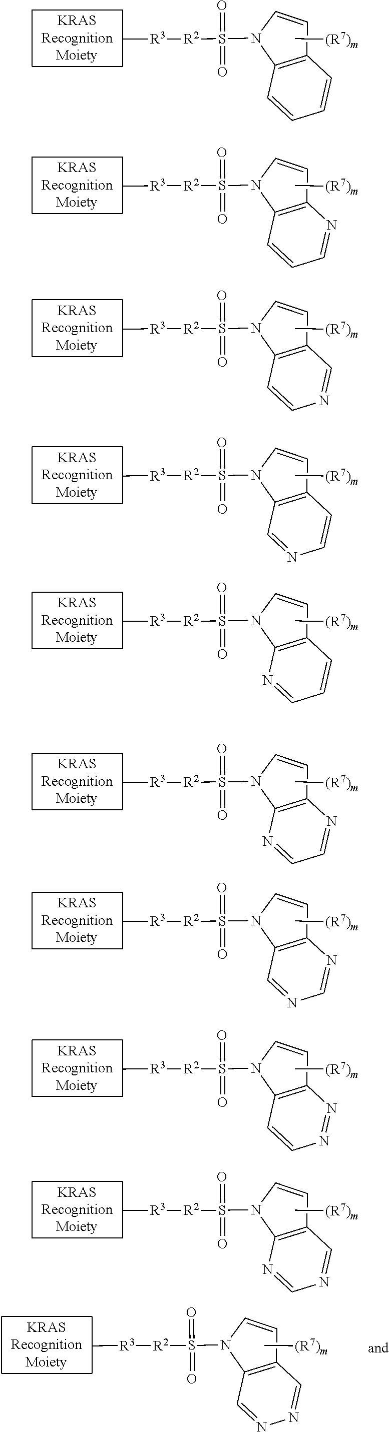

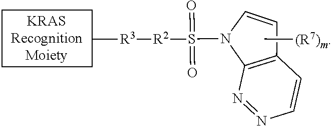

In certain embodiments, the heteroaryl sulfonyl compound of the present invention is selected from:

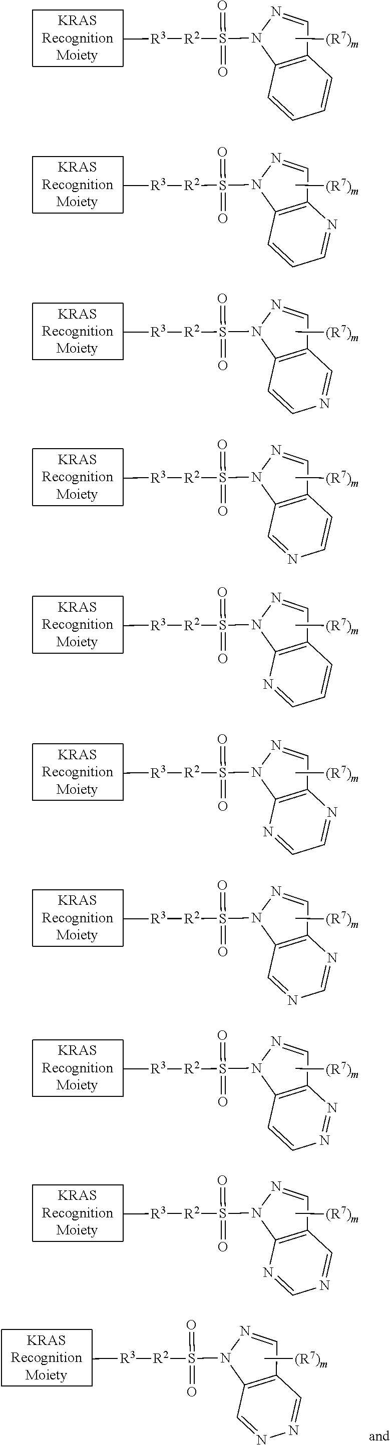

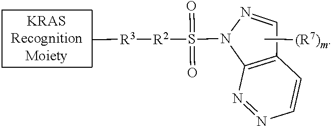

In certain embodiments, the heteroaryl sulfonyl compound of the present invention is selected from:

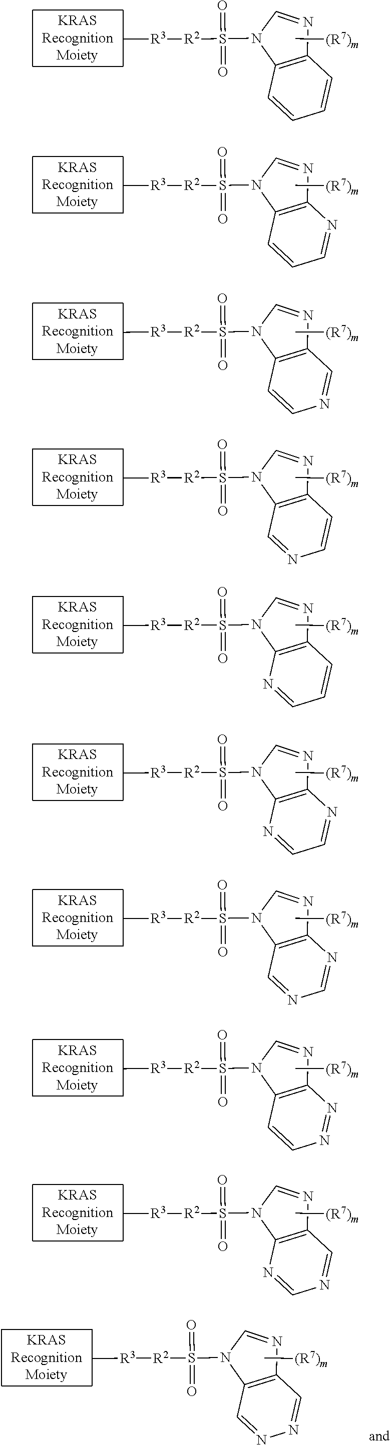

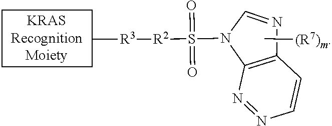

In certain embodiments, the heteroaryl sulfonyl compound of the present invention is selected from:

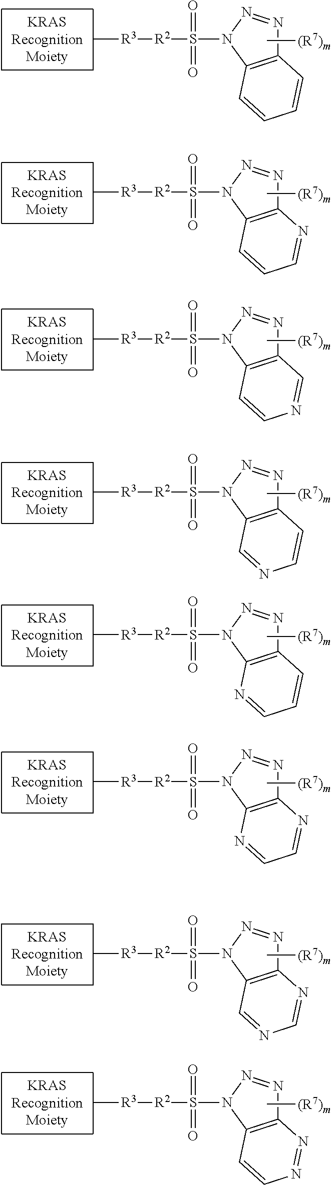

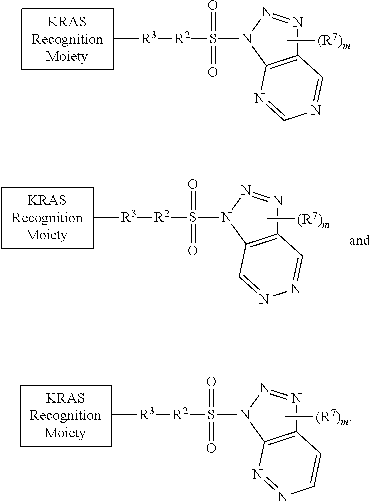

In certain embodiments, the heteroaryl sulfonyl compound of the present invention is selected from:

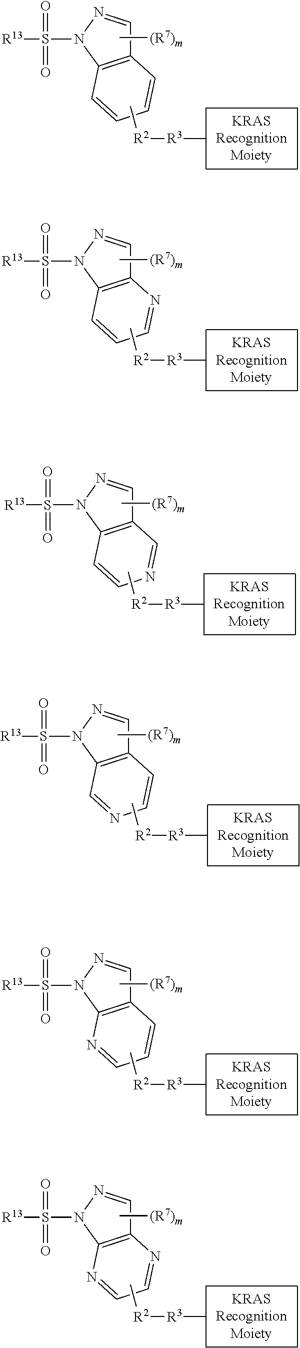

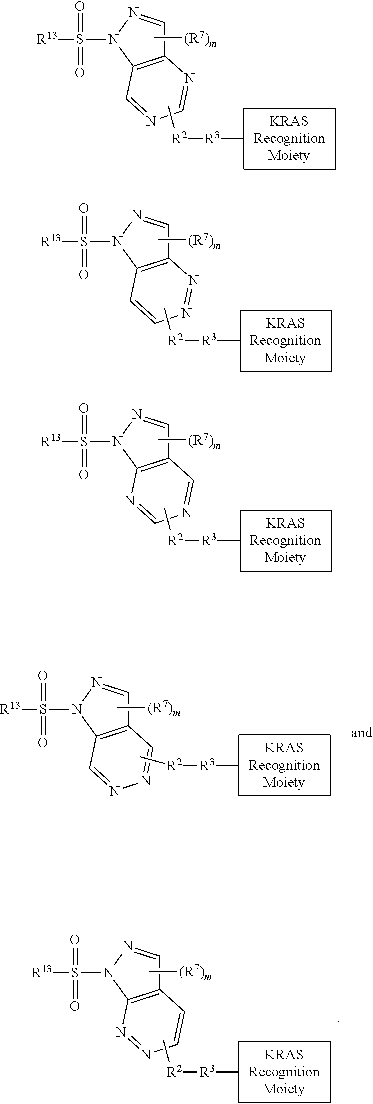

In certain embodiments, the heteroaryl sulfonyl compound of the present invention is selected from:

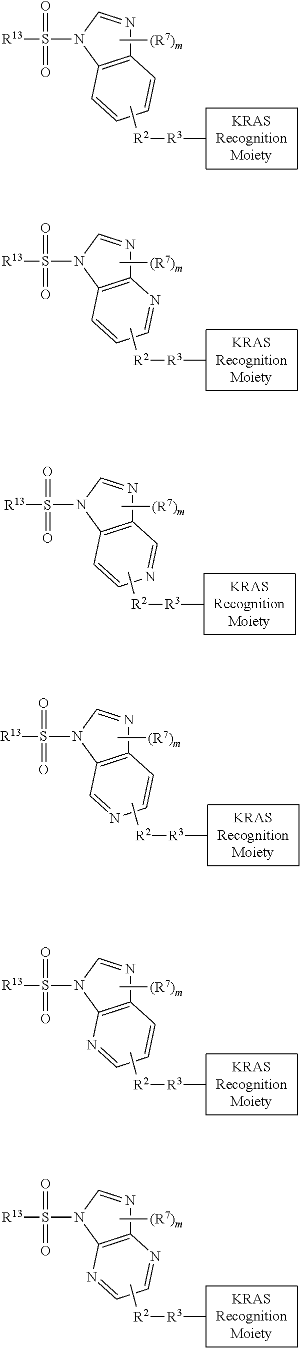

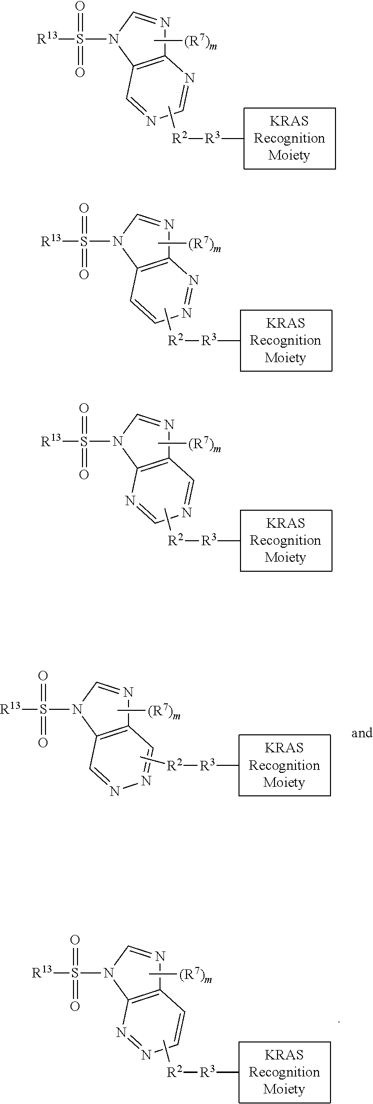

In certain embodiments, the heteroaryl sulfonyl compound of the present invention is selected from:

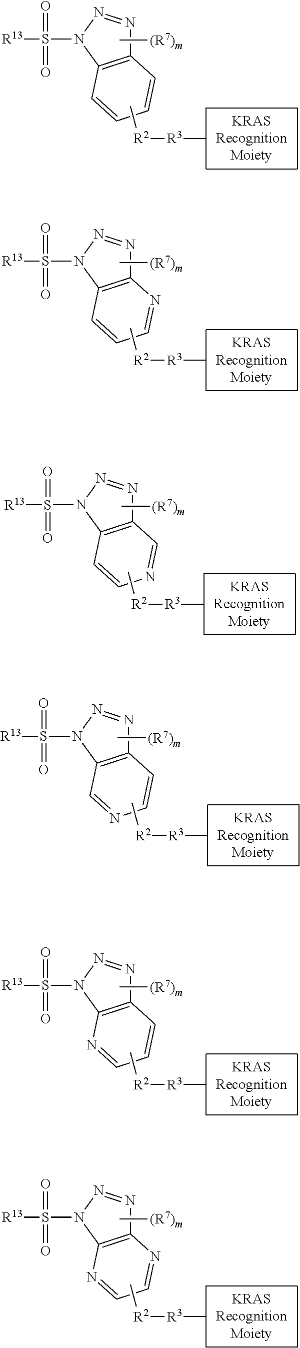

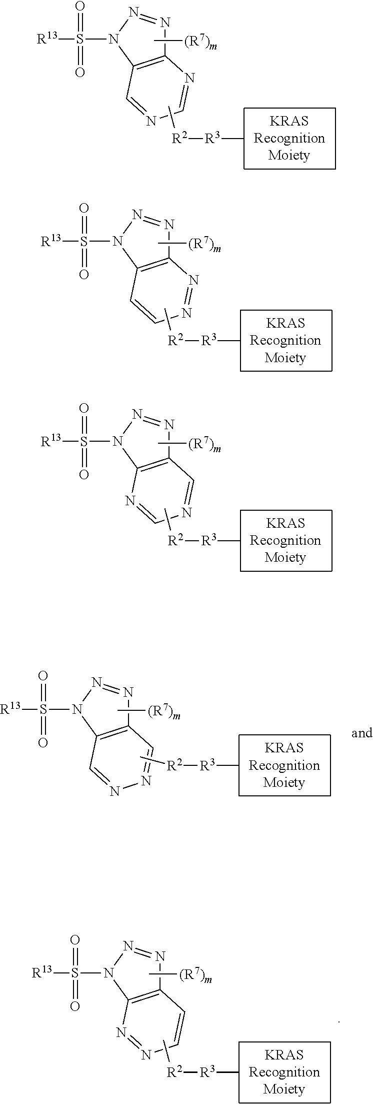

In certain embodiments, the heteroaryl sulfonyl compound of the present invention is selected from:

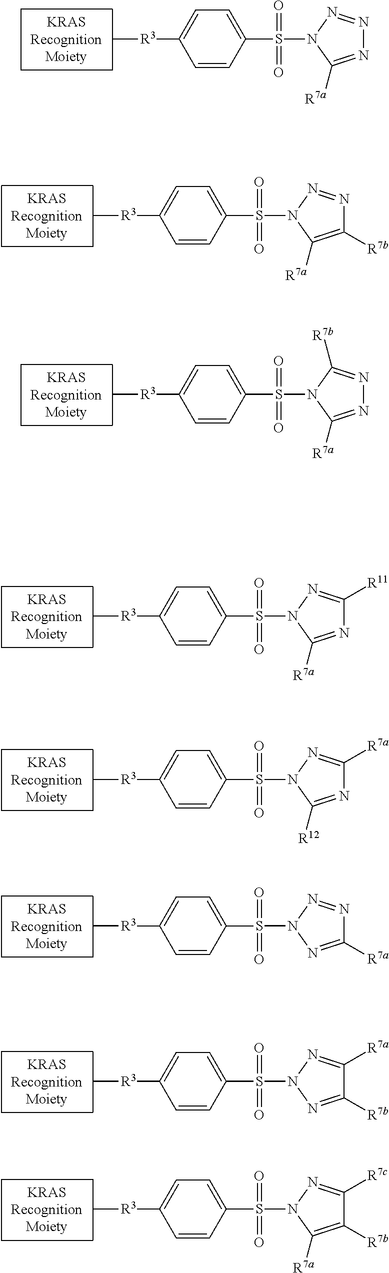

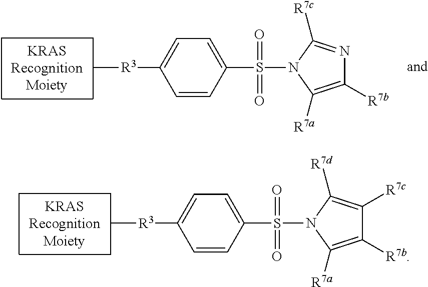

In certain embodiments, the heteroaryl sulfonyl compound of the present invention is selected from:

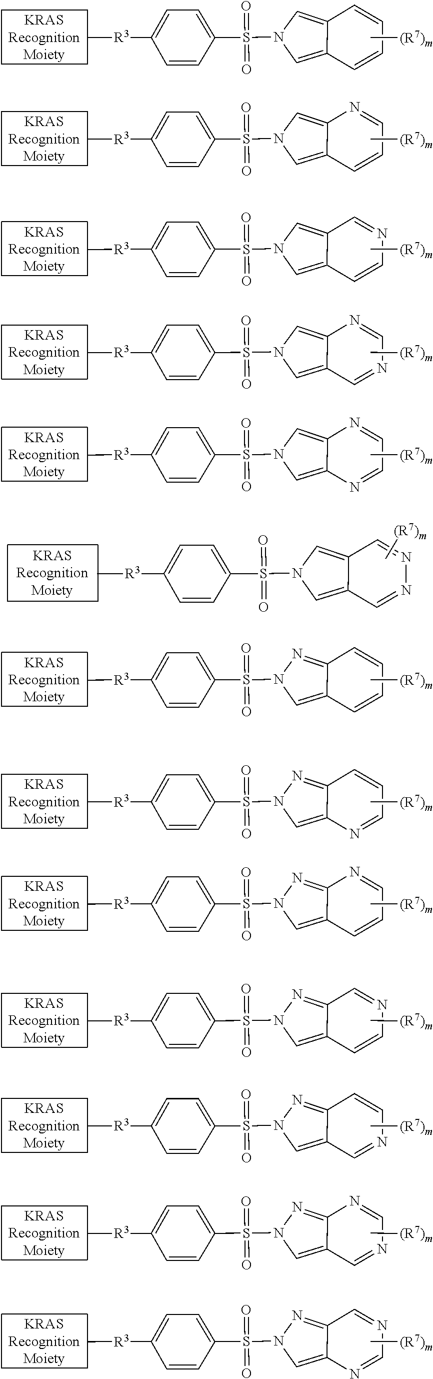

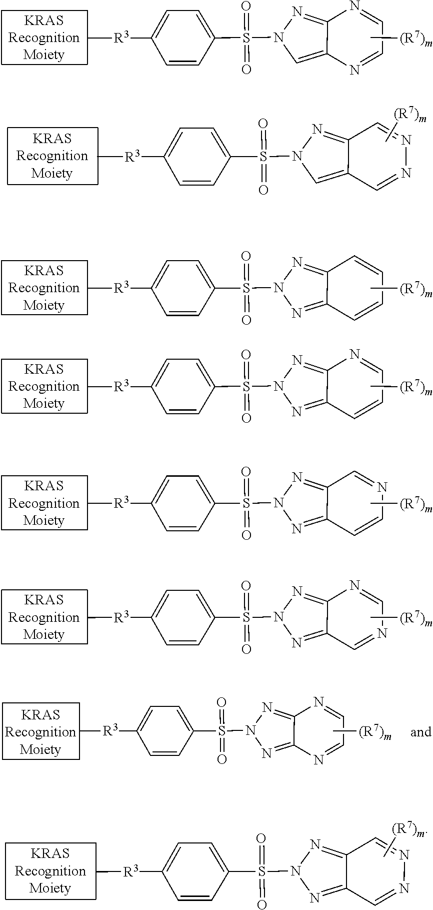

In certain embodiments, the heteroaryl sulfonyl compound of the present invention is selected from:

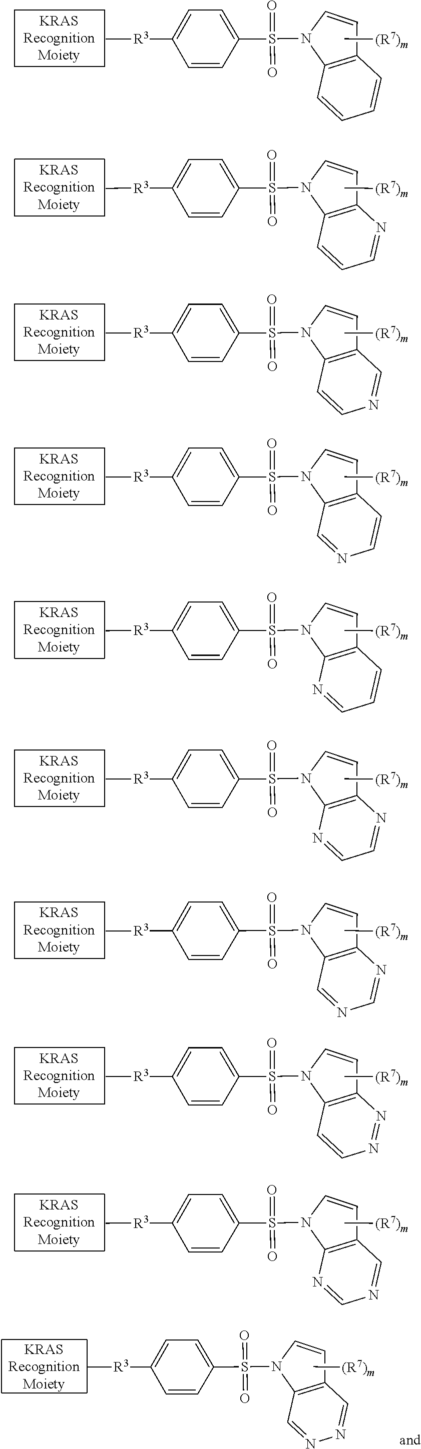

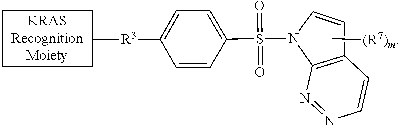

In certain embodiments, the heteroaryl sulfonyl compound of the present invention is selected from:

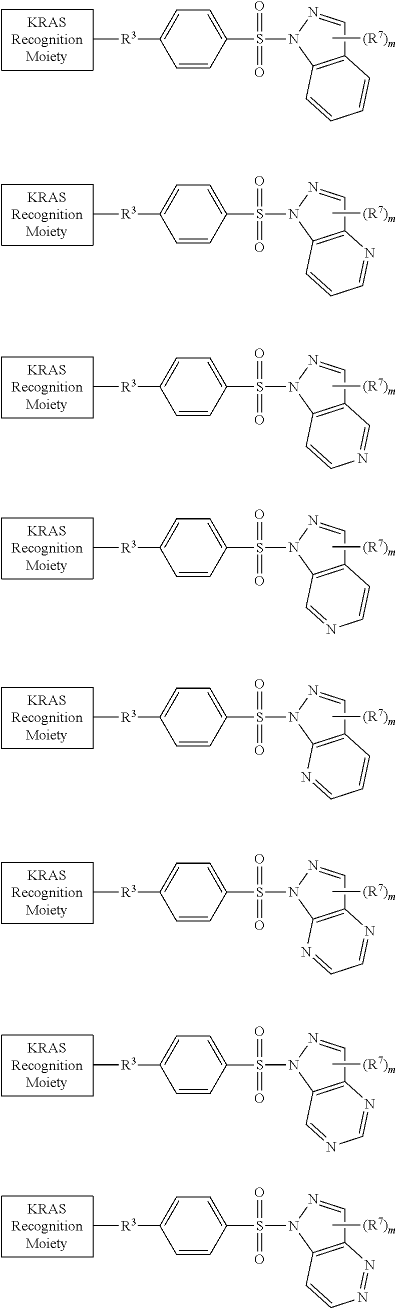

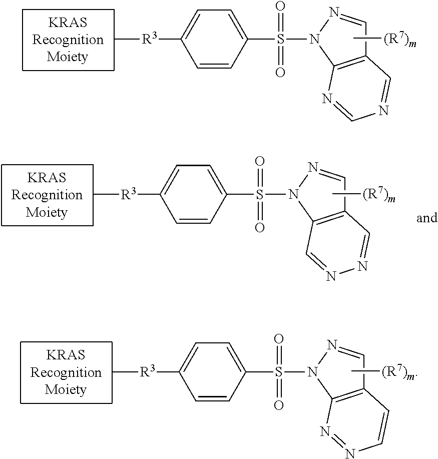

In certain embodiments, the heteroaryl sulfonyl compound of the present invention is selected from:

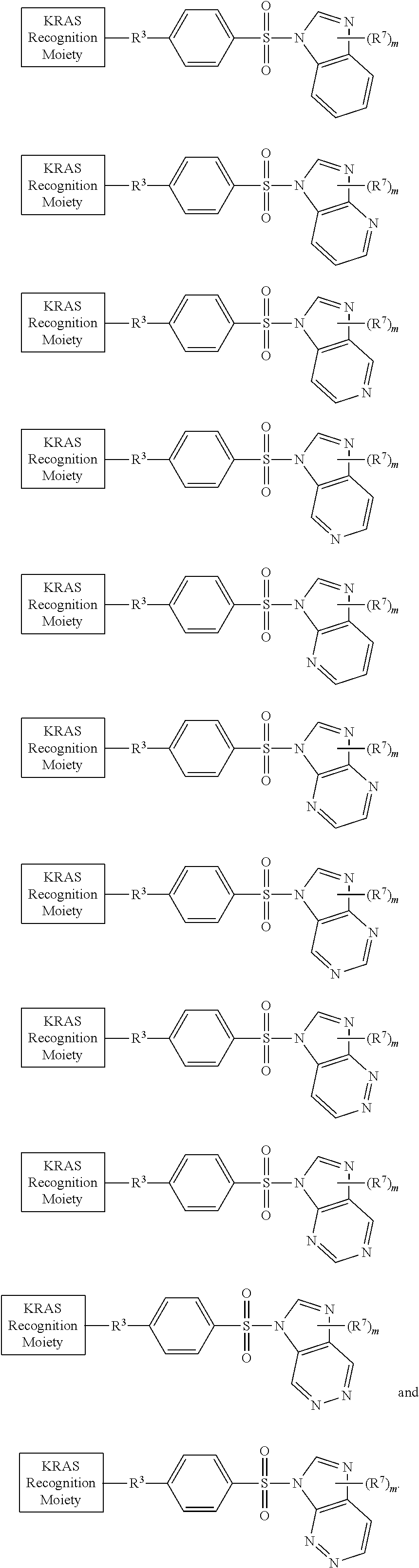

In certain embodiments, the heteroaryl sulfonyl compound of the present invention is selected from:

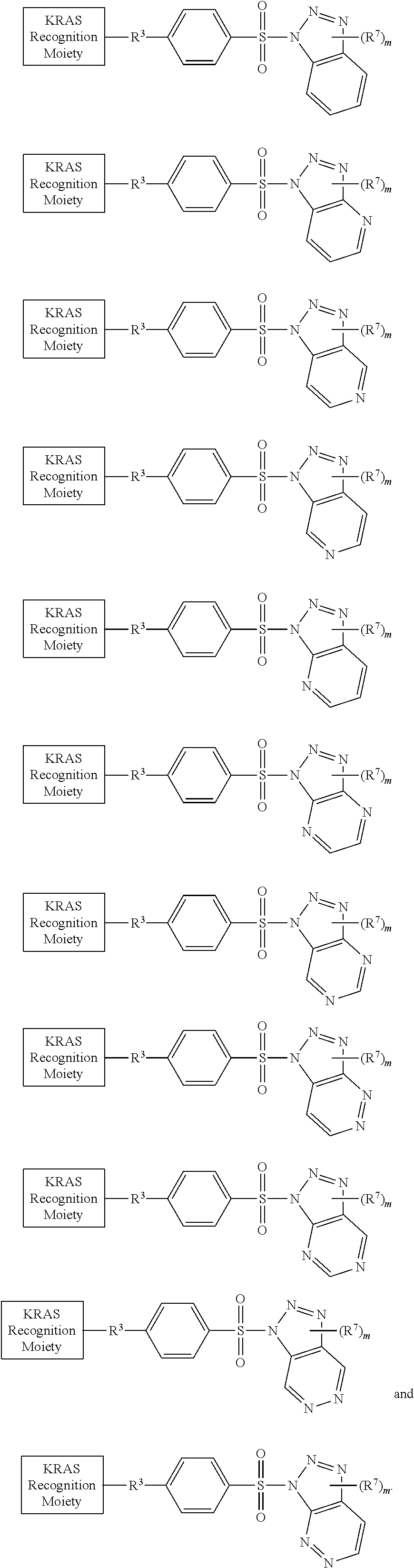

In certain embodiments, the heteroaryl sulfonyl compound of the present invention is selected from:

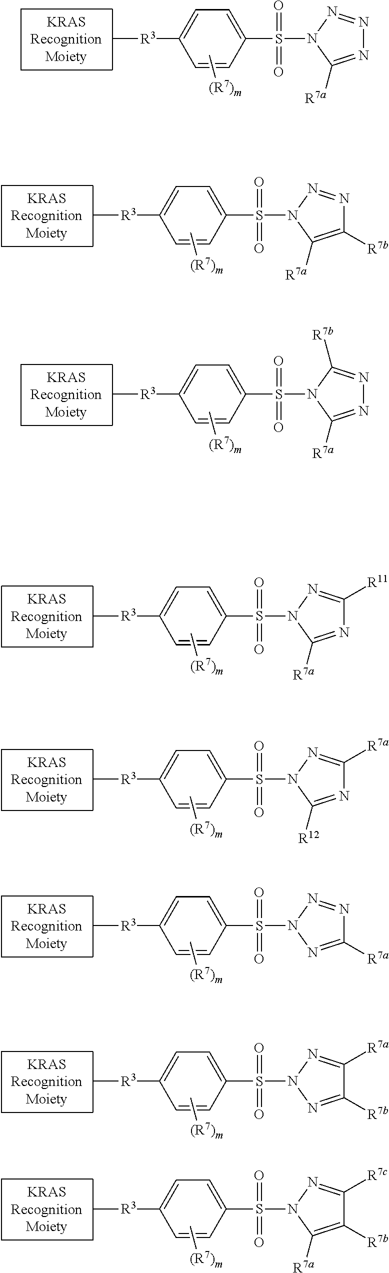

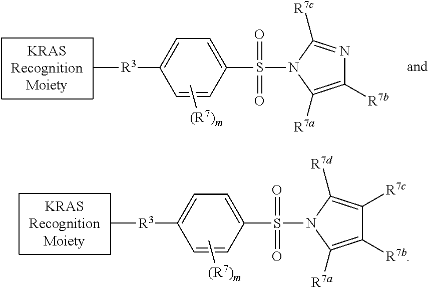

In certain embodiments, the heteroaryl sulfonyl compound of the present invention is selected from:

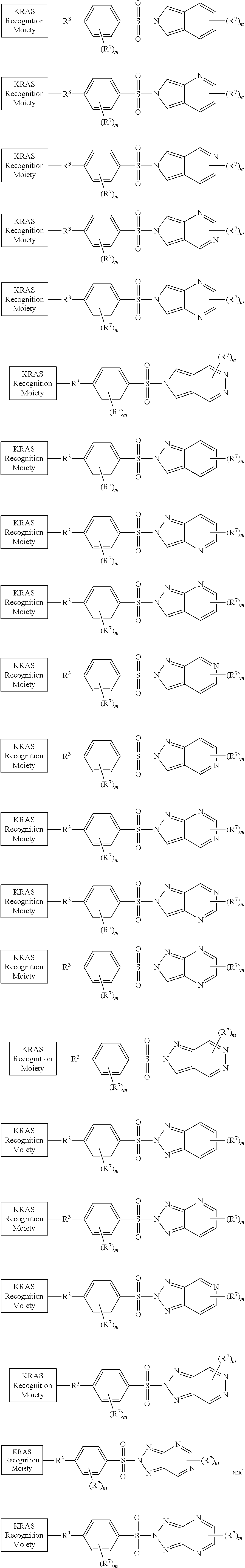

In certain embodiments, the heteroaryl sulfonyl compound of the present invention is selected from:

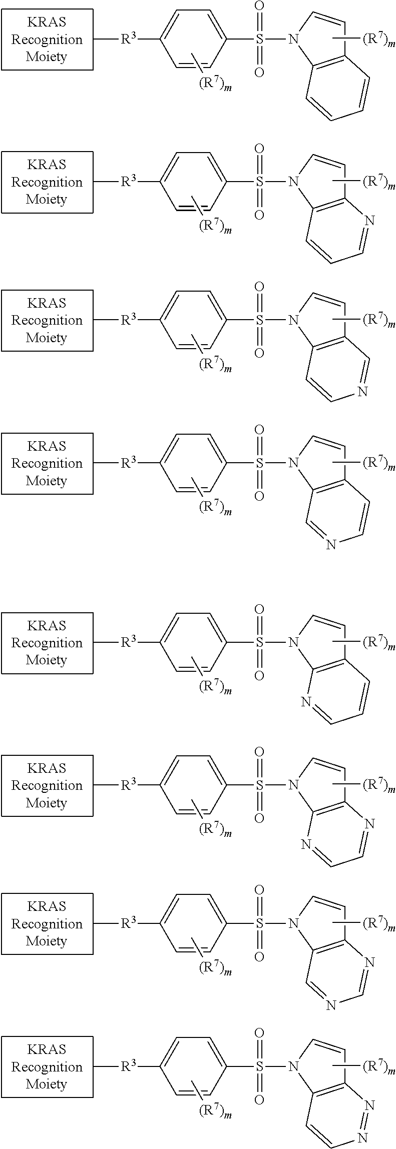

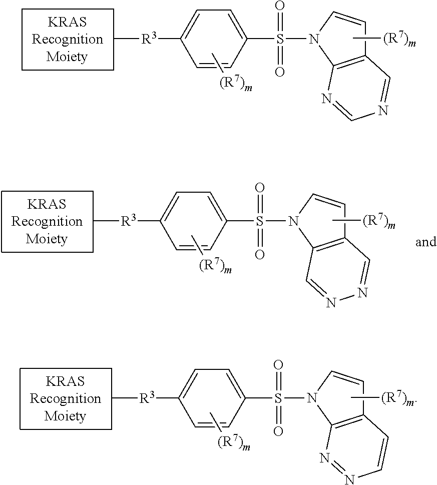

In certain embodiments, the heteroaryl sulfonyl compound of the present invention is selected from:

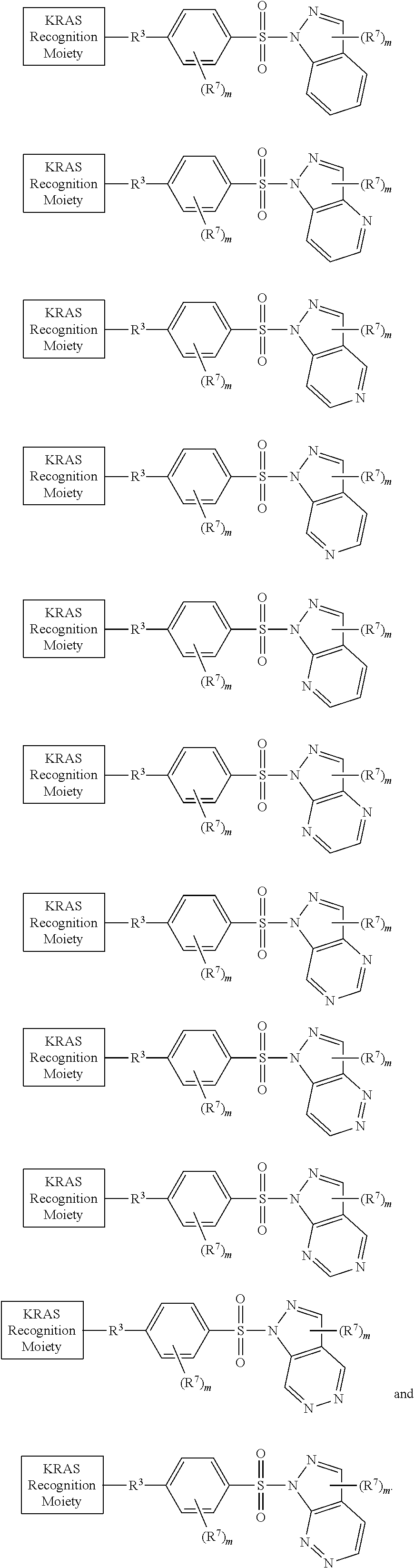

In certain embodiments, the heteroaryl sulfonyl compound of the present invention is selected from:

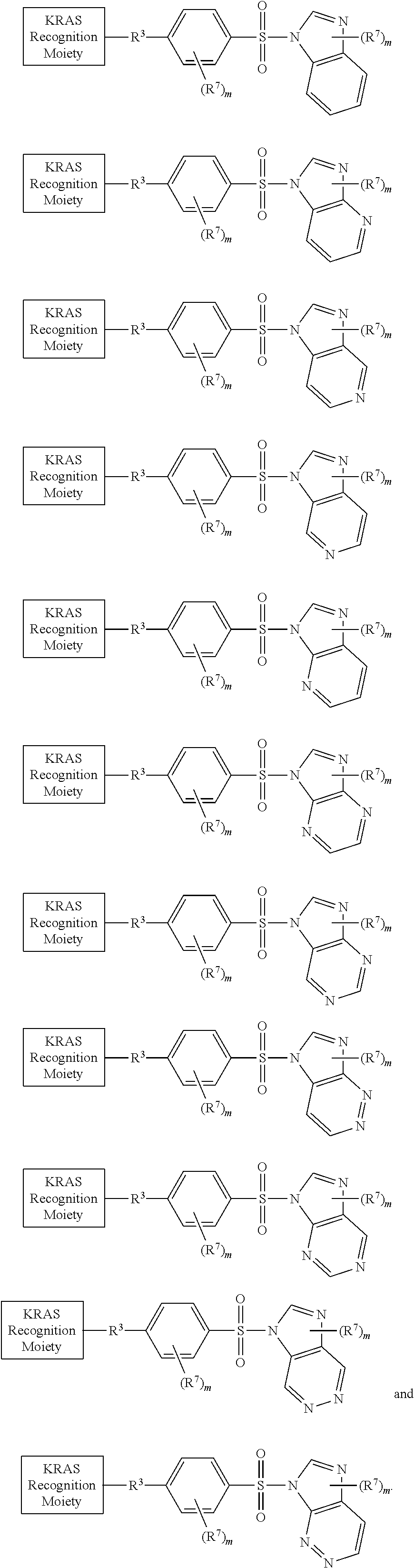

In certain embodiments, the heteroaryl sulfonyl compound of the present invention is selected from:

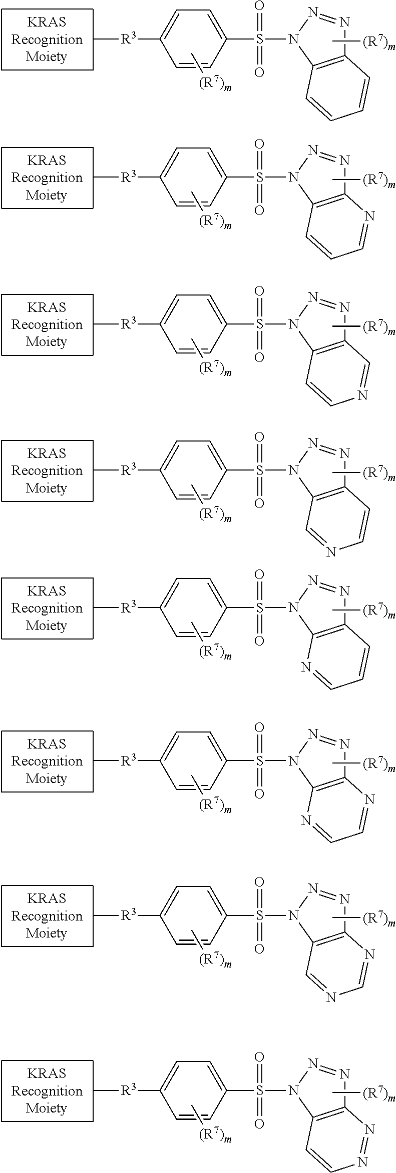

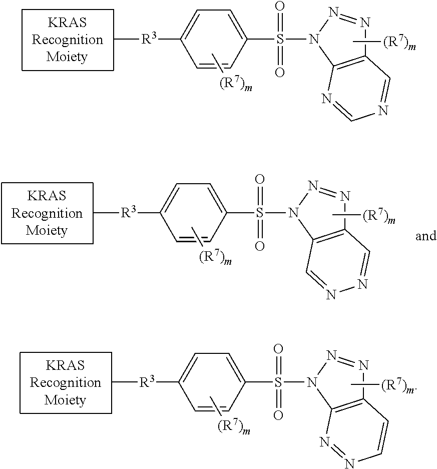

In certain embodiments, the heteroaryl sulfonyl compound of the present invention is selected from:

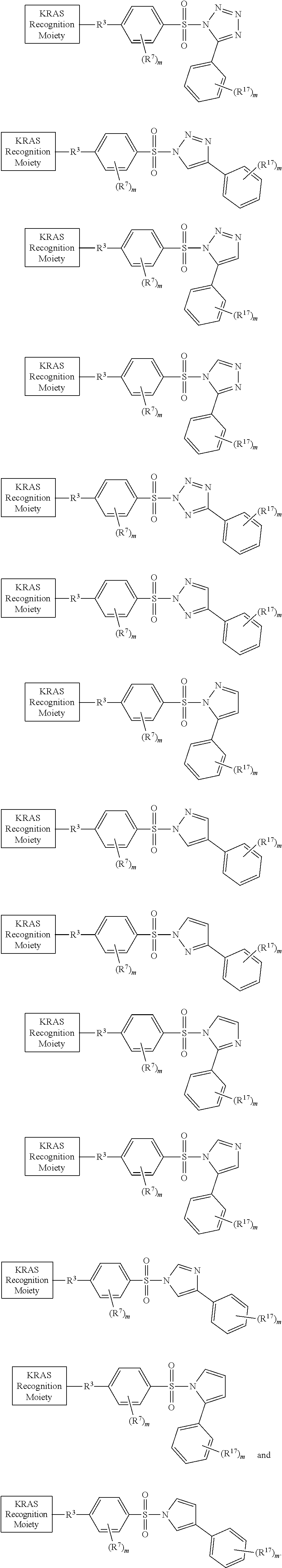

In certain embodiments, the heteroaryl sulfonyl compound of the present invention is selected from:

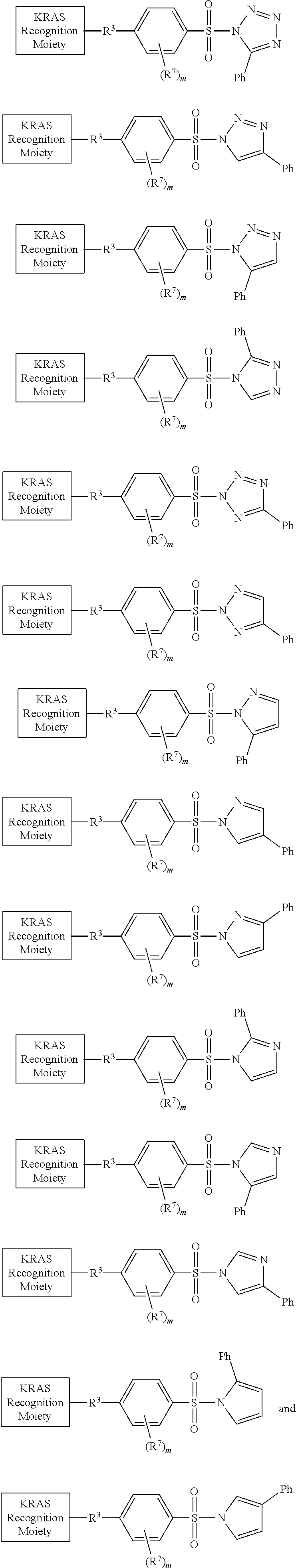

In certain embodiments, the heteroaryl sulfonyl compound of the present invention is selected from:

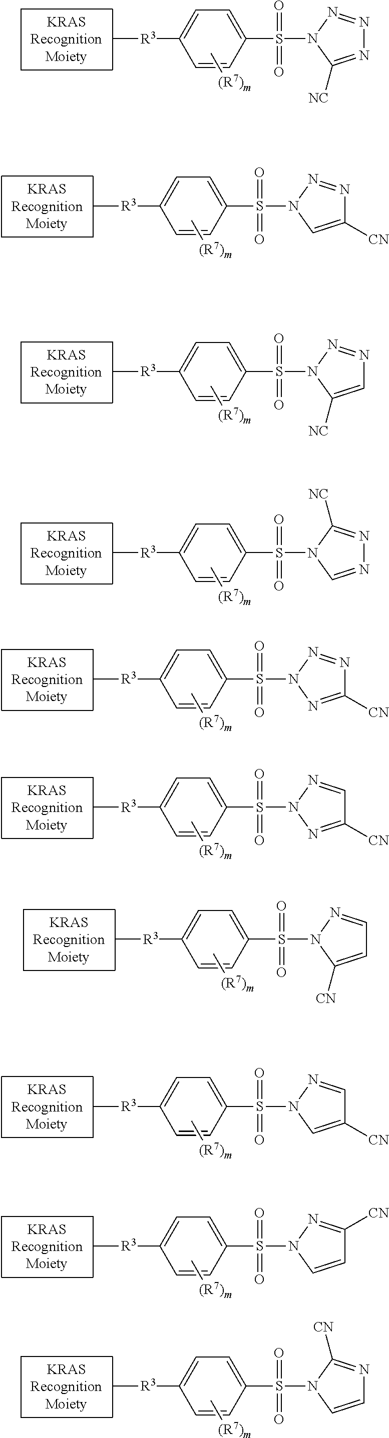

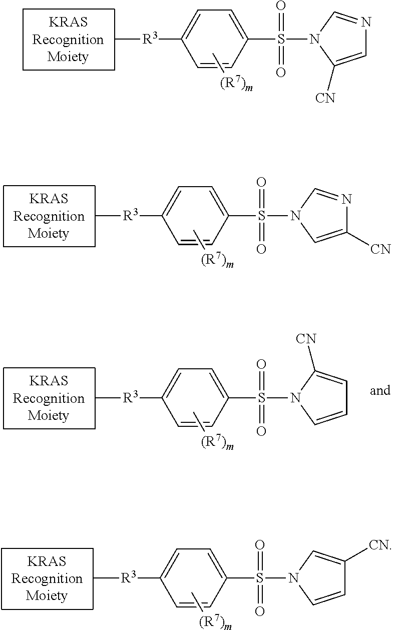

In certain embodiments, the heteroaryl sulfonyl compound of the present invention is selected from:

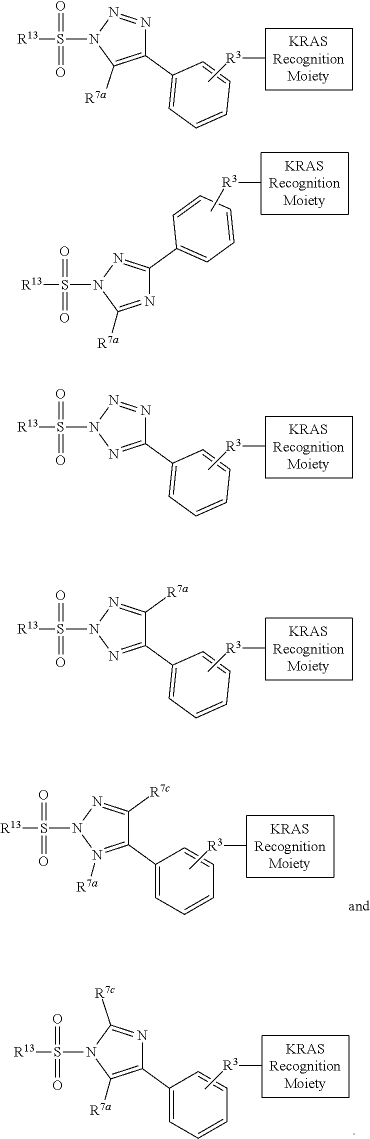

In certain embodiments, the heteroaryl sulfonyl compound of the present invention is selected from:

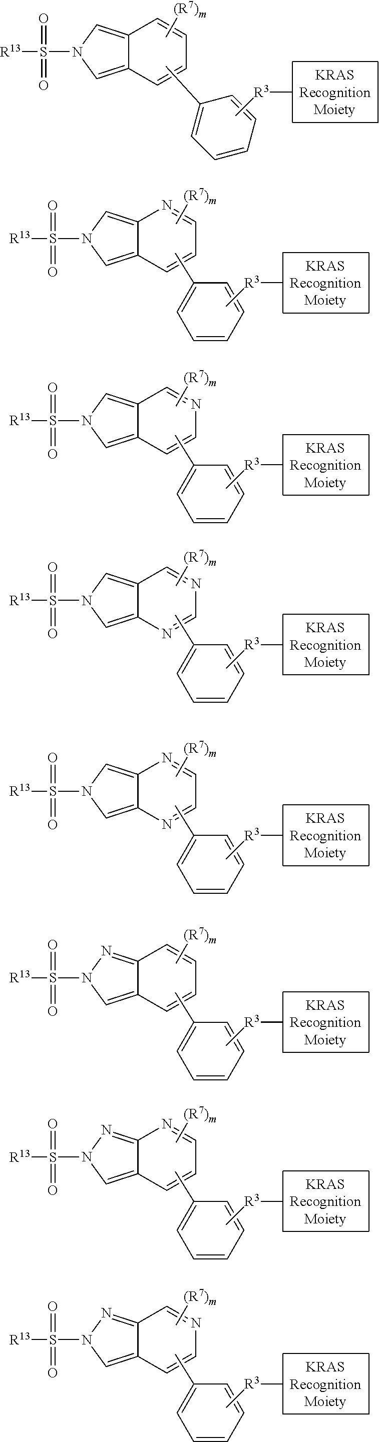

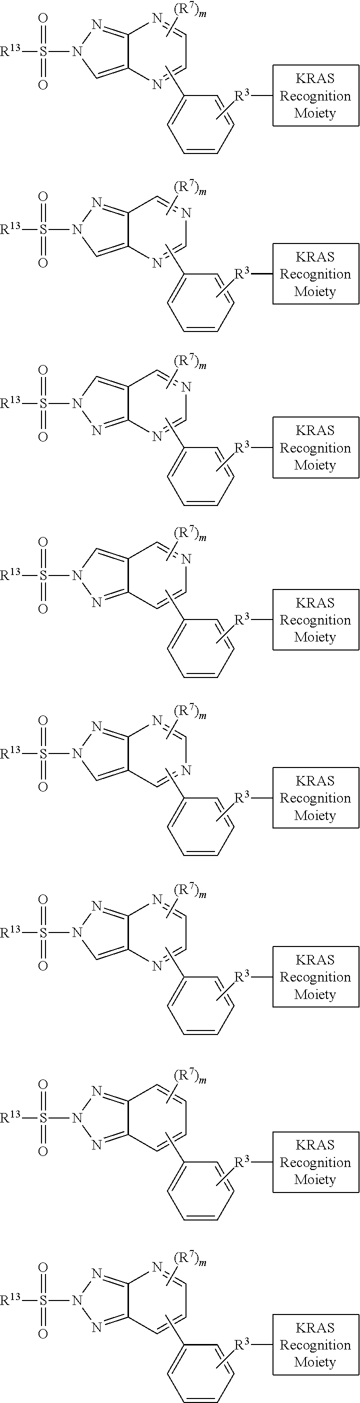

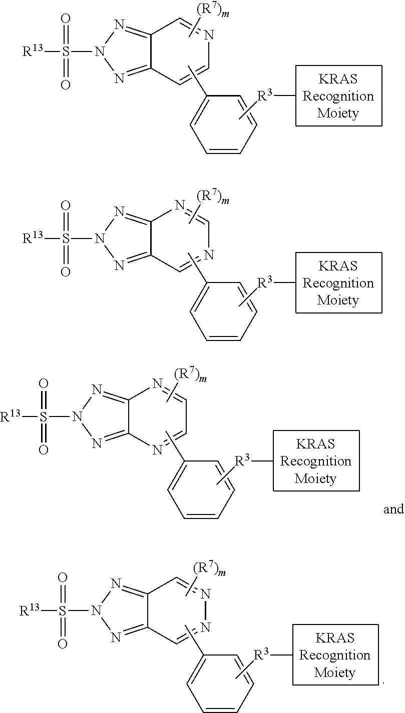

In certain embodiments, the heteroaryl sulfonyl compound of the present invention is selected from:

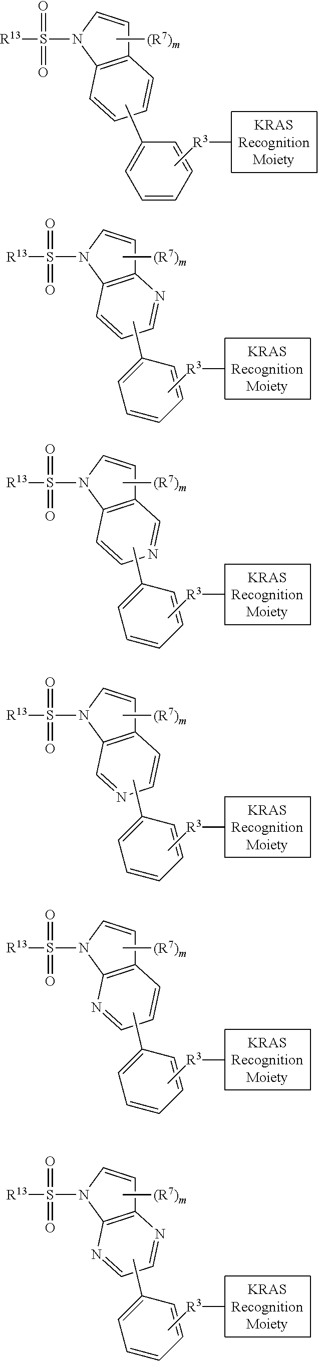

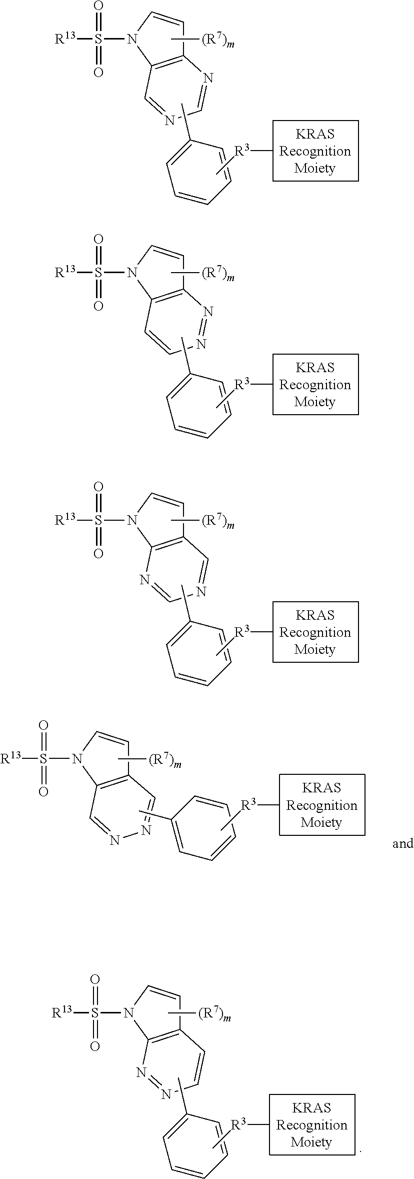

In certain embodiments, the heteroaryl sulfonyl compound of the present invention is selected from:

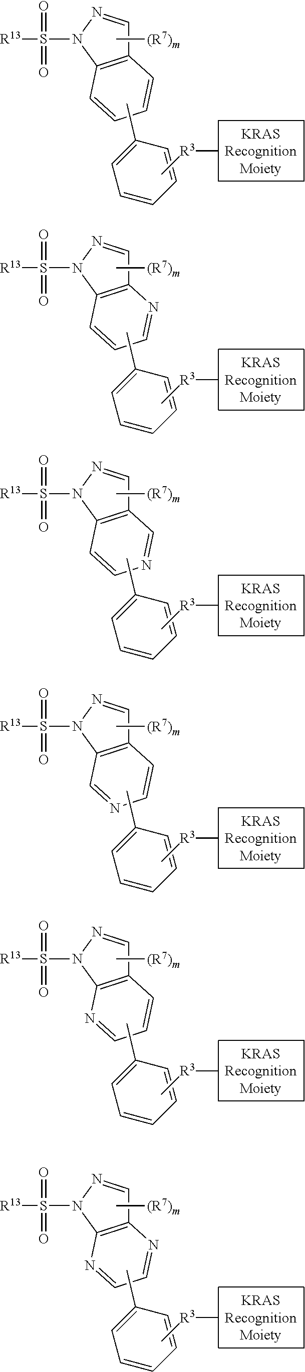

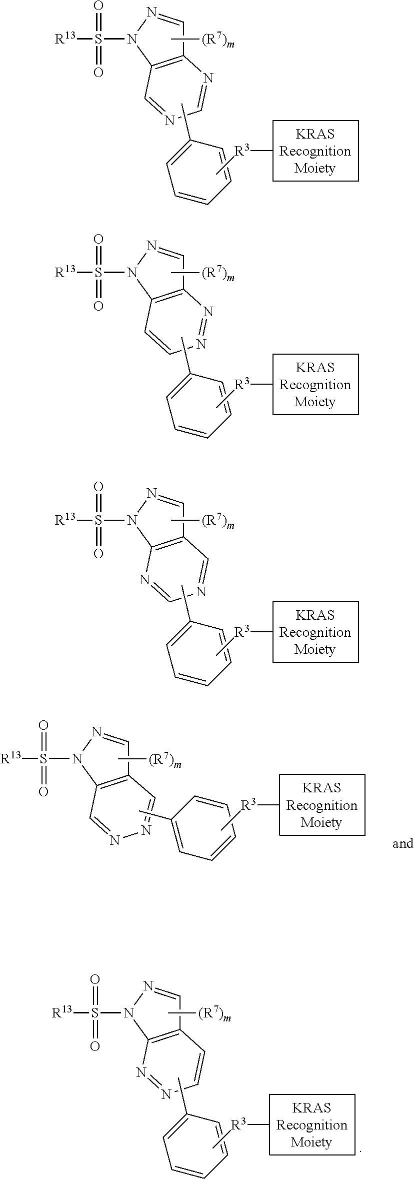

In certain embodiments, the heteroaryl sulfonyl compound of the present invention is selected from:

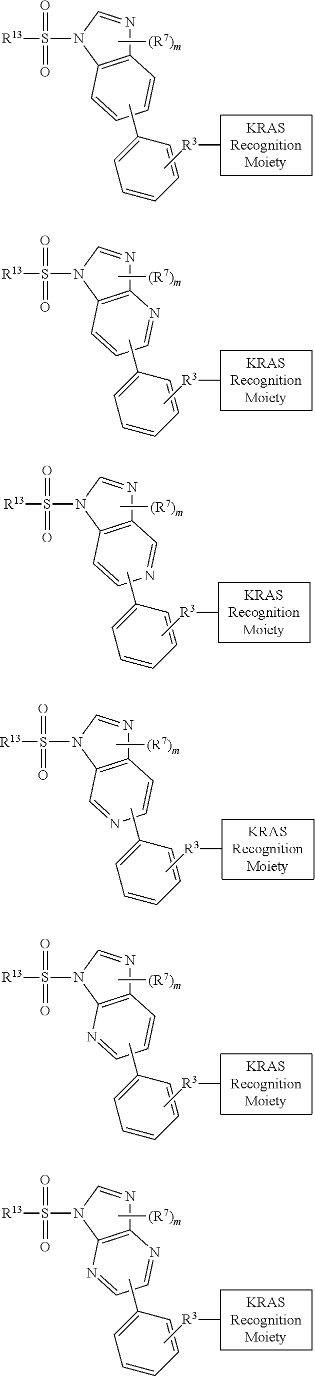

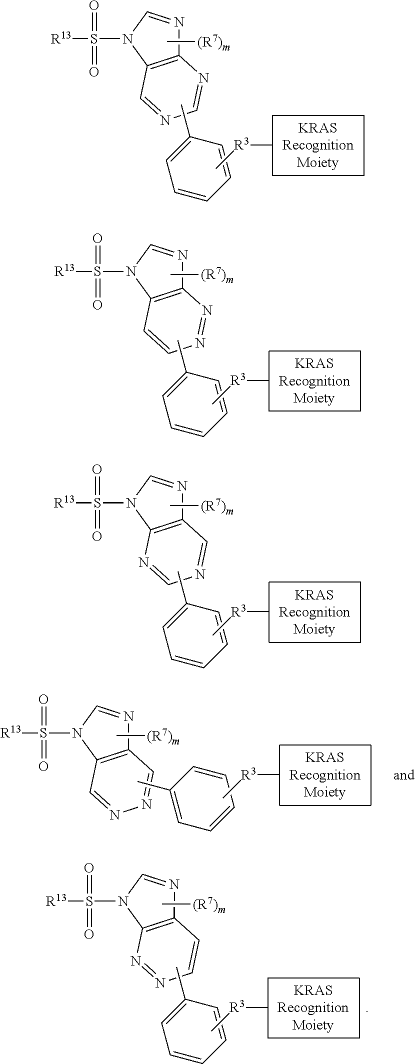

In certain embodiments, the heteroaryl sulfonyl compound of the present invention is selected from:

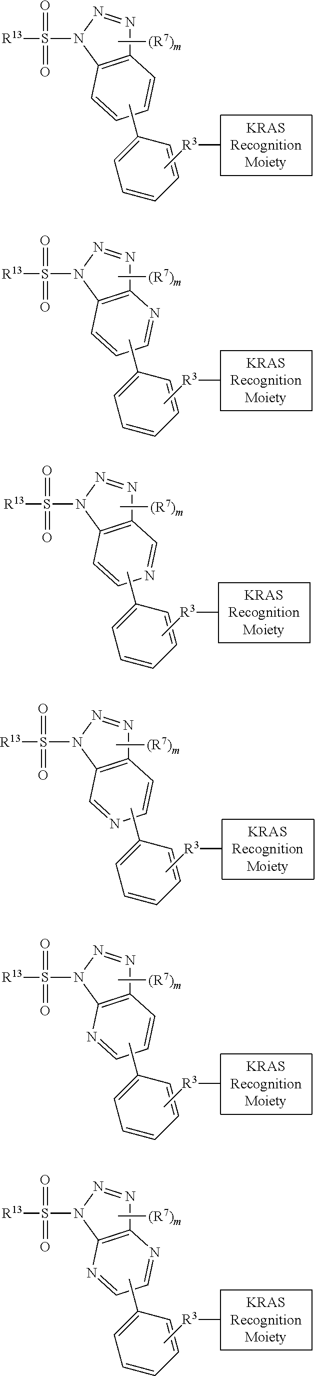

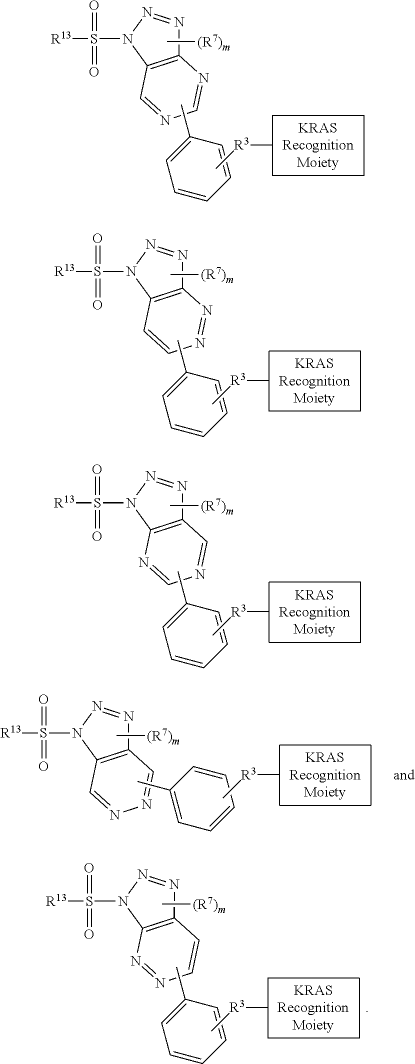

### Embodiments of R¹

In certain embodiments R¹ is

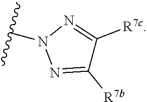

In certain embodiments R¹ is

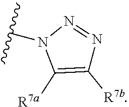

In certain embodiments R¹ is

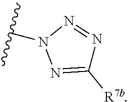

In certain embodiments R¹ is

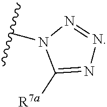

In certain embodiments R¹ is

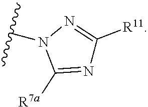

In certain embodiments R¹ is

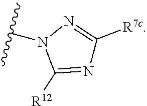

In certain embodiments R¹ is

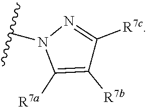

In certain embodiments R¹ is

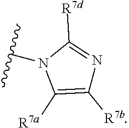

In certain embodiments R¹ is a fused bicyclic heteroaryl.

In certain embodiments R¹ or R⁴ is

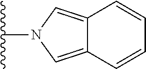 [SMILES: *n1cc2ccccc2c1]

optionally substituted with 1 2, 3, or 4 R⁷ substituents.

In certain embodiments R¹ or R⁴ is

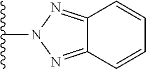 [SMILES: *n1nc2ccccc2n1]

optionally substituted with 1, 2, 3, or 4 R⁷ substituents.

In certain embodiments R¹ or R⁴ is

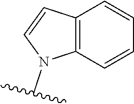 [SMILES: *n1ccc2ccccc21]

optionally substituted with 1, 2, 3, or 4 R⁷ substituents.

In certain embodiments R¹ or R⁴ is

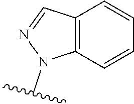 [SMILES: *n1ncc2ccccc21]

optionally substituted with 1, 2, 3, or 4 R⁷ substituents.

In certain embodiments R¹ or R⁴ is

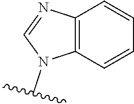 [SMILES: *n1cnc2ccccc21]

optionally substituted with 1, 2, 3, or 4 R⁷ substituents.

In certain embodiments R¹ or R⁴ is

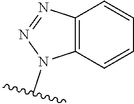 [SMILES: *n1nnc2ccccc21]

optionally substituted with 1, 2, 3, or 4 R⁷ substituents.

In certain embodiments R¹ or R⁴ is

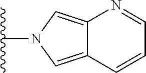 [SMILES: *n1cc2cccnc2c1]

optionally substituted with 1, 2, 3, or 4 R⁷ substituents.

In certain embodiments R¹ or R⁴ is

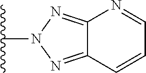 [SMILES: *n1nc2cccnc2n1]

optionally substituted with 1, 2, or 3, R⁷ substituents.

In certain embodiments R¹ or R⁴ is

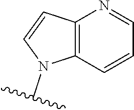 [SMILES: *n1ccc2ncccc21]

optionally substituted with 1, 2, 3, or 4 R⁷ substituents.

In certain embodiments R¹ or R⁴ is

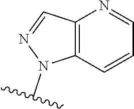 [SMILES: *n1ncc2ncccc21]

optionally substituted with 1, 2, 3, or 4 R⁷ substituents.

In certain embodiments R¹ or R⁴ is

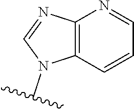 [SMILES: *n1cnc2ncccc21]

optionally substituted with 1, 2, 3, or 4 R⁷ substituents.

In certain embodiments R¹ or R⁴ is

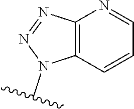 [SMILES: *n1nnc2ncccc21]

optionally substituted with 1, 2, or 3, R⁷ substituents.

In certain embodiments R¹ or R⁴ is

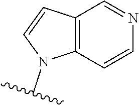 [SMILES: *n1ccc2cnccc21]

optionally substituted with 1, 2, 3, or 4 R⁷ substituents.

In certain embodiments R¹ or R⁴ is

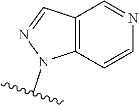 [SMILES: *n1ncc2cnccc21]

optionally substituted with 1, 2, 3, or 4 R⁷ substituents.

In certain embodiments R¹ or R⁴ is

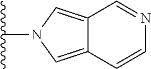 [SMILES: *n1cc2ccncc2c1]

optionally substituted with 1, 2, 3, or 4 R⁷ substituents.

In certain embodiments R¹ or R⁴ is

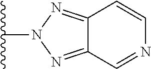 [SMILES: *n1nc2ccncc2n1]

optionally substituted with 1, 2, or 3, R⁷ substituents.

In certain embodiments R¹ or R⁴ is

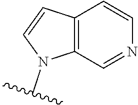 [SMILES: *n1ccc2ccncc21]

optionally substituted with 1, 2, 3, or 4 R⁷ substituents.

In certain embodiments R¹ or R⁴ is

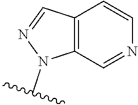 [SMILES: *n1ncc2ccncc21]

optionally substituted with 1, 2, 3, or 4 R⁷ substituents.

In certain embodiments R¹ or R⁴ is

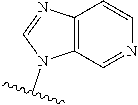 [SMILES: *n1cnc2ccncc21]

optionally substituted with 1, 2, 3, or 4 R⁷ substituents.

In certain embodiments R¹ or R⁴ is

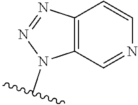 [SMILES: *n1nnc2ccncc21]

optionally substituted with 1, 2, or 3, R⁷ substituents.

In certain embodiments R¹ or R⁴ is

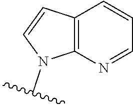 [SMILES: *n1ccc2cccnc21]

optionally substituted with 1, 2, 3, or 4 R⁷ substituents.

In certain embodiments R¹ or R⁴ is

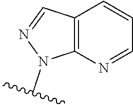 [SMILES: *n1ncc2cccnc21]

optionally substituted with 1, 2, 3, or 4 R⁷ substituents.

In certain embodiments R¹ or R⁴ is

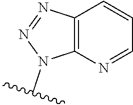 [SMILES: *n1nnc2cccnc21]

optionally substituted with 1, 2, or 3, R⁷ substituents.

In certain embodiments R¹ or R⁴ is

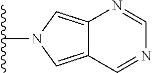 [SMILES: *n1cc2cncnc2c1]

optionally substituted with 1, 2, 3, or 4 R⁷ substituents.

In certain embodiments R¹ or R⁴ is

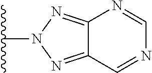 [SMILES: *n1nc2cncnc2n1]

optionally substituted with 1 or 2 R⁷ substituents.

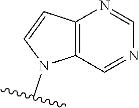 [SMILES: *n1ccc2ncncc21]

In certain embodiments R¹ or R⁴ is

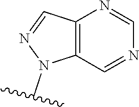 [SMILES: *n1ncc2ncncc21]

optionally substituted with 1, 2, 3, or 4 R⁷ substituents.

In certain embodiments R¹ or R⁴ is

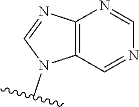 [SMILES: *n1cnc2ncncc21]

optionally substituted with 1, 2, or 3 R⁷ substituents.

In certain embodiments R¹ or R⁴ is

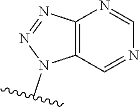 [SMILES: *n1nnc2ncncc21]

optionally substituted with 1 or 2 R⁷ substituents.

In certain embodiments R¹ or R⁴ is

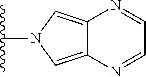 [SMILES: *n1cc2nccnc2c1]

optionally substituted with 1, 2, 3, or 4 R⁷ substituents.

In certain embodiments R¹ or R⁴ is

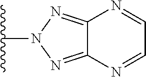 [SMILES: *n1nc2nccnc2n1]

optionally substituted with 1 or 2 R⁷ substituents.

In certain embodiments R¹ or R⁴ is

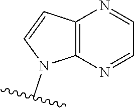 [SMILES: *n1ccc2nccnc21]

optionally substituted with 1, 2, 3, or 4 R⁷ substituents.

In certain embodiments R¹ or R⁴ is

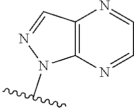 [SMILES: *n1ncc2nccnc21]

optionally substituted with 1, 2, or 3 R⁷ substituents.

In certain embodiments R¹ or R⁴ is

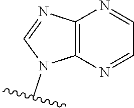 [SMILES: *n1cnc2nccnc21]

optionally substituted with 1, 2, or 3 R⁷ substituents.

In certain embodiments R¹ or R⁴ is

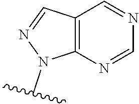 [SMILES: *n1ncc2cncnc21]

optionally substituted with 1, 2, or 3 R⁷ substituents.

In certain embodiments R¹ or R⁴ is

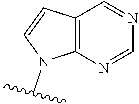 [SMILES: *n1ccc2cncnc21]

optionally substituted with 1, 2, 3, or 4 R⁷ substituents.

In certain embodiments R¹ or R⁴ is

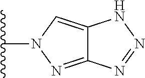 [SMILES: *n1cc2[nH]nnc2n1]

optionally substituted with 1 or 2 R⁷ substituents.

In certain embodiments R¹ or R⁴ is

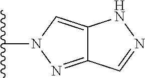 [SMILES: *n1cc2[nH]ncc2n1]

optionally substituted with 1, 2, or 3 R⁷ substituents.

In certain embodiments R¹ or R⁴ is

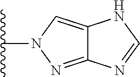 [SMILES: *n1cc2[nH]cnc2n1]

optionally substituted with 1, 2, or 3 R⁷ substituents.

In certain embodiments R¹ or R⁴ is

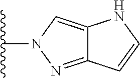 [SMILES: *n1cc2[nH]ccc2n1]

optionally substituted with 1, 2, or 3 R⁷ substituents.

In certain embodiments R¹ or R⁴ is

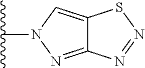 [SMILES: *n1cc2snnc2n1]

optionally substituted with 1 R⁷ substituents.

In certain embodiments R¹ or R⁴ is

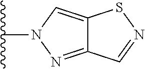 [SMILES: *n1cc2sncc2n1]

optionally substituted with 1 or 2 R⁷ substituents.

In certain embodiments R¹ or R⁴ is

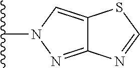 [SMILES: *n1cc2scnc2n1]

optionally substituted with 1 or 2 R⁷ substituents.

In certain embodiments R¹ or R⁴ is

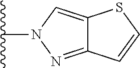 [SMILES: *n1cc2sccc2n1]

optionally substituted with 1, 2, or 3 R⁷ substituents.

In certain embodiments R¹ or R⁴ is

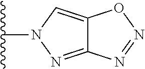 [SMILES: *n1cc2onnc2n1]

optionally substituted with 1 R⁷ substituents.

In certain embodiments R¹ or R⁴ is

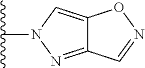 [SMILES: *n1cc2oncc2n1]

optionally substituted with 1 or 2 R⁷ substituents.

In certain embodiments R¹ or R⁴ is

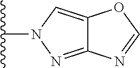 [SMILES: *n1cc2ocnc2n1]

optionally substituted with 1 or 2 R⁷ substituents.

In certain embodiments R¹ or R⁴ is

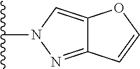 [SMILES: *n1cc2occc2n1]

optionally substituted with 1, 2 or 3 R⁷ substituents.

In certain embodiments R¹ or R⁴ is

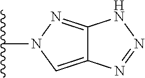 [SMILES: *n1cc2nn[nH]c2n1]

optionally substituted with 1 or 2 R⁷ substituents.

In certain embodiments R¹ or R⁴ is

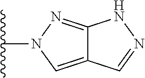 [SMILES: *n1cc2cn[nH]c2n1]

optionally substituted with 1, 2, or 3 R⁷ substituents.

In certain embodiments R¹ or R⁴ is

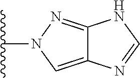 [SMILES: *n1cc2nc[nH]c2n1]

optionally substituted with 1, 2, or 3 R⁷ substituents.

In certain embodiments R¹ or R⁴ is

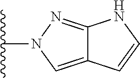 [SMILES: *n1cc2cc[nH]c2n1]

optionally substituted with 1, 2, 3, or 4 R⁷ substituents.

In certain embodiments R¹ or R⁴ is

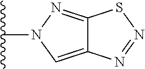 [SMILES: *n1cc2nnsc2n1]

optionally substituted with 1 R⁷ substituents.

In certain embodiments R¹ or R⁴ is

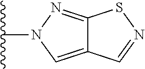 [SMILES: *n1cc2cnsc2n1]

optionally substituted with 1 or 2 R⁷ substituents.

In certain embodiments R¹ or R⁴ is

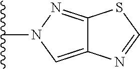 [SMILES: *n1cc2ncsc2n1]

optionally substituted with 1 or 2 R⁷ substituents.

In certain embodiments R¹ or R⁴ is

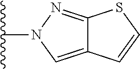 [SMILES: *n1cc2ccsc2n1]

optionally substituted with 1, 2, or 3 R⁷ substituents.

In certain embodiments R¹ or R⁴ is

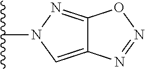 [SMILES: *n1cc2nnoc2n1]

optionally substituted with 1 R⁷ substituents.

In certain embodiments R¹ or R⁴ is

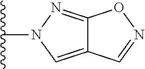 [SMILES: *n1cc2cnoc2n1]

optionally substituted with 1 or 2 R⁷ substituents.

In certain embodiments R¹ or R⁴ is

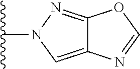 [SMILES: *n1cc2ncoc2n1]

optionally substituted with 1 or 2 R⁷ substituents.

In certain embodiments R¹ or R⁴ is

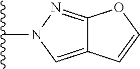 [SMILES: *n1cc2ccoc2n1]

optionally substituted with 1, 2, or 3 R⁷ substituents.

### Embodiments of R² and R³

Bivalent substituents described herein can be either attached in a left to right fashion or a right to left fashion except as excluded by context. For example, where R² is -aryl-C(O)—NR⁶— either the aryl or nitrogen side is attached to the sulfonyl group. For example, when R² is -aryl-C(O)—NR⁶—, Formula I can be

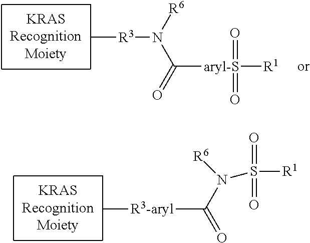

similarly, when —R³— is

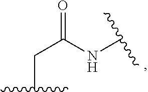 [SMILES: *CC(=O)N*]

### Formula I can be

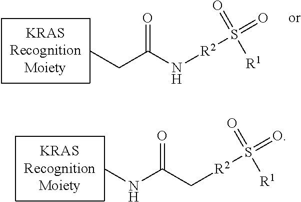

In certain embodiments —R²—R³— and —R³—R²— are selected from:

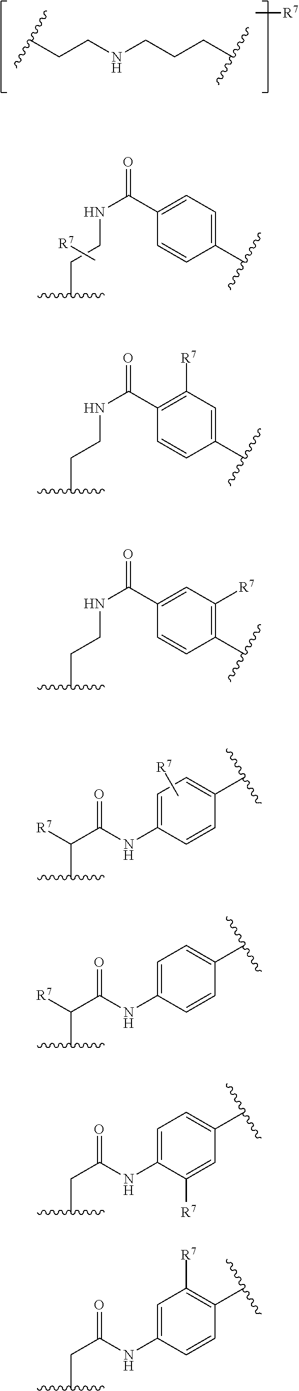

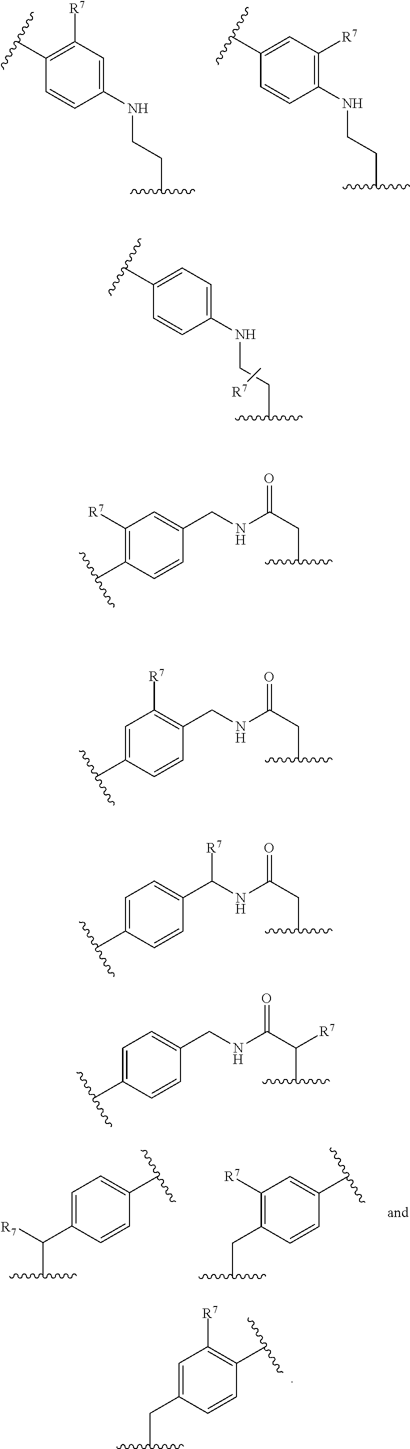

In certain embodiments —R²—R³— and —R³—R²— are selected from:

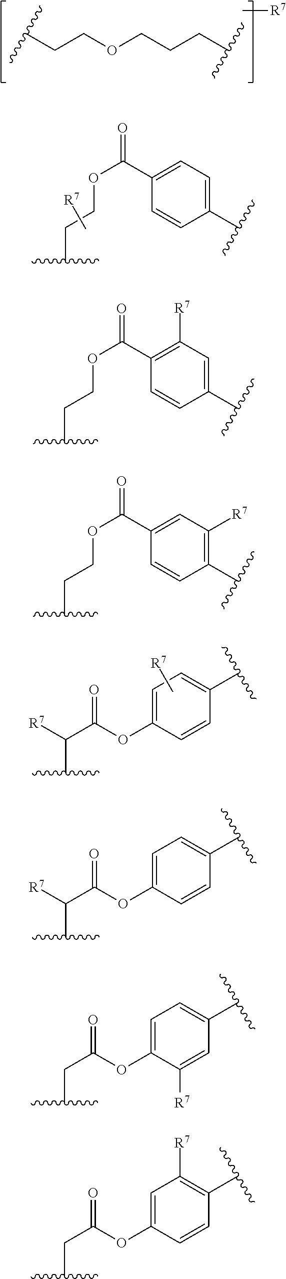

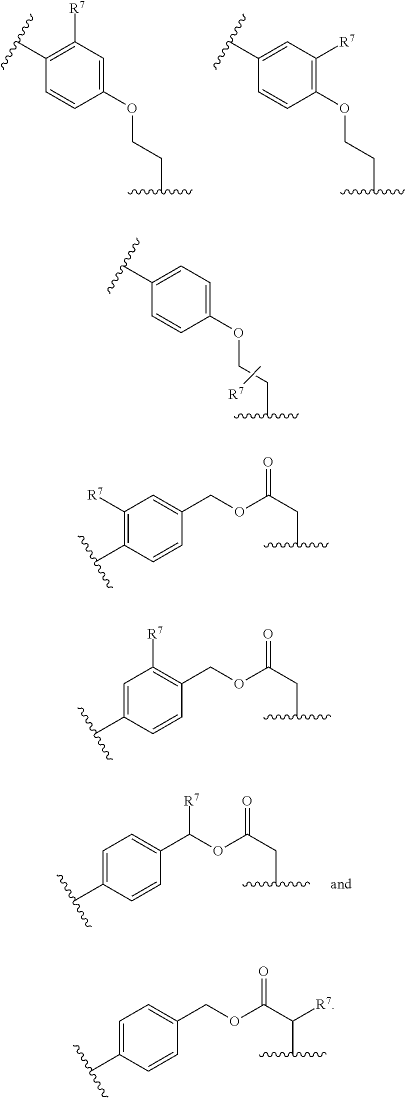

In certain embodiments —R²—R³— and —R³—R²— are selected from:

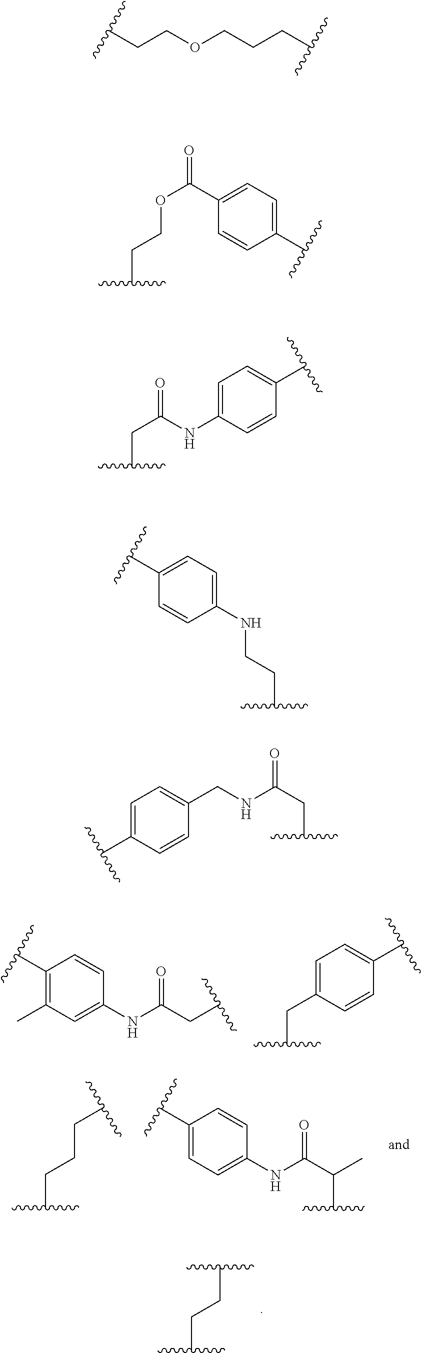

In certain embodiments R² is selected from:

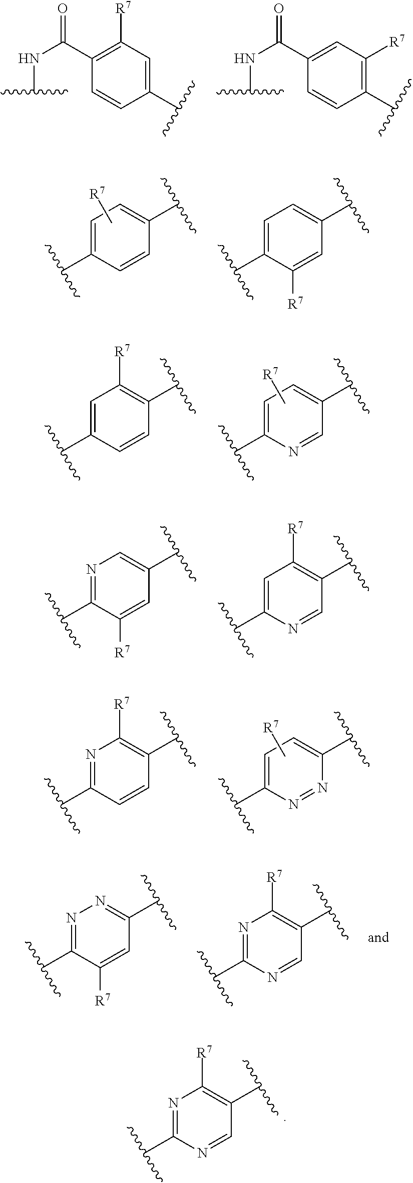

In certain embodiments R² is selected from

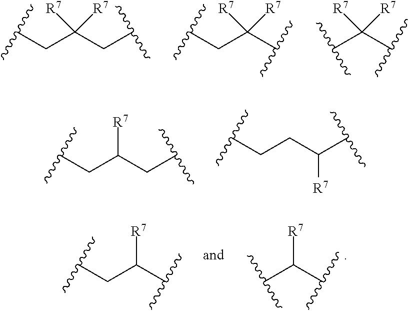

In certain embodiments R² is selected from:

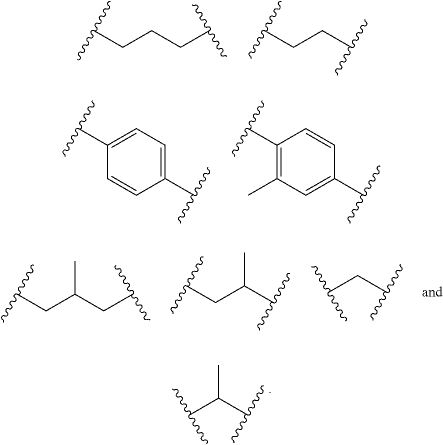

In certain embodiments R² is selected from:

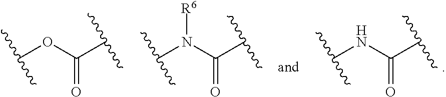

In certain embodiments R³ is selected from

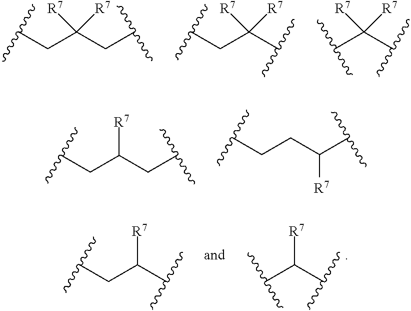

In certain embodiments R³ is selected from:

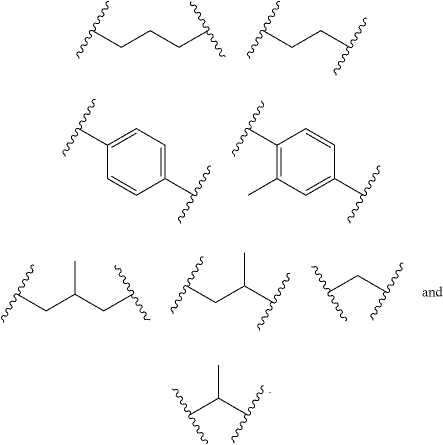

In certain embodiments R³ is selected from:

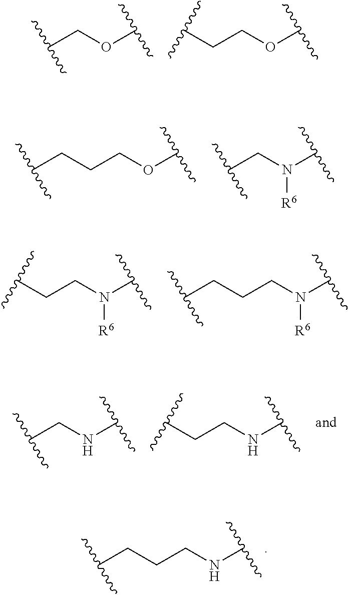

In certain embodiments R³ is selected from:

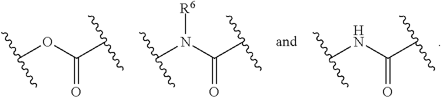

In certain embodiments R³ is selected from:

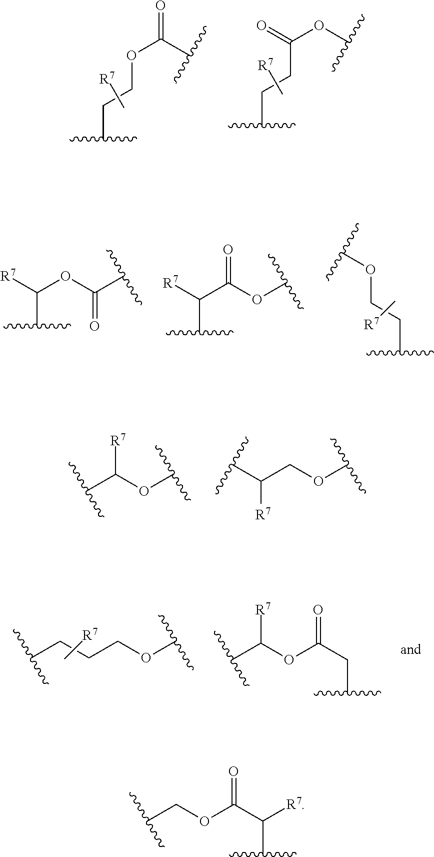

In certain embodiments R³ is selected from:

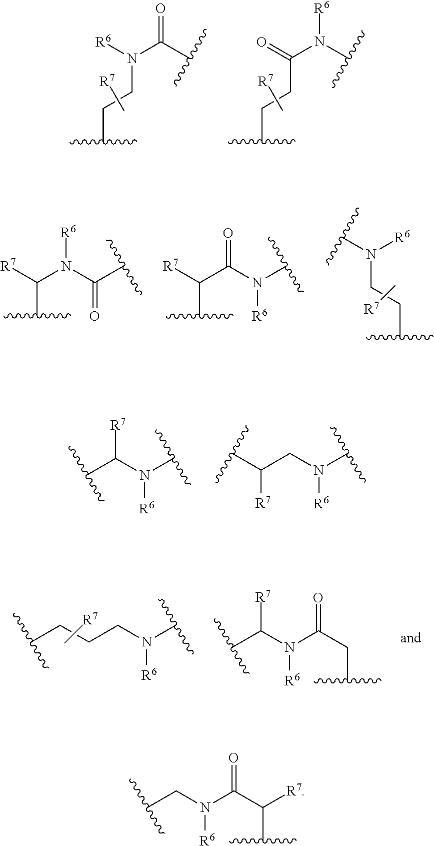

In certain embodiments R³ is selected from:

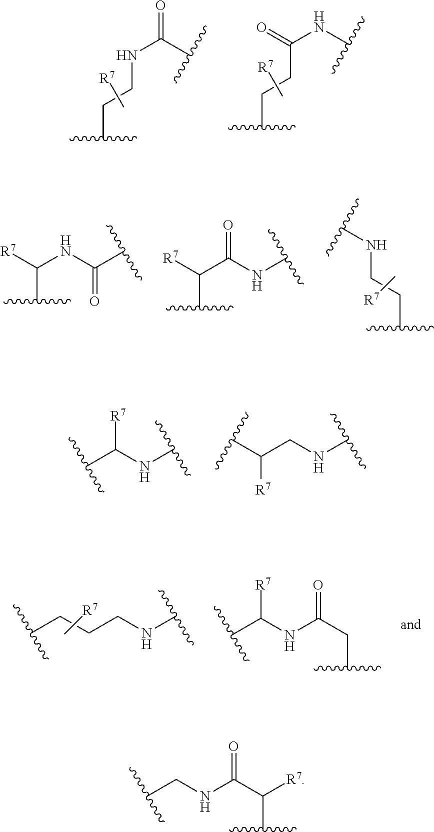

In certain embodiments R² is bond.

In certain embodiments R³ is bond.

In certain embodiments R² and R³ are both bond.

In certain embodiments one of R² and R³ is bond and the other is selected from alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl, aryl-C(O)—NR⁶—, heteroaryl-C(O)—NR⁶—, and heteroaryl, each of which is optionally substituted as allowed by valence with 1, 2, 3, or 4 substituents selected from R⁷.

In certain embodiments p is 1.

In certain embodiments p is 2.

In certain embodiments p is 3.

In certain embodiments p is 4.

In certain embodiments p is 5.

In certain embodiments p is 6.

In certain embodiments R² is phenyl.

In certain embodiments R² is phenyl substituted with 1 substituent selected from R⁷.

In certain embodiments R² is phenyl substituted with 2 substituents selected from R⁷.

In certain embodiments R² is phenyl substituted with 3 substituents selected from R⁷.

In certain embodiments R² is phenyl substituted with 4 substituents selected from R⁷.

In certain embodiments R² is phenyl substituted with 1 substituent selected from R^{7EWG}In certain embodiments R² is phenyl substituted with 2 substituents selected from R^{7EWG}

In certain embodiments R² is phenyl substituted with 3 substituents selected from R^{7EWG}.

In certain embodiments R² is phenyl substituted with 4 substituents selected from R^{7EWG}.

R^{7EWG} is independently selected at each instance from halogen, haloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, with each haloalkyl, heterocycle, aryl, and heteroaryl optionally substituted as allowed by valence with 1, 2, 3, or 4 substituents selected from R¹⁷.

In certain embodiments R³ is phenyl substituted with 1 substituent selected from R⁷.

In certain embodiments R³ is phenyl substituted with 2 substituents selected from R⁷.

In certain embodiments R³ is phenyl substituted with 3 substituents selected from R⁷.

In certain embodiments R³ is phenyl substituted with 4 substituents selected from R⁷.

In certain embodiments R² is heteroaryl.

In certain embodiments R² is heteroaryl substituted with 1 substituent selected from R⁷.

In certain embodiments R² is heteroaryl substituted with 2 substituents selected from R⁷.

In certain embodiments R² is heteroaryl substituted with 3 substituents selected from R⁷.

In certain embodiments R² is heteroaryl substituted with 4 substituents selected from R⁷.

### Embodiments of R⁴

In certain embodiments R⁴ is

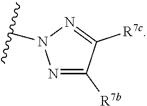

In certain embodiments R⁴ is

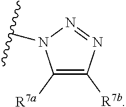

In certain embodiments R⁴ is

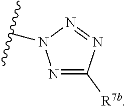

In certain embodiments R⁴ is

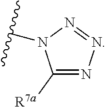

In certain embodiments R⁴ is

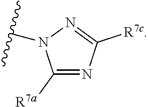

In certain embodiments R⁴ is

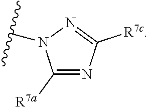

In certain embodiments R⁴ is

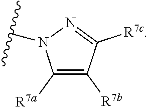

In certain embodiments R⁴ is

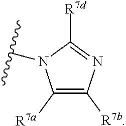

In certain embodiments R⁴ is a 5-membered heteroaryl.

In certain embodiments R⁴ is a fused bicyclic heteroaryl.

In certain embodiments each R⁴ is independently selected from R^{7a}, R^{7b}, R^{7c} and R^{7d}.

In certain embodiments R⁴ is a bicyclic heteroaryl optionally substituted with 1, 2, 3, or 4 R⁷ substituents.

### Embodiments of R⁵

In certain embodiments R⁵ is selected from

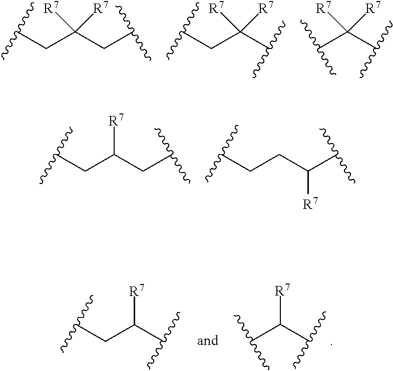

In certain embodiments R⁵ is selected from:

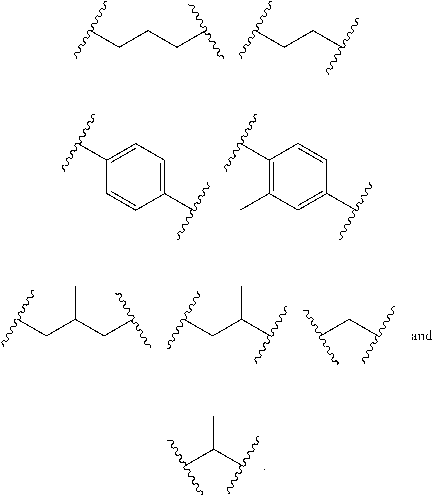

In certain embodiments R⁵ is bond.

In certain embodiments R⁵ and R³ are both bond.

In certain embodiments one of R⁵ and R³ is bond and the other is selected from alkyl, alkenyl, haloalkyl, cycloalkyl, heterocycle, naphthyl, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl, heteroaryl-C(O)—NR⁶—, and heteroaryl, each of which except bond is optionally substituted as allowed by valence with 1, 2, 3, or 4 substituents selected from R⁷.

### Embodiments of R⁶

In certain embodiments one R⁶ is hydrogen.

In certain embodiments one R⁶ is alkyl.

In certain embodiments one R⁶ is haloalkyl.

In certain embodiments one R⁶ is cycloalkyl.

In certain embodiments one R⁶ is aryl.

In certain embodiments one R⁶ is heterocycle.

In certain embodiments one R⁶ is heteroaryl.

### Embodiments of R⁷

In certain embodiments R⁷ is independently selected at each instance from R^{7EWG}.

In certain embodiments R^{7a} is independently selected at each instance from R^{7EWG}.

In certain embodiments R^{7b} is independently selected at each instance from R^{7EWG}.

In certain embodiments R^{7c} is independently selected at each instance from R^{7EWG}.

In certain embodiments R^{7d} is independently selected at each instance from R^{7EWG}.

R^{7EWG} is independently selected at each instance from halogen, haloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, with each haloalkyl, heterocycle, aryl, and heteroaryl optionally substituted as allowed by valence with 1, 2, 3, or 4 substituents selected from R¹⁷.

In certain embodiments R^{7a} is hydrogen.

In certain embodiments R^{7a} is cyano.

In certain embodiments R^{7a} is halogen.

In certain embodiments R^{7a} is fluoro.

In certain embodiments R^{7a} is haloalkyl.

In certain embodiments R^{7a} is —CF₃.

In certain embodiments R^{7a} is aryl optionally substituted as allowed by valence with 1, 2, 3, or 4 substituents selected from R¹⁷.

In certain embodiments R^{7a} is phenyl optionally substituted as allowed by valence with 1, 2, 3, or 4 substituents selected from R¹⁷.

In certain embodiments R^{7a} is aryl.

In certain embodiments R^{7a} is phenyl.

In certain embodiments R^{7b} is hydrogen.

In certain embodiments R^{7b} is cyano.

In certain embodiments R^{7b} is halogen.

In certain embodiments R^{7b} is fluoro.

In certain embodiments R^{7b} is haloalkyl.

In certain embodiments R^{7b} is —CF₃.

In certain embodiments R^{7b} is aryl optionally substituted as allowed by valence with 1, 2, 3, or 4 substituents selected from R¹⁷.

In certain embodiments R^{7b} is phenyl optionally substituted as allowed by valence with 1, 2, 3, or 4 substituents selected from R¹⁷.

In certain embodiments R^{7b} is aryl.

In certain embodiments R^{7b} is phenyl.

In certain embodiments R^{7c} is hydrogen.

In certain embodiments R^{7c} is cyano.

In certain embodiments R^{7c} is halogen.

In certain embodiments R^{7c} is fluoro.

In certain embodiments R^{7c} is haloalkyl.

In certain embodiments R^{7c} is —CF₃.

In certain embodiments R^{7c} is aryl optionally substituted as allowed by valence with 1, 2, 3, or 4 substituents selected from R¹⁷.

In certain embodiments R^{7c} is phenyl optionally substituted as allowed by valence with 1, 2, 3, or 4 substituents selected from R¹⁷.

In certain embodiments R^{7c} is aryl.

In certain embodiments R^{7c} is phenyl.

In certain embodiments R^{7d} is hydrogen.

In certain embodiments R^{7d} is cyano.

In certain embodiments R^{7d} is halogen.

In certain embodiments R^{7d} is fluoro.

In certain embodiments R^{7d} is haloalkyl.

In certain embodiments R^{7d} is —CF₃.

In certain embodiments R^{7d} is aryl optionally substituted as allowed by valence with 1, 2, 3, or 4 substituents selected from R¹⁷.

In certain embodiments R^{7d} is phenyl optionally substituted as allowed by valence with 1, 2, 3, or 4 substituents selected from R¹⁷.

In certain embodiments R^{7d} is aryl.

In certain embodiments R^{7d} is phenyl.

### Embodiments of R^{8a}, R^{8b}, R^{8c}, and R^{8d}

In certain embodiments R^{8a} is R¹².

In certain embodiments R^{8b} is R¹².

In certain embodiments R^{8c} is R¹².

In certain embodiments R^{8d} is R¹².

In certain embodiments R^{8a} is R¹² and R^{8b}, R^{8c}, and R^{8d} are hydrogen.

In certain embodiments R^{8b} is R¹² and R^{8c}, R^{8d}, and R^{8a} are hydrogen.

In certain embodiments R^{8c} is R¹² and R^{8b}, R^{8d}, and R^{8a} are hydrogen.

In certain embodiments R^{8d} is R¹² and R^{8b}, R^{8c}, and R^{8a} are hydrogen.

In certain embodiments R^{8a} is cyano.

In certain embodiments R^{8a} is halogen.

In certain embodiments R^{8a} is fluoro.

In certain embodiments R^{8a} is haloalkyl.

In certain embodiments R^{8a} is —CF₃.

In certain embodiments R^{8a} is aryl optionally substituted as allowed by valence with 1, 2, 3, or 4 substituents selected from R¹⁷.

In certain embodiments R^{8a} is phenyl optionally substituted as allowed by valence with 1, 2, 3, or 4 substituents selected from R¹⁷.

In certain embodiments R^{8a} is aryl.

In certain embodiments R^{8a} is phenyl.

In certain embodiments R^{8a} is OR⁶.

In certain embodiments R^{8a} is N(R⁶)₂.

In certain embodiments R^{8b} is cyano.

In certain embodiments R^{8b} is halogen.

In certain embodiments R^{8b} is fluoro.

In certain embodiments R^{8b} is haloalkyl.

In certain embodiments R^{8b} is —CF₃.

In certain embodiments R^{8b} is aryl optionally substituted as allowed by valence with 1, 2, 3, or 4 substituents selected from R¹⁷.

In certain embodiments R^{8b} is phenyl optionally substituted as allowed by valence with 1, 2, 3, or 4 substituents selected from R¹⁷.

In certain embodiments R^{8b} is aryl.

In certain embodiments R^{8b} is phenyl.

In certain embodiments R^{8b} is OR⁶.

In certain embodiments R^{8b} is N(R⁶)₂.

In certain embodiments R^{8c} is cyano.

In certain embodiments R^{8c} is halogen.

In certain embodiments R^{8c} is fluoro.

In certain embodiments R^{8c} is haloalkyl.

In certain embodiments R^{8c} is —CF₃.

In certain embodiments R^{8c} is aryl optionally substituted as allowed by valence with 1, 2, 3, or 4 substituents selected from R¹⁷.

In certain embodiments R^{8c} is phenyl optionally substituted as allowed by valence with 1, 2, 3, or 4 substituents selected from R¹⁷.

In certain embodiments R^{8c} is aryl.

In certain embodiments R^{8c} is phenyl.

In certain embodiments R^{8c} is OR⁶.

In certain embodiments R^{8c} is N(R⁶)₂.

In certain embodiments R^{8d} is cyano.

In certain embodiments R^{8d} is halogen.

In certain embodiments R^{8d} is fluoro.

In certain embodiments R^{8d} is haloalkyl.

In certain embodiments R^{8d} is —CF₃.

In certain embodiments R^{8d} is aryl optionally substituted as allowed by valence with 1, 2, 3, or 4 substituents selected from R¹⁷.

In certain embodiments R^{8d} is phenyl optionally substituted as allowed by valence with 1, 2, 3, or 4 substituents selected from R¹⁷.

In certain embodiments R^{8d} is aryl.

In certain embodiments R^{8d} is phenyl.

In certain embodiments R^{8d} is OR⁶.

In certain embodiments R^{8d} is N(R⁶)₂.

### Embodiments of R⁹

In certain embodiments —R²—R⁹— and —R⁹—R²— are selected from:

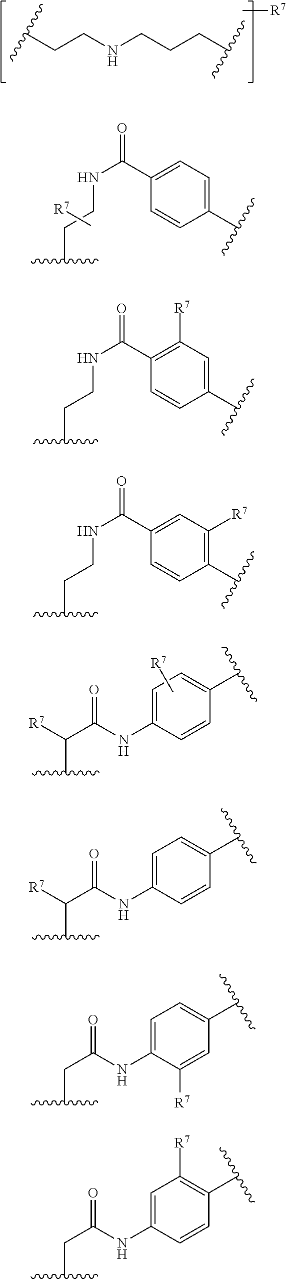

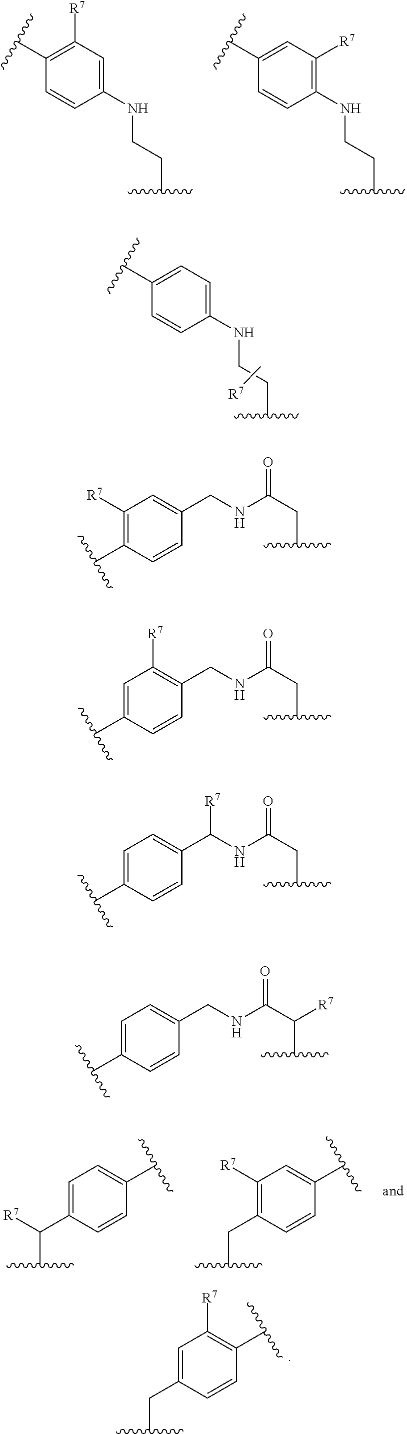

In certain embodiments —R²—R⁹— and —R⁹—R²— are selected from:

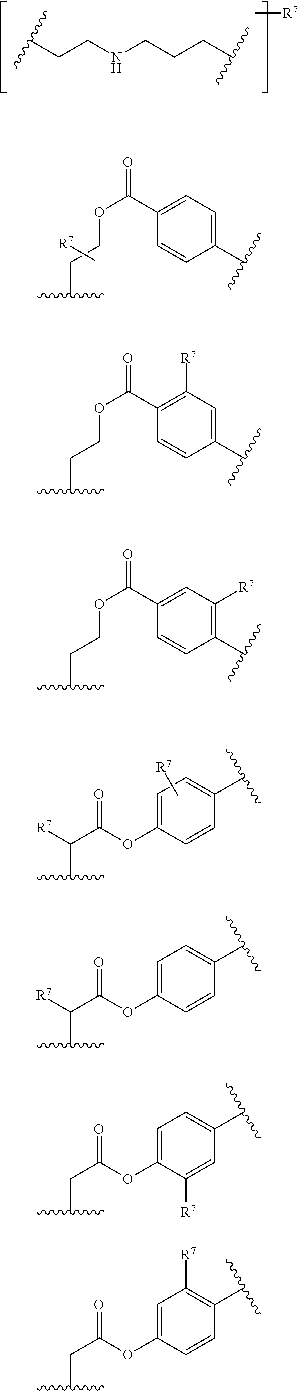

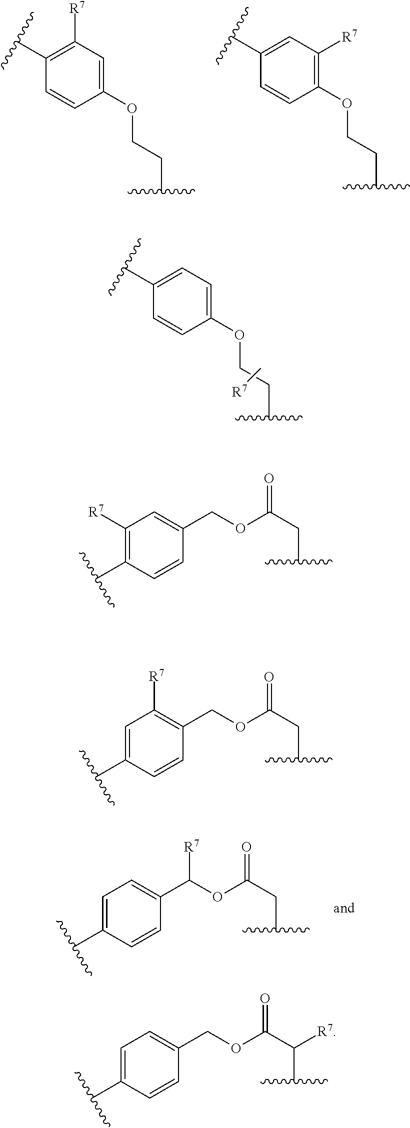

In certain embodiments —R⁹—R³— and —R⁹—R²— are selected from:

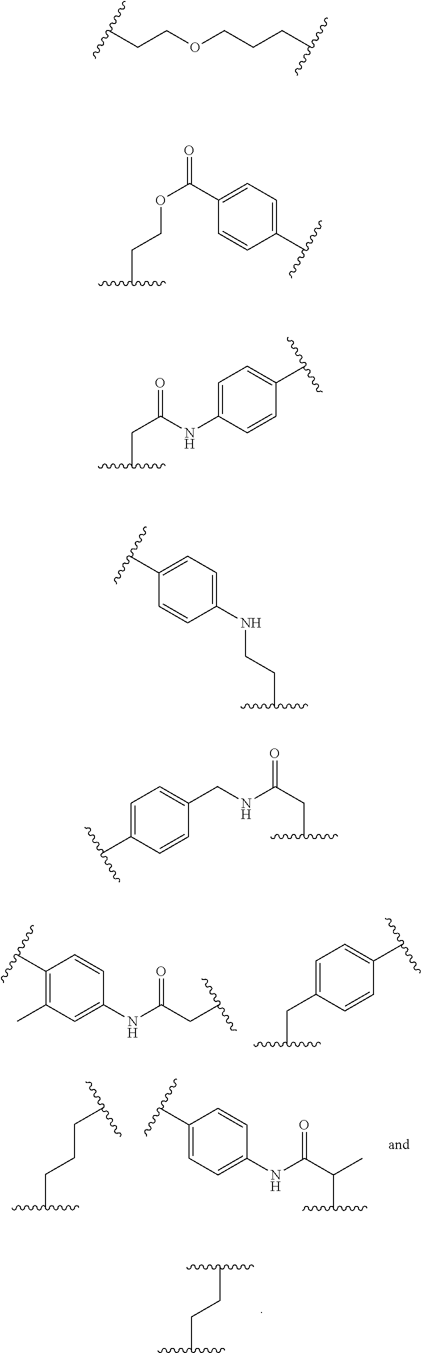

In certain embodiments R⁹ is selected from

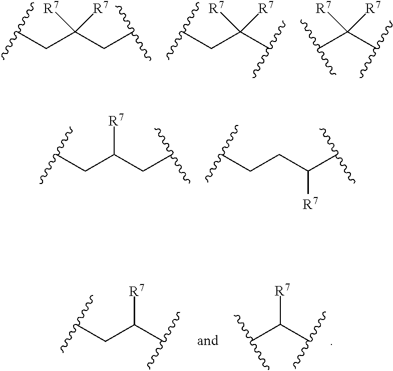

In certain embodiments R⁹ is selected from:

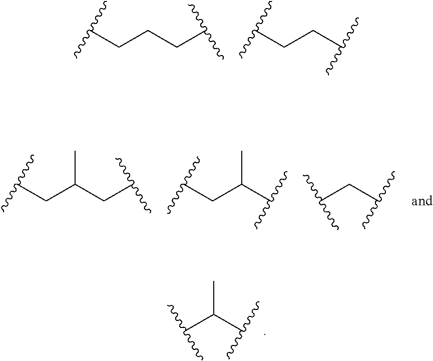

In certain embodiments R⁹ is selected from:

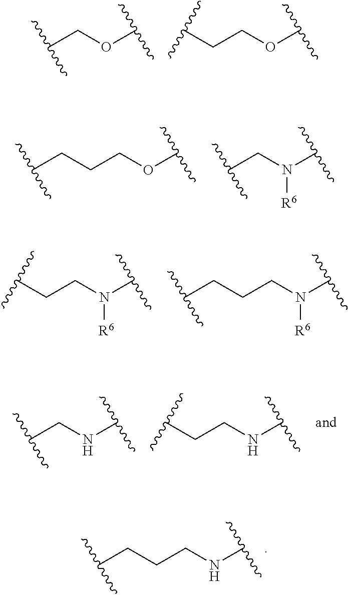

In certain embodiments R⁹ is selected from:

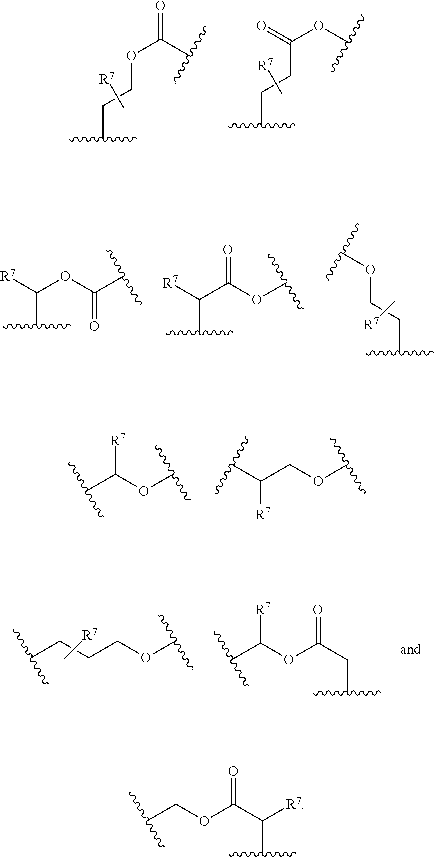

In certain embodiments R⁹ is selected from:

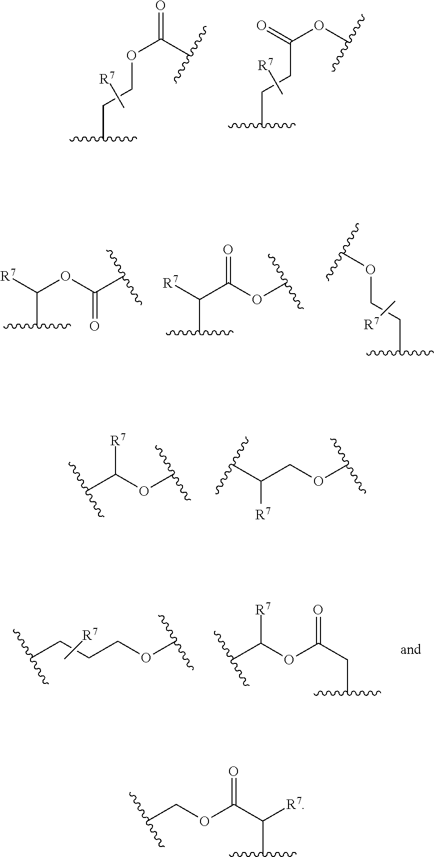

In certain embodiments R² is bond and R⁹ is selected from alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, and heteroaryl, each of which is optionally substituted as allowed by valence with 1, 2, 3, or 4 substituents selected from R⁷.

### Embodiments of R¹¹

In certain embodiments R¹¹ is hydrogen.

In certain embodiments R¹¹ is cyano.

In certain embodiments R¹¹ is halogen.

In certain embodiments R¹¹ is fluoro.

In certain embodiments R¹¹ is haloalkyl.

In certain embodiments R¹¹ is —CF₃.

In certain embodiments R¹¹ is naphthyl optionally substituted as allowed by valence with 1, 2, 3, or 4 substituents selected from R¹⁷.

In certain embodiments R¹¹ is naphthyl.

### Embodiments of R¹²

In certain embodiments R¹² is cyano.

In certain embodiments R¹² is halogen.

In certain embodiments R¹² is fluoro.

In certain embodiments R¹² is haloalkyl.

In certain embodiments R¹² is —CF₃.

In certain embodiments R¹² is aryl optionally substituted as allowed by valence with 1, 2, 3, or 4 substituents selected from R¹⁷.

In certain embodiments R¹² is aryl.

In certain embodiments R¹² is phenyl optionally substituted as allowed by valence with 1, 2, 3, or 4 substituents selected from R¹⁷.

In certain embodiments R¹² is phenyl.

### Embodiments of R³

In certain embodiments R¹³ is cycloalkyl.

In certain embodiments R¹³ is cyclopropyl.

In certain embodiments R¹³ is aryl optionally substituted as allowed by valence with 1, 2, 3, or 4 substituents selected from R⁷.

In certain embodiments R¹³ is phenyl optionally substituted as allowed by valence with 1, 2, 3, or 4 substituents selected from R⁷.

In certain embodiments R¹³ is heteroaryl optionally substituted as allowed by valence with 1, 2, 3, or 4 substituents selected from R⁷.

In certain embodiments R¹³ is aryl.

In certain embodiments R¹³ is phenyl.

In certain embodiments R¹³ is heteroaryl.

In certain embodiments R¹³ is haloalkyl.

### Embodiments of R¹⁵

In certain embodiments R¹⁵ is selected from

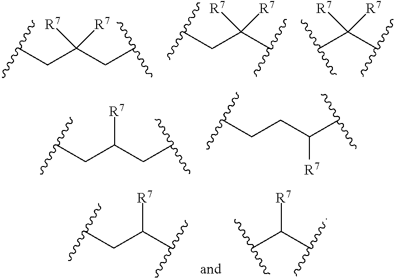

In certain embodiments R¹⁵ is selected from:

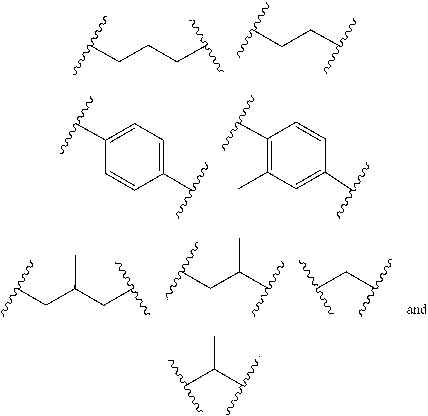

In certain embodiments R¹⁵ is selected from:

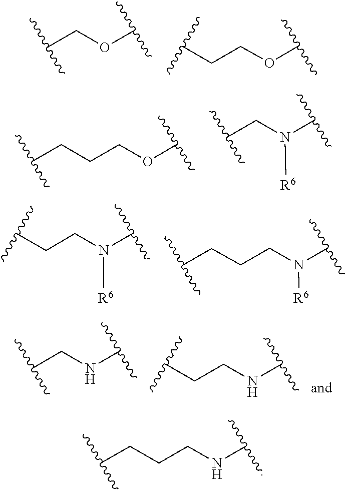

In certain embodiments R¹⁵ is selected from:

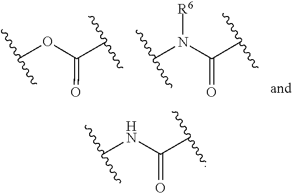

In certain embodiments R¹⁵ is selected from:

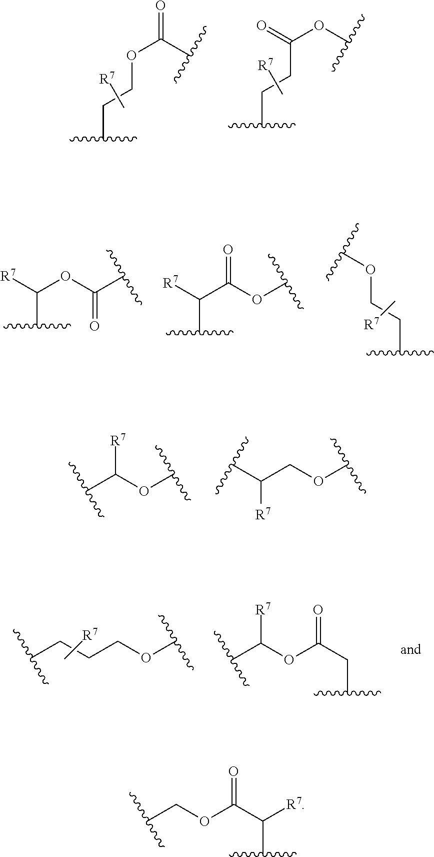

In certain embodiments R¹⁵ is selected from:

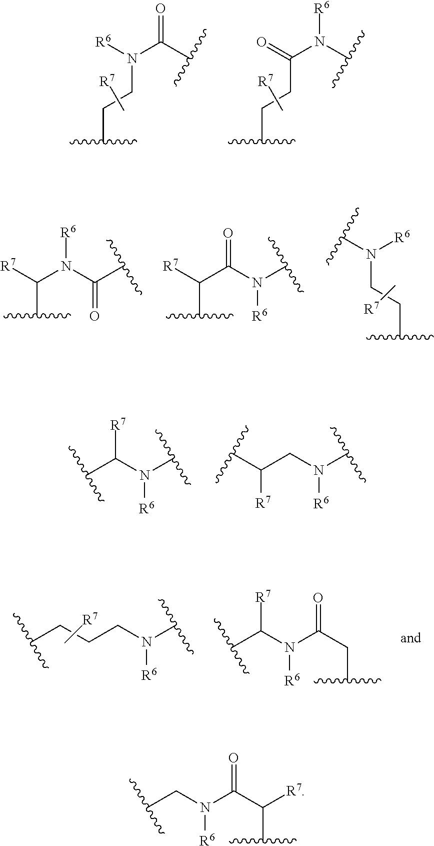

In certain embodiments R¹⁵ is selected from:

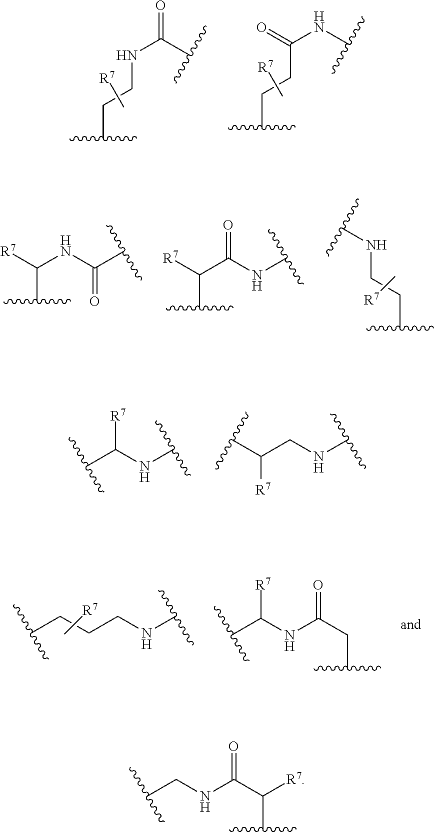

In certain embodiments R¹⁵ is phenyl.

In certain embodiments R¹⁵ is phenyl substituted with 1 substituent selected from R⁷.

In certain embodiments R¹⁵ is phenyl substituted with 2 substituents selected from R⁷.

In certain embodiments R¹⁵ is phenyl substituted with 3 substituents selected from R⁷.

In certain embodiments R¹⁵ is phenyl substituted with 4 substituents selected from R⁷.

In certain embodiments R¹⁵ is heteroaryl.

In certain embodiments R¹⁵ is heteroaryl substituted with 1 substituent selected from R⁷.

In certain embodiments R¹⁵ is heteroaryl substituted with 2 substituents selected from R⁷.

In certain embodiments R¹⁵ is heteroaryl substituted with 3 substituents selected from R⁷.

In certain embodiments R¹⁵ is heteroaryl substituted with 4 substituents selected from R⁷.

### Embodiments of R¹⁶

In certain embodiments R¹⁶ is

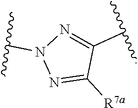

In certain embodiments R¹⁶ is

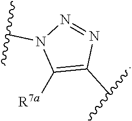

In certain embodiments R¹⁶ is

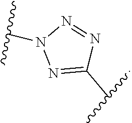

In certain embodiments R¹⁶ is

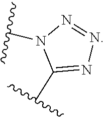 [SMILES: *c1nnnn1*]

In certain embodiments R¹⁶ is

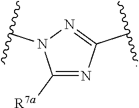

In certain embodiments R¹⁶ is

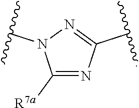

In certain embodiments R¹⁶ is

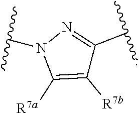

In certain embodiments R¹⁶ is

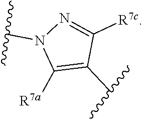

In certain embodiments R¹⁶ is

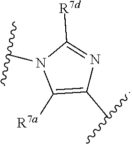

In certain embodiments R¹⁶ is a fused bicyclic heteroaryl.

### Embodiments of R¹⁷

In certain embodiments R¹⁷ is hydrogen.

In certain embodiments R¹⁷ is cyano.

In certain embodiments R¹⁷ is halogen.

In certain embodiments R¹⁷ is fluoro.

In certain embodiments R¹⁷ is haloalkyl.

In certain embodiments R¹⁷ is —CF₃.

In certain embodiments R¹⁷ is aryl.

In certain embodiments R¹⁷ is phenyl.

In certain embodiments one R¹⁷ is hydrogen.

In certain embodiments one R¹⁷ is cyano.

In certain embodiments one R¹⁷ is halogen.

In certain embodiments one R¹⁷ is fluoro.

In certain embodiments one R¹⁷ is haloalkyl.

In certain embodiments one R¹⁷ is —CF₃.

In certain embodiments one R¹⁷ is aryl.

In certain embodiments one R¹⁷ is phenyl.

### Embodiments of R¹⁸

In certain embodiments R¹⁸ is hydrogen.

In certain embodiments R¹⁸ is cyano.

In certain embodiments R¹⁸ is halogen.

In certain embodiments R¹⁸ is fluoro.

In certain embodiments R¹⁸ is haloalkyl.

In certain embodiments R¹⁸ is —CF₃.

In certain embodiments R¹⁸ is aryl.

In certain embodiments R¹⁸ is phenyl.

### Embodiments of R¹⁹

In certain embodiments one R¹⁹ is hydrogen.

In certain embodiments one R¹⁹ is alkyl.

In certain embodiments one R¹⁹ is haloalkyl.

In certain embodiments one R¹⁹ is cycloalkyl.

In certain embodiments one R¹⁹ is aryl.

In certain embodiments one R¹⁹ is heterocycle.

In certain embodiments one R¹⁹ is heteroaryl.

### Embodiments of R²⁷

In certain embodiments R²⁷ is hydrogen.

In certain embodiments R²⁷ is cyano.

In certain embodiments R²⁷ is halogen.

In certain embodiments R²⁷ is fluoro.

In certain embodiments R²⁷ is haloalkyl.

In certain embodiments R²⁷ is —CF₃.

In certain embodiments R²⁷ is aryl.

In certain embodiments R²⁷ is phenyl.

In certain embodiments one R²⁷ is hydrogen.

In certain embodiments one R²⁷ is cyano.

In certain embodiments one R²⁷ is halogen.

In certain embodiments one R²⁷ is fluoro.

In certain embodiments one R²⁷ is haloalkyl.

In certain embodiments one R²⁷ is —CF₃.

In certain embodiments one R²⁷ is aryl.

In certain embodiments one R²⁷ is phenyl.

### Embodiments of “Alkyl”

In one embodiment “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In one embodiment “alkyl” has one carbon.

In one embodiment “alkyl” has two carbons.

In one embodiment “alkyl” has three carbons.

In one embodiment “alkyl” has four carbons.

In one embodiment “alkyl” has five carbons.

In one embodiment “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In an alternative embodiment the “alkyl” group is optionally substituted.

In an alternative embodiment the “alkenyl” group is optionally substituted.

### Embodiments of “Haloalkyl”

In one embodiment “haloalkyl” is a C₁-C₁₀haloalkyl, C₁-C₉haloalkyl, C₁-C₈haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In one embodiment “haloalkyl” has one carbon.

In one embodiment “haloalkyl” has one carbon and one halogen.

In one embodiment “haloalkyl” has one carbon and two halogens.

In one embodiment “haloalkyl” has one carbon and three halogens.

In one embodiment “haloalkyl” has two carbons.

In one embodiment “haloalkyl” has three carbons.

In one embodiment “haloalkyl” has four carbons.

In one embodiment “haloalkyl” has five carbons.

In one embodiment “haloalkyl” has six carbons.

Non-limiting examples of “haloalkyl” include:

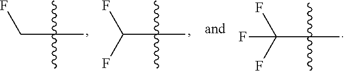

Additional non-limiting examples of “haloalkyl” include:

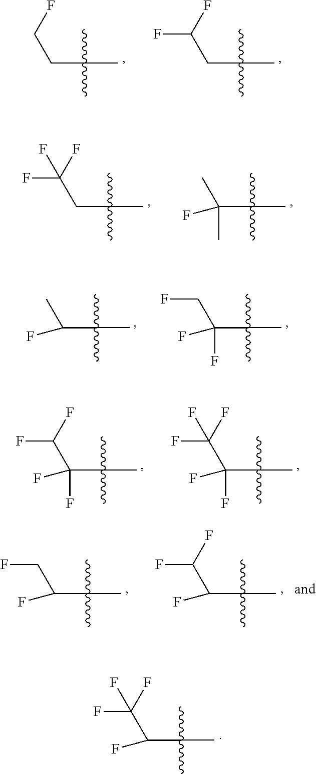

Additional non-limiting examples of “haloalkyl” include:

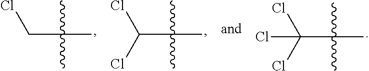

Additional non-limiting examples of “haloalkyl” include:

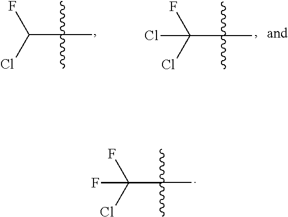

### Embodiments of “Heteroaryl”

Non-limiting examples of 5 membered “heteroaryl” groups include pyrrole, furan, thiophene, pyrazole, imidazole, triazole, isoxazole, oxazole, oxadiazole, oxatriazole, isothiazole, thiazole, thiadiazole, and thiatriazole.

Additional non-limiting examples of 5 membered “heteroaryl” groups include:

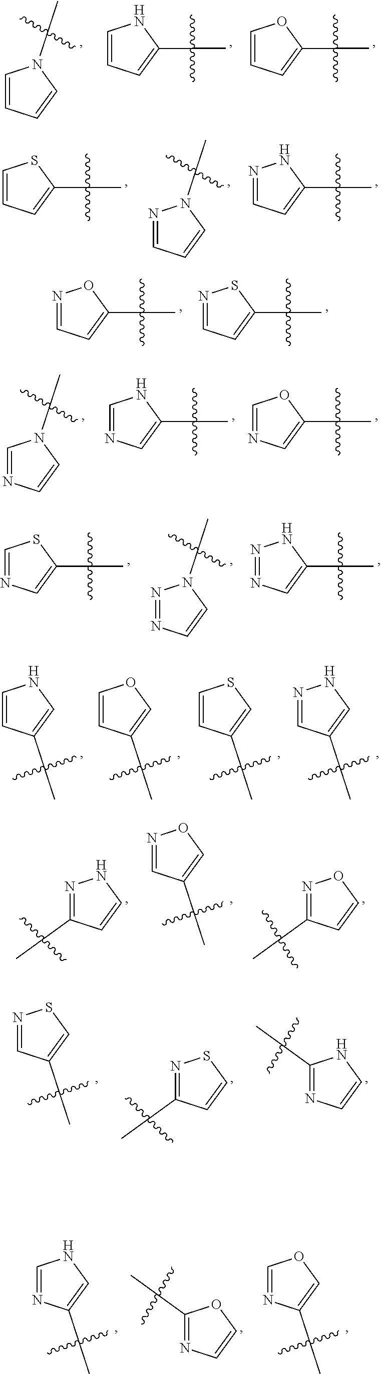

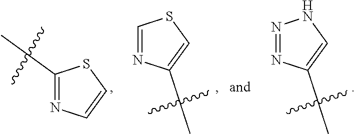

In one embodiment “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

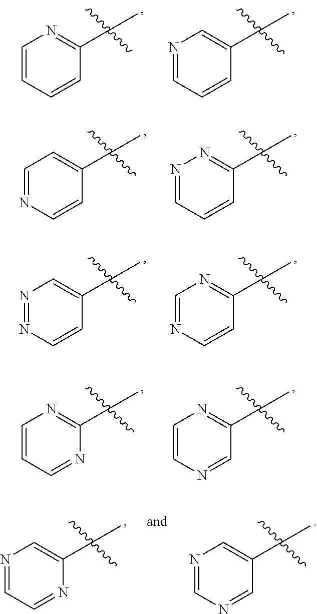

In one embodiment “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

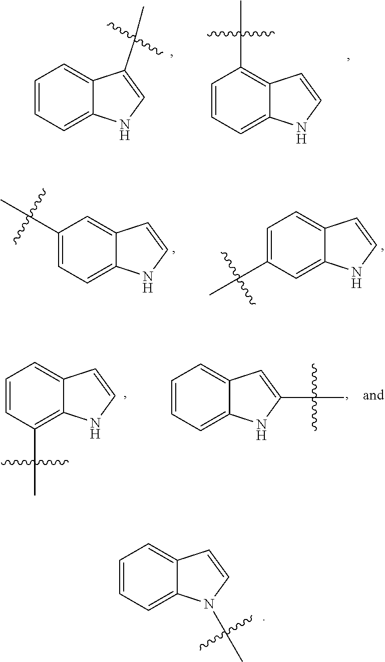

Additional non-limiting examples of “heteroaryl” groups that are bicyclic include:

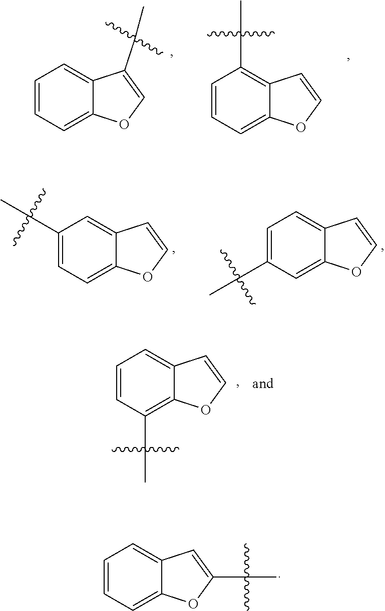

Additional non-limiting examples of “heteroaryl” groups that are bicyclic include:

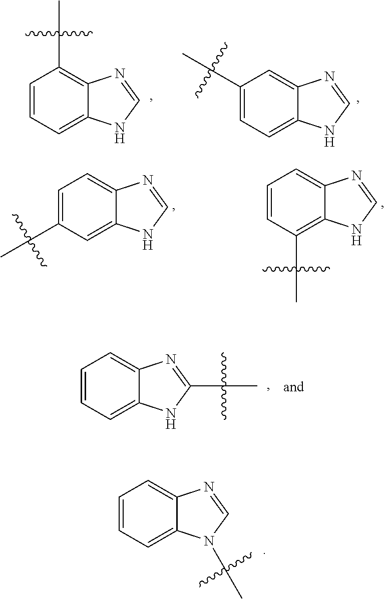

In one embodiment “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

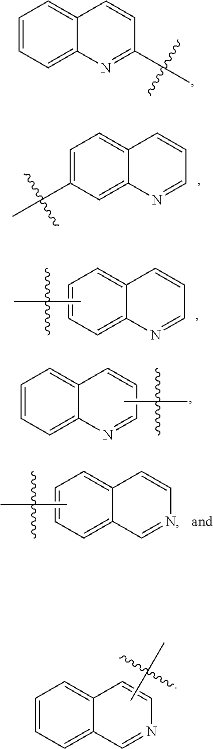

### Embodiments of “Heterocycle”

In one embodiment “heterocycle” refers to a cyclic ring with one nitrogen and 3, 4, 5, 6, 7, or 8 carbon atoms.

In one embodiment “heterocycle” refers to a cyclic ring with one nitrogen and one oxygen and 3, 4, 5, 6, 7, or 8 carbon atoms.

In one embodiment “heterocycle” refers to a cyclic ring with two nitrogens and 3, 4, 5, 6, 7, or 8 carbon atoms.

In one embodiment “heterocycle” refers to a cyclic ring with one oxygen and 3, 4, 5, 6, 7, or 8 carbon atoms.

In one embodiment “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

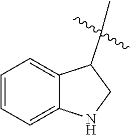 [SMILES: *C1CNc2ccccc21]

is a “heterocycle” group.

However,

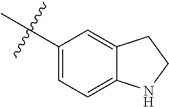 [SMILES: *c1ccc2c(c1)CCN2]

is an “aryl” group.

Non-limiting examples of “heterocycle” also include:

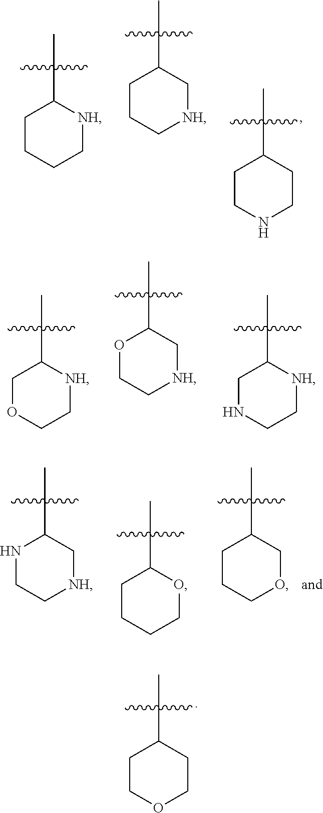

Additional non-limiting examples of “heterocycle” include:

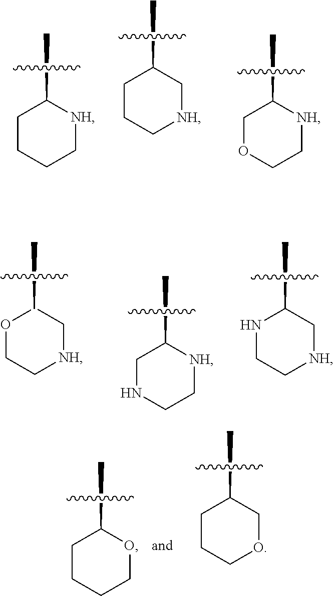

Additional non-limiting examples of “heterocycle” include:

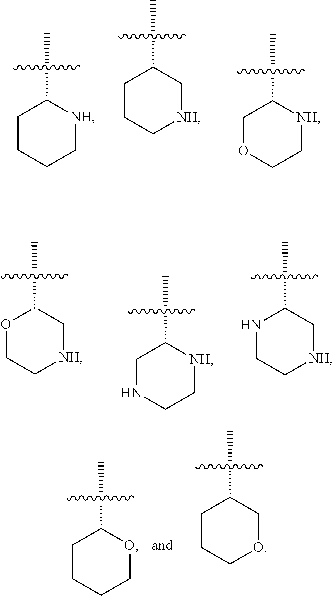

Non-limiting examples of “heterocycle” also include:

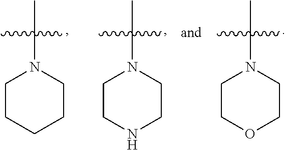

Non-limiting examples of “heterocycle” also include:

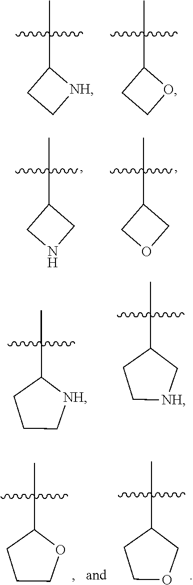

Additional non-limiting examples of “heterocycle” include:

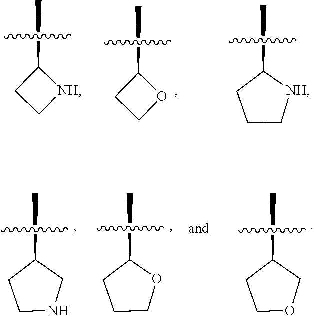

Additional non-limiting examples of “heterocycle” include:

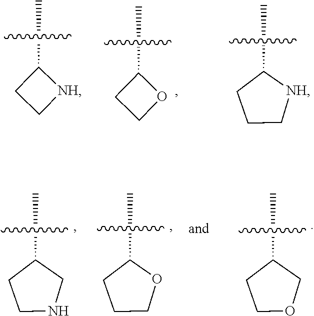

### Embodiments of “Aryl”

In one embodiment “aryl” is a 6 carbon aromatic group (phenyl).

In one embodiment “aryl” is a 10 carbon aromatic group (naphthyl).

In one embodiment “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example

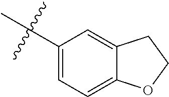 [SMILES: *c1ccc2c(c1)CCO2]

is an “aryl” group.

However,

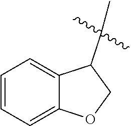 [SMILES: *C1COc2ccccc21]

is a “heterocycle” group.

1. In one embodiment a compound selected from Formula I, Formula II, Formula III, Formula IV Formula V, Formula VI, Formula VII, and Formula VIII is provided:

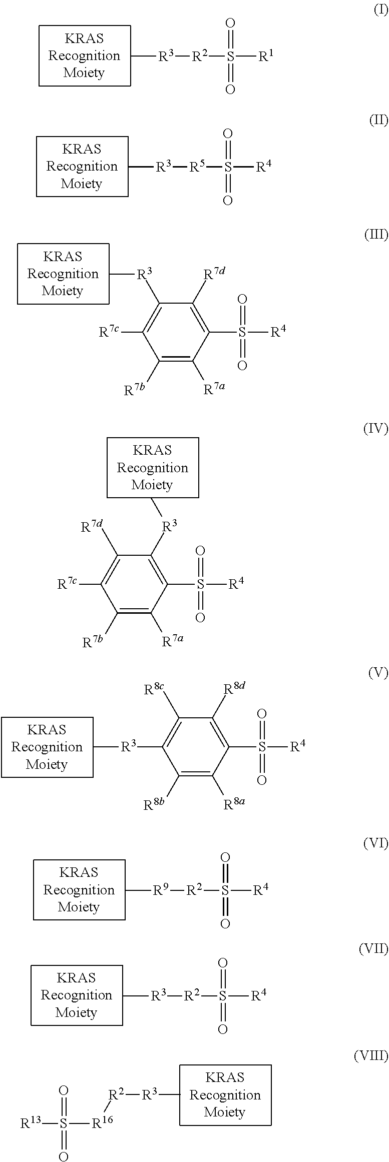

or a pharmaceutically acceptable salt, N-oxide, isotopic derivative, or prodrug thereof; wherein:

R¹ is selected from:

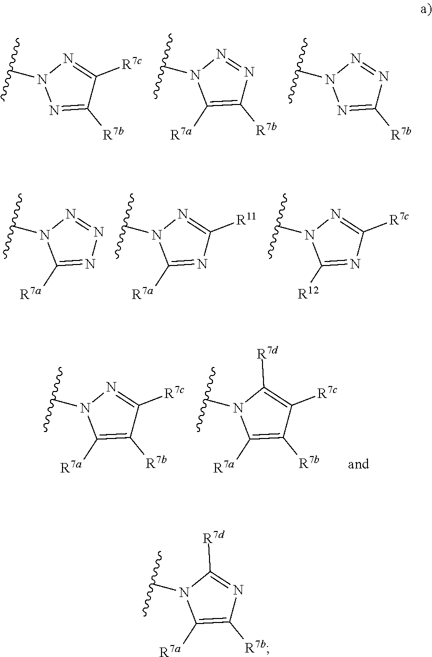

b) a bicyclic heteroaryl or tricyclic heteroaryl, each of which is optionally substituted as allowed by valence with 1, 2, 3, or 4 substituents selected from R⁷;
   R² is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which except bond is optionally substituted as allowed by valence with 1, 2, 3, or 4 substituents selected from R⁷;
   R³ is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which except bond is optionally substituted as allowed by valence with 1, 2, 3, or 4 substituents selected from R⁷;
   and wherein R² and R³ are selected such that a suitably stable and suitably nontoxic compound for in vivo administration in a host is achieved, and in certain embodiments, in a way that avoids undesired repetition of atoms or moieties, such as in nonlimiting examples, S, O, or a combination thereof (i.e., that would otherwise form a disulfide or peroxide bond), as well known to skilled artisans;
   p is independently selected from 1, 2, 3, 4, 5, and 6;
   R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;
   R⁵ is selected from alkyl, alkenyl, haloalkyl, cycloalkyl, naphthyl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;
   and wherein R³ and R⁵ are selected such that a suitably stable and suitably nontoxic compound for in vivo administration in a host is achieved, and in certain embodiments, in a way that avoids undesired repetition of atoms or moieties, such as in nonlimiting examples, S, O, or a combination thereof (i.e., that would otherwise form a disulfide or peroxide bond), as well known to skilled artisans;
   R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;
   R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
   R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶;
   R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;
   R¹⁹ is independently selected at each instance from hydrogen, alkyl, haloalkyl, cycloalkyl, aryl, heterocycle, and heteroaryl;
   R^{8a}, R^{8b}, R^{8c}, and R^{8d} are independently selected at each instance from R⁷ and R¹² wherein at least one of R^{8a}, R^{8b}, R^{8c}, and R^{8d} is R¹²;
   R⁹ is selected from alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;
   R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R¹⁷; and
   R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷; and
   R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;
   R¹⁶ is a heteroaryl group, where the bond to the sulfur atom is through one of the nitrogen atoms present in the cycle, and R¹⁶ is optionally substituted as allowed by valence with 1, 2, 3, or 4 substituents selected from R⁷;
   KRAS Recognition Moiety is a molecule which can bind to or otherwise anchors too KRAS.

2. The compound of embodiment 1, wherein the compound is of Formula:

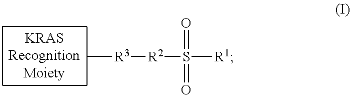

or a pharmaceutically acceptable salt thereof.

3. The compound of embodiment 1, wherein the compound is of Formula:

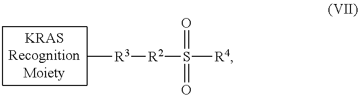

or a pharmaceutically acceptable salt thereof.

4. The compound of embodiment 1, wherein the compound is of Formula:

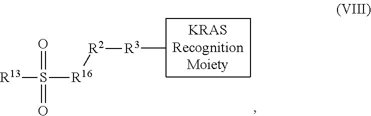

or a pharmaceutically acceptable salt thereof.

5. The compound of embodiment 4, wherein R¹³ is phenyl optionally substituted as allowed by valence with 1, 2, 3, or 4 substituents selected from R⁷.

6. The compound of embodiment 4, wherein R¹³ is alkyl optionally substituted as allowed by valence with 1, 2, 3, or 4 substituents selected from R⁷.

7. The compound of embodiment 4, wherein R¹³ is cycloalkyl optionally substituted as allowed by valence with 1, 2, 3, or 4 substituents selected from R⁷.

8. The compound of embodiment 4, wherein R¹³ is aryl optionally substituted as allowed by valence with 1, 2, 3, or 4 substituents selected from R⁷.

9. The compound of embodiment 4, wherein R¹³ is cyclopropyl optionally substituted as allowed by valence with 1, 2, 3, or 4 substituents selected from R⁷.

10. The compound of embodiment 4, wherein R¹³ is heterocycle optionally substituted as allowed by valence with 1, 2, 3, or 4 substituents selected from R⁷.

11. The compound of embodiment 4, wherein R¹³ is haloalkyl optionally substituted as allowed by valence with 1, 2, 3, or 4 substituents selected from R⁷.

12. The compound of any one of embodiments 5-11, wherein R¹⁶ is a triazole.

13. The compound of any one of embodiments 5-12, wherein R¹⁶ is

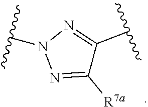

14. The compound of any one of embodiments 5-12, wherein R¹⁶ is

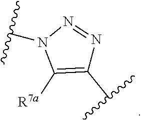

15. The compound of any one of embodiments 5-11, wherein R¹⁶ is

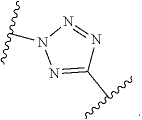 [SMILES: *c1nnn(*)n1]

16. The compound of any one of embodiments 5-11, wherein R¹⁶ is

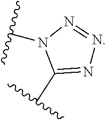 [SMILES: *c1nnnn1*]

17. The compound of any one of embodiments 5-12, wherein R¹⁶ is

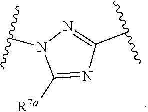

18. The compound of any one of embodiments 5-11, wherein R¹⁶ is

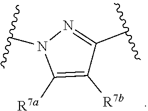

19. The compound of any one of embodiments 5-11, wherein R¹⁶ is

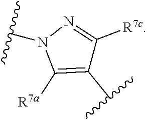

20. The compound of any one of embodiments 5-11, wherein R¹⁶ is

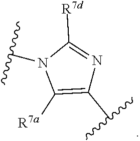

21. The compound of embodiment 1, wherein the compound is of Formula:

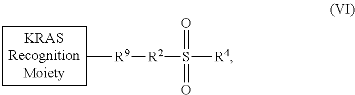

or a pharmaceutically acceptable salt thereof.

22. The compound of embodiment 21, wherein R⁹ is selected alkyl, alkenyl, haloalkyl, cycloalkyl, bicycle, tricycle, and heteroaryl, each of which except bond is optionally substituted as allowed by valence with 1, 2, 3, or 4 substituents selected from R⁷.

23. The compound of embodiment 22, wherein R⁹ is alkyl optionally substituted as allowed by valence with 1, 2, 3, or 4 substituents selected from R⁷.

24. The compound of embodiment 22, wherein R⁹ is alkenyl optionally substituted as allowed by valence with 1, 2, 3, or 4 substituents selected from R⁷.

25. The compound of embodiment 22, wherein R⁹ is haloalkyl optionally substituted as allowed by valence with 1, 2, 3, or 4 substituents selected from R⁷.

26. The compound of embodiment 22, wherein R⁹ is cycloalkyl optionally substituted as allowed by valence with 1, 2, 3, or 4 substituents selected from R⁷.

27. The compound of embodiment 22, wherein R⁹ is heteroaryl optionally substituted as allowed by valence with 1, 2, 3, or 4 substituents selected from R⁷.

28. The compound of embodiment 22, wherein R⁹ is bicycle optionally substituted as allowed by valence with 1, 2, 3, or 4 substituents selected from R⁷.

29. The compound of any one of embodiments 1-20, wherein R³ is bond.

30. The compound of any one of embodiments 1-20, wherein R³ is phenyl optionally substituted as allowed by valence with 1, 2, 3, or 4 substituents selected from R⁷.

31. The compound of any one of embodiments 1-20, wherein R³ is alkyl optionally substituted as allowed by valence with 1, 2, 3, or 4 substituents selected from R⁷.

32. The compound of any one of embodiments 1-20, wherein R³ is alkenyl optionally substituted as allowed by valence with 1, 2, 3, or 4 substituents selected from R⁷.

33. The compound of any one of embodiments 1-20, wherein R³ is haloalkyl optionally substituted as allowed by valence with 1, 2, 3, or 4 substituents selected from R⁷.

34. The compound of any one of embodiments 1-20, wherein R³ is cycloalkyl optionally substituted as allowed by valence with 1, 2, 3, or 4 substituents selected from R⁷.

35. The compound of any one of embodiments 1-20, wherein R³ is aryl optionally substituted as allowed by valence with 1, 2, 3, or 4 substituents selected from R⁷.

36. The compound of any one of embodiments 1-20, wherein R³ is heteroaryl optionally substituted as allowed by valence with 1, 2, 3, or 4 substituents selected from R⁷.

37. The compound of any one of embodiments 1-20, wherein R³ is bicycle optionally substituted as allowed by valence with 1, 2, 3, or 4 substituents selected from R⁷.

38. The compound of any one of embodiments 1-37, wherein R² is selected from bond, alkyl, alkenyl, haloalkyl, cycloalkyl, aryl, bicycle, tricycle, and heteroaryl, each of which except bond is optionally substituted as allowed by valence with 1, 2, 3, or 4 substituents selected from R⁷.

39. The compound of any one of embodiments 1-38, wherein R² is bond.

40. The compound of any one of embodiments 1-38, wherein R² is phenyl optionally substituted as allowed by valence with 1, 2, 3, or 4 substituents selected from R⁷.

41. The compound of any one of embodiments 1-38, wherein R² is alkyl optionally substituted as allowed by valence with 1, 2, 3, or 4 substituents selected from R⁷.

42. The compound of any one of embodiments 1-38, wherein R² is alkenyl optionally substituted as allowed by valence with 1, 2, 3, or 4 substituents selected from R⁷.

43. The compound of any one of embodiments 1-38, wherein R² is haloalkyl optionally substituted as allowed by valence with 1, 2, 3, or 4 substituents selected from R⁷.

44. The compound of any one of embodiments 1-38, wherein R² is cycloalkyl optionally substituted as allowed by valence with 1, 2, 3, or 4 substituents selected from R⁷.

45. The compound of any one of embodiments 1-38, wherein R² is aryl optionally substituted as allowed by valence with 1, 2, 3, or 4 substituents selected from R⁷.

46. The compound of any one of embodiments 1-38, wherein R² is heteroaryl optionally substituted as allowed by valence with 1, 2, 3, or 4 substituents selected from R⁷.

47. The compound of any one of embodiments 1-38, wherein R² is bicycle optionally substituted as allowed by valence with 1, 2, 3, or 4 substituents selected from R⁷.

48. The compound of any one of embodiments 1-47, wherein R² is not substituted.

49. The compound of any one of embodiments 1-47, wherein R² is substituted as allowed by valence with 1 substituent selected from R⁷.

50. The compound of any one of embodiments 1-47, wherein R² is substituted as allowed by valence with 2 substituents selected from R⁷.

51. The compound of any one of embodiments 1-47, wherein R² is substituted as allowed by valence with 3 substituents selected from R⁷.

52. The compound of embodiment 1, wherein the compound is of Formula:

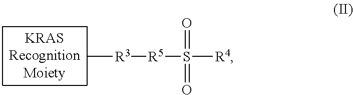

or a pharmaceutically acceptable salt thereof.

53. The compound of embodiment 1 wherein the compound is Formula:

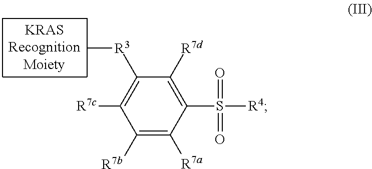

or a pharmaceutically acceptable salt thereof.

54. The compound of embodiment 1 wherein the compound is Formula:

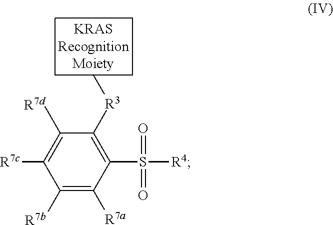

or a pharmaceutically acceptable salt thereof.

55. The compound of embodiment 1 wherein the compound is Formula:

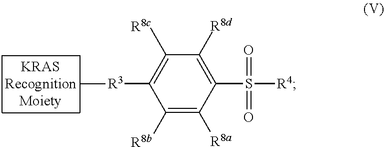

or a pharmaceutically acceptable salt thereof.

56. The compound of any one of embodiments 52-55, wherein R³ is bond.

57. The compound of any one of embodiments 52-55, wherein R³ is phenyl optionally substituted as allowed by valence with 1, 2, 3, or 4 substituents selected from R⁷.

58. The compound of any one of embodiments 52-55, wherein R³ is alkyl optionally substituted as allowed by valence with 1, 2, 3, or 4 substituents selected from R⁷.

59. The compound of any one of embodiments 52-55, wherein R³ is alkenyl optionally substituted as allowed by valence with 1, 2, 3, or 4 substituents selected from R⁷.

60. The compound of any one of embodiments 52-55, wherein R³ is haloalkyl optionally substituted as allowed by valence with 1, 2, 3, or 4 substituents selected from R⁷.

61. The compound of any one of embodiments 52-55, wherein R³ is cycloalkyl optionally substituted as allowed by valence with 1, 2, 3, or 4 substituents selected from R⁷.

62. The compound of any one of embodiments 52-55, wherein R³ is aryl optionally substituted as allowed by valence with 1, 2, 3, or 4 substituents selected from R⁷.

63. The compound of any one of embodiments 52-55, wherein R³ is heteroaryl optionally substituted as allowed by valence with 1, 2, 3, or 4 substituents selected from R⁷.

64. The compound of any one of embodiments 52-55, wherein R³ is bicycle optionally substituted as allowed by valence with 1, 2, 3, or 4 substituents selected from R⁷.

65. The compound of any one of embodiments 1-3, or 21-64, wherein R¹ and R⁴ are

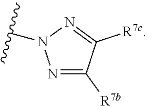

66. The compound of any one of embodiments 1-3, or 21-64, wherein R¹ and R⁴ are

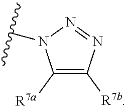

67. The compound of any one of embodiments 1-3, or 21-64, wherein R¹ and R⁴ are

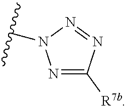

68. The compound of any one of embodiments 1-3, or 21-64, wherein R¹ and R⁴ are

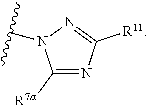

69. The compound of any one of embodiments 1-3, or 21-64, wherein R¹ and R⁴ are

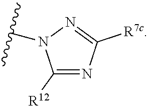

70. The compound of any one of embodiments 1-3, or 21-64, wherein R¹ and R⁴ are

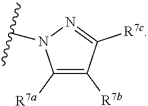

71. The compound of any one of embodiments 1-3, or 21-64, wherein R¹ and R⁴ are

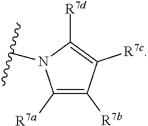

72. The compound of any one of embodiments 1-3, or 21-64, wherein R¹ and R⁴ are

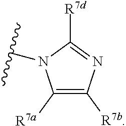

73. The compound of any one of embodiments 1-3, or 21-64, wherein R¹ and R⁴ are a bicyclic heteroaryl which is optionally substituted as allowed by valence with 1, 2, 3, or 4 substituents selected from R⁷.

74. The compound of any one of embodiments 1-3, or 21-64, wherein R⁴ is a heteroaryl group, where the bond to the sulfur atom is through the nitrogen present in the cycle, and each heteroaryl is optionally substituted as allowed by valence with 1, 2, 3, or 4 substituents selected from R⁷.

75. The compound of any one of embodiments 1-74, wherein R⁶ is independently selected at each instance from alkyl, haloalkyl, cycloalkyl, aryl, heterocycle, and heteroaryl; each of which is optionally substituted as allowed by valence with 1, 2, 3, or 4 substituents selected from R¹⁸.

76. The compound of embodiment 75, wherein R⁶ is alkyl optionally substituted as allowed by valence with 1, 2, 3, or 4 substituents selected from R¹⁸.

77. The compound of embodiment 75, wherein R⁶ is haloalkyl optionally substituted as allowed by valence with 1, 2, 3, or 4 substituents selected from R¹⁸.

78. The compound of embodiment 75, wherein R⁶ is cycloalkyl optionally substituted as allowed by valence with 1, 2, 3, or 4 substituents selected from R¹⁸.

79. The compound of embodiment 75, wherein R⁶ is aryl optionally substituted as allowed by valence with 1, 2, 3, or 4 substituents selected from R¹⁸.

80. The compound of embodiment 75, wherein R⁶ is heterocycle optionally substituted as allowed by valence with 1, 2, 3, or 4 substituents selected from R¹⁸.

81. The compound of embodiment 75, wherein R⁶ is heteroaryl optionally substituted as allowed by valence with 1, 2, 3, or 4 substituents selected from R¹⁸.

82. The compound of any one of embodiments 75-81, wherein R⁶ is not substituted with R¹⁸.

83. The compound of any one of embodiments 75-81, wherein R⁶ is substituted with 1 substituent selected from R¹⁸.

84. The compound of any one of embodiments 75-81, wherein R⁶ is substituted with 2 substituents independently selected from R¹⁸.

85. The compound of any one of embodiments 75-81, wherein R⁶ is substituted with 3 substituents independently selected from R¹⁸.

86. The compound of any one of embodiments 1-85, wherein R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, and nitro, wherein each alkyl, haloalkyl, alkenyl, cycloalkyl, heterocycle, aryl, and heteroaryl is optionally substituted as allowed by valence with 1, 2, 3, or 4 substituents selected from R¹⁷

87. The compound of any one of embodiments 1-86, wherein one of R⁷, R^{7a}, R^{7b}, R^{7c}, and R^{7d} is alkyl optionally substituted as allowed by valence with 1, 2, 3, or 4 substituents selected from R¹⁷.

88. The compound of any one of embodiments 1-86, wherein one of R⁷, R^{7a}, R^{7b}, R^{7c}, and R^{7d} is haloalkyl optionally substituted as allowed by valence with 1, 2, 3, or 4 substituents selected from R¹⁷.

89. The compound of any one of embodiments 1-86, wherein one of R⁷, R^{7a}, R^{7b}, R^{7c}, and R^{7d} is phenyl optionally substituted as allowed by valence with 1, 2, 3, or 4 substituents selected from R¹⁷.

90. The compound of any one of embodiments 1-86, wherein one of R⁷, R^{7a}, R^{7b}, R^{7c}, and R^{7d} is heteroaryl optionally substituted as allowed by valence with 1, 2, 3, or 4 substituents selected from R¹⁷.

91. The compound of any one of embodiments 1-86, wherein one of R⁷, R^{7a}, R^{7b}, R^{7c}, and R^{7d} is haloalkyl optionally substituted as allowed by valence with 1, 2, 3, or 4 substituents selected from R¹⁷.

92. The compound of any one of embodiments 1-86, wherein one of R⁷, R^{7a}, R^{7b}, R^{7c}, and R^{7d} is heterocycle optionally substituted as allowed by valence with 1, 2, 3, or 4 substituents selected from R¹⁷.

93. The compound of any one of embodiments 1-86, wherein one of R⁷, R^{7a}, R^{7b}, R^{7c}, and R^{7d} is cycloalkyl optionally substituted as allowed by valence with 1, 2, 3, or 4 substituents selected from R¹⁷.

94. The compound of any one of embodiments 87-93, wherein R⁷, R^{7a}, R^{7b}, R^{7c}, and R^{7d} are not substituted.

95. The compound of any one of embodiments 87-93, wherein R⁷, R^{7a}, R^{7b}, R^{7c}, and R^{7d} are optionally substituted with 1 or 2 substituents selected from R¹⁷.

96. The compound of any one of embodiments 1-86, wherein one of R⁷, R^{7a}, R^{7b}, R^{7c}, and R^{7d} is halogen.

97. The compound of any one of embodiments 1-86, wherein one of R⁷, R^{7a}, R^{7b}, R^{7c}, and R^{7d} is —OR⁶.

98. The compound of any one of embodiments 1-86, wherein one of R⁷, R^{7a}, R^{7b}, R^{7c}, and R^{7d} is ═O.

99. The compound of any one of embodiments 1-86, wherein one of R⁷, R^{7a}, R^{7b}, R^{7c}, and R^{7d} is cyano.

100. The compound of any one of embodiments 1-86, wherein one of R⁷, R^{7a}, R^{7b}, R^{7c}, and R^{7d} is nitro.

101. The compound of any one of embodiments 1-100, wherein R¹⁷ is selected in each instance from halogen, alkyl, haloalkyl, alkenyl, and cyano.

102. In one embodiment a pharmaceutical composition comprising a compound of any one of embodiments 1-101 or a pharmaceutically acceptable salt thereof and a pharmaceutically acceptable excipient is provided.

103. In one embodiment a method of treating a disorder mediated by KRAS comprising administering an effective amount of a compound of any one of embodiments 1-102 or a pharmaceutically acceptable salt thereof to a patient in need thereof is provided.

104. The method of embodiment 103 wherein the patient is a human.

In other embodiments a compound, pharmaceutical composition, or method is provided as described below:

1. A compound of Formula:

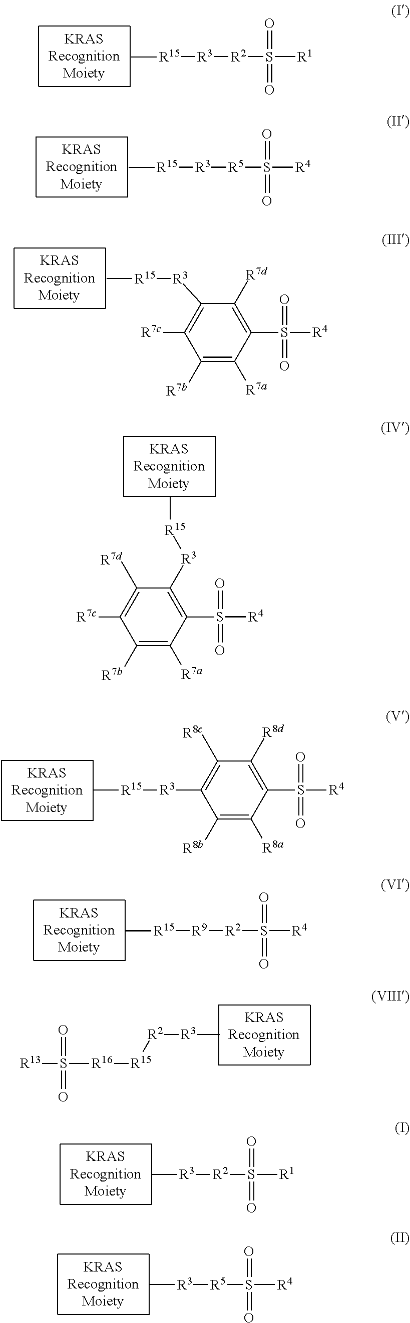

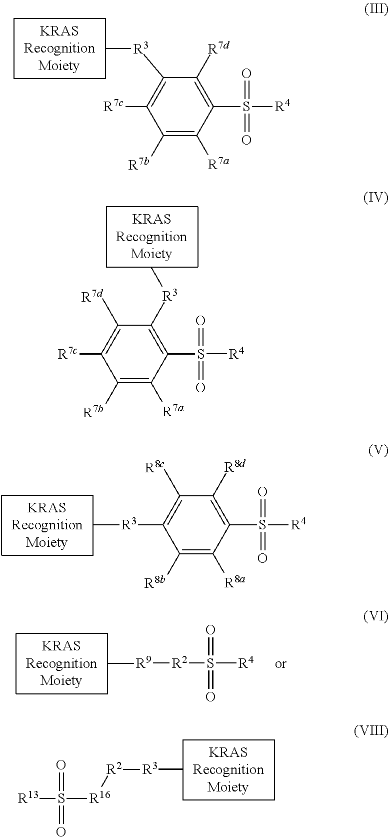

or a pharmaceutically acceptable salt thereof;
wherein:
R¹ is selected from:

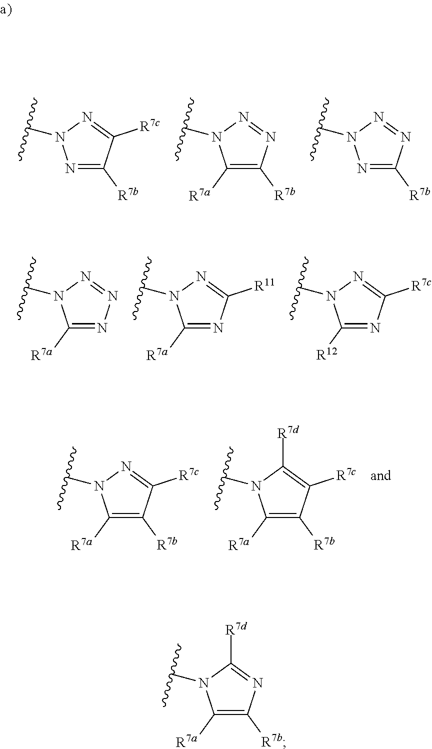

or
b) a bicyclic heteroaryl or tricyclic heteroaryl, optionally substituted as allowed by valence with 1, 2, 3, or 4 substituents selected from R⁷;
R² is a bivalent moiety independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;
R³ is a bivalent moiety independently selected at each instance from bond, alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents selected from R⁷;
and wherein R² and R³ are selected such that a suitably stable and suitably nontoxic compound for in vivo administration in a host is achieved;
each p is independently selected from 1, 2, 3, 4, 5, and 6;
R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;
R⁵ is a bivalent moiety selected from alkyl, alkenyl, haloalkyl, cycloalkyl, naphthyl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;
and wherein R³ and R⁵ are selected such that a suitably stable and suitably nontoxic compound for in vivo administration in a host is achieved;
R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;
R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
R¹⁵ is a bivalent moiety selected from the group consisting of alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents independently selected from R⁷;
R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶;
R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;
R¹⁹ is independently selected at each instance from hydrogen, alkyl, haloalkyl, cycloalkyl, aryl, heterocycle, and heteroaryl;
R^{8a}, R^{8b}, R^{8c}, and R^{8d} are independently selected at each instance from R⁷ and R¹² wherein at least one of R⁸, R^{8b}, R^{8c}, and R^{8d} is R¹²;
R⁹ is a bivalent moiety selected from alkyl, alkenyl, haloalkyl, cycloalkyl, heterocycle, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;
R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷; and
R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;
R¹⁶ is a heteroaryl group, where the bond to the sulfur atom is through one of the nitrogen atoms present in the cycle, and R¹⁶ is optionally substituted as allowed by valence with 1, 2, 3, or 4 substituents selected from R⁷; and
KRAS Recognition Moiety is a molecule which can bind to or otherwise anchors too KRAS.

2. A compound of Formula:

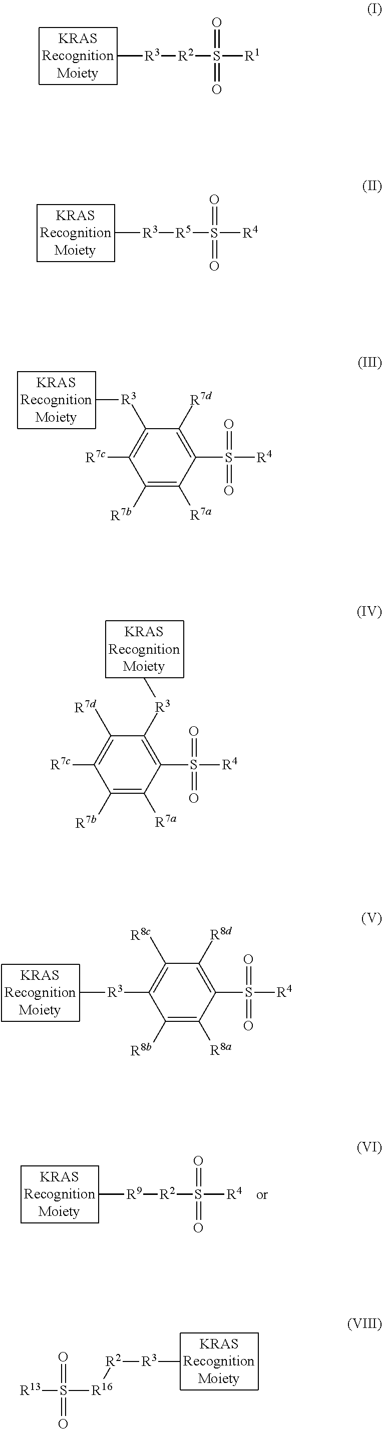

or a pharmaceutically acceptable salt thereof,
wherein:
R¹ is selected from:

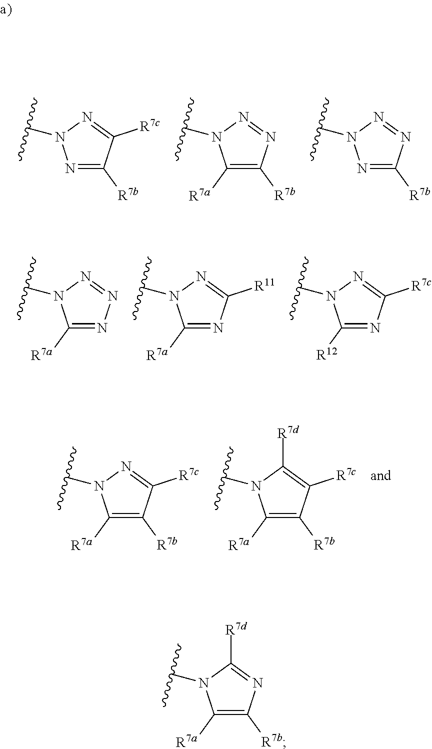

b) a bicyclic heteroaryl or tricyclic heteroaryl, each of which is optionally substituted as allowed by valence with 1, 2, 3, or 4 substituents selected from R⁷;
R² is a bivalent moiety independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;
R³ is a bivalent moiety independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;
and wherein R² and R³ are selected such that a suitably stable and suitably nontoxic compound for in vivo administration in a host is achieved;
p is independently selected from 1, 2, 3, 4, 5, and 6;
R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;
R⁵ is a bivalent moiety selected from alkyl, alkenyl, haloalkyl, cycloalkyl, naphthyl, heterocycle, —S—, —O—, —NR¹—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;
and wherein R³ and R⁵ are selected such that a suitably stable and suitably nontoxic compound for in vivo administration in a host is achieved;
R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;
R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶;
R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;
R¹⁹ is independently selected at each instance from hydrogen, alkyl, haloalkyl, cycloalkyl, aryl, heterocycle, and heteroaryl;
R^{8a}, R^{8b}, R^{8c}, and R^{8d} are independently selected at each instance from R⁷ and R¹² wherein at least one of R^{8a}, R^{8b}, R^{8c}, and R^{8d} is R¹²;
R⁹ is a bivalent moiety selected from alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;
R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R¹⁷; and
R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷; and
R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;
R¹⁶ is a heteroaryl group, where the bond to the sulfur atom is through one of the nitrogen atoms present in the cycle, and R¹⁶ is optionally substituted as allowed by valence with 1, 2, 3, or 4 substituents selected from R⁷;
KRAS Recognition Moiety is a molecule which can bind to or otherwise anchors to KRAS.

3. The compound of embodiment 1 or embodiment 2, wherein the compound is of Formula:

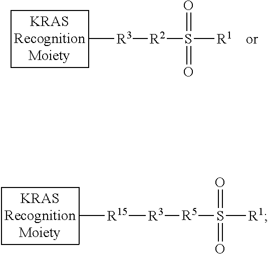

or a pharmaceutically acceptable salt thereof.

4. The compound of embodiment 1 or embodiment 2, wherein the compound is of Formula:

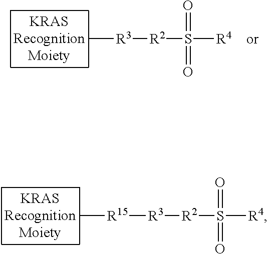

or a pharmaceutically acceptable salt thereof.

5. The compound of embodiment 1 or embodiment 2, wherein the compound is of Formula:

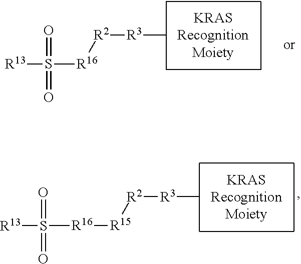

or a pharmaceutically acceptable salt thereof.

6. The compound of embodiment 5, wherein R¹³ is phenyl optionally substituted as allowed by valence with 1, 2, 3, or 4 substituents selected from R⁷.

7. The compound of embodiment 5, wherein R¹³ is alkyl optionally substituted as allowed by valence with 1, 2, 3, or 4 substituents selected from R⁷.

8. The compound of embodiment 5, wherein R¹³ is cycloalkyl optionally substituted as allowed by valence with 1, 2, 3, or 4 substituents selected from R⁷.

9. The compound of embodiment 5, wherein R¹³ is aryl optionally substituted as allowed by valence with 1, 2, 3, or 4 substituents selected from R⁷.

10. The compound of embodiment 5, wherein R¹³ is cyclopropyl optionally substituted as allowed by valence with 1, 2, 3, or 4 substituents selected from R⁷.

11. The compound of embodiment 5, wherein R¹³ is heterocycle optionally substituted as allowed by valence with 1, 2, 3, or 4 substituents selected from R⁷.

12. The compound of embodiment 5, wherein R¹³ is haloalkyl optionally substituted as allowed by valence with 1, 2, 3, or 4 substituents selected from R⁷.

13. The compound of any one of embodiments 5-12, wherein R¹⁶ is a triazole.

14. The compound of any one of embodiments 5-12, wherein R¹⁶ is

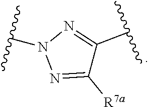

15. The compound of any one of embodiments 5-12, wherein R¹⁶ is

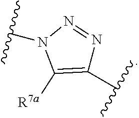

16. The compound of any one of embodiments 5-12, wherein R¹⁶ is

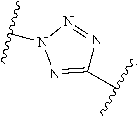 [SMILES: *c1nnn(*)n1]

17. The compound of any one of embodiments 5-12, wherein R¹⁶ is

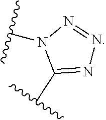 [SMILES: *c1nnnn1*]

18. The compound of any one of embodiments 5-12, wherein R¹⁶ is

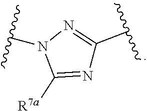

19. The compound of any one of embodiments 5-12, wherein R¹⁶ is

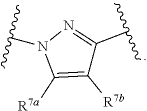

20. The compound of any one of embodiments 5-12, wherein R¹⁶ is

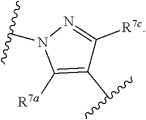

21. The compound of any one of embodiments 5-12, wherein R¹⁶ is

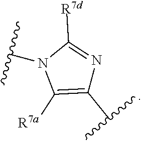

22. The compound of embodiment 1 or 2, wherein the compound is of Formula:

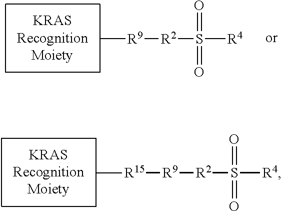

or a pharmaceutically acceptable salt thereof.

23. The compound of embodiment 22, wherein R⁹ is selected from alkyl, alkenyl, haloalkyl, cycloalkyl, bicycle, tricycle, and heteroaryl, each of which except bond is optionally substituted as allowed by valence with 1, 2, 3, or 4 substituents selected from R⁷.

24. The compound of embodiment 22, wherein R⁹ is alkyl optionally substituted as allowed by valence with 1, 2, 3, or 4 substituents selected from R⁷.

25. The compound of embodiment 22, wherein R⁹ is alkenyl optionally substituted as allowed by valence with 1, 2, 3, or 4 substituents selected from R⁷.

26. The compound of embodiment 22, wherein R⁹ is haloalkyl optionally substituted as allowed by valence with 1, 2, 3, or 4 substituents selected from R⁷.

27. The compound of embodiment 22, wherein R⁹ is cycloalkyl optionally substituted as allowed by valence with 1, 2, 3, or 4 substituents selected from R⁷.

28. The compound of embodiment 22, wherein R⁹ is heteroaryl optionally substituted as allowed by valence with 1, 2, 3, or 4 substituents selected from R⁷.

29. The compound of embodiment 22, wherein R⁹ is bicycle optionally substituted as allowed by valence with 1, 2, 3, or 4 substituents selected from R⁷.

30. The compound of any one of embodiments 1-29, wherein R³ is bond.

31. The compound of any one of embodiments 1-29, wherein R³ is phenyl optionally substituted as allowed by valence with 1, 2, 3, or 4 substituents selected from R⁷.

32. The compound of any one of embodiments 1-29, wherein R³ is alkyl optionally substituted as allowed by valence with 1, 2, 3, or 4 substituents selected from R⁷.

33. The compound of any one of embodiments 1-29, wherein R³ is alkenyl optionally substituted as allowed by valence with 1, 2, 3, or 4 substituents selected from R⁷.

34. The compound of any one of embodiments 1-29, wherein R³ is haloalkyl optionally substituted as allowed by valence with 1, 2, 3, or 4 substituents selected from R⁷.

35. The compound of any one of embodiments 1-29, wherein R³ is cycloalkyl optionally substituted as allowed by valence with 1, 2, 3, or 4 substituents selected from R⁷.

36. The compound of any one of embodiments 1-29, wherein R³ is aryl optionally substituted as allowed by valence with 1, 2, 3, or 4 substituents selected from R⁷.

37. The compound of any one of embodiments 1-29, wherein R³ is heteroaryl optionally substituted as allowed by valence with 1, 2, 3, or 4 substituents selected from R⁷.

38. The compound of any one of embodiments 1-29, wherein R³ is bicycle optionally substituted as allowed by valence with 1, 2, 3, or 4 substituents selected from R⁷.

39. The compound of any one of embodiments 1-38, wherein R² is selected from bond, alkyl, alkenyl, haloalkyl, cycloalkyl, aryl, bicycle, tricycle, and heteroaryl, each of which except bond is optionally substituted as allowed by valence with 1, 2, 3, or 4 substituents selected from R⁷.

40. The compound of any one of embodiments 1-38, wherein R² is bond.

41. The compound of any one of embodiments 1-38, wherein R² is phenyl optionally substituted as allowed by valence with 1, 2, 3, or 4 substituents selected from R⁷.

42. The compound of any one of embodiments 1-38, wherein R² is alkyl optionally substituted as allowed by valence with 1, 2, 3, or 4 substituents selected from R⁷.

43. The compound of any one of embodiments 1-38, wherein R² is alkenyl optionally substituted as allowed by valence with 1, 2, 3, or 4 substituents selected from R⁷.

44. The compound of any one of embodiments 1-38, wherein R² is haloalkyl optionally substituted as allowed by valence with 1, 2, 3, or 4 substituents selected from R⁷.

45. The compound of any one of embodiments 1-38, wherein R² is cycloalkyl optionally substituted as allowed by valence with 1, 2, 3, or 4 substituents selected from R⁷.

46. The compound of any one of embodiments 1-38, wherein R² is aryl optionally substituted as allowed by valence with 1, 2, 3, or 4 substituents selected from R⁷.

47. The compound of any one of embodiments 1-38, wherein R² is heteroaryl optionally substituted as allowed by valence with 1, 2, 3, or 4 substituents selected from R⁷.

48. The compound of any one of embodiments 1-38, wherein R² is bicycle optionally substituted as allowed by valence with 1, 2, 3, or 4 substituents selected from R⁷.

49. The compound of any one of embodiments 1-48, wherein R² is not substituted.

50. The compound of any one of embodiments 1-48, wherein R² is substituted as allowed by valence with 1 substituent selected from R⁷.

51. The compound of any one of embodiments 1-48, wherein R² is substituted as allowed by valence with 2 substituents selected from R⁷.

52. The compound of any one of embodiments 1-48, wherein R² is substituted as allowed by valence with 3 substituents selected from R⁷.

53. The compound of embodiment 1, wherein the compound is of Formula:

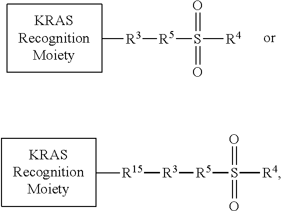

or a pharmaceutically acceptable salt thereof.

54. The compound of embodiment 1, wherein the compound is Formula:

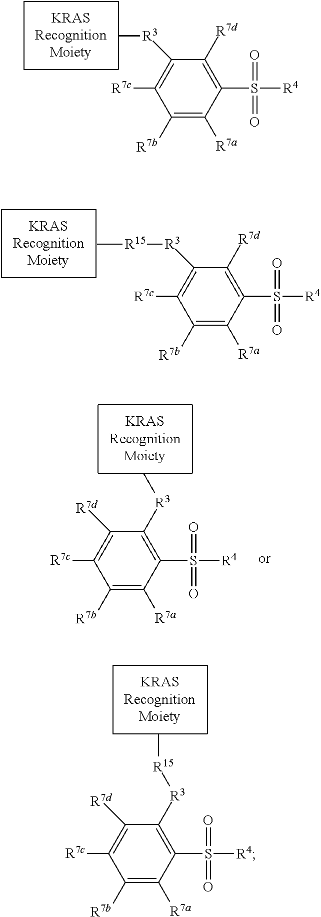

or a pharmaceutically acceptable salt thereof.

55. A compound of Formula

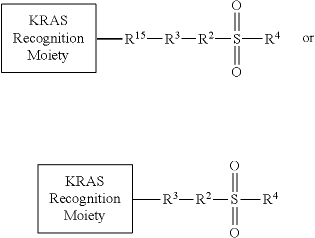

or a pharmaceutically acceptable salt thereof;
wherein:
R² is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;
R³ is independently selected at each instance from bond, alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents selected from R⁷;
and wherein R² and R³ are selected such that a suitably stable and suitably nontoxic compound for in vivo administration in a host is achieved;
each p is independently selected from 1, 2, 3, 4, 5, and 6;
R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;
R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;
R⁷ is independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
R¹⁵ is selected from the group consisting of alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents independently selected from R⁷;
R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶;
R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;
R¹⁹ is independently selected at each instance from hydrogen, alkyl, haloalkyl, cycloalkyl, aryl, heterocycle, and heteroaryl; and
KRAS Recognition Moiety is a molecule which can bind to or otherwise anchors too KRAS.

56. The compound of embodiment 1, wherein the compound is Formula:

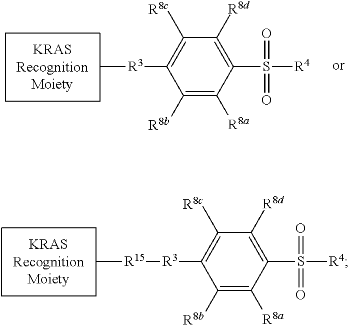

or a pharmaceutically acceptable salt thereof.

57. The compound of any one of embodiments 53-56, wherein R³ is bond.

58. The compound of any one of embodiments 53-56, wherein R³ is phenyl optionally substituted as allowed by valence with 1, 2, 3, or 4 substituents selected from R⁷.

59. The compound of any one of embodiments 53-56, wherein R³ is alkyl optionally substituted as allowed by valence with 1, 2, 3, or 4 substituents selected from R⁷.

60. The compound of any one of embodiments 53-56, wherein R³ is alkenyl optionally substituted as allowed by valence with 1, 2, 3, or 4 substituents selected from R⁷.

61. The compound of any one of embodiments 53-56, wherein R³ is haloalkyl optionally substituted as allowed by valence with 1, 2, 3, or 4 substituents selected from R⁷.

62. The compound of any one of embodiments 53-56, wherein R³ is cycloalkyl optionally substituted as allowed by valence with 1, 2, 3, or 4 substituents selected from R⁷.

63. The compound of any one of embodiments 53-56, wherein R³ is aryl optionally substituted as allowed by valence with 1, 2, 3, or 4 substituents selected from R⁷.

64. The compound of any one of embodiments 53-56, wherein R³ is heteroaryl optionally substituted as allowed by valence with 1, 2, 3, or 4 substituents selected from R⁷.

65. The compound of any one of embodiments 53-56, wherein R³ is bicycle optionally substituted as allowed by valence with 1, 2, 3, or 4 substituents selected from R⁷.

66. The compound of any one of embodiments 1-4 or 22-65, wherein R¹ and R⁴ are

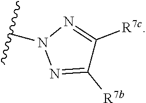

67. The compound of any one of embodiments 1-4 or 22-65, wherein R¹ and R⁴ are

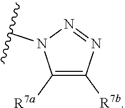

68. The compound of any one of embodiments 1-4 or 22-65, wherein R¹ and R⁴ are

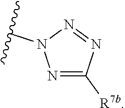

69. The compound of any one of embodiments 1-4 or 22-65, wherein R¹ and R⁴ are

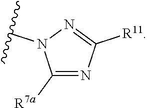

70. The compound of any one of embodiments 1-4 or 22-65, wherein R¹ and R⁴ are

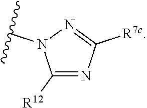

71. The compound of any one of embodiments 1-4 or 22-65, wherein R¹ and R⁴ are

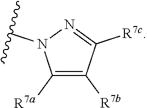

72. The compound of any one of embodiments 1-4 or 22-65, wherein R¹ and R⁴ are

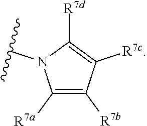

73. The compound of any one of embodiments 1-4 or 22-65, wherein R¹ and R⁴ are

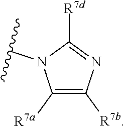

74. The compound of any one of embodiments 1-4 or 22-65, wherein R¹ and R⁴ are a bicyclic heteroaryl which is optionally substituted as allowed by valence with 1, 2, 3, or 4 substituents selected from R⁷.

75. The compound of any one of embodiments 1-4 or 22-65, wherein R⁴ is a heteroaryl group, where the bond to the sulfur atom is through the nitrogen present in the cycle, and each heteroaryl is optionally substituted as allowed by valence with 1, 2, 3, or 4 substituents selected from R⁷.

76. The compound of any one of embodiments 1-75, wherein R⁶ is independently selected at each instance from alkyl, haloalkyl, cycloalkyl, aryl, heterocycle, and heteroaryl; each of which is optionally substituted as allowed by valence with 1, 2, 3, or 4 substituents selected from R¹⁸.

77. The compound of any one of embodiments 1-75, wherein R⁶ is alkyl optionally substituted as allowed by valence with 1, 2, 3, or 4 substituents selected from R¹⁸.

78. The compound of any one of embodiments 1-75, wherein R⁶ is haloalkyl optionally substituted as allowed by valence with 1, 2, 3, or 4 substituents selected from R¹⁸.

79. The compound of any one of embodiments 1-75, wherein R⁶ is cycloalkyl optionally substituted as allowed by valence with 1, 2, 3, or 4 substituents selected from R¹⁸.

80. The compound of any one of embodiments 1-75, wherein R⁶ is aryl optionally substituted as allowed by valence with 1, 2, 3, or 4 substituents selected from R¹⁸.

81. The compound of any one of embodiments 1-75, wherein R⁶ is heterocycle optionally substituted as allowed by valence with 1, 2, 3, or 4 substituents selected from R¹⁸.

82. The compound of any one of embodiments 1-75, wherein R⁶ is heteroaryl optionally substituted as allowed by valence with 1, 2, 3, or 4 substituents selected from R¹⁸.

83. The compound of any one of embodiments 76-82, wherein R⁶ is not substituted with R¹⁸.

84. The compound of any one of embodiments 76-82, wherein R⁶ is substituted with 1 substituent selected from R¹⁸.

85. The compound of any one of embodiments 76-82, wherein R⁶ is substituted with 2 substituents independently selected from R¹⁸.

86. The compound of any one of embodiments 76-82, wherein R⁶ is substituted with 3 substituents independently selected from R¹⁸.

87. The compound of any one of embodiments 1-86, wherein R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, and nitro, wherein each alkyl, haloalkyl, alkenyl, cycloalkyl, heterocycle, aryl, and heteroaryl is optionally substituted as allowed by valence with 1, 2, 3, or 4 substituents selected from R¹⁷.

88. The compound of any one of embodiments 1-87, wherein one of R⁷, R^{7a}, R^{7b}, R^{7c}, and R^{7d} is alkyl optionally substituted as allowed by valence with 1, 2, 3, or 4 substituents selected from R¹⁷.

89. The compound of any one of embodiments 1-87, wherein one of R⁷, R^{7a}, R^{7b}, R^{7c}, and R^{7d} is haloalkyl optionally substituted as allowed by valence with 1, 2, 3, or 4 substituents selected from R¹⁷.

90. The compound of any one of embodiments 1-87, wherein one of R⁷, R^{7a}, R^{7b}, R^{7c}, and R^{7d} is phenyl optionally substituted as allowed by valence with 1, 2, 3, or 4 substituents selected from R¹⁷.

91. The compound of any one of embodiments 1-87, wherein one of R⁷, R^{7a}, R^{7b}, R^{7c}, and R^{7d} is heteroaryl optionally substituted as allowed by valence with 1, 2, 3, or 4 substituents selected from R¹⁷.

92. The compound of any one of embodiments 1-87, wherein one of R⁷, R^{7a}, R^{7b}, R^{7c}, and R^{7d} is haloalkyl optionally substituted as allowed by valence with 1, 2, 3, or 4 substituents selected from R¹⁷.

93. The compound of any one of embodiments 1-87, wherein one of R⁷, R^{7a}, R^{7b}, R^{7c}, and R^{7d} is heterocycle optionally substituted as allowed by valence with 1, 2, 3, or 4 substituents selected from R¹⁷.

94. The compound of any one of embodiments 1-87, wherein one of R⁷, R^{7a}, R^{7b}, R^{7c}, and R^{7d} is cycloalkyl optionally substituted as allowed by valence with 1, 2, 3, or 4 substituents selected from R¹⁷.

95. The compound of any one of embodiments 87-94, wherein R⁷, R^{7a}, R^{7b}, R^{7c}, and R^{7d} are not substituted.

96. The compound of any one of embodiments 87-94, wherein R⁷, R^{7a}, R^{7b}, R^{7c}, and R^{7d} are optionally substituted with 1 or 2 substituents selected from R¹⁷.

97. The compound of any one of embodiments 1-87, wherein one of R⁷, R^{7a}, R^{7b}, R^{7c}, and R^{7d} is halogen.

98. The compound of any one of embodiments 1-87, wherein one of R⁷, R^{7a}, R^{7b}, R^{7c}, and R^{7d} is —OR⁶.

99. The compound of any one of embodiments 1-87, wherein one of R⁷, R^{7a}, R^{7b}, R^{7c}, and R^{7d} is ═O.

100. The compound of any one of embodiments 1-87, wherein one of R⁷, R^{7a}, R^{7b}, R^{7c}, and R^{7d} is cyano.

101. The compound of any one of embodiments 1-87, wherein one of R⁷, R^{7a}, R^{7b}, R^{7c}, and R^{7d} is nitro.

102. The compound of any one of embodiments 1-101, wherein R¹⁷ is selected in each instance from halogen, alkyl, haloalkyl, alkenyl, and cyano.

103. A compound selected from Table 1.

104. A compound selected from Table 2.

105. A pharmaceutical composition comprising a compound of any one of embodiments 1-104 or a pharmaceutically acceptable salt thereof and a pharmaceutically acceptable excipient.

106. The pharmaceutical composition of embodiment 105, wherein the composition is suitable for oral delivery.

107. The pharmaceutical composition of embodiment 105, wherein the composition is suitable for intravenous delivery.

108. The pharmaceutical composition of embodiment 105, wherein the composition is suitable for parental delivery.

109. A method of treating a disorder mediated by KRAS comprising administering an effective amount of a compound of any one of embodiments 1-104 or a pharmaceutically acceptable salt thereof or a pharmaceutical composition of any one of embodiments 105-108 to a patient in need thereof.

110. The method of embodiment 109, wherein the patient is a human.

111. The method of embodiment 109 or 110, wherein the disorder is a cancer.

112. The method of embodiment 111, wherein the cancer is a solid cancer.

113. The method of embodiment 111, wherein the cancer is a hematological cancer.

114. The method of embodiment 111, wherein the disorder is lung cancer.

115. The method of embodiment 111, wherein the disorder is pancreatic cancer.

116. The method of embodiment 111, wherein the disorder is colorectal cancer.

117. The method of embodiment 111, wherein the disorder is ovarian or endometrial cancer.

118. The method of embodiment 111, wherein the disorder is breast cancer.

119. The method of any one of embodiments 111-119, wherein the cancer is metastatic.

120. The method of any one of embodiments 111-120, wherein the cancer is relapsed.

121. The method of any one of embodiments 111-121, wherein the cancer is refractory.

122. The method of embodiment 109 or 110, wherein the disorder is a tumor.

123. The method any one of embodiments 109-122, wherein the disorder is mediated by mutant KRAS.

124. Use of a compound of any one of embodiments 1-104 or a pharmaceutically acceptable salt thereof or a pharmaceutical composition of any one of embodiments 105-108 to treat a KRAS mediated disorder in a patient in need thereof.

125. Use of a compound of any one of embodiments 1-104 or a pharmaceutically acceptable salt thereof or a pharmaceutical composition of any one of embodiments 105-108 in the manufacture of a medicament to treat a KRAS mediated disorder in a patient in need thereof.

126. The use of embodiment 124 or 125, wherein the patient is a human.

127. The use of any one of embodiments 124-126, wherein the disorder is a cancer.

128. The use of embodiment 127, wherein the cancer is a solid cancer.

129. The use of embodiment 127, wherein the cancer is a hematological cancer.

130. The use of embodiment 127, wherein the disorder is lung cancer.

131. The use of embodiment 127, wherein the disorder is pancreatic cancer.

132. The use of embodiment 127, wherein the disorder is colorectal cancer.

133. The use of embodiment 127, wherein the disorder is breast cancer.

134. The use of embodiment 127, wherein the disorder is ovarian or endometrial cancer.

135. The use of any one of embodiments 127-134, wherein the cancer is metastatic.

136. The use of any one of embodiments 127-135, wherein the cancer is relapsed.

137. The use of any one of embodiments 127-136, wherein the cancer is refractory.

138. The use of any one of embodiments 124-126, wherein the disorder is a tumor.

139. The use of any one of embodiments 125-138, wherein the disorder is mediated by mutant KRAS.

140. A compound of any one of embodiments 1-104 or a pharmaceutically acceptable salt thereof or a pharmaceutical composition of any one of embodiments 105-108 for use in the treatment of a KRAS mediated disorder in a patient in need thereof.

141. The compound or pharmaceutical composition of embodiment 140, wherein the patient is a human.

142. The compound or pharmaceutical composition of embodiment 140 or 141, wherein the disorder is a cancer.

143. The compound or pharmaceutical composition of embodiment 142, wherein the cancer is a solid cancer.

144. The compound or pharmaceutical composition of embodiment 142, wherein the cancer is a hematological cancer.

145. The compound or pharmaceutical composition of embodiment 142, wherein the disorder is lung cancer.

146. The compound or pharmaceutical composition of embodiment 142, wherein the disorder is pancreatic cancer.

147. The compound or pharmaceutical composition of embodiment 142, wherein the disorder is breast cancer.

148. The compound or pharmaceutical composition of embodiment 142, wherein the disorder is ovarian or endometrial cancer.

149. The compound or pharmaceutical composition of embodiment 142, wherein the disorder is fallopian tube cancer.

150. The compound or pharmaceutical composition of any one of embodiments 142-149, wherein the cancer is metastatic.

151. The compound or pharmaceutical composition of any one of embodiments 142-150, wherein the cancer is relapsed.

152. The compound or pharmaceutical composition of any one of embodiments 142-151, wherein the cancer is refractory.

153. The compound or pharmaceutical composition of embodiment 140 or 141, wherein the disorder is a tumor.

154. The compound or pharmaceutical composition of any one of embodiments 140-153, wherein the disorder is mediated by mutant KRAS.

Additional Heteroaryl Sulfonyl Compounds of the Present Invention

In certain embodiments the heteroaryl sulfonyl compound of the present invention is selected from:

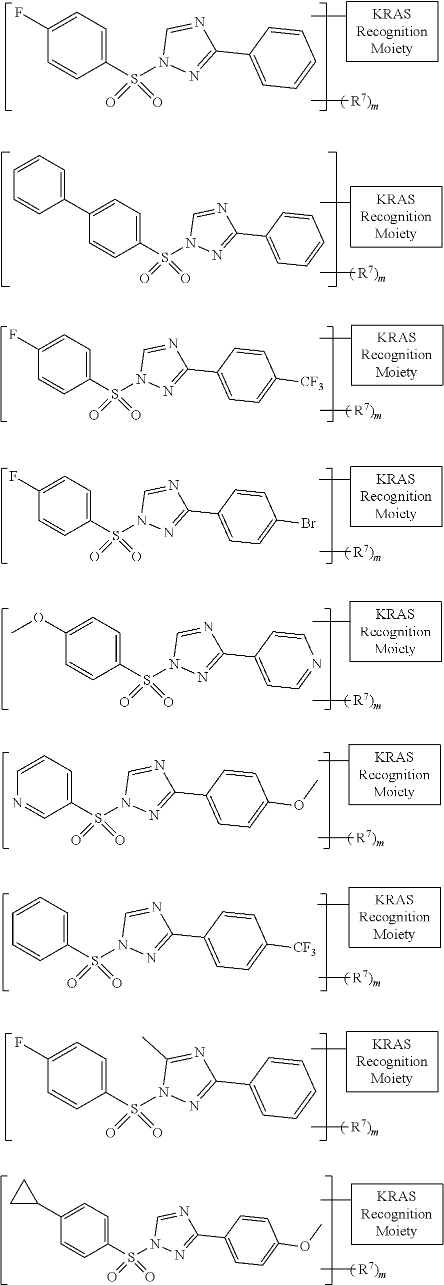

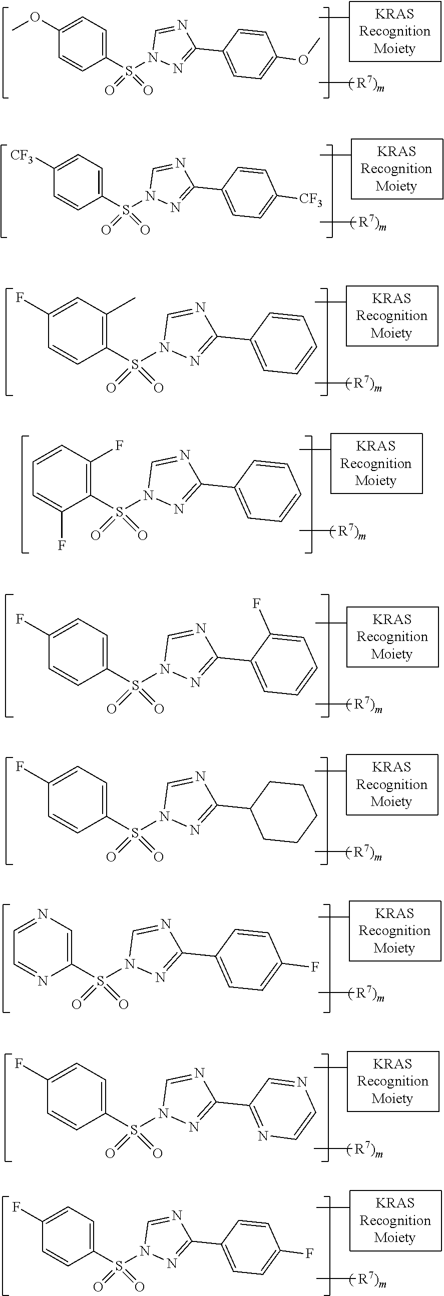

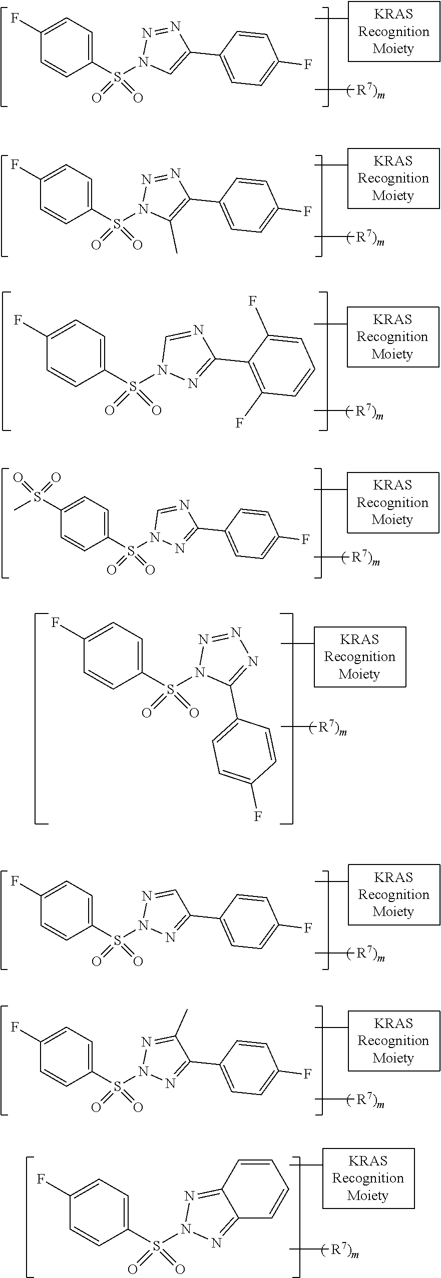

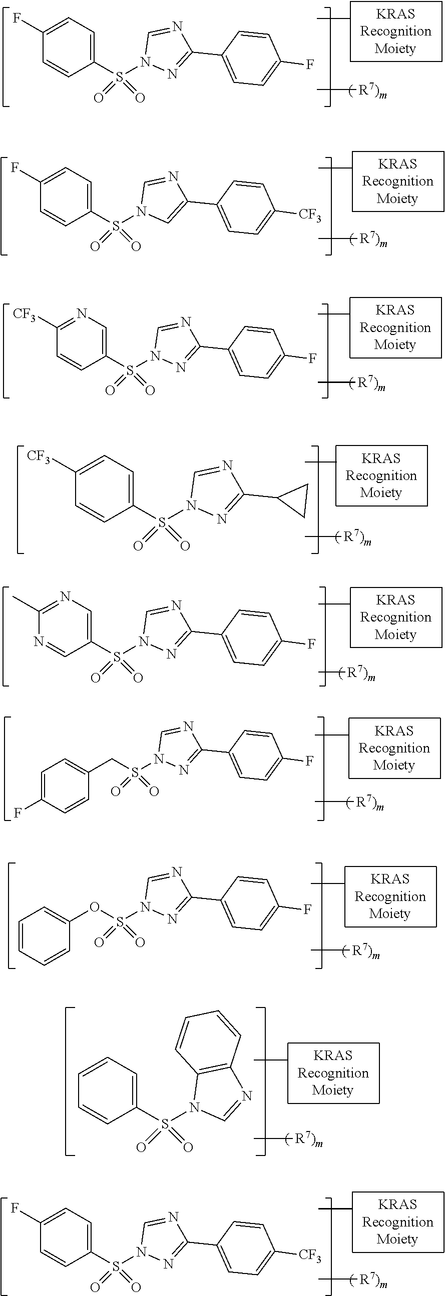

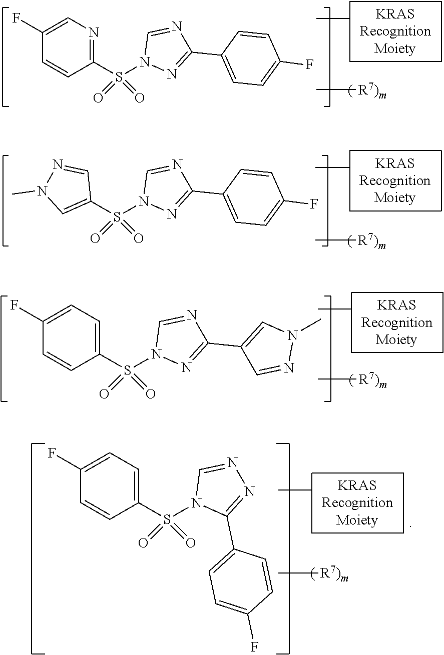

In certain embodiments the heteroaryl sulfonyl compound of the present invention is selected from:

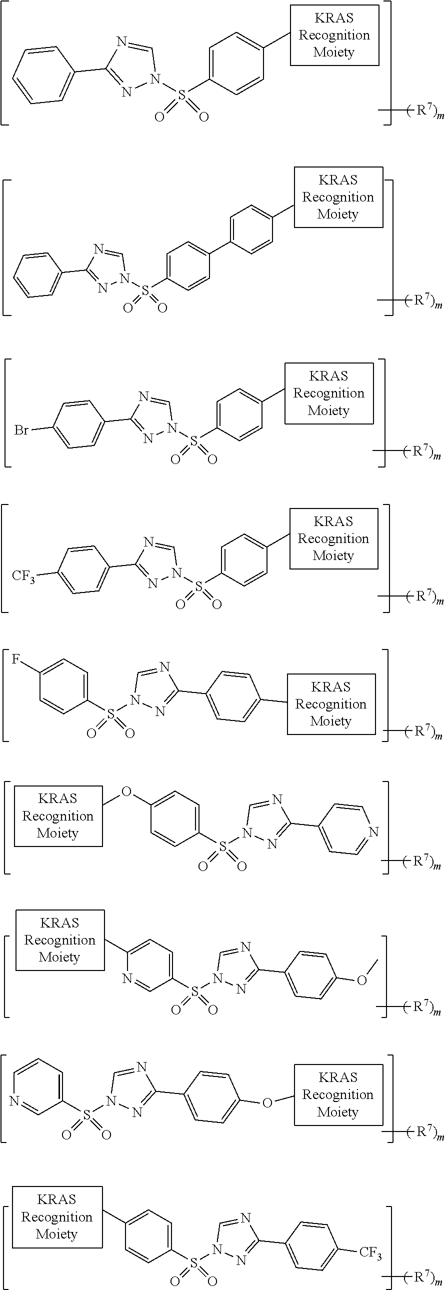

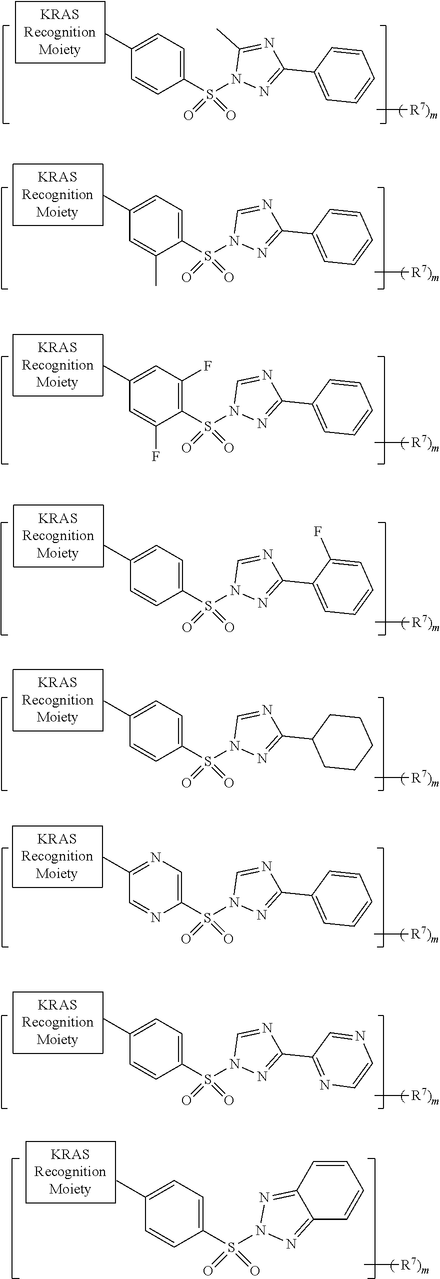

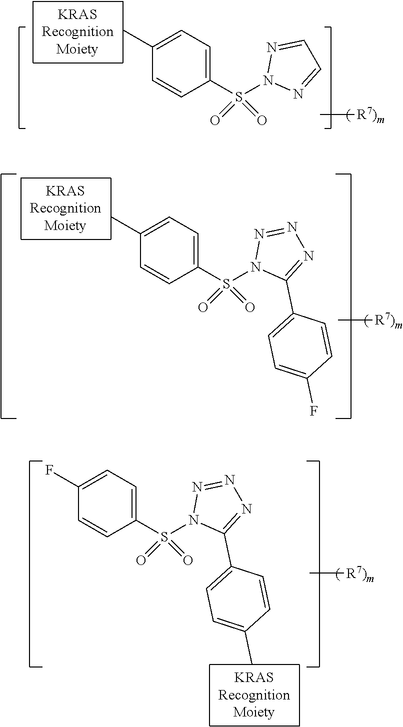

Non-limiting examples of heteroaryl sulfonyl compounds of the present invention include:

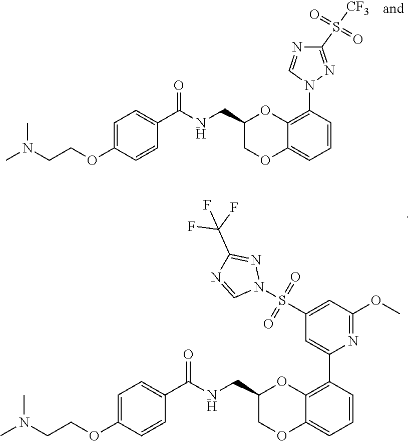

Non-limiting examples of heteroaryl sulfonyl compounds of the present invention include:

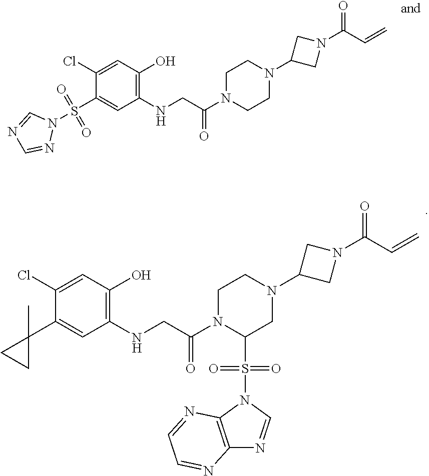 [SMILES: C=CC(=O)N1CC(N2CCN(C(=O)CNc3cc(S(=O)(=O)n4cncn4)c(Cl)cc3O)CC2)C1 ; C=CC(=O)N1CC(N2CCN(C(=O)CNc3cc(C4(C)CC4)c(Cl)cc3O)C(S(=O)(=O)n3cnc4nccnc43)C2)C1]

Non-limiting examples of heteroaryl sulfonyl compounds of the present invention include:

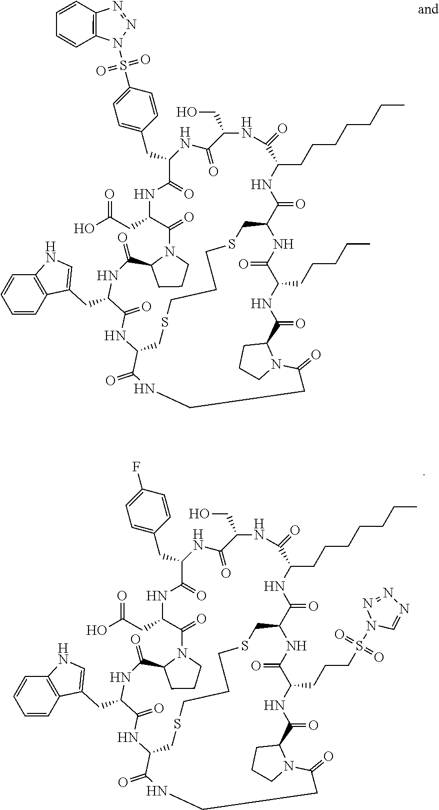 [SMILES: CCCCCCC[C@@H]1NC(=O)[C@@H]2CSCCCSC[C@@H](NC(=O)[C@H](Cc3c[nH]c4ccccc34)NC(=O)[C@@H]3CCCN3C(=O)[C@H](CC(=O)O)NC(=O)[C@H](Cc3ccc(S(=O)(=O)n4nnc5ccccc54)cc3)NC(=O)[C@H](CO)NC1=O)C(=O)NCCC(=O)N1CCC[C@H]1C(=O)N[C@@H](CCCCC)C(=O)N2 ; CCCCCCC[C@@H]1NC(=O)[C@@H]2CSCCCSC[C@@H](NC(=O)[C@H](Cc3c[nH]c4ccccc34)NC(=O)[C@@H]3CCCN3C(=O)[C@H](CC(=O)O)NC(=O)[C@H](Cc3ccc(F)cc3)NC(=O)[C@H](CO)NC1=O)C(=O)NCCC(=O)N1CCC[C@H]1C(=O)N[C@@H](CCCS(=O)(=O)n1cnnn1)C(=O)N2]

Non-limiting examples of heteroaryl sulfonyl compounds of the present invention include:

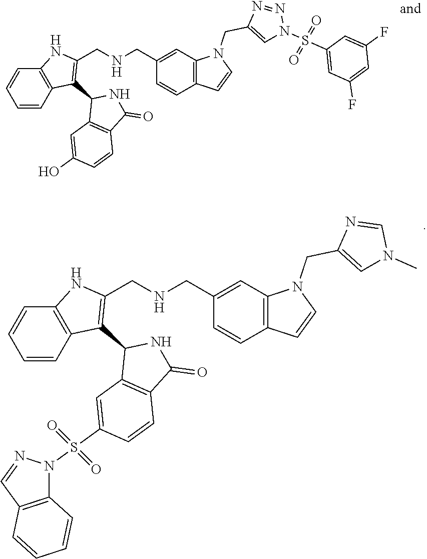 [SMILES: O=C1N[C@H](c2c(CNCc3ccc4ccn(Cc5cn(S(=O)(=O)c6cc(F)cc(F)c6)nn5)c4c3)[nH]c3ccccc23)c2cc(O)ccc21 ; Cn1cnc(Cn2ccc3ccc(CNCc4[nH]c5ccccc5c4[C@H]4NC(=O)c5ccc(S(=O)(=O)n6ncc7ccccc76)cc54)cc32)c1]

Non-limiting examples of heteroaryl sulfonyl compounds of the present invention include:

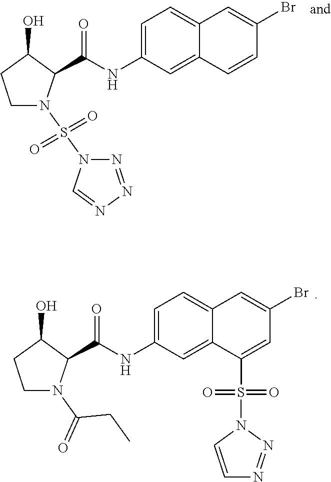 [SMILES: O=C(Nc1ccc2cc(Br)ccc2c1)[C@@H]1[C@H](O)CCN1S(=O)(=O)n1cnnn1 ; CCC(=O)N1CC[C@@H](O)[C@H]1C(=O)Nc1ccc2cc(Br)cc(S(=O)(=O)n3ccnn3)c2c1]

Non-limiting examples of heteroaryl sulfonyl compounds of the present invention include:

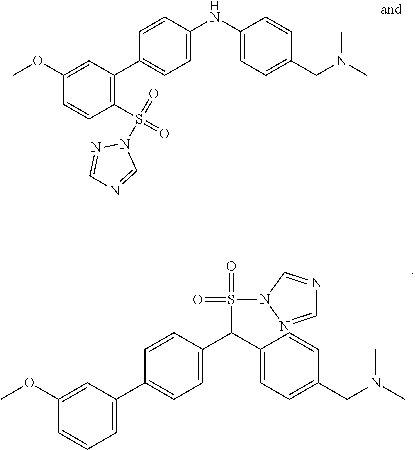

Non-limiting examples of heteroaryl sulfonyl compounds of the present invention include:

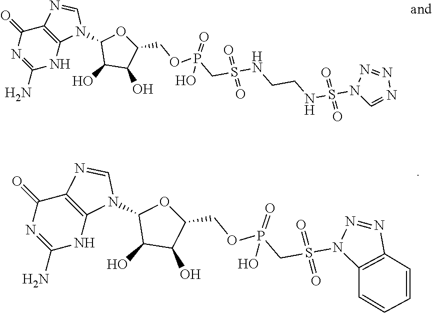 [SMILES: Nc1nc(=O)c2ncn([C@@H]3O[C@H](COP(=O)(O)CS(=O)(=O)NCCNS(=O)(=O)n4cnnn4)[C@@H](O)[C@H]3O)c2[nH]1 ; Nc1nc(=O)c2ncn([C@@H]3O[C@H](COP(=O)(O)CS(=O)(=O)n4nnc5ccccc54)[C@@H](O)[C@H]3O)c2[nH]1]

Non-limiting examples of heteroaryl sulfonyl compounds of the present invention include:

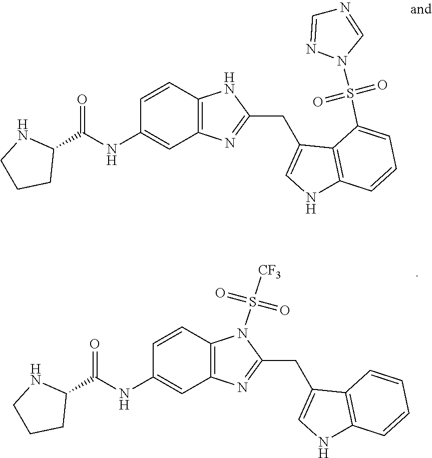 [SMILES: O=C(Nc1ccc2[nH]c(Cc3c[nH]c4cccc(S(=O)(=O)n5cncn5)c34)nc2c1)[C@@H]1CCCN1 ; O=C(Nc1ccc2c(c1)nc(Cc1c[nH]c3ccccc13)n2S(=O)(=O)C(F)(F)F)[C@@H]1CCCN1]

Non-limiting examples of heteroaryl sulfonyl compounds of the present invention include:

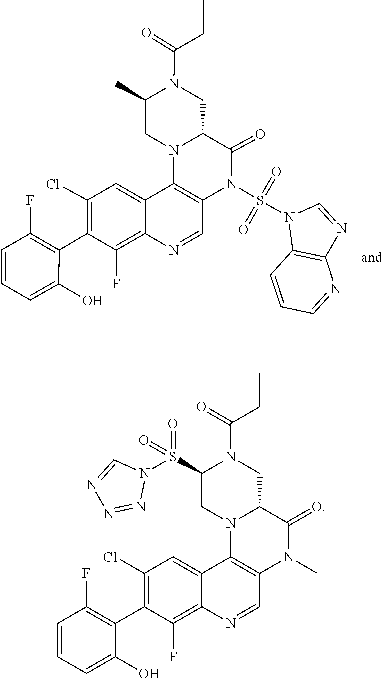 [SMILES: CCC(=O)N1C[C@@H]2C(=O)N(S(=O)(=O)n3cnc4ncccc43)c3cnc4c(F)c(-c5c(O)cccc5F)c(Cl)cc4c3N2C[C@H]1C ; CCC(=O)N1C[C@@H]2C(=O)N(C)c3cnc4c(F)c(-c5c(O)cccc5F)c(Cl)cc4c3N2C[C@H]1S(=O)(=O)n1cnnn1]

Non-limiting examples of heteroaryl sulfonyl compounds of the present invention include:

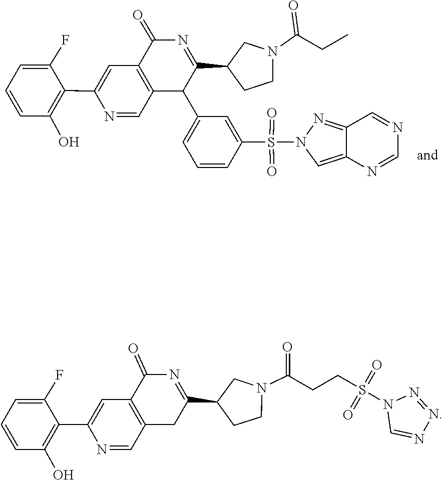 [SMILES: CCC(=O)N1CC[C@@H](C2=NC(=O)c3cc(-c4c(O)cccc4F)ncc3C2c2cccc(S(=O)(=O)n3cc4ncncc4n3)c2)C1 ; O=C1N=C([C@@H]2CCN(C(=O)CCS(=O)(=O)n3cnnn3)C2)Cc2cnc(-c3c(O)cccc3F)cc21]

Non-limiting examples of heteroaryl sulfonyl compounds of the present invention include:

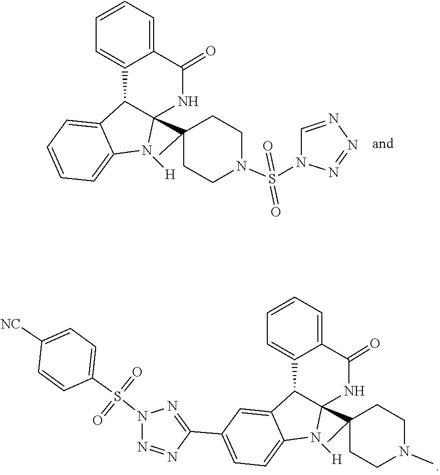 [SMILES: CC1([C@]23NC(=O)c4ccccc4[C@H]2c2ccccc2N3)CCN(S(=O)(=O)n2cnnn2)CC1 ; CN1CCC(C)([C@]23NC(=O)c4ccccc4[C@H]2c2cc(-c4nnn(S(=O)(=O)c5ccc(C#N)cc5)n4)ccc2N3)CC1]

Non-limiting examples of heteroaryl sulfonyl compounds of the present invention include:

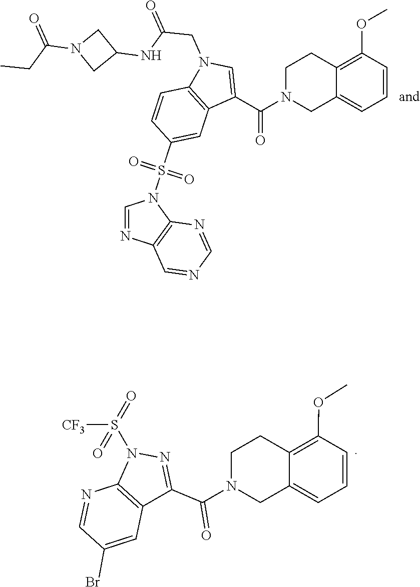 [SMILES: CCC(=O)N1CC(NC(=O)Cn2cc(C(=O)N3CCc4c(cccc4OC)C3)c3cc(S(=O)(=O)n4cnc5cncnc54)ccc32)C1 ; COc1cccc2c1CCN(C(=O)c1nn(S(=O)(=O)C(F)(F)F)c3ncc(Br)cc13)C2]

Non-limiting examples of heteroaryl sulfonyl compounds of the present invention include:

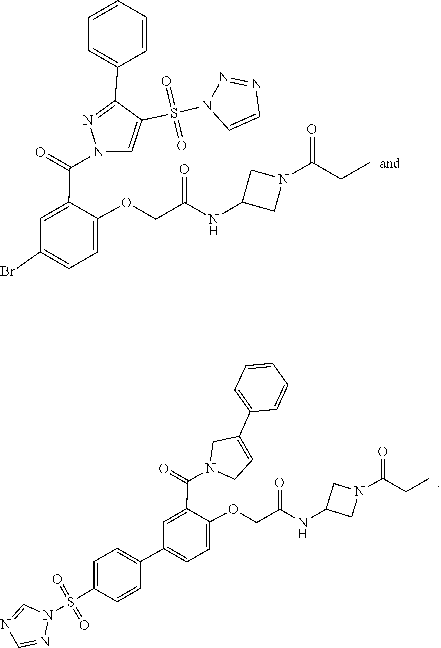 [SMILES: CCC(=O)N1CC(NC(=O)COc2ccc(Br)cc2C(=O)n2cc(S(=O)(=O)n3ccnn3)c(-c3ccccc3)n2)C1 ; CCC(=O)N1CC(NC(=O)COc2ccc(-c3ccc(S(=O)(=O)n4cncn4)cc3)cc2C(=O)N2CC=C(c3ccccc3)C2)C1]

Non-limiting examples of heteroaryl sulfonyl compounds of the present invention include:

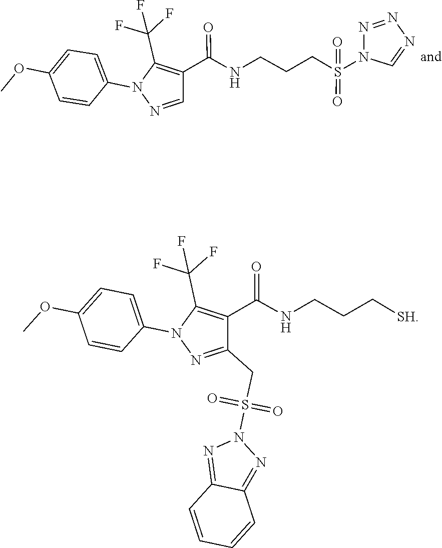 [SMILES: COc1ccc(-n2ncc(C(=O)NCCCS(=O)(=O)n3cnnn3)c2C(F)(F)F)cc1 ; COc1ccc(-n2nc(CS(=O)(=O)n3nc4ccccc4n3)c(C(=O)NCCCS)c2C(F)(F)F)cc1]

Non-limiting examples of heteroaryl sulfonyl compounds of the present invention include:

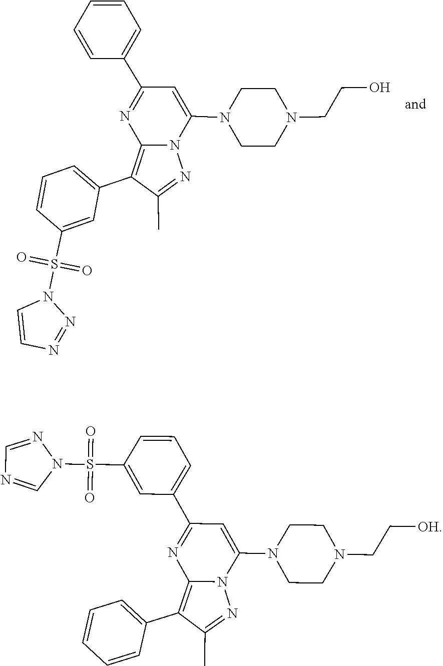 [SMILES: Cc1nn2c(N3CCN(CCO)CC3)cc(-c3ccccc3)nc2c1-c1cccc(S(=O)(=O)n2ccnn2)c1 ; Cc1nn2c(N3CCN(CCO)CC3)cc(-c3cccc(S(=O)(=O)n4cncn4)c3)nc2c1-c1ccccc1]

### Terminology

As used herein, Anchor Bond is defined as the chemical bond between the KRAS Recognition Moiety and the rest of the molecule for example a bond to R³, R⁹, or R¹⁶, as appropriate. Non-limiting examples of Anchor Bonds are shown in bold in the following structures:

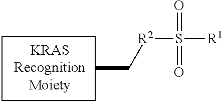

where R³ is methylene,

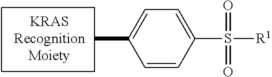

where R³ is bond and R² is phenyl,

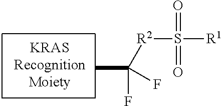

where R⁹ is difluoromethylene, and

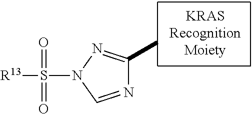

where R¹⁶ is 1,2,4-triazole.

Compounds are described using standard nomenclature. Unless defined otherwise, all technical and scientific terms used herein have the same meaning as is commonly understood by one of skill in the art to which this invention belongs.

The heteroaryl sulfonyl compounds in any of the Formulas described herein include enantiomers, mixtures of enantiomers, diastereomers, tautomers, racemates and other isomers, such as rotamers, as if each is specifically described, unless otherwise indicated or otherwise excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

In certain embodiments the present invention includes heteroaryl sulfonyl compounds with at least one desired isotopic substitution of an atom, at an amount above the natural abundance of the isotope, i.e., enriched. In certain embodiments the heteroaryl sulfonyl compounds of the present invention are not isotopically enriched.

Examples of isotopes that can be incorporated into heteroaryl sulfonyl compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁷O, ¹⁸O, ¹⁸F ³¹P, ³²P, ³⁵S, ³⁶CI, and ¹²⁵I respectively. In one embodiment, isotopically labelled heteroaryl sulfonyl compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. For example, a ¹⁸F labeled heteroaryl sulfonyl compound may be desirable for PET or SPECT studies. Isotopically labeled heteroaryl sulfonyl compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may optionally be used anywhere in described structures that achieves the desired result. Alternatively, or in addition, isotopes of carbon, e.g., ¹³C and ¹⁴C, may be used. In one embodiment, the isotopic substitution is replacing hydrogen with a deuterium at one or more locations on the molecule to improve the performance of the drug, for example, the pharmacodynamics, pharmacokinetics, biodistribution, half-life, stability, AUC, Tmax, Cmax, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95 or 99% or more enriched in an isotope at any location of interest. In certain embodiments deuterium is 80, 85, 90, 95 or 99% enriched at a desired location. Unless otherwise stated, the enrichment at any point is above natural abundance, and in an embodiment is enough to alter a detectable property of the drug in a human.

In one embodiment, the substitution of a hydrogen atom for a deuterium atom occurs within any variable group. For example, when any variable group is, or contain for example through substitution, methyl, ethyl, or methoxy, the alkyl residue may be deuterated (in nonlimiting embodiments, CDH₂, CD₂H, CD₃, CD₂CD₃, CHDCH₂D, CH₂CD₃, CHDCHD₂, OCDH₂, OCD₂H, or OCD₃ etc.).

The heteroaryl sulfonyl compound of the present invention may form a solvate with solvents (including water). Therefore, in one embodiment, the invention includes a solvated form of the active heteroaryl sulfonyl compound. The term “solvate” refers to a molecular complex of a heteroaryl sulfonyl compound of the present invention (including a salt thereof) with one or more solvent molecules. Nonlimiting examples of solvents are water, ethanol, dimethyl sulfoxide, acetone and other common organic solvents. The term “hydrate” refers to a molecular complex comprising a heteroaryl sulfonyl compound of the invention and water. Pharmaceutically acceptable solvates in accordance with the invention include those wherein the solvent of crystallization may be isotopically substituted, e.g. D₂O, d₆-acetone, d₆-DMSO. A solvate can be in a liquid or solid form.

A dash (“-”) that is not between two letters or symbols is used to indicate a point of attachment for a substituent. For example, —(C═O)NH₂ is attached through carbon of the keto (C═O) group.

The term “substituted”, as used herein, means that any one or more hydrogens on the designated atom or group is replaced with a moiety selected from the indicated group, provided that the designated atom's normal valence is not exceeded and the resulting heteroaryl sulfonyl compound is stable. For example, when the substituent is oxo (i.e., ═O) then two hydrogens on the atom are replaced. For example a pyridyl group substituted by oxo is a pyridone. Combinations of substituents and/or variables are permissible only if such combinations result in stable heteroaryl sulfonyl compounds or useful synthetic intermediates.

“Alkyl” is a branched, straight chain, or cyclic saturated aliphatic hydrocarbon group. In one embodiment, the alkyl contains from 1 to about 12 carbon atoms, more generally from 1 to about 6 carbon atoms, from 1 to about 4 carbon atoms, or from 1 to 3 carbon atoms. In one embodiment, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having from 1, 2, 3, 4, 5, or 6 carbon atoms and also a carbocyclic alkyl group of 3, 4, 5, or 6 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl.

When a term is used that includes “alk” it should be understood that “cycloalkyl” or “carbocyclic” can be considered part of the definition, unless unambiguously excluded by the context. For example and without limitation, the terms alkyl, alkenyl, alkynyl, alkoxy, alkanoyl, alkenloxy, haloalkyl, etc. can all be considered to include the cyclic forms of alkyl, unless unambiguously excluded by context.

“Alkenyl” is a branched or straight chain aliphatic hydrocarbon group having one or more carbon-carbon double bonds that may occur at a stable point along the chain. Nonlimiting examples are C₂-C₅alkenyl, C₂-C₇alkenyl, C₂-C₆alkenyl, C₂-C₅alkenyl and C₂-C₄alkenyl. The specified ranges as used herein indicate an alkenyl group having each member of the range described as an independent species, as described above for the alkyl moiety. Examples of alkenyl include, but are not limited to, ethenyl and propenyl.

“Alkynyl” is a branched or straight chain aliphatic hydrocarbon group having one or more carbon-carbon triple bonds that may occur at any stable point along the chain, for example, C₂-C₈alkynyl or C₂-C₆alkynyl. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “alkylthio” or a “thioalkyl” group is an alkyl group as defined above with the indicated number of carbon atoms covalently bound through a sulfur bridge (—S—). In one embodiment, the alkoxy group is optionally substituted as described above.

“Haloalkyl” indicates both branched and straight-chain alkyl groups substituted with 1 or more halogen atoms, up to the maximum allowable number of halogen atoms. Examples of haloalkyl include, but are not limited to, trifluoromethyl, monofluoromethyl, difluoromethyl, 2-fluoroethyl, and penta-fluoroethyl.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. The term “aryl” includes groups where a saturated or partially unsaturated carbocycle group is fused with an aromatic ring. The term “aryl” also includes groups where a saturated or partially unsaturated heterocycle group is fused with an aromatic ring so long as the attachment point is the aromatic ring. Such heteroaryl sulfonyl compounds may include aryl rings fused to a 4 to 7 or a 5 to 7-membered saturated or partially unsaturated cyclic group that optionally contains 1, 2 or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group.

The term “heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems). It does not include rings containing —O—O— or —S—S— portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused or bridged with a carbocycle radical. For example partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

Non-limiting examples of bicyclic heterocycles include:

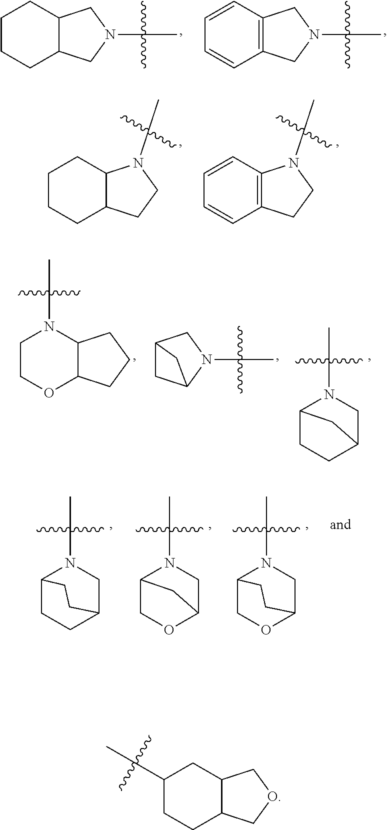

Unless otherwise drawn or clear from the context, the term “bicyclic heterocycle” includes cis and trans diastereomers. Non-limiting examples of chiral bicyclic heterocycles include:

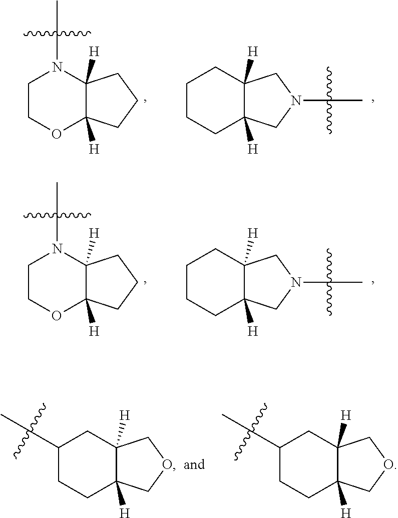

In certain alternative embodiments the term “heterocycle” refers to saturated and partially saturated heteroatom-containing ring radicals, where the heteroatoms may be selected from N, S, O, B, Si, and P.

The term “bicycle” refers to a ring system wherein two rings are fused together and each ring is independently selected from carbocycle, heterocycle, aryl, and heteroaryl. Non-limiting examples of bicycle groups include:

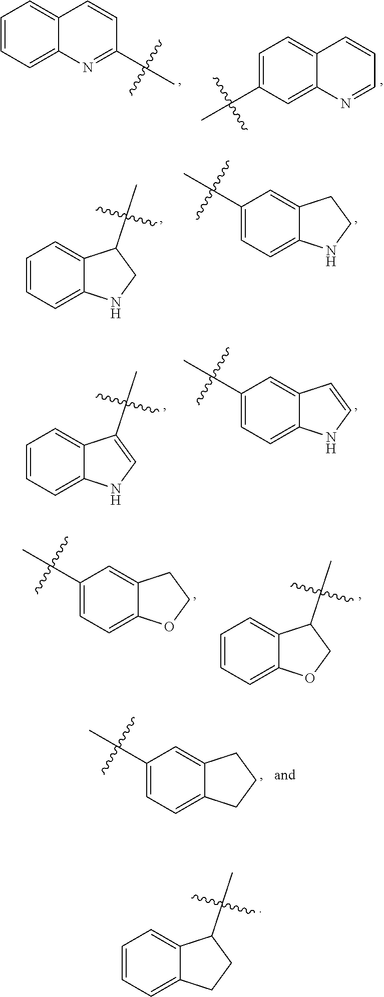

When the term “bicycle” is used in the context of a bivalent residue such as R², R³, or R⁵, the attachment points can be on separate rings or on the same ring. In certain embodiments both attachment points are on the same ring. In certain embodiments both attachment points are on different rings. Non-limiting examples of bivalent bicycle groups include:

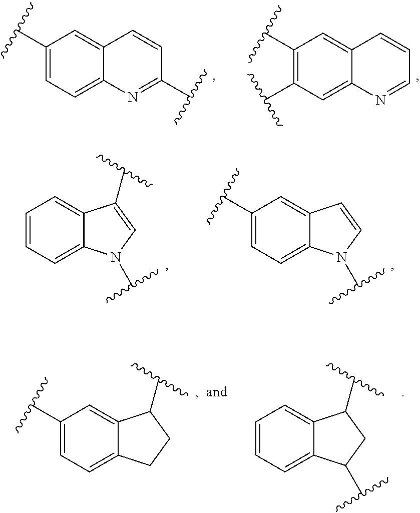 [SMILES: *c1ccc2nc(*)ccc2c1 ; *c1cc2cccnc2cc1* ; *c1cn(*)c2ccccc12 ; *c1ccc2c(ccn2*)c1 ; *c1ccc2c(c1)C(*)CC2 ; *C1CC(*)c2ccccc21]

The term “tricycle” refers to a ring system wherein three rings are fused together and each ring is independently selected from carbocycle, heterocycle, aryl, and heteroaryl. Non-limiting examples of bicycle groups include:

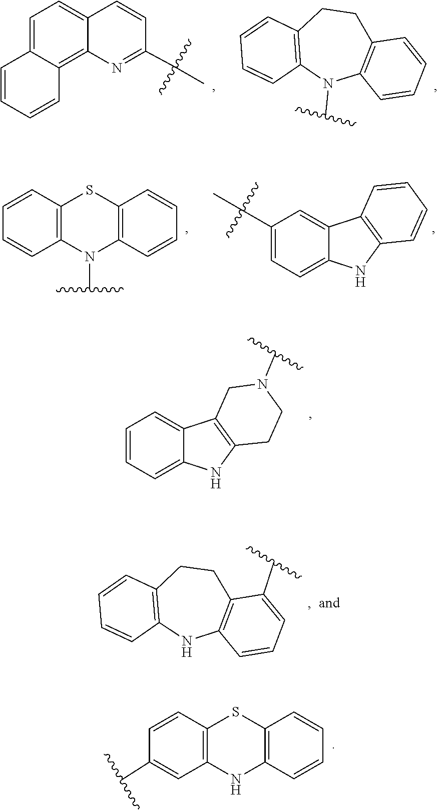

When the term “tricycle” is used in the context of a bivalent residue such as R², R³, or R⁵, the attachment points can be on separate rings or on the same ring. In certain embodiments both attachment points are on the same ring. In certain embodiments both attachment points are on different rings. Non-limiting examples of bivalent tricycle groups include:

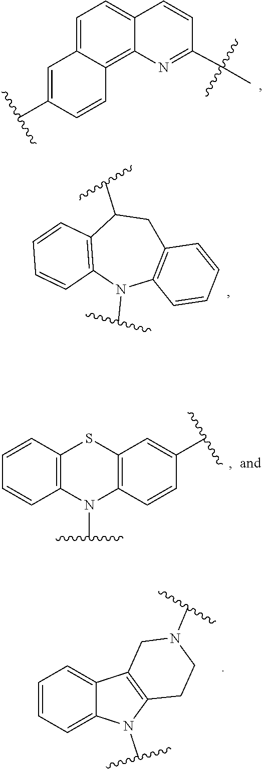 [SMILES: *c1ccc2c(ccc3ccc(*)nc32)c1 ; *C1Cc2ccccc2N(*)c2ccccc21 ; *c1ccc2c(c1)Sc1ccccc1N2* ; *N1CCc2c(c3ccccc3n2*)C1]

“Heteroaryl” refers to a stable monocyclic, bicyclic, or multicyclic aromatic ring which contains from 1 to 5, or in some embodiments from 1, 2, 3, 4, or 5 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5 or 6 ring atoms. In some embodiments bicyclic heteroaryl groups are 8- to 10-membered heteroaryl groups, that is, groups containing 8 or 10 ring atoms in which one 5, 6, or 7-member aromatic ring is fused to a second aromatic or non-aromatic ring wherein the point of attachment is the aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl. Heteroaryl groups are optionally substituted independently with one or more substituents described herein.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

“Pharmaceutical compositions” are compositions comprising at least one active agent, and at least one other substance, such as a carrier. The present invention includes pharmaceutical compositions of the described heteroaryl sulfonyl compounds.

“Pharmaceutical combinations” are combinations of at least two active agents which may be combined in a single dosage form or provided together in separate dosage forms with instructions that the active agents are to be used together to treat any disorder described herein.

A “pharmaceutically acceptable salt” is a derivative of the disclosed heteroaryl sulfonyl compound in which the parent heteroaryl sulfonyl compound is modified by making inorganic and organic, pharmaceutically acceptable, acid or base addition salts thereof. The salts of the present heteroaryl sulfonyl compounds can be synthesized from a parent heteroaryl sulfonyl compound that contains a basic or acidic moiety by conventional chemical methods. Generally, such salts can be prepared by reacting free acid forms of these heteroaryl sulfonyl compounds with a stoichiometric amount of the appropriate base (such as Na, Ca, Mg, or K hydroxide, carbonate, bicarbonate, or the like), or by reacting free base forms of these heteroaryl sulfonyl compounds with a stoichiometric amount of the appropriate acid. Such reactions are typically carried out in water or in an organic solvent, or in a mixture of the two. Salts of the present heteroaryl sulfonyl compounds further include solvates of the heteroaryl sulfonyl compounds and of the heteroaryl sulfonyl compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent heteroaryl sulfonyl compound formed, for example, from inorganic or organic acids. Examples, of such salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active heteroaryl sulfonyl compound is provided.

A “pharmaceutically acceptable excipient” means an excipient that is useful in preparing a pharmaceutical composition/combination that is generally safe, acceptable for human consumption, and neither biologically nor otherwise inappropriate for administration to a host, typically a human. In one embodiment, an excipient is used that is acceptable for veterinary use.

A “patient” or “host” or “subject” is a human or non-human animal in need of treatment or prevention of any of the disorders as specifically described herein. Typically, the host is a human. A “patient” or “host” or “subject” also refers to for example, a mammal, primate (e.g., human), cow, sheep, goat, horse, dog, cat, rabbit, rat, mice, bird and the like.

A “therapeutically effective amount” of a compound, pharmaceutical composition, or combination of this invention means an amount effective, when administered to a host, provides a therapeutic benefit such as an amelioration of symptoms or reduction or diminution of the disease itself.

KRAS Recognition Moiety

The KRAS Recognition Moiety is typically a ligand or a portion of a ligand that binds to the KRAS. Non-limiting examples of KRAS Recognition Moieties are provided below. The skilled artisan will recognize additional KRAS Recognition Moieties that are known in the art and will know where to link the moiety to provide the desired effect. For example, the skilled artisan can look up the crystal structure for KRAS on https://www.rcsb.org/ and then pull a list of ligands that bind that crystal structure. The skilled artisan can also determine where to attach the sulfur-heteroaryl group of the present invention based on the crystal structure provided which will allow identification of where in the binding pocket the sulfur-heteroaryl group can fit and which functional groups on the ligand are essential for activity.

As used herein codes referring to crystal structures correspond to the crystal structure available on the Protein Data Bank (PDB, https://www.rcsb.org). For example, non-limiting examples of crystal structures of KRAS include 6OIM, 5V9U, 6FA2, 6GQX, 6TAM, 7ACQ, 4EPW, 6VC8, 6ZL5, 5KYK, 6MTA, 4LDJ, 4TQ9, 4DSN, 4QL3, 3GFT, and 5YY1, where these codes correspond to crystal structures in the PDB database and are available online at https://www.rcsb.org.

As used herein R²⁷ is independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R¹⁷;

As used herein n is 0, 1, 2, 3, or 4.

As used herein

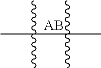 [SMILES: **]

is an Anchor Bond. Anchor Bond is the chemical bond between the KRAS Recognition Moiety and the rest of the molecule for example a bond to R³, R⁹, or R¹⁶, as appropriate.

In certain embodiments the KRAS Recognition Moiety is

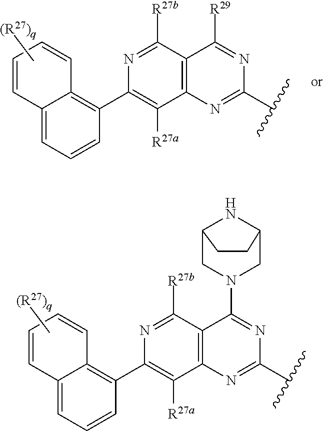

wherein
R²⁹ is independently selected from hydrogen, alkyl, haloalkyl, cycloalkyl, aryl, heterocycle, halogen and heteroaryl each of which is optionally substituted with 1 or 2 substituents independently selected from R¹⁷;
q is 0, 1, 2, 3, 4, or 5;
and wherein all other variables are as defined herein.

In certain embodiments q is 0, 1, 2, or 3. In certain embodiments q is 0. In certain embodiments q is 1. In certain embodiments q is 2.

In certain embodiments R²⁹ is a bicyclic heterocycle;

In certain embodiments the KRAS Recognition Moiety is

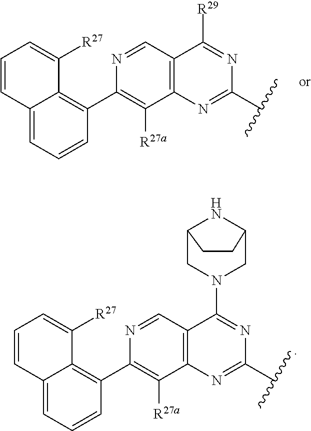

In certain embodiments the KRAS Recognition Moiety is

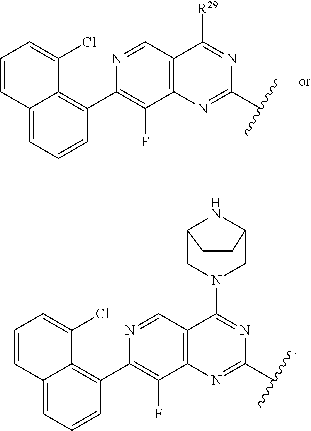

In certain embodiments R^{27a} is halogen.

In certain embodiments R^{27a} is haloalkyl.

In certain embodiments R^{27a} is alkyl.

In certain embodiments R^{27a} is hydrogen.

In certain embodiments R^{27b} is halogen.

In certain embodiments R^{27b} is haloalkyl.

In certain embodiments R^{27b} is alkyl.

In certain embodiments there is one R²⁷ and it is halogen.

In certain embodiments there is one R²⁷ and it is haloalkyl.

In certain embodiments there is one R²⁷ and it is alkyl.

In certain embodiments there is one R²⁷ and it is hydrogen.

In certain embodiments the KRAS Recognition Moiety is

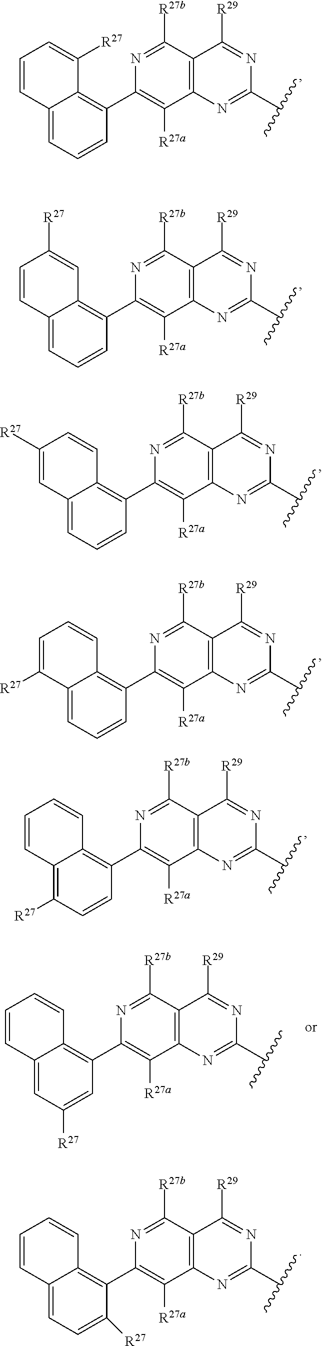

In certain embodiments the KRAS Recognition Moiety is

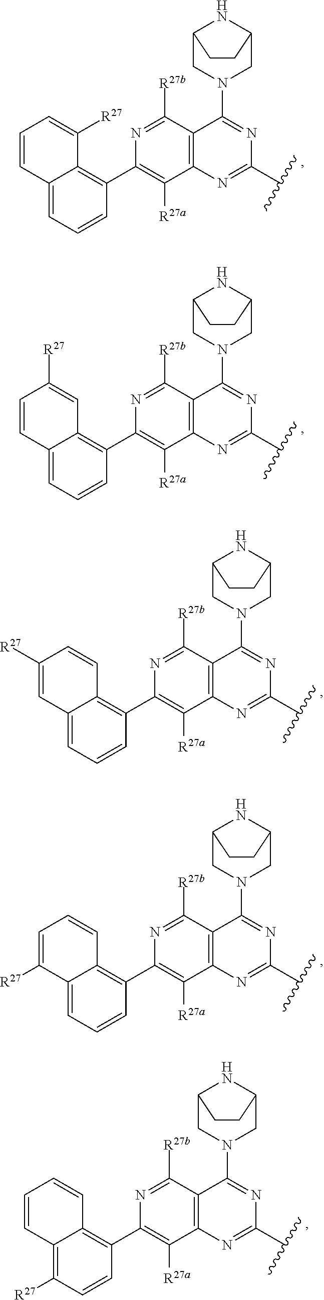

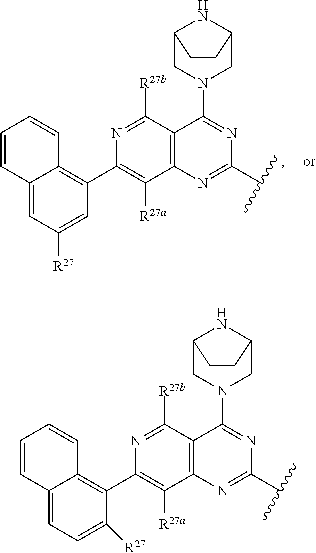

In certain embodiments the KRAS Recognition Moiety is adagrasib or a fragment or derivative thereof. For example, in certain embodiments, the KRAS Recognition Moiety is

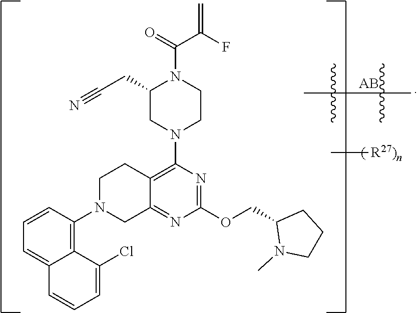

In certain embodiments the KRAS Recognition Moiety is MRTX1257 or a fragment or derivative thereof. For example, in certain embodiments, the KRAS Recognition Moiety is

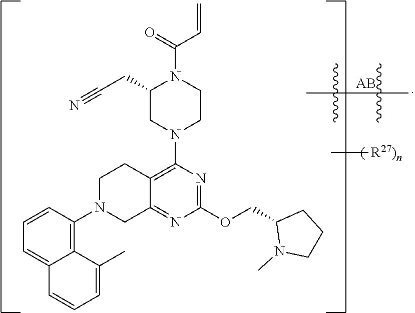

In certain embodiments the KRAS Recognition Moiety is MRTX1133 or a fragment or derivative thereof.

In certain embodiments the KRAS Recognition Moiety is sotorasib or a fragment or derivative thereof. For example, in certain embodiments, the KRAS Recognition Moiety is selected from:

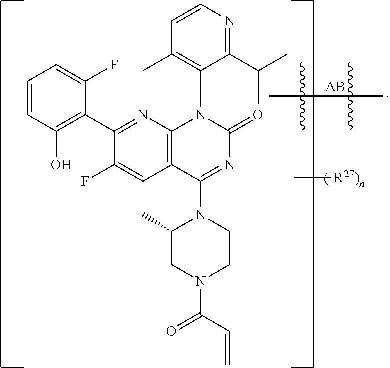

In certain embodiments the KRAS Recognition Moiety is ARS-1620 or a fragment or derivative thereof. For example, in certain embodiments, the KRAS Recognition Moiety is

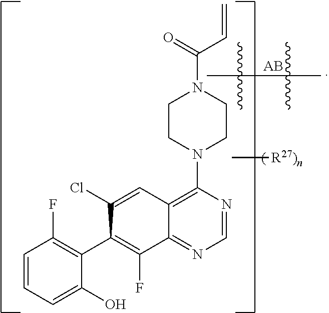

In certain embodiments the KRAS Recognition Moiety is BI-2852 or a fragment or derivative thereof. For example, in certain embodiments, the KRAS Recognition Moiety is selected from:

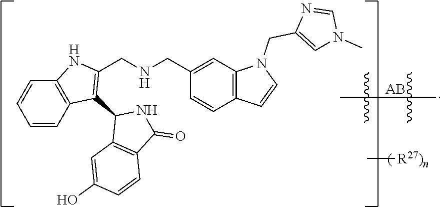

In certain embodiments the KRAS Recognition Moiety is KS-58 or a fragment or derivative thereof. For example, in certain embodiments, the KRAS Recognition Moiety is selected from:

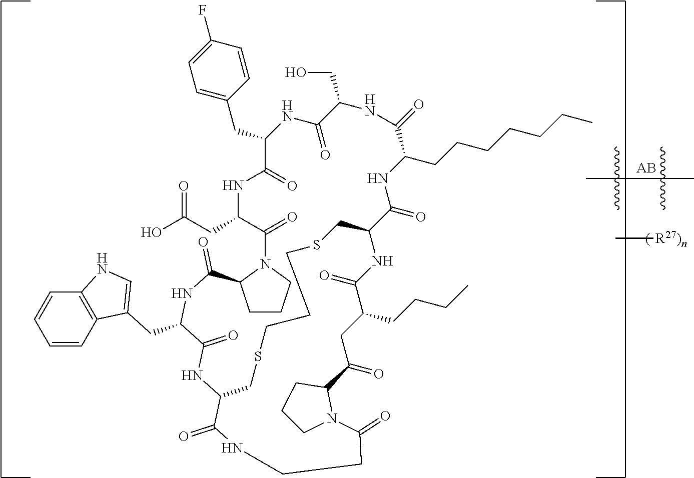

In certain embodiments the KRAS Recognition Moiety is Compound 12 from Ostrem et al. Nature 503(7477), 548-551 (2013) or a fragment or derivative thereof. For example, in certain embodiments, the KRAS Recognition Moiety is selected from:

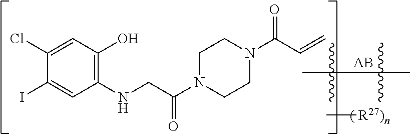

In certain embodiments the KRAS Recognition Moiety is ARS-853 or a fragment or derivative thereof. For example, in certain embodiments, the KRAS Recognition Moiety is selected from:

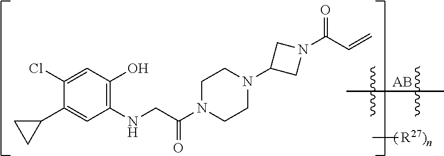

In certain embodiments the KRAS Recognition Moiety is ARS-1323 or a fragment or derivative thereof. For example, in certain embodiments, the KRAS Recognition Moiety is selected from:

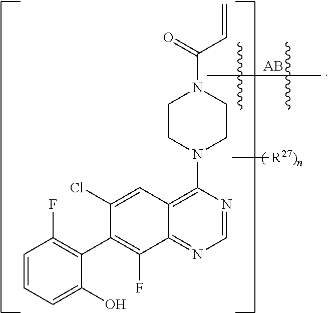

In certain embodiments the KRAS Recognition Moiety is ARS-107 or a fragment or derivative thereof. For example, in certain embodiments, the KRAS Recognition Moiety is selected from:

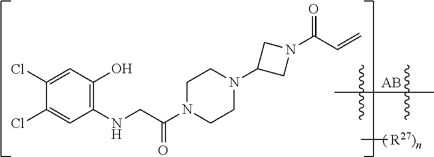

In certain embodiments the KRAS Recognition Moiety is ARS-917 or a fragment or derivative thereof. For example, in certain embodiments, the KRAS Recognition Moiety is selected from:

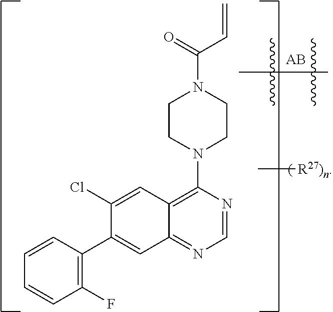

In certain embodiments the KRAS Recognition Moiety is (R)-4-(2-(dimethylamino)ethoxy)-N-((8-(6-methoxypyridin-2-yl)-2,3-dihydrobenzo[b][1,4]dioxin-2-yl)methyl)benzamide or a fragment or derivative thereof. For example, in certain embodiments, the KRAS Recognition Moiety is selected from:

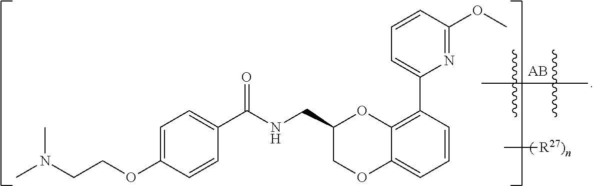

In certain embodiments the KRAS Recognition Moiety is (R)—N-((2,3-dihydrobenzo[b][1,4]dioxin-2-yl)methyl)-4-(2-(dimethylamino)ethoxy)benzamide or a fragment or derivative thereof. For example, in certain embodiments, the KRAS Recognition Moiety is selected from:

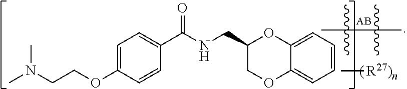

In certain embodiments the KRAS Recognition Moiety is N-(4-((dimethylamino)methyl)phenyl)-3′-methoxy-[1,1′-biphenyl]-4-amine or a fragment or derivative thereof. For example, in certain embodiments, the KRAS Recognition Moiety is selected from:

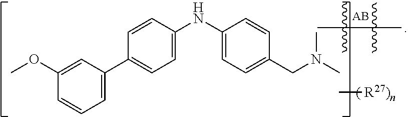

In certain embodiments the KRAS Recognition Moiety is (3S,3'S)-3,3′-(((hexane-1,6-diylbis(azanediyl))bis(methylene))bis(1H-indole-2,3-diyl))bis(5-hydroxyisoindolin-1-one) or a fragment or derivative thereof. For example, in certain embodiments, the KRAS Recognition Moiety is selected from:

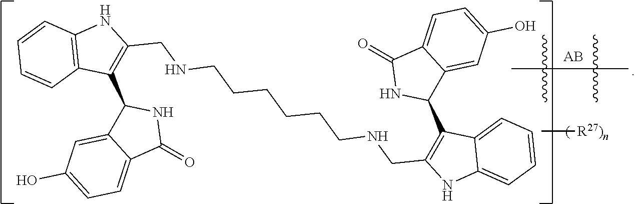

In certain embodiments the KRAS Recognition Moiety is (R)-7-(2,4-difluorophenyl)-3-(1-propionylpyrrolidin-3-yl)isoquinolin-1(4H)-one or a fragment or derivative thereof. For example, in certain embodiments, the KRAS Recognition Moiety is selected from:

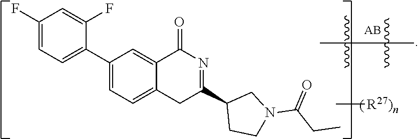

In certain embodiments the KRAS Recognition Moiety is 3-amino-1-(4-(6-chloro-8-fluoro-7-(5-methyl-1H-indazol-4-yl)quinazolin-4-yl)piperazin-1-yl)propan-1-one or a fragment or derivative thereof. For example, in certain embodiments, the KRAS Recognition Moiety is selected from:

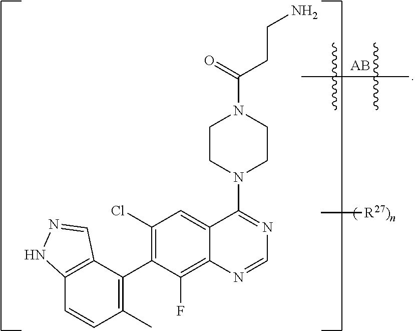

In certain embodiments the KRAS Recognition Moiety is (2S,3R)—N-(6-bromonaphthalen-2-yl)-3-hydroxy-1-propionylpyrrolidine-2-carboxamide or a fragment or derivative thereof. For example, in certain embodiments, the KRAS Recognition Moiety is selected from:

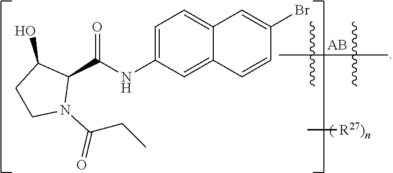

In certain embodiments the KRAS Recognition Moiety is N-{1-[(2,4-dichlorophenoxy)acetyl]piperidin-4-yl}ethanesulfonamide or a fragment or derivative thereof. For example, in certain embodiments, the KRAS Recognition Moiety is selected from:

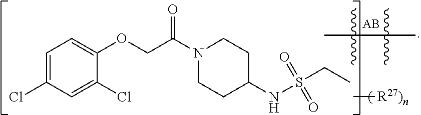

In certain embodiments the KRAS Recognition Moiety is (2R,4aR)-11-chloro-9-fluoro-10-(2-fluoro-6-hydroxyphenyl)-2,6-dimethyl-3-propionyl-2,3,4,4a-tetrahydro-1H-pyrazino[1′,2′:4,5]pyrazino[2,3-c]quinolin-5(6H)-one or a fragment or derivative thereof. For example, in certain embodiments, the KRAS Recognition Moiety is selected from:

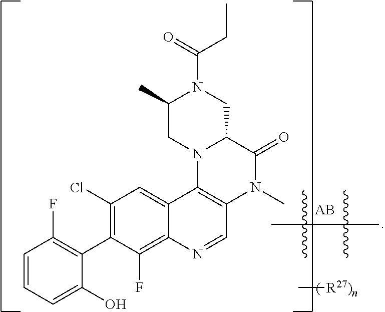

In certain embodiments the KRAS Recognition Moiety is (S)—N-(2-((1H-indol-3-yl)methyl)-1H-benzo[d]imidazol-5-yl)pyrrolidine-2-carboxamide or a fragment or derivative thereof. For example, in certain embodiments, the KRAS Recognition Moiety is selected from:

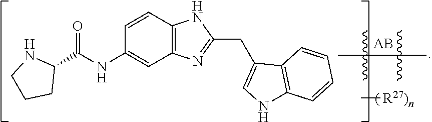

In certain embodiments the KRAS Recognition Moiety is ((2R,3S,4R,5R)-5-(2-amino-6-oxo-3,6-dihydro-9H-purin-9-yl)-3,4-dihydroxytetrahydrofuran-2-yl)methyl hydrogen ((N-(2-(2-chloroacetamido)ethyl)sulfamoyl)methyl)phosphonate or a fragment or derivative thereof. For example, in certain embodiments, the KRAS Recognition Moiety is selected from:

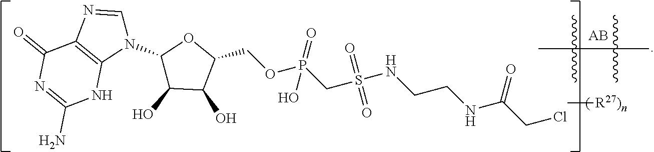

In certain embodiments the KRAS Recognition Moiety is (R)-7-(2-fluoro-6-hydroxyphenyl)-3-(1-propionylpyrrolidin-3-yl)-2,6-naphthyridin-1(4H)-one or a fragment or derivative thereof. For example, in certain embodiments, the KRAS Recognition Moiety is selected from:

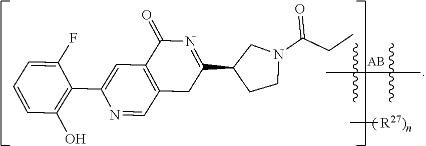

In certain embodiments the KRAS Recognition Moiety is (6aR,11bS)-6a-(1,4-dimethylpiperidin-4-yl)-6,6a,7,11b-tetrahydro-5H-indolo[2,3-c]isoquinolin-5-one or a fragment or derivative thereof. For example, in certain embodiments, the KRAS Recognition Moiety is selected from:

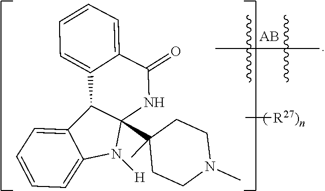

In certain embodiments the KRAS Recognition Moiety is 2-(5-bromo-3-(5-methoxy-1,2,3,4-tetrahydroisoquinoline-2-carbonyl)-1H-indol-1-yl)-N-(1-propionylazetidin-3-yl)acetamide or a fragment or derivative thereof. For example, in certain embodiments, the KRAS Recognition Moiety is selected from:

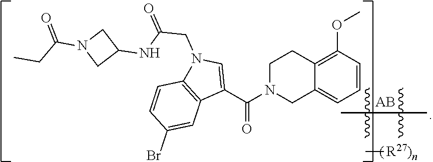

In certain embodiments the KRAS Recognition Moiety is 2-(4-bromo-2-(3-phenyl-2,5-dihydro-1H-pyrrole-1-carbonyl)phenoxy)-N-(1-propionylazetidin-3-yl)acetamide or a fragment or derivative thereof. For example, in certain embodiments, the KRAS Recognition Moiety is selected from:

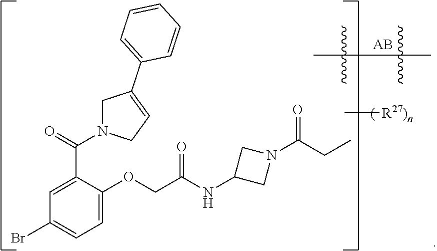

In certain embodiments the KRAS Recognition Moiety is N-(5-bromo-2-(2-oxo-2-((1-propionylazetidin-3-yl)amino)ethoxy)phenyl)-3-methylisoxazole-5-carboxamide or a fragment or derivative thereof. For example, in certain embodiments, the KRAS Recognition Moiety is selected from:

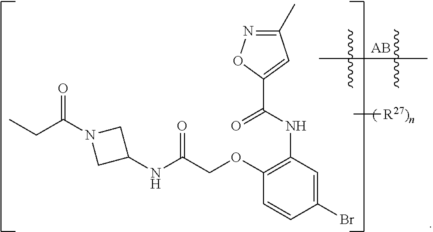

In certain embodiments the KRAS Recognition Moiety is N-(3-mercaptopropyl)-1-(4-methoxyphenyl)-5-(trifluoromethyl)-1H-pyrazole-4-carboxamide or a fragment or derivative thereof. For example, in certain embodiments, the KRAS Recognition Moiety is selected from:

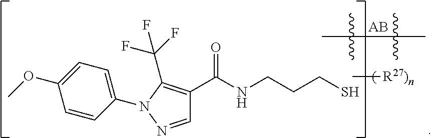

In certain embodiments the KRAS Recognition Moiety is (R)-1-(11-chloro-10-(5-methyl-2H-indazol-4-yl)-1,2,4a,5-tetrahydropyrazino[1′,2′:4,5][1,4]oxazino[2,3-c]quinolin-3(4H)-yl)propan-1-one or a fragment or derivative thereof. For example, in certain embodiments, the KRAS Recognition Moiety is selected from:

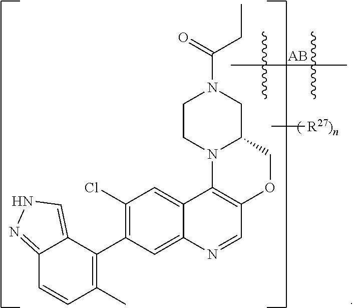

In certain embodiments the KRAS Recognition Moiety is 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((S)-2-fluoropropanoyl)piperazin-2-yl)acetonitrile or a fragment or derivative thereof. For example, in certain embodiments, the KRAS Recognition Moiety is selected from:

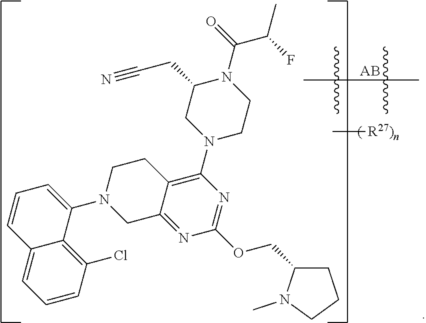

In certain embodiments the KRAS Recognition Moiety is 2-(4-(2-methyl-3,5-diphenylpyrazolo[1,5-a]pyrimidin-7-yl)piperazin-1-yl)ethan-1-ol or a fragment or derivative thereof. For example, in certain embodiments, the KRAS Recognition Moiety is selected from:

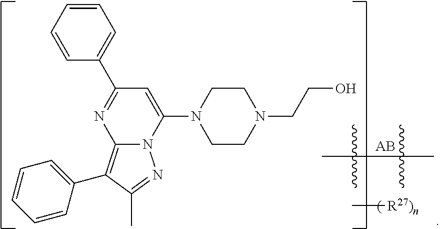

In certain embodiments the KRAS Recognition Moiety is 2E07 or a fragment or derivative thereof. For example, in certain embodiments, the KRAS Recognition Moiety is selected from:

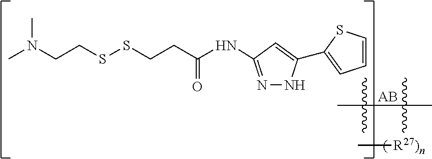

In certain embodiments the KRAS Recognition Moiety is 6H05 or a fragment or derivative thereof. For example, in certain embodiments, the KRAS Recognition Moiety is selected from:

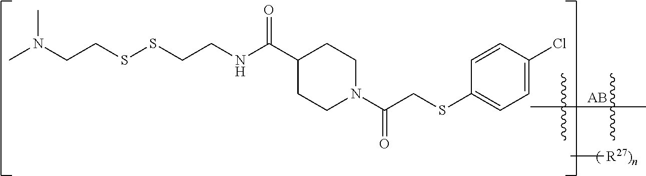

In certain embodiments the KRAS Recognition Moiety is an RNA that binds KRAS. The RNA can be a fragment, SiRNA, a sequence of naturally occurring RNA, a sequence of unnatural RNA, or a combination thereof. In certain embodiments the RNA binds a viral target (see, for example, the paper by Bader Alhatlani, “In silico identification of conserved cis-acting RNA elements in the SARS COV-2 genome” Future Virology 15(7) 409-417). In another embodiment the RNA binds a protein that mediates a non-viral disorder such as a cancer or a tumor (see, for example, the paper by Xiangping Liang, et. al., “RNA-based pharmacotherapy for tumors: From bench to clinic and back” Biomedicine and Pharmacotherapy Volume 125, 2020, 109997).

In certain embodiments the KRAS Recognition Moiety is a DNA that binds KRAS. The DNA can be a fragment, a sequence of naturally occurring DNA, a sequence of unnatural DNA, or a combination thereof (see, for example, the paper by Siddhesh D Patil, et al. “DNA-based therapeutics and DNA delivery systems: a comprehensive review” AAPS J. 2005 8; 7(1)).

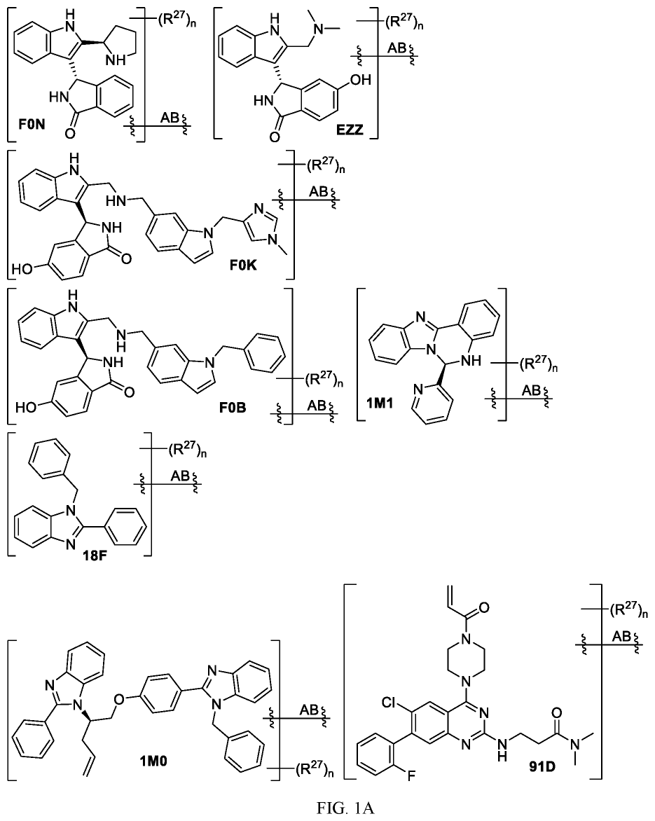

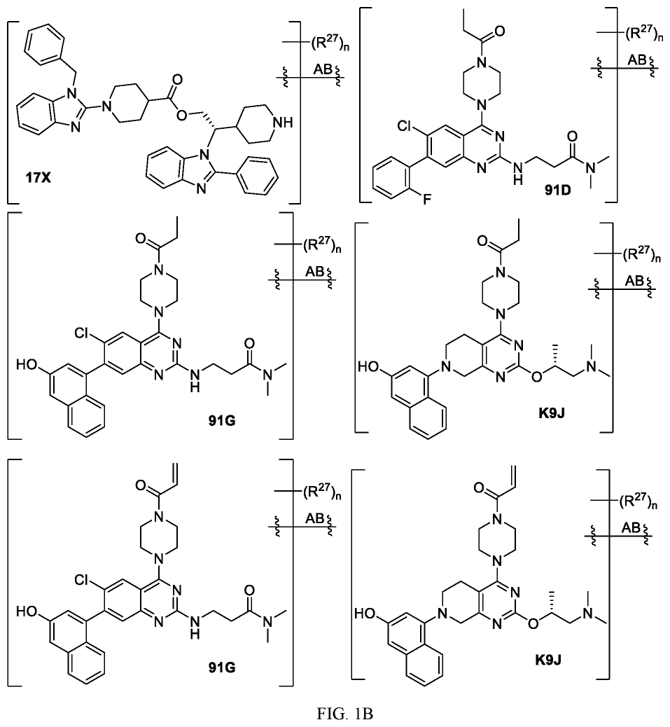

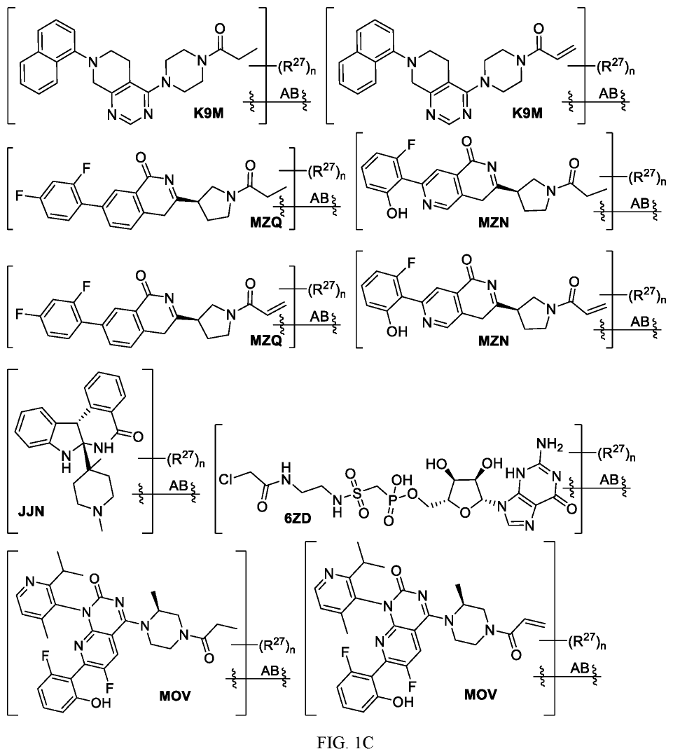

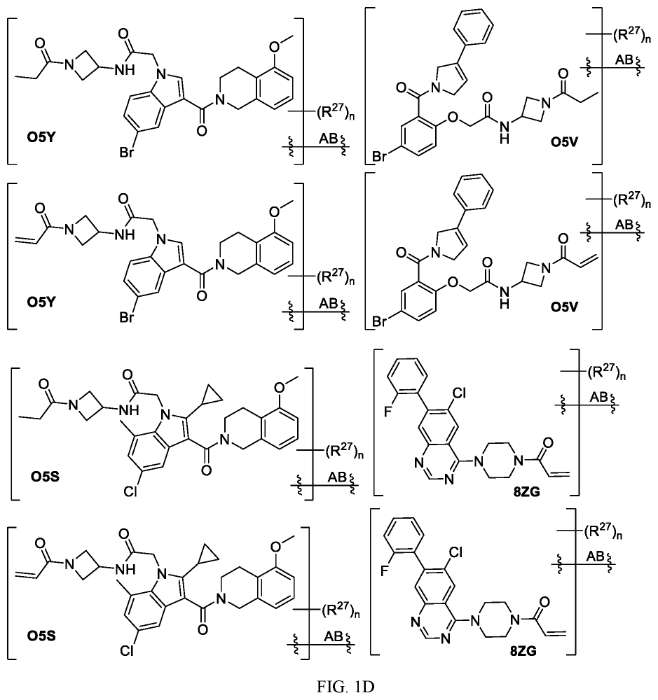

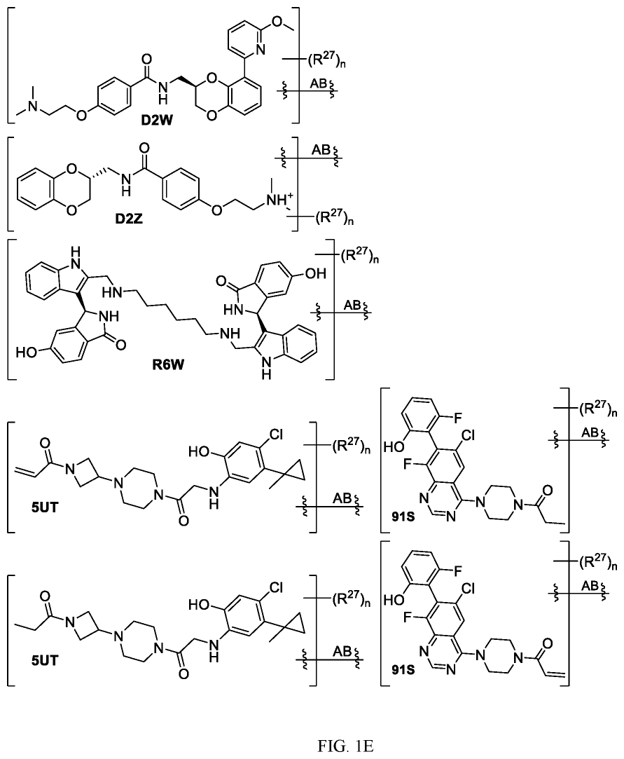

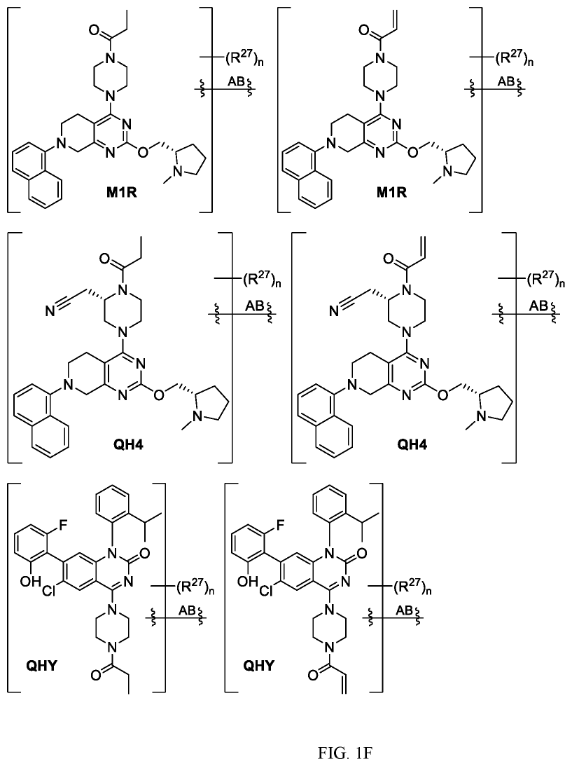

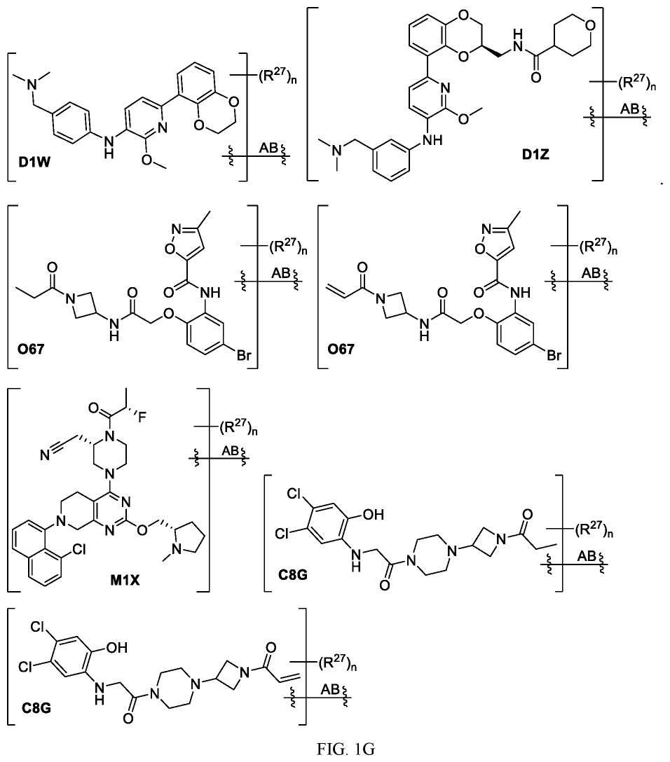

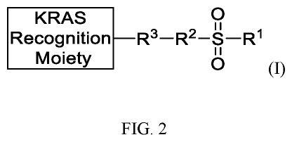

The Protein Data Bank website provides the crystal structure of KRAS searchable by 6N65 (Ou et la., “High resolution crystal structure of a KRAS promoter G-quadruplex reveals a dimer with extensive poly-A pi-stacking interactions for small-molecule recognition”, Nucleic Acids Res., 2020, 48: 5766-5776); as well as the crystal structure of KRAS bound to various compounds searchable by 6MNX, 6BOF, 6GJ6, 6GJ5, 6GJ8, 6GJ7, 6CU6, 4JV8, 4JV6, 4JVB, 4JVF, 5V9L, 5V90, 6N2K, 6N2J, 6TAM, 6TAN, 6QUV, 601M, 5ML3, 5KYK, 6P8W, 6P8Y, 6P8X, 6P8Z, 6BOV, 6BOY, 6FA2, 6FA1, 7ACA, 5F2E, 5V9U, 6UTO, 6USX, 6USZ, 6PGP, 6FA4, and 6FA3. Representative KRAS Targeting Ligands are provided in FIG. 1.

In certain embodiments the KRAS Recognition Moiety binds fewer than 80, 70, 60, 50, 40, 30, 20, 15, 10, or 5 endogenous proteins with a KD50 of 10 μM or less.

In certain embodiments the KRAS Recognition Moiety binds fewer than 80, 70, 60, 50, 40, 30, 20, 15, 10, or 5 endogenous proteins with a KD50 of 5 μM or less.

In certain embodiments the KRAS Recognition Moiety binds fewer than 80, 70, 60, 50, 40, 30, 20, 15, 10, or 5 endogenous proteins with a KD50 of 2 μM or less.

In certain embodiments the KRAS Recognition Moiety binds fewer than 80, 70, 60, 50, 40, 30, 20, 15, 10, or 5 endogenous proteins with a KD50 of 1 μM or less.

In certain embodiments the KRAS Recognition Moiety binds fewer than 80, 70, 60, 50, 40, 30, 20, 15, 10, or 5 endogenous proteins with a KD50 of 0.5 μM or less.

Exemplary Methods of Treatment of Diseases Mediated by KRAS

The present invention can be used to treat any disorder that is mediated by KRAS, for example mutant KRAS. Typically, the KRAS Recognition Moiety is a targeting ligand or portion of a targeting ligand that binds or is bound by KRAS, for example mutant KRAS. Nonlimiting examples of disorders that can be treated with a heteroaryl sulfonyl compound of the present invention include abnormal cellular proliferation disorders such as a cancer or a tumor.

In certain embodiments a heteroaryl sulfonyl compound of the present invention is used to treat a pancreatic cancer.

In certain embodiments a heteroaryl sulfonyl compound of the present invention is used to treat a colon cancer.

In certain embodiments a heteroaryl sulfonyl compound of the present invention is used to treat a lung cancer for example small cell lung cancer.

In certain embodiments, diseases associated with KRAS include, but are not limited to, cancer (for example, non-small cell lung cancer (NSCLC), pancreatic cancer, Adenocarcinoma, lung adenocarcinoma, mucinous adenoma, ductal carcinoma of the pancreas, colorectal carcinoma, breast cancer, Bile Duct Cancer, colon cancer, ovarian cancer, solid tumor, urothelial carcinoma, rectal cancer, gastrointestinal cancer, lymphoma, hematopoietic and lymphoid cell neoplasm, peritoneal carcinomatosis, head and neck squamous cell carcinoma, bone metastases, advanced malignant neoplasm, melanoma, braf mutated melanoma, liver metastases, NUT midline carcinoma, stomach cancer, oral cancer, endometrial cancer, leukemia, cholangiocarcinoma, acute myeloid leukemia, thyroid cancer, juvenile myelomonocytic leukemia (JMML), myelodysplastic syndromes (MDS), human hepatic vascular cavernomas), Oculoectodermal Syndrome, ocular disease (for example, Noonan Syndrome 3, Noonan-like CBL syndrome), Adnexal Diseases, Steatohepatitis, KRAS Gene Mutation (for example, Cardio-facio-cutaneous (CFC) syndrome, neurofibromatosis type I, and Legius syndrome), sporadic arteriovenous malformations, cardio-skeletal muscle disease associated with syndromes, autoimmune lymphoproliferative syndrome, epidermal nevus, costello syndrome (CS), inflammatory disease (for example, inflammatory and immune modulation specifically in KRAS-mutant cancers, Acute graft-versus-host disease), and autoimmune disease (for example, immune thrombocytopenia, recurrent HenochSchönlein purpura and intestinal Behget disease). (Nikolaev et al., “Somatic activating KRAS mutations in arteriovenous malformations of the brain”, New England Journal of Medicine (2018), 378(3), 250-261; Bollag et al., “Biochemical characterization of a novel KRAS insertion mutation from a human leukemia”, Journal of Biological Chemistry, 1996, 271(51), 32491-32494; Kim et al., “MicroRNA-mediated regulation of KRAS in cancer”, Journal of Hematology & Oncology (2014), 7, 84/1-84/8; Jancik et al., “Clinical relevance of KRAS in human cancers”, Journal of Biomedicine and Biotechnology (2010); McCormick, Frank, “KRAS as a Therapeutic Target”, Clinical Cancer Research, 2015, 21(8), 1797-1801; Gremer et al., “Germline KRAS Mutations Cause Aberrant Biochemical and Physical Properties Leading to Developmental Disorders”, Hum Mutat., 2011, January; 32(1): 33-43; Moritake et al., “Autoimmunity Including Intestinal Behçet Disease Bearing the KRAS Mutation in Lymphocytes: A Case Report”, Pediatrics, 2016, 137(3), :e 20152891; Niemeyer, Charlotte, “RAS diseases in children”, Hematologica, 2014, 99(11);)

Non-limiting examples of cancers that can be treated according to the present invention include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CVL), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In certain embodiments, the cancer is a hematopoietic cancer. In certain embodiments, the hematopoietic cancer is a lymphoma. In certain embodiments, the hematopoietic cancer is a leukemia. In certain embodiments, the leukemia is acute myelocytic leukemia (AML).

In certain embodiments, the proliferative disorder is a myeloproliferative neoplasm. In certain embodiments, the myeloproliferative neoplasm (MPN) is primary myelofibrosis (PMF).

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas, as described above herein. Additional examples of solid tumors include, but are not limited to, squamous cell carcinoma, colon cancer, breast cancer, prostate cancer, lung cancer, liver cancer, pancreatic cancer, and melanoma.

Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

Exemplary cancers which may be treated by the disclosed heteroaryl sulfonyl compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the disclosed heteroaryl sulfonyl compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

Pharmaceutical Compositions

A heteroaryl sulfonyl compound of the present invention or a pharmaceutically acceptable salt, solvate or prodrug thereof as disclosed herein can be administered as a neat chemical, but is more typically administered as a pharmaceutical composition that includes an effective amount for a host, typically a human, in need of such treatment to treat a disorder mediated by the target extracellular protein, as described herein or otherwise well-known for that extracellular protein.

The heteroaryl sulfonyl compounds of the present invention can be administered in any manner that allows the heteroaryl sulfonyl compound to covalently modify KRAS, for example mutant KRAS. As such, examples of methods to deliver a heteroaryl sulfonyl compound of the present invention include, but are not limited to, oral, intravenous, sublingual, subcutaneous, parenteral, buccal, rectal, intra-aortal, intracranial, subdermal, transdermal, controlled drug delivery, intramuscular, or transnasal, or by other means, in dosage unit formulations containing one or more conventional pharmaceutically acceptable carriers, as appropriate. In certain embodiments, a heteroaryl sulfonyl compound of the present invention is provided in a liquid dosage form, a solid dosage form, a gel, particle, etc.

In certain embodiments the heteroaryl sulfonyl compound of the present invention is administered subcutaneously. Typically, the heteroaryl sulfonyl compound will be formulated in a liquid dosage form for subcutaneous injection, such as a buffered solution. Non-limiting examples of solutions for subcutaneous injection include phosphate buffered solution and saline buffered solution. In certain embodiments the solution is buffered with multiple salts.

In certain embodiments the heteroaryl sulfonyl compound of the present invention is administered intravenously. Typically, if administered intravenously, the heteroaryl sulfonyl compound will be formulated in a liquid dosage form for intravenous injection, such as a buffered solution. Non-limiting examples of solutions for intravenous injection include phosphate buffered solution and saline buffered solution. In certain embodiments the solution is buffered with multiple salts.

Therefore, the disclosure provides pharmaceutical compositions comprising an effective amount of heteroaryl sulfonyl compound or its pharmaceutically acceptable salt together with at least one pharmaceutically acceptable carrier for any appropriate use thereof. The pharmaceutical composition may contain a heteroaryl sulfonyl compound or salt as the only active agent, or, in an alternative embodiment, the heteroaryl sulfonyl compound and at least one additional active agent.

The term “pharmaceutically acceptable salt” as used herein refers to a salt of the described heteroaryl sulfonyl compound which is, within the scope of sound medical judgment, suitable for administration to a host such as a human without undue toxicity, irritation, allergic response, and the like, commensurate with a reasonable benefit/risk ratio, and effective for its intended use. Thus, the term “pharmaceutically acceptable salt” refers to the relatively non-toxic, inorganic and organic acid addition salts of the presently disclosed heteroaryl sulfonyl compounds. These salts can be prepared during the final isolation and purification of the heteroaryl sulfonyl compounds or by separately reacting the purified heteroaryl sulfonyl compound in its free form with a suitable organic or inorganic acid and then isolating the salt thus formed. Basic heteroaryl sulfonyl compounds are capable of forming a wide variety of different salts with various inorganic and organic acids. Acid addition salts of the basic heteroaryl sulfonyl compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents. Pharmaceutically acceptable base addition salts may be formed with a metal or amine, such as alkali and alkaline earth metal hydroxide, or an organic amine. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic heteroaryl sulfonyl compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Any dosage form can be used that achieves the desired results. In certain embodiments the pharmaceutical composition is in a dosage form that contains from about 0.1 mg to about 1500 mg, from about 10 mg to about 1000 mg, from about 100 mg to about 800 mg, or from about 200 mg to about 600 mg of the active heteroaryl sulfonyl compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 25, 50, 100, 200, 250, 300, 400, 500, 600, 700, or 750 mg of active heteroaryl sulfonyl compound, or its salt.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

In some embodiments, heteroaryl sulfonyl compounds disclosed herein or used as described are administered once a day (QD), twice a day (BID), or three times a day (TID). In some embodiments, heteroaryl sulfonyl compounds disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In certain embodiments the heteroaryl sulfonyl compound of the present invention is administered once a day, twice a day, three times a day, or four times a day.

The pharmaceutical composition may be formulated as any pharmaceutically useful form, e.g., a pill, capsule, tablet, an injection or infusion solution, a syrup, an inhalation formulation, a suppository, a buccal or sublingual formulation, a parenteral formulation, or in a medical device. Some dosage forms, such as tablets and capsules, can be subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the heteroaryl sulfonyl compound is sufficient to provide a practical quantity of material for administration per unit dose of the heteroaryl sulfonyl compound. If provided as in a liquid, it can be a solution or a suspension.

Representative carriers include phosphate buffered saline, water, solvent(s), diluents, pH modifying agents, preservatives, antioxidants, suspending agents, wetting agent, viscosity agents, tonicity agents, stabilizing agents, and combinations thereof. In some embodiments, the carrier is an aqueous carrier. Examples of aqueous carries include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, Provisc® diluted with PBS, Krebs buffer, Dulbecco's PBS, normal PBS, sodium hyaluronate solution (HA, 5 mg/mL in PBS), citrate buffer, simulated body fluids, plasma platelet concentrate and tissue culture medium or an aqueous solution or suspension comprising an organic solvent. Acceptable solutions include, for example, water, Ringer's solution and isotonic sodium chloride solutions. The formulation may also be a sterile solution, suspension, or emulsion in a non-toxic diluent or solvent such as 1,3-butanediol.

Viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextin, polysaccharides, polyacrylamide, polyvinyl alcohol (including partially hydrolyzed polyvinyl acetate), polyvinyl acetate, derivatives thereof and mixtures thereof.

Solutions, suspensions, or emulsions for administration may be buffered with an effective amount necessary to maintain a pH suitable for the selected administration. Suitable buffers are well known by those skilled in the art. Some examples of useful buffers are acetate, borate, carbonate, citrate, and phosphate buffers. Solutions, suspensions, or emulsions for topical, for example, ocular administration may also contain one or more tonicity agents to adjust the isotonic range of the formulation. Suitable tonicity agents are well known in the art. Some examples include glycerin, mannitol, sorbitol, sodium chloride, and other electrolytes.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the heteroaryl sulfonyl compound of the present invention.

The pharmaceutical compositions/combinations can be formulated for oral administration. These compositions can contain any amount of active heteroaryl sulfonyl compound that achieves the desired result, for example between 0.1 and 99 weight % (wt. %) of the heteroaryl sulfonyl compound and usually at least about 1 wt. % of the heteroaryl sulfonyl compound. Some embodiments contain from about 25 wt. % to about 50 wt. % or from about 5 wt. % to about 75 wt. % of the heteroaryl sulfonyl compound. Enteric coated oral tablets may also be used to enhance bioavailability of the heteroaryl sulfonyl compounds for an oral route of administration.

Formulations suitable for rectal administration are typically presented as unit dose suppositories. These may be prepared by admixing the active heteroaryl sulfonyl compound with one or more conventional solid carriers, for example, cocoa butter, and then shaping the resulting mixture.

Processes of Manufacture:

The heteroaryl sulfonyl compounds of the present invention can be manufactured according to routes described in the Working Examples below or as otherwise known in the patent or scientific literature and if appropriate supported by the knowledge of the ordinary worker or common general knowledge.

Some of the carbons in the heteroaryl sulfonyl compounds described herein are drawn with designated stereochemistry. Other carbons are drawn without stereochemical designation. When drawn without designated stereochemistry, that carbon can be in any desired stereochemical configuration that achieves the desired purpose. One skilled in the art will recognize that pure enantiomers, enantiomerically enriched heteroaryl sulfonyl compounds, racemates and diastereomers can be prepared by methods known in the art as guided by the information provided herein. Examples of methods to obtain optically active materials include at least the following:
i) chiral liquid chromatography—a technique whereby diastereomers are separated in a liquid mobile phase by virtue of their differing interactions with a stationary phase (including vial chiral HPLC). The stationary phase can be made of chiral material or the mobile phase can contain an additional chiral material to provoke the differing interactions;
ii) non-chiral chromatography of diastereomers-Often diastereomers can be separated using normal non-chiral column conditions;
iii) chiral gas chromatography—a technique whereby the racemate is volatilized and enantiomers are separated by virtue of their differing interactions in the gaseous mobile phase with a column containing a fixed non-racemic chiral adsorbent phase;
iv) simultaneous crystallization—a technique whereby the individual diastereomers are separately crystallized from a solution;
v) enzymatic resolutions—a technique whereby partial or complete separation of diastereomers are separated by virtue of differing rates of reaction with an enzyme;
vi) chemical asymmetric synthesis—a synthetic technique whereby the desired diastereomer is synthesized from an achiral precursor under conditions that produce asymmetry (i.e. chirality) in the product, which may be achieved by chiral catalysts or chiral auxiliaries;
vii) diastereomer separations—a technique whereby a racemic heteroaryl sulfonyl compound is reacted with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer; and
viii) extraction with chiral solvents—a technique whereby diastereomers are separated by virtue of preferential dissolution of one over the others in a particular chiral solvent.

### Synthesis of Compound 101

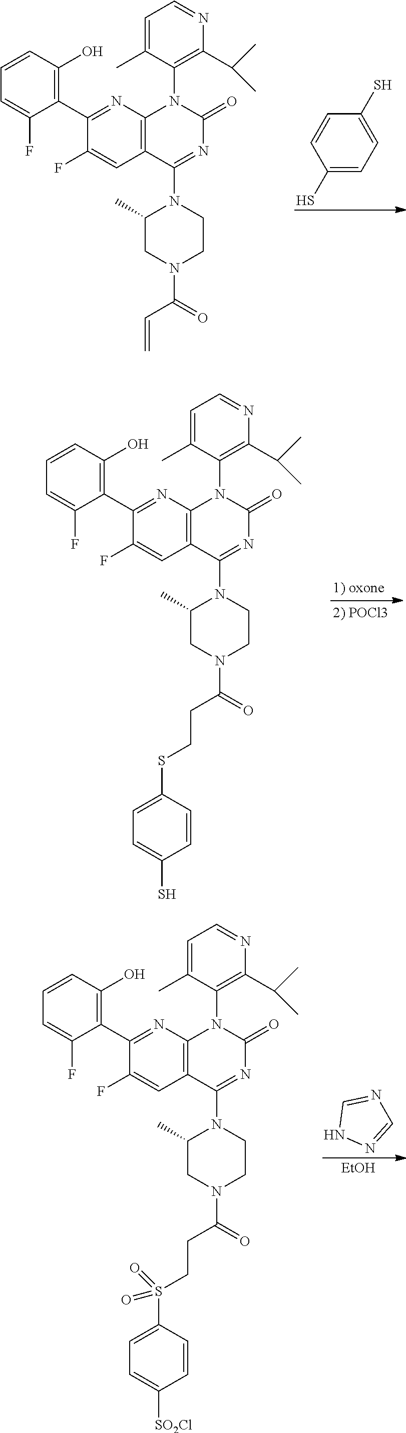 [SMILES: C=CC(=O)N1CCN(c2nc(=O)n(-c3c(C)ccnc3C(C)C)c3nc(-c4c(O)cccc4F)c(F)cc23)[C@@H](C)C1 ; Sc1ccc(S)cc1 ; "EtOH" ; Cc1ccnc(C(C)C)c1-n1c(=O)nc(N2CCN(C(=O)CCSc3ccc(S)cc3)C[C@@H]2C)c2cc(F)c(-c3c(O)cccc3F)nc21 ; Cc1ccnc(C(C)C)c1-n1c(=O)nc(N2CCN(C(=O)CCS(=O)(=O)c3ccc(S(=O)(=O)Cl)cc3)C[C@@H]2C)c2cc(F)c(-c3c(O)cccc3F)nc21 ; c1nc[nH]n1]

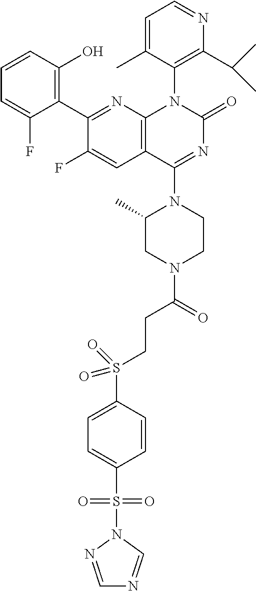 [SMILES: Cc1ccnc(C(C)C)c1-n1c(=O)nc(N2CCN(C(=O)CCS(=O)(=O)c3ccc(S(=O)(=O)n4cncn4)cc3)C[C@@H]2C)c2cc(F)c(-c3c(O)cccc3F)nc21]

### Synthesis of Compound 102

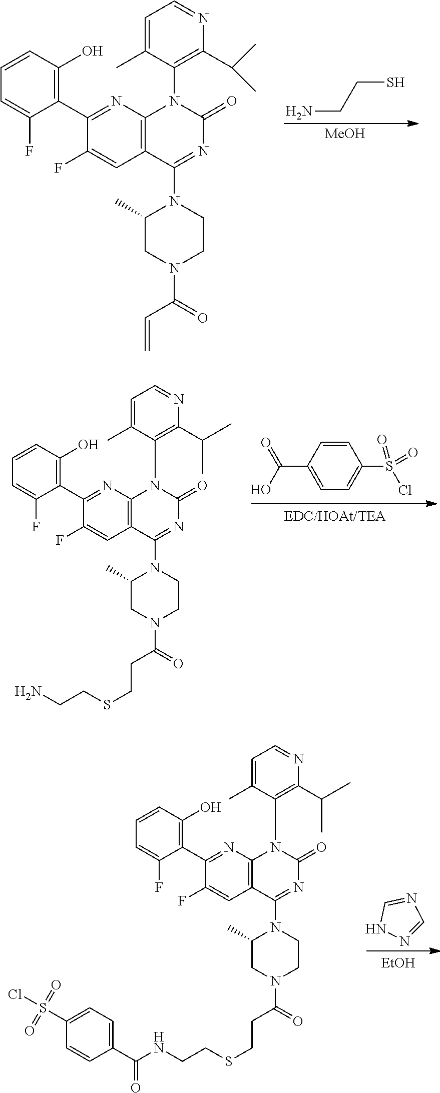

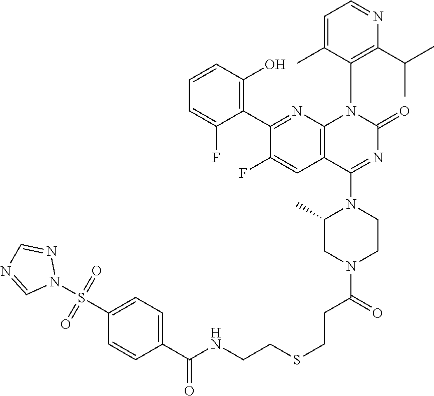 [SMILES: Cc1ccnc(C(C)C)c1-n1c(=O)nc(N2CCN(C(=O)CCSCCNC(=O)c3ccc(S(=O)(=O)n4cncn4)cc3)C[C@@H]2C)c2cc(F)c(-c3c(O)cccc3F)nc21]

### Synthesis of Compound 103

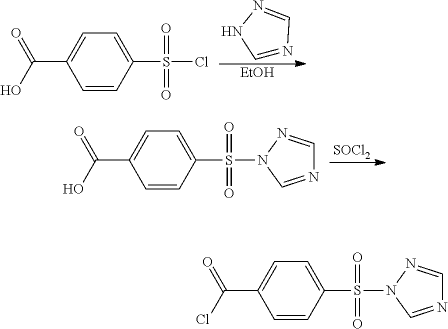

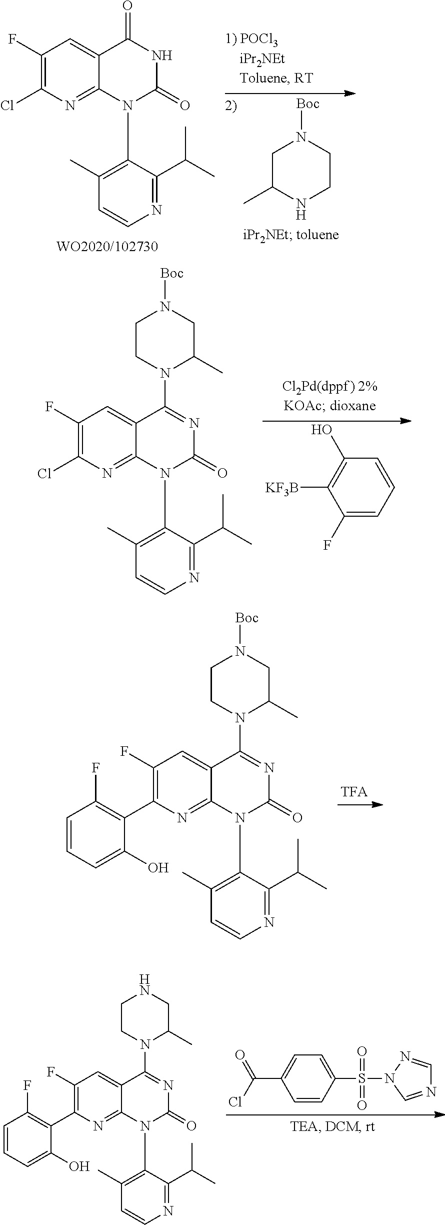

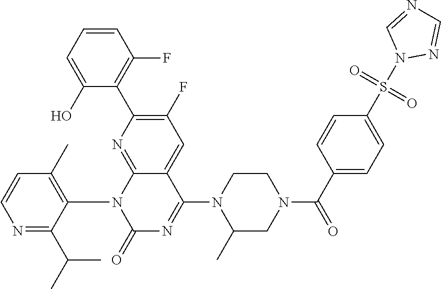 [SMILES: Cc1ccnc(C(C)C)c1-n1c(=O)nc(N2CCN(C(=O)c3ccc(S(=O)(=O)n4cncn4)cc3)CC2C)c2cc(F)c(-c3c(O)cccc3F)nc21]

### Synthesis of Compound 104

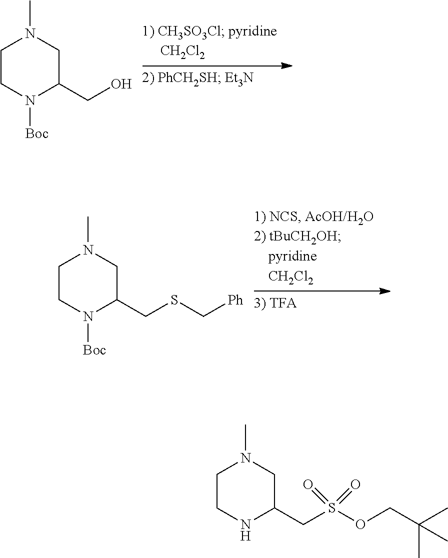 [SMILES: CN1CCN(C(=O)OC(C)(C)C)C(CO)C1 ; CN1CCN(C(=O)OC(C)(C)C)C(CSCc2ccccc2)C1 ; CN1CCNC(CS(=O)(=O)OCC(C)(C)C)C1]

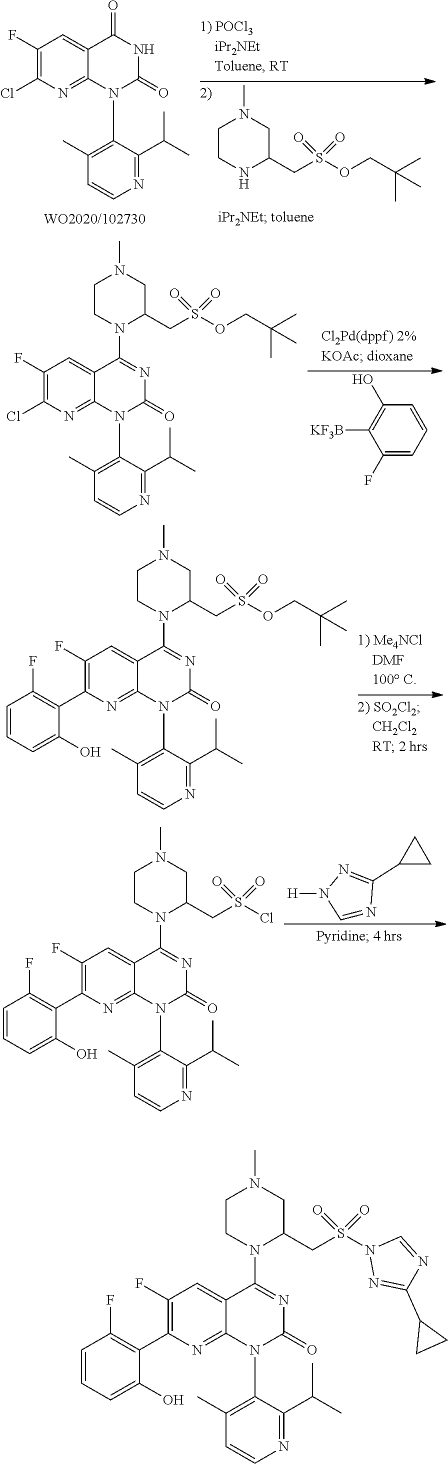

### Synthesis of Compound 105

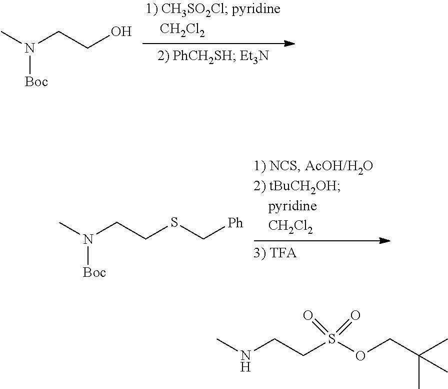 [SMILES: CN(CCO)C(=O)OC(C)(C)C ; CN(CCSCc1ccccc1)C(=O)OC(C)(C)C ; CNCCS(=O)(=O)OCC(C)(C)C]

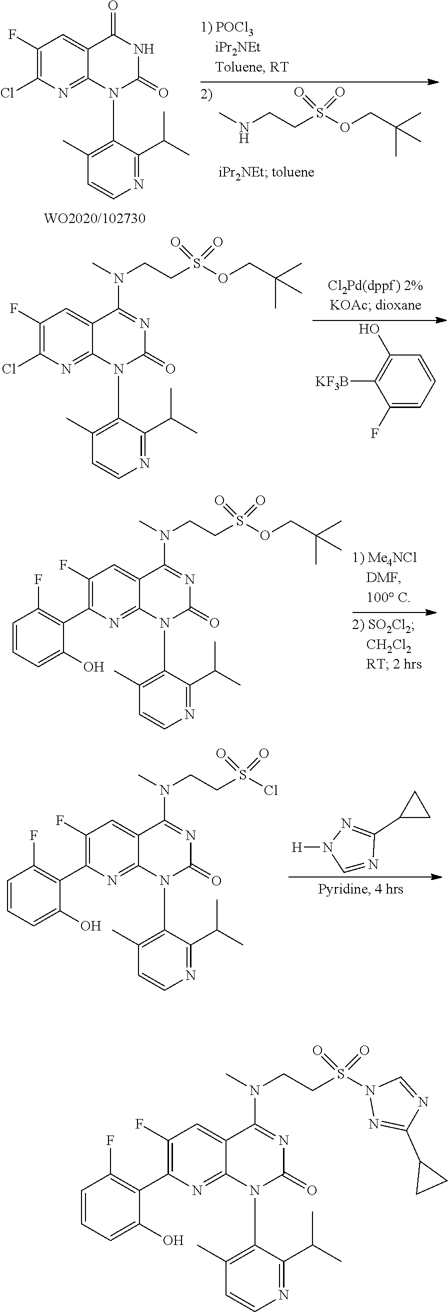

### Synthesis of Compound 106

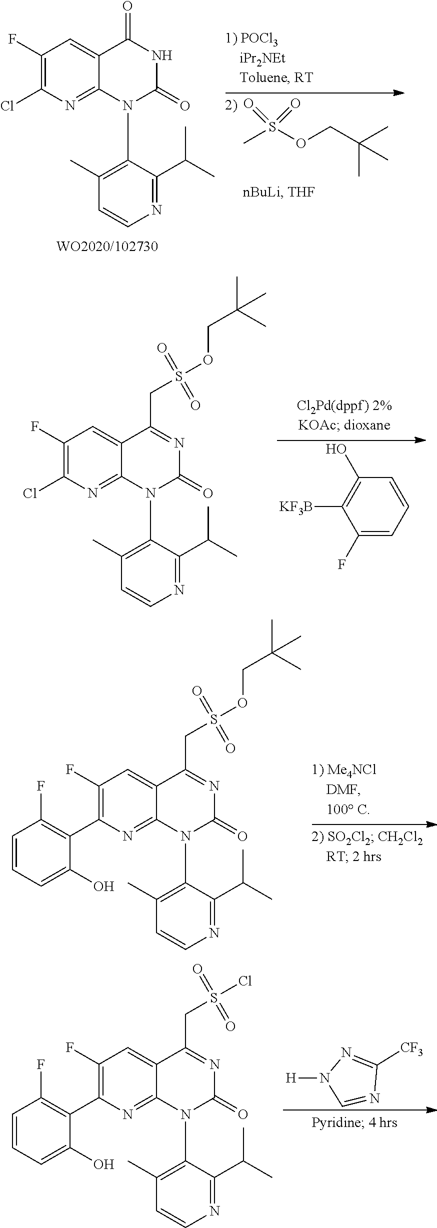

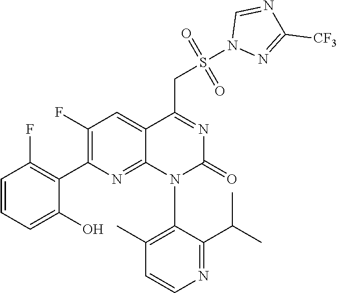 [SMILES: Cc1ccnc(C(C)C)c1-n1c(=O)nc(CS(=O)(=O)n2cnc(C(F)(F)F)n2)c2cc(F)c(-c3c(O)cccc3F)nc21]

### Synthesis of Compound 107

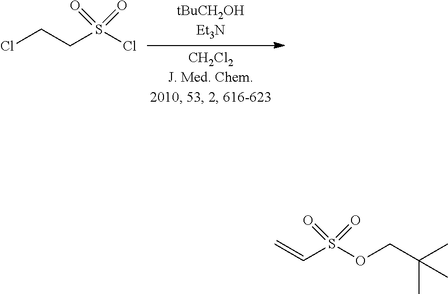

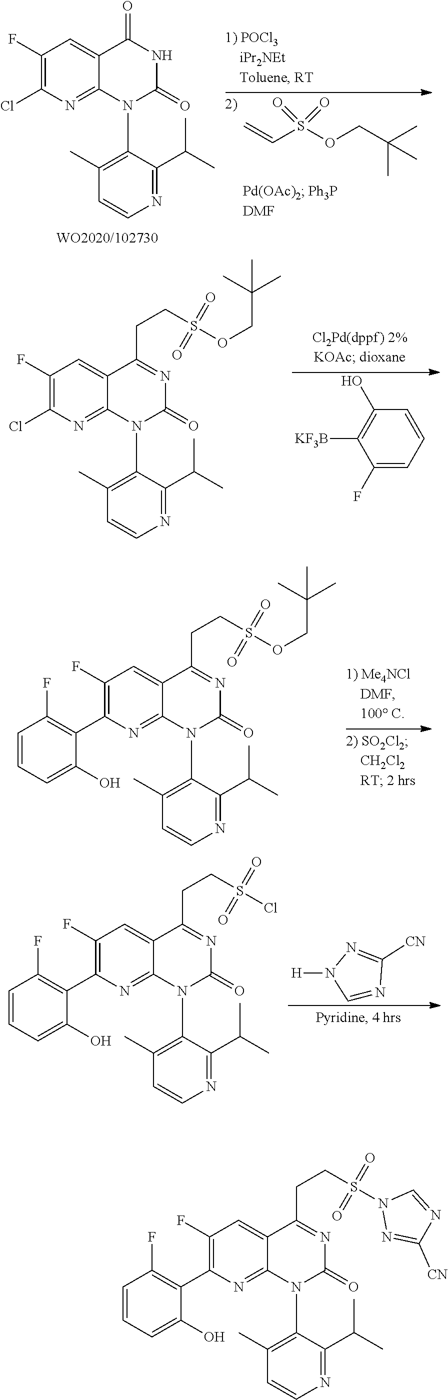

### Synthesis of Compound 108

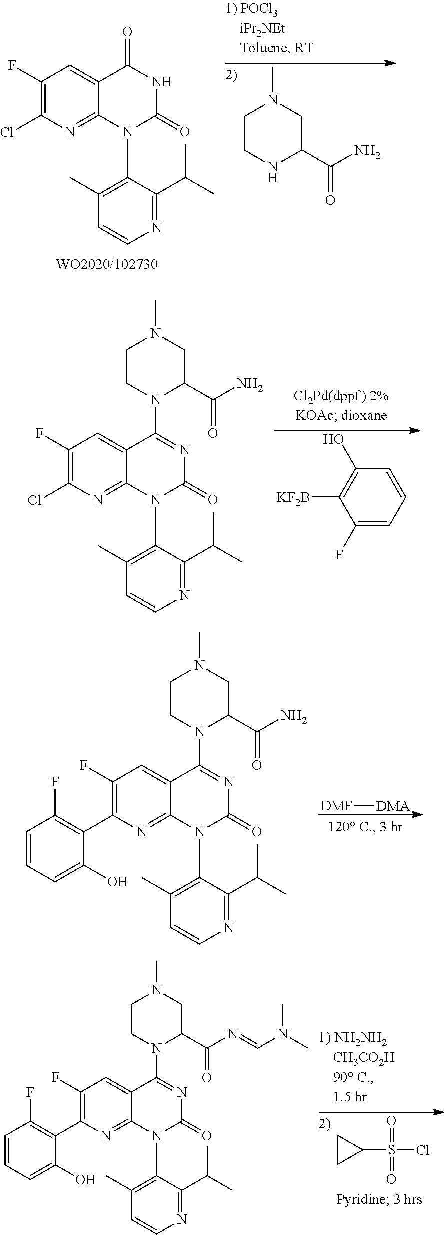

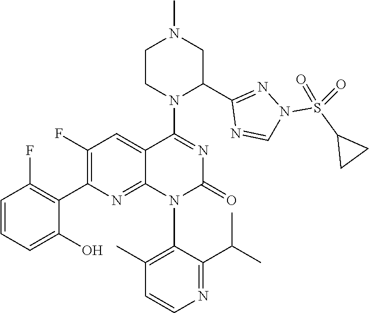 [SMILES: Cc1ccnc(C(C)C)c1-n1c(=O)nc(N2CCN(C)CC2c2ncn(S(=O)(=O)C3CC3)n2)c2cc(F)c(-c3c(O)cccc3F)nc21]

### Synthesis of Compound 109

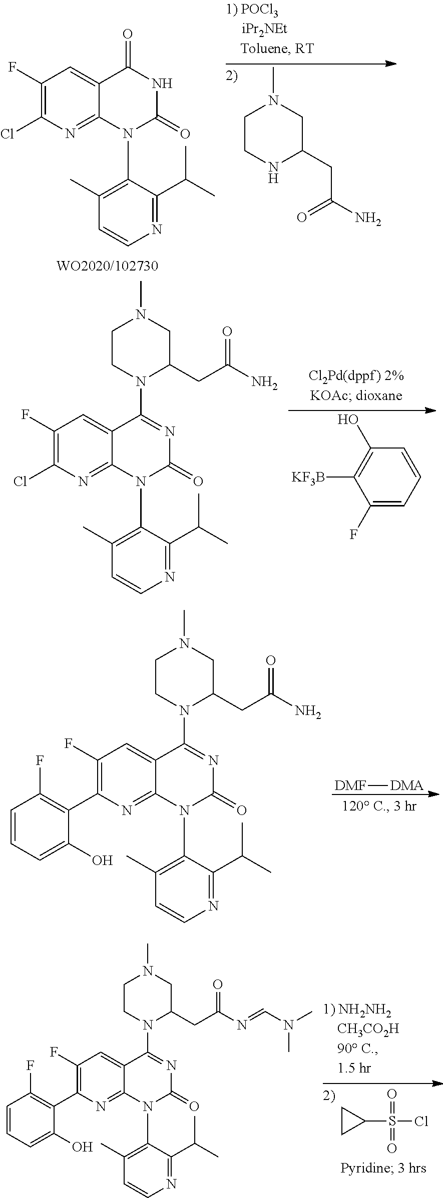

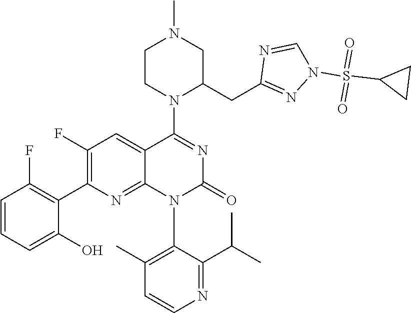 [SMILES: Cc1ccnc(C(C)C)c1-n1c(=O)nc(N2CCN(C)CC2Cc2ncn(S(=O)(=O)C3CC3)n2)c2cc(F)c(-c3c(O)cccc3F)nc21]

### Synthesis of Compound 110

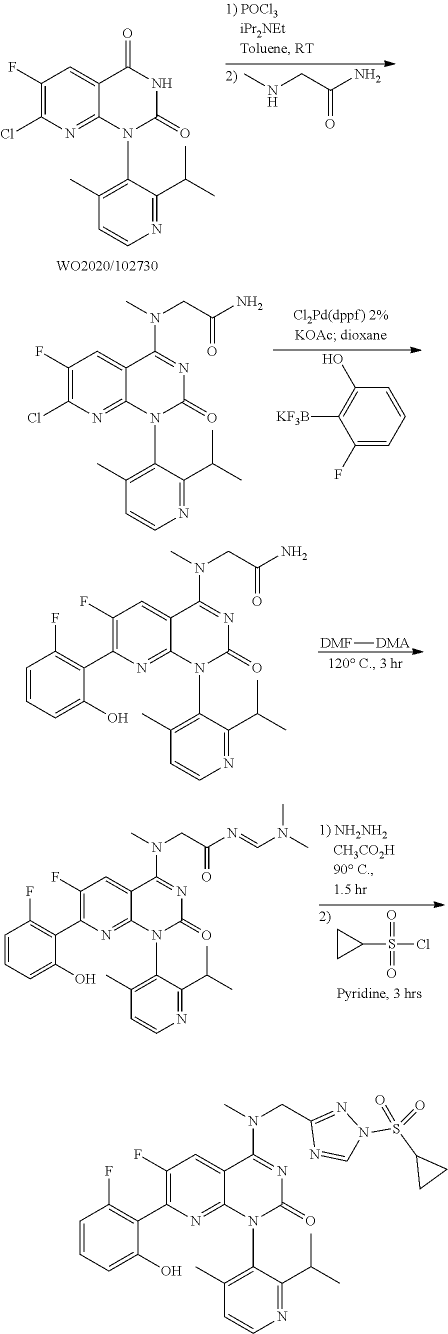

### Synthesis of Compound 111

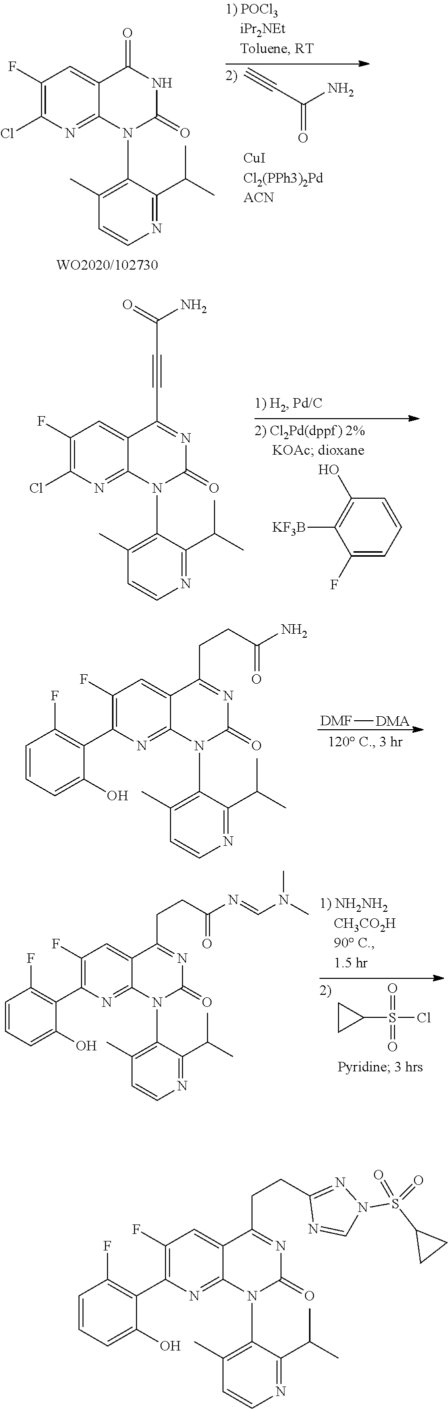

### Synthesis of Compound 112

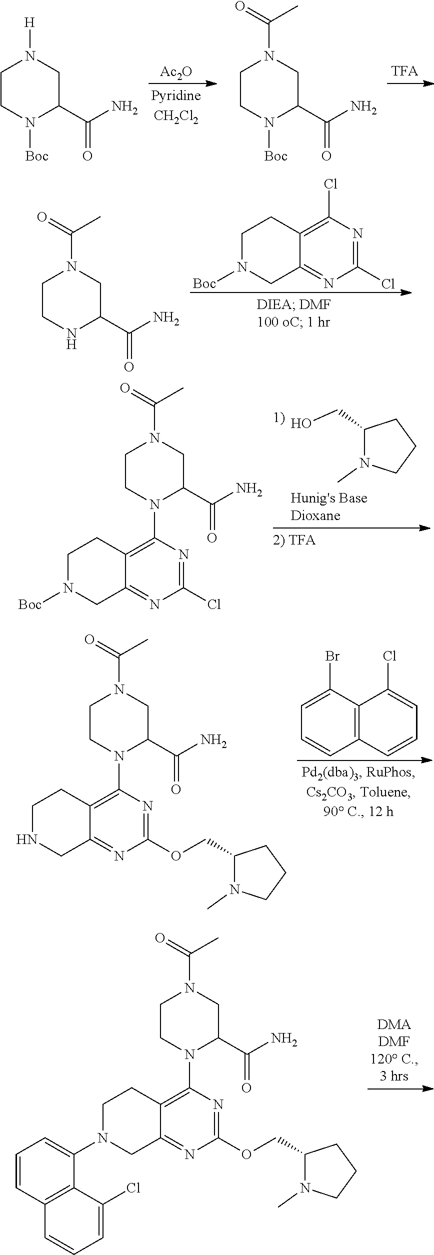

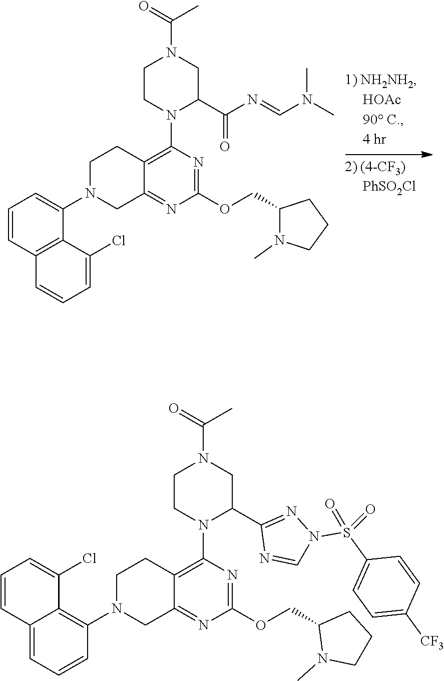 [SMILES: CC(=O)N1CCN(c2nc(OC[C@@H]3CCCN3C)nc3c2CCN(c2cccc4cccc(Cl)c24)C3)C(C(=O)/N=C/N(C)C)C1 ; CC(=O)N1CCN(c2nc(OC[C@@H]3CCCN3C)nc3c2CCN(c2cccc4cccc(Cl)c24)C3)C(c2ncn(S(=O)(=O)c3ccc(C(F)(F)F)cc3)n2)C1]

### Synthesis of Compound 113

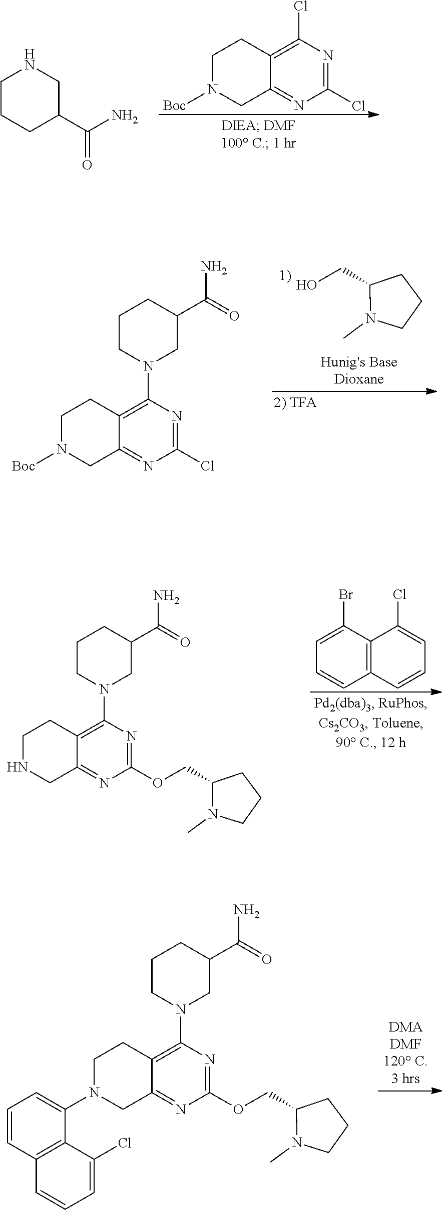

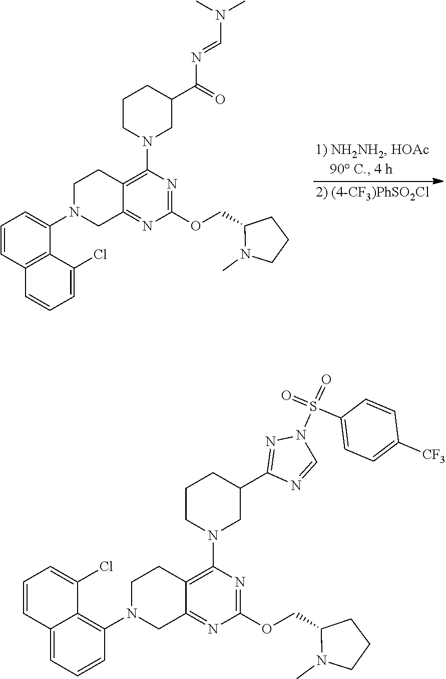 [SMILES: CN(C)/C=N/C(=O)C1CCCN(c2nc(OC[C@@H]3CCCN3C)nc3c2CCN(c2cccc4cccc(Cl)c24)C3)C1 ; CN1CCC[C@H]1COc1nc2c(c(N3CCCC(c4ncn(S(=O)(=O)c5ccc(C(F)(F)F)cc5)n4)C3)n1)CCN(c1cccc3cccc(Cl)c13)C2]

### Synthesis of Compound 114

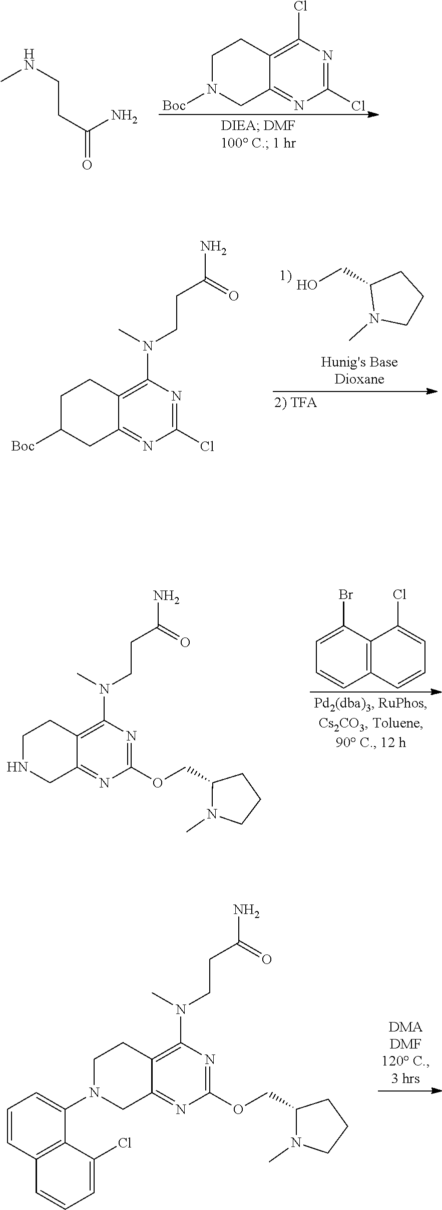

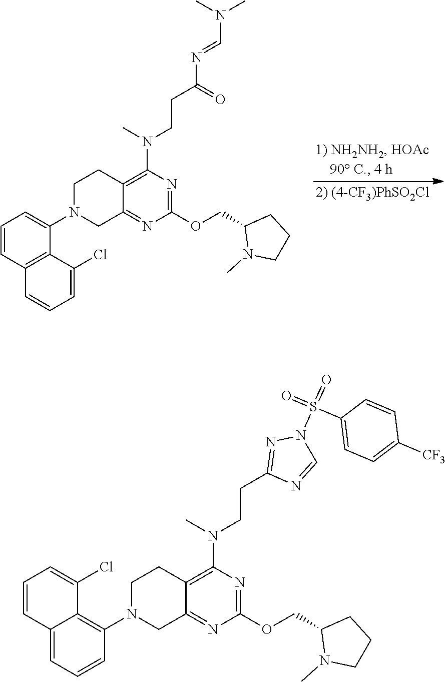 [SMILES: CN(C)/C=N/C(=O)CCN(C)c1nc(OC[C@@H]2CCCN2C)nc2c1CCN(c1cccc3cccc(Cl)c13)C2 ; CN(CCc1ncn(S(=O)(=O)c2ccc(C(F)(F)F)cc2)n1)c1nc(OC[C@@H]2CCCN2C)nc2c1CCN(c1cccc3cccc(Cl)c13)C2]

### Synthesis of Compound 115

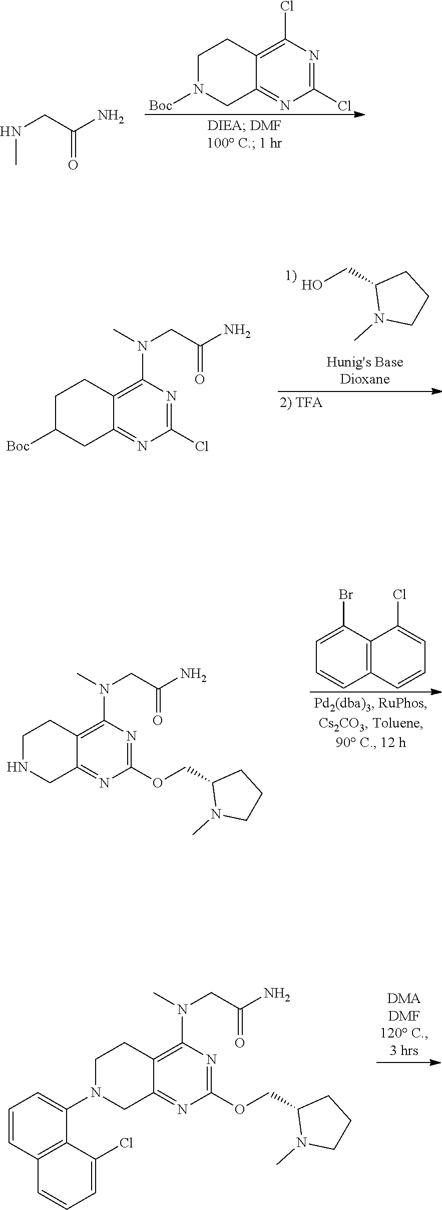

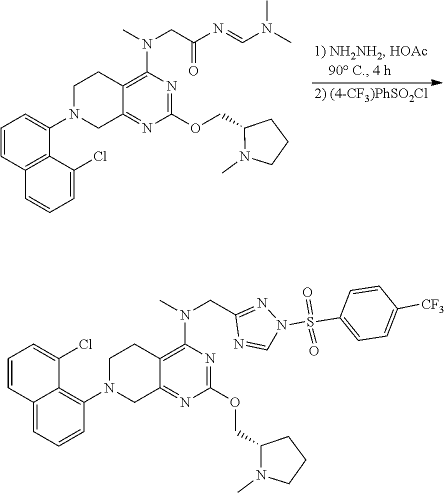 [SMILES: CN(C)/C=N/C(=O)CN(C)c1nc(OC[C@@H]2CCCN2C)nc2c1CCN(c1cccc3cccc(Cl)c13)C2 ; CN(Cc1ncn(S(=O)(=O)c2ccc(C(F)(F)F)cc2)n1)c1nc(OC[C@@H]2CCCN2C)nc2c1CCN(c1cccc3cccc(Cl)c13)C2]

### Synthesis of Compound 116

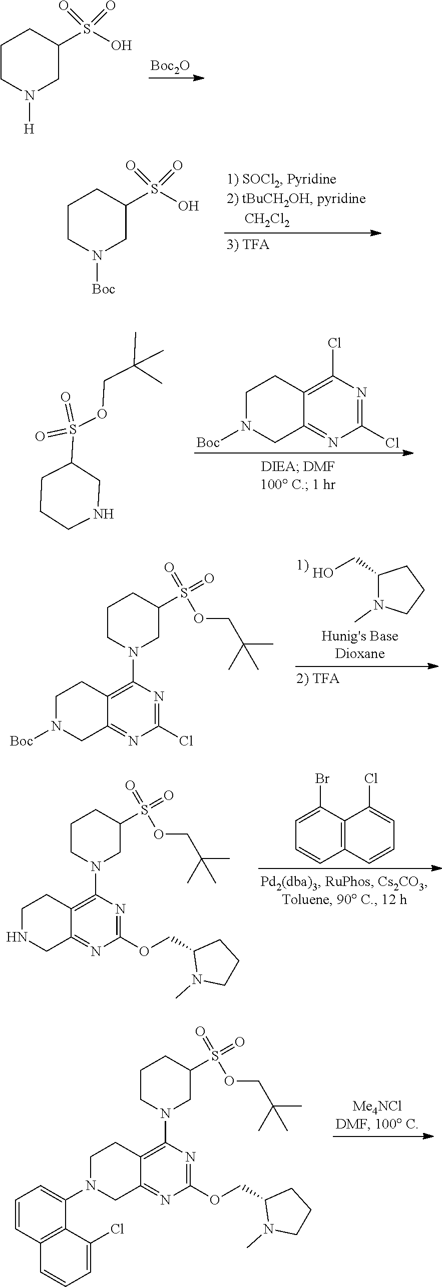

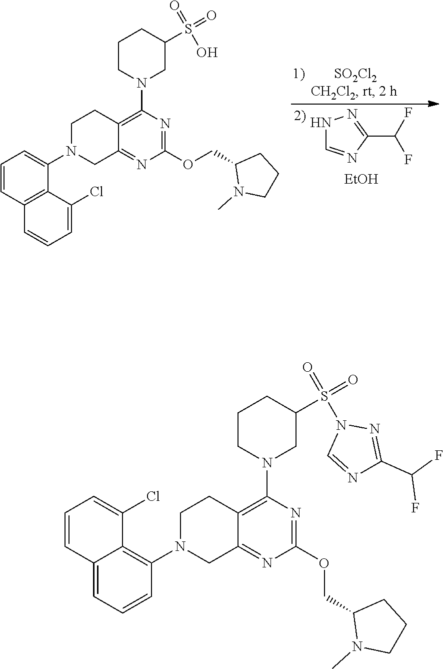 [SMILES: "EtOH" ; CN1CCC[C@H]1COc1nc2c(c(N3CCCC(S(=O)(=O)O)C3)n1)CCN(c1cccc3cccc(Cl)c13)C2 ; FC(F)c1nc[nH]n1 ; CN1CCC[C@H]1COc1nc2c(c(N3CCCC(S(=O)(=O)n4cnc(C(F)F)n4)C3)n1)CCN(c1cccc3cccc(Cl)c13)C2]

### Synthesis of Compound 117

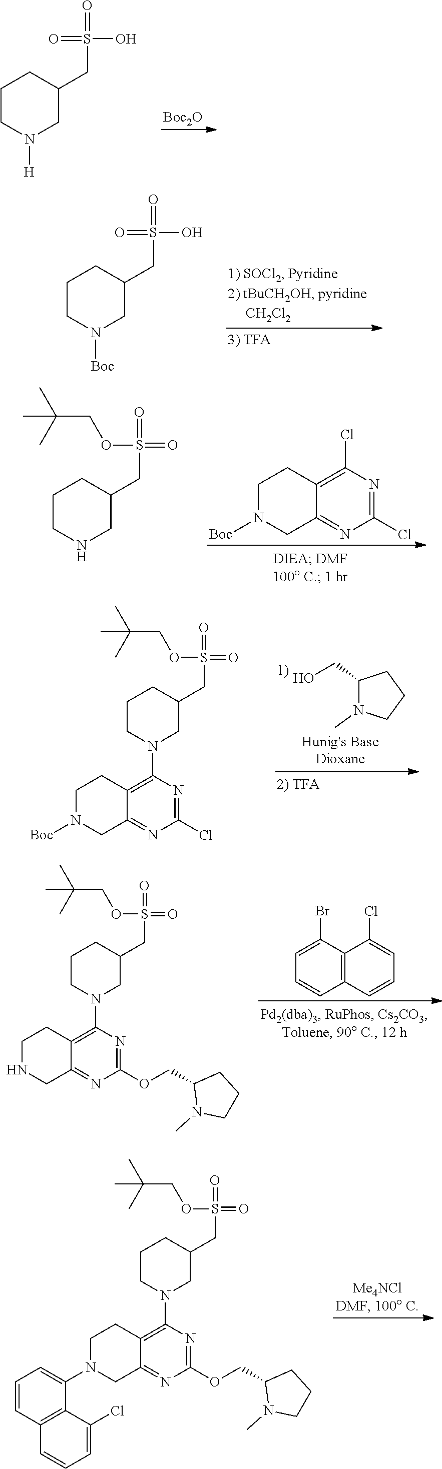

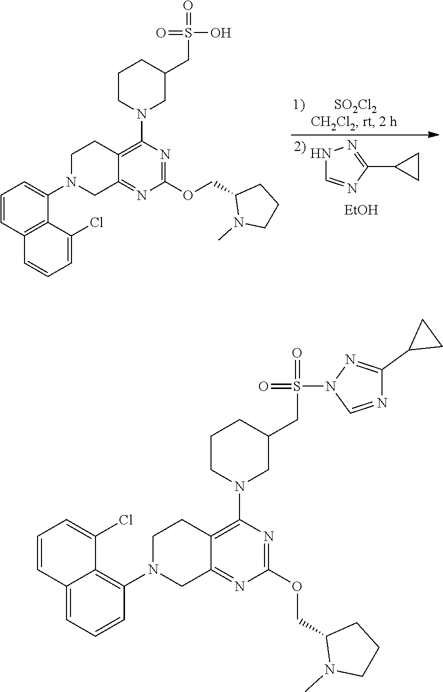 [SMILES: "EtOH" ; CN1CCC[C@H]1COc1nc2c(c(N3CCCC(CS(=O)(=O)O)C3)n1)CCN(c1cccc3cccc(Cl)c13)C2 ; c1nc(C2CC2)n[nH]1 ; CN1CCC[C@H]1COc1nc2c(c(N3CCCC(CS(=O)(=O)n4cnc(C5CC5)n4)C3)n1)CCN(c1cccc3cccc(Cl)c13)C2]

### Synthesis of Compound 118

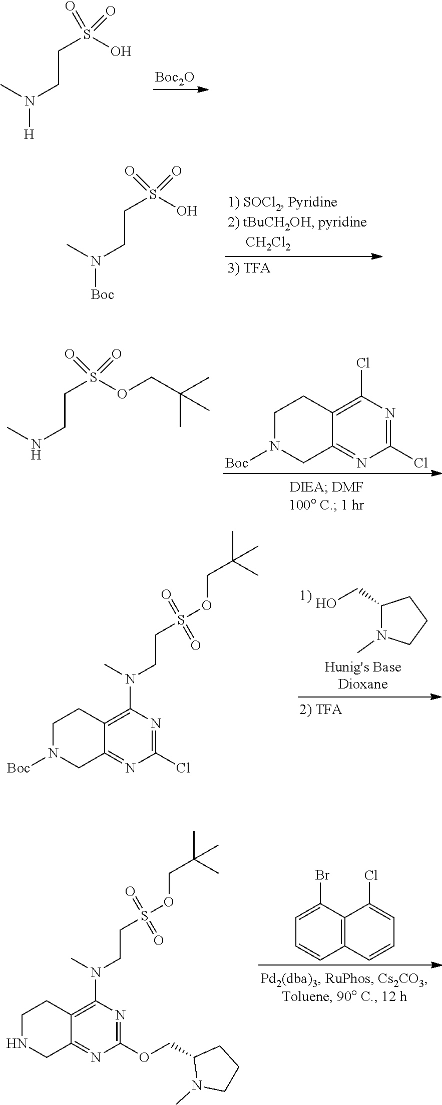

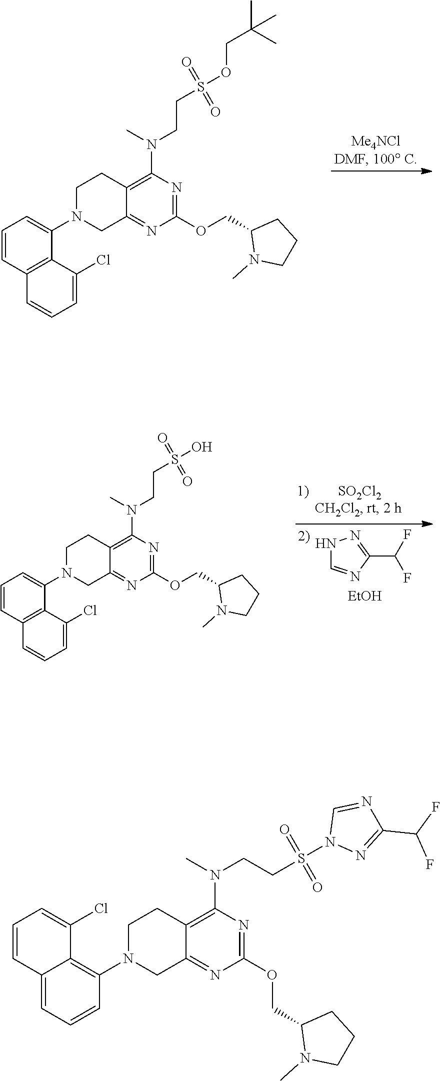

### Synthesis of Compound 119

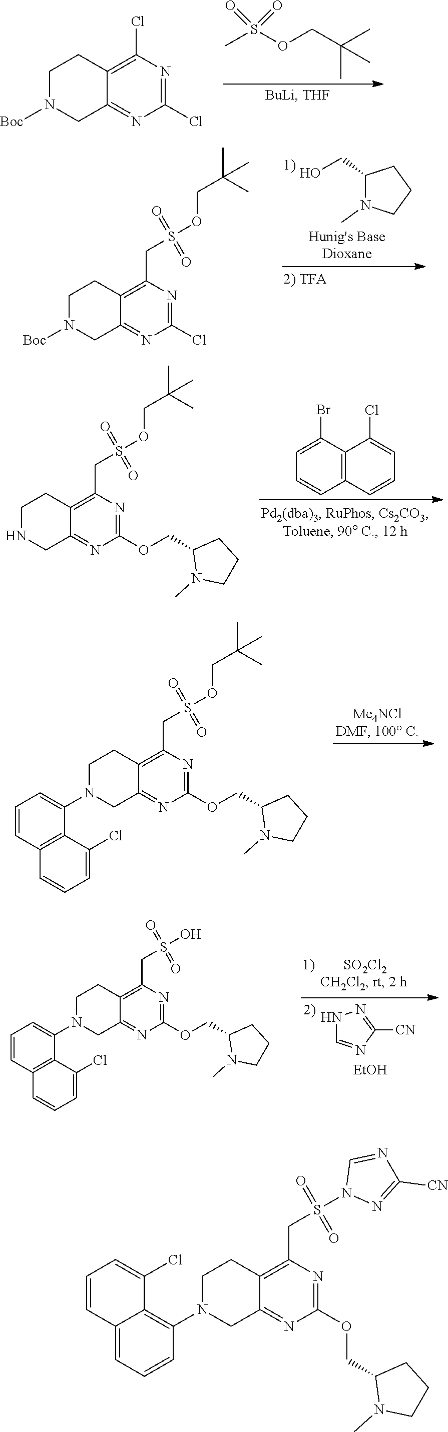

### Synthesis of Compound 120

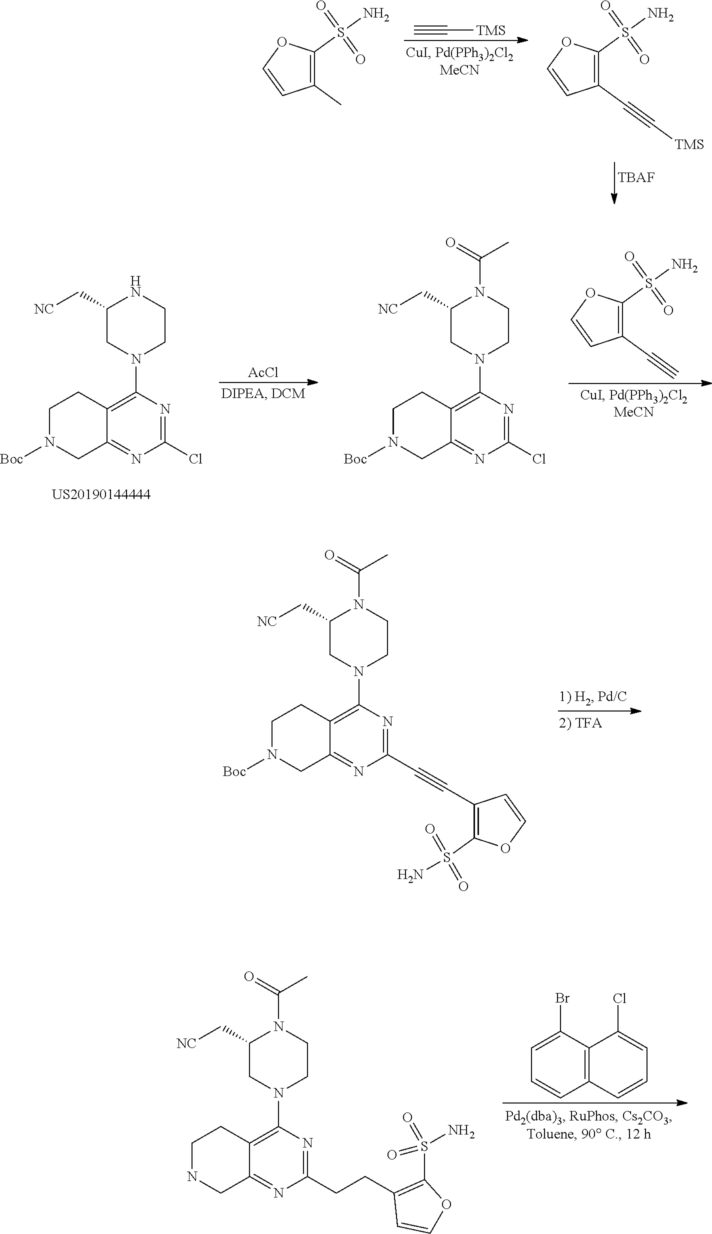

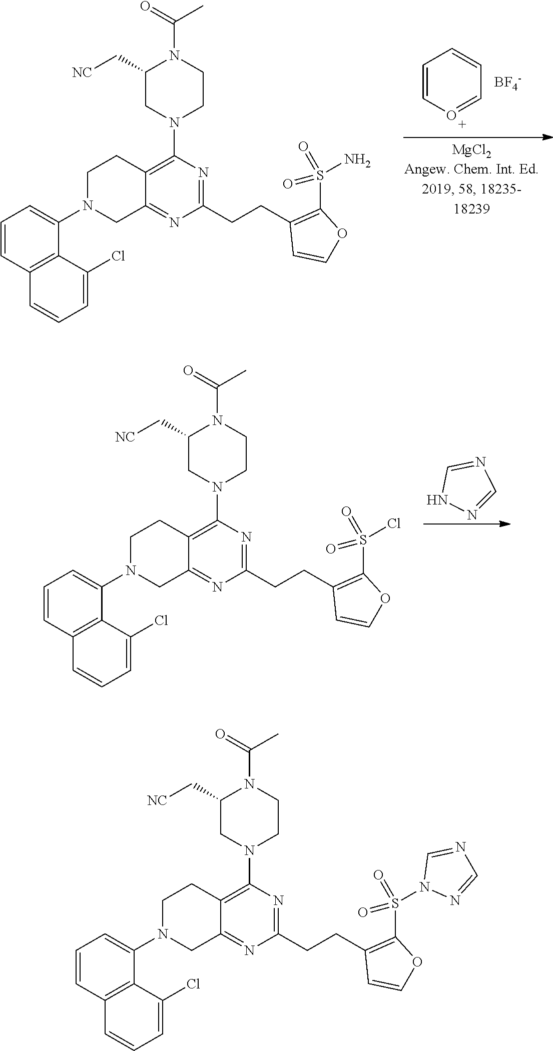

### Synthesis of Compound 121

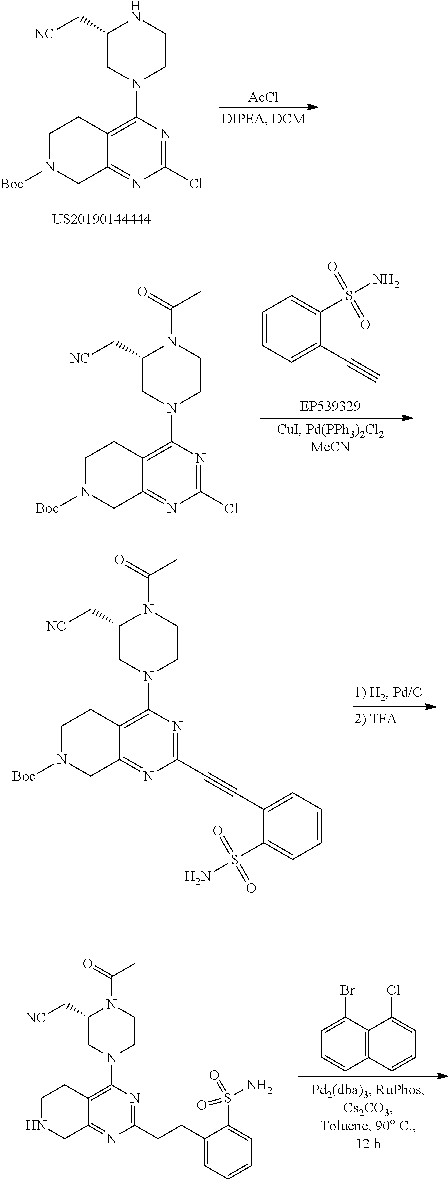

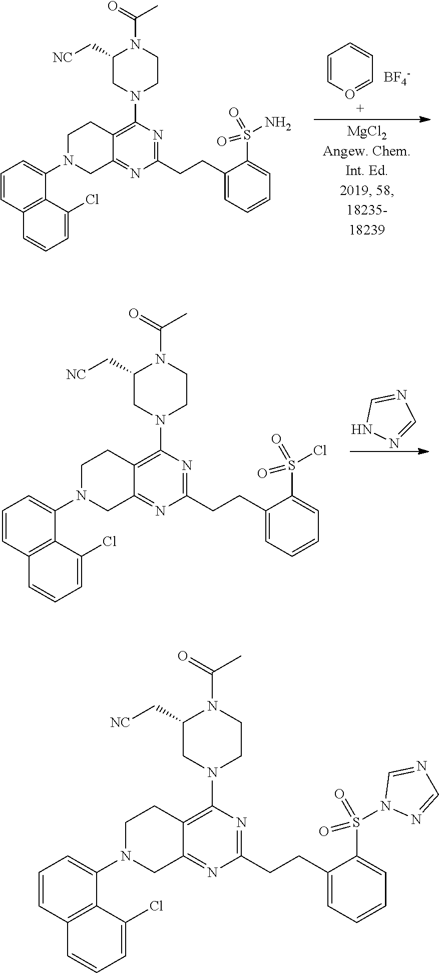

### Synthesis of Compound 122

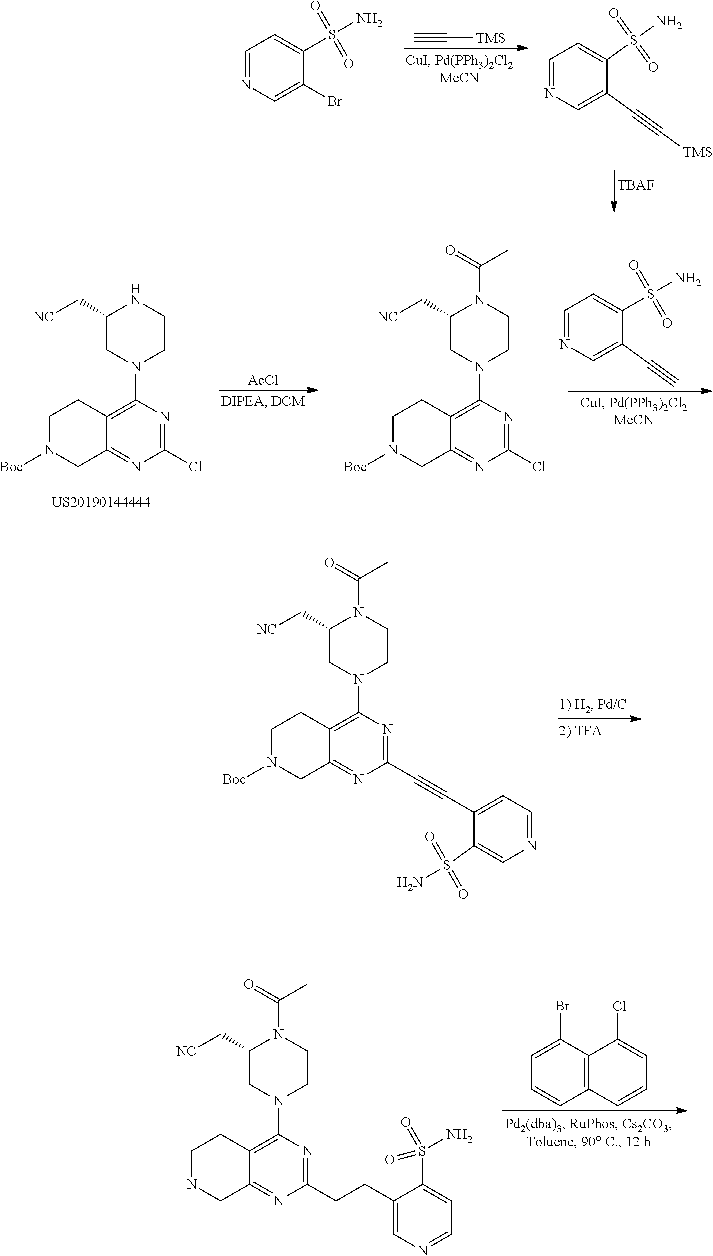

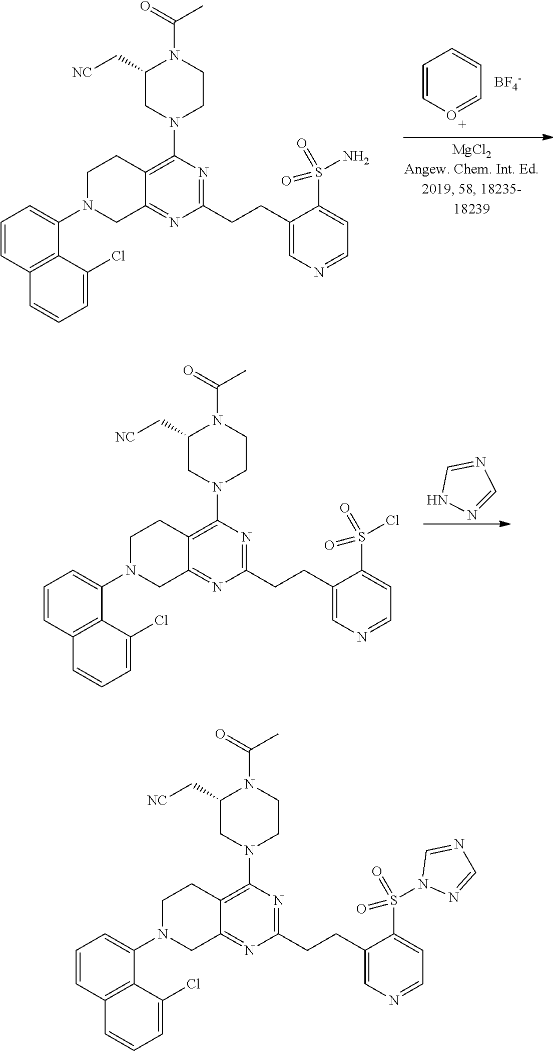

### Synthesis of Compound 123

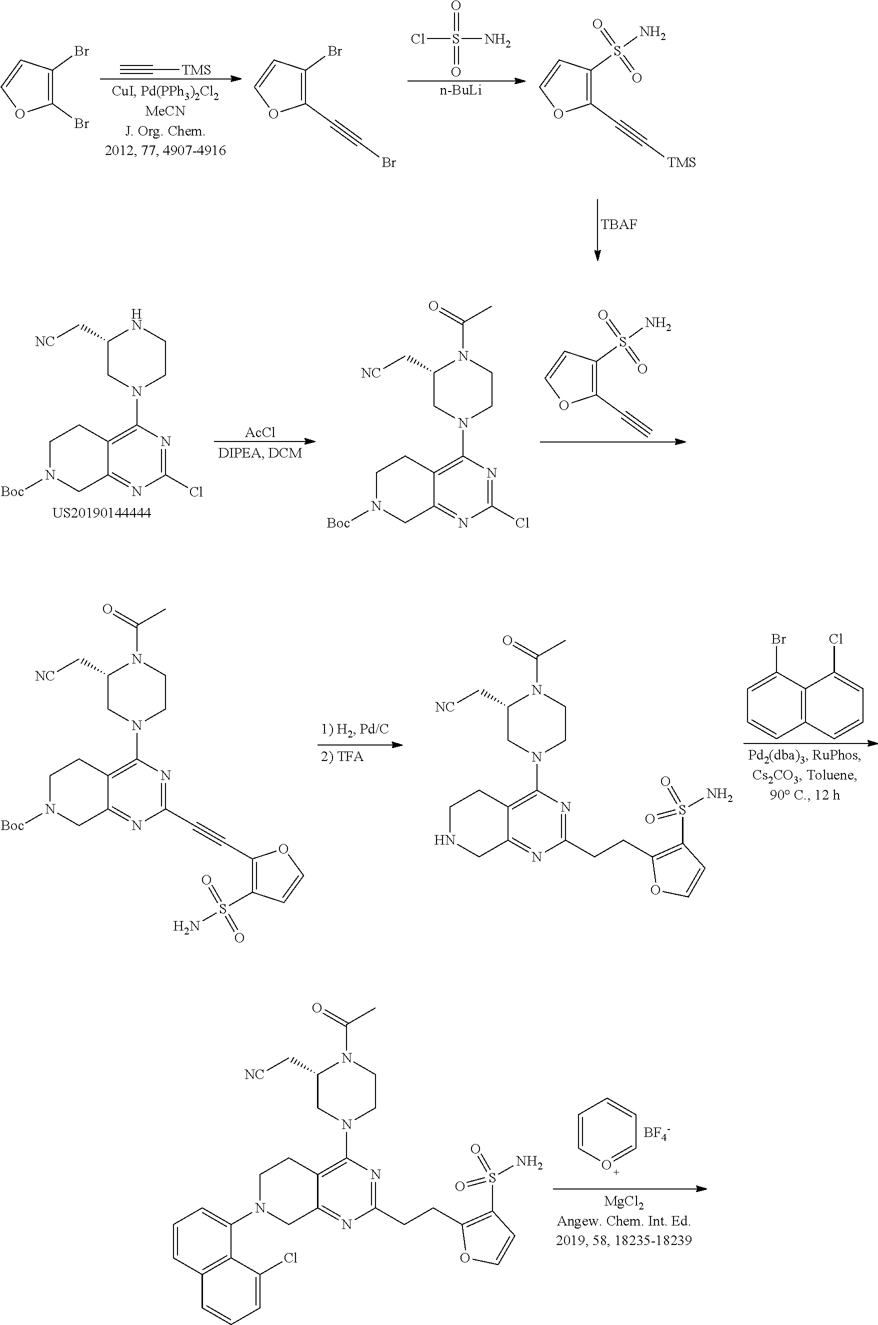

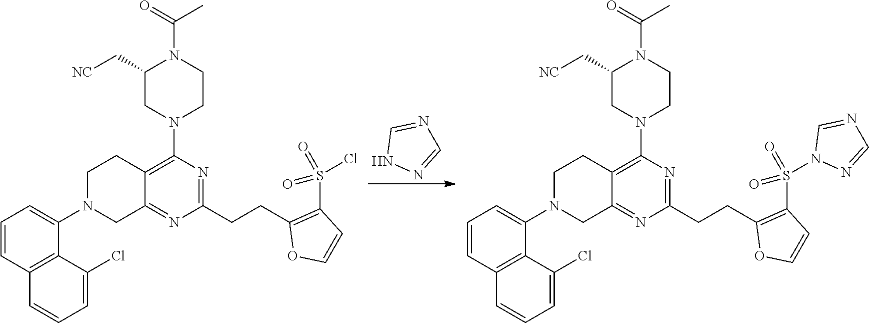

### Synthesis of Compound 124

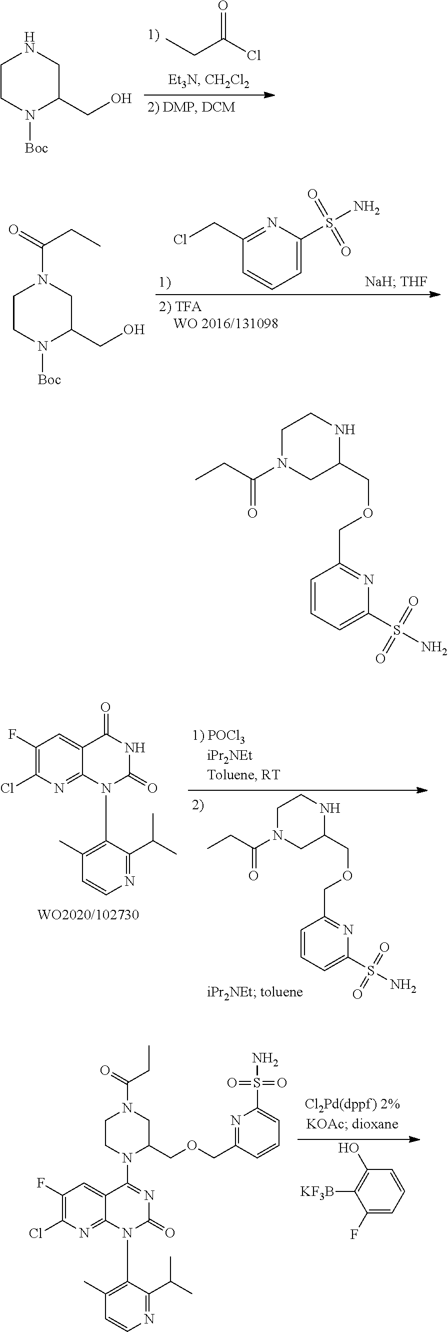

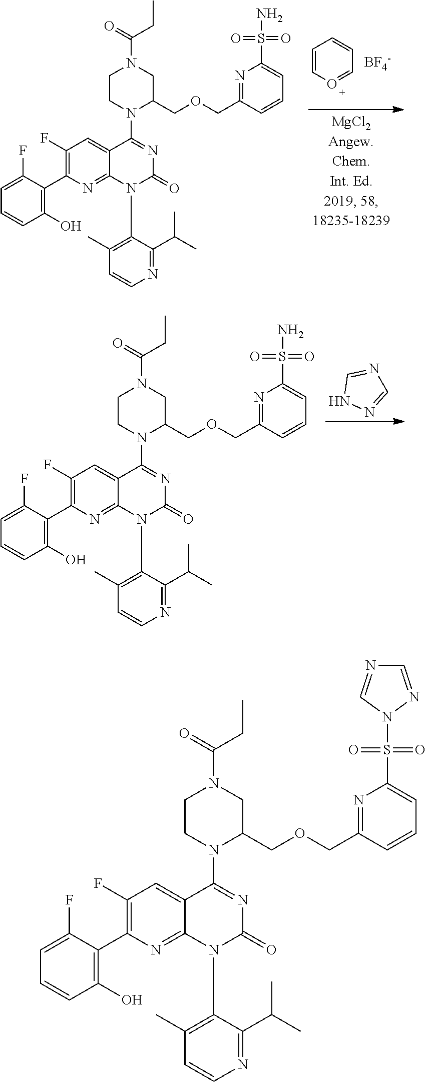

### Synthesis of Compound 125

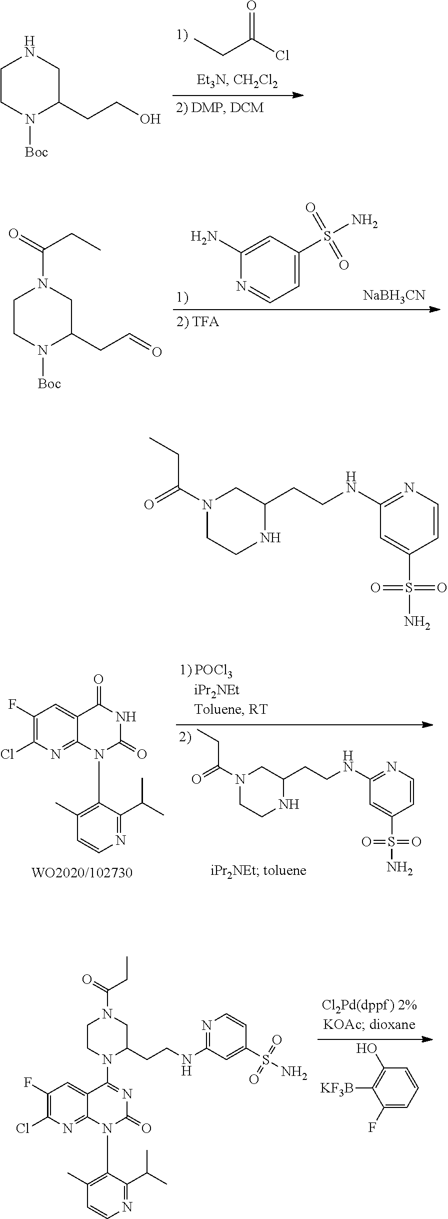

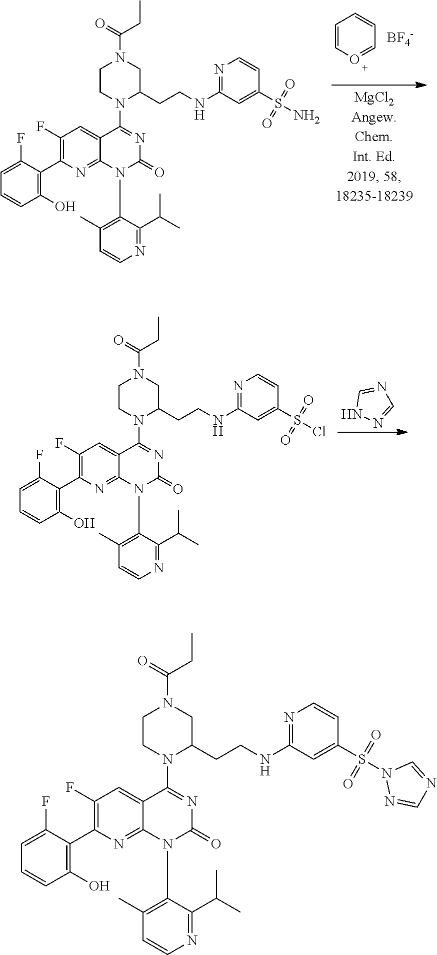

### Synthesis of Compound 126

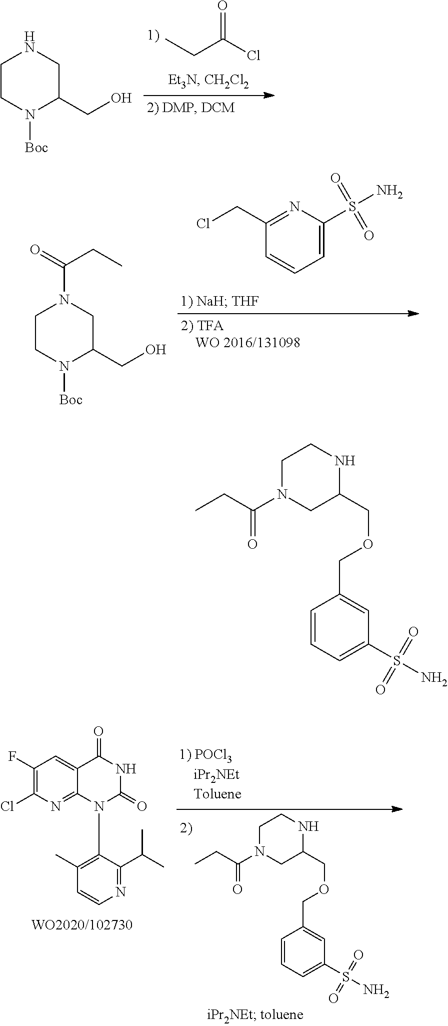

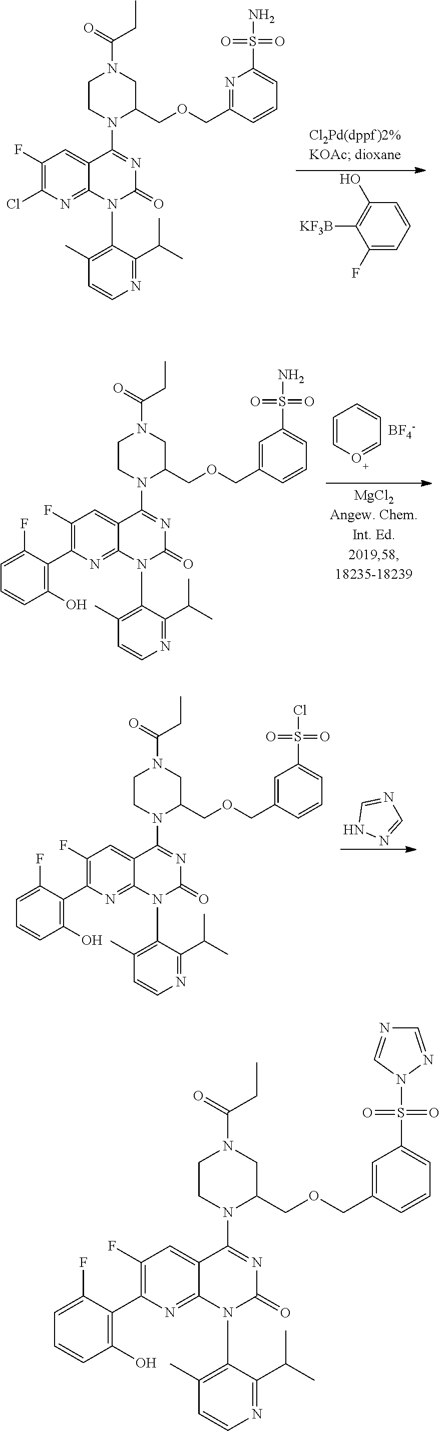

### Synthesis of Compound 127

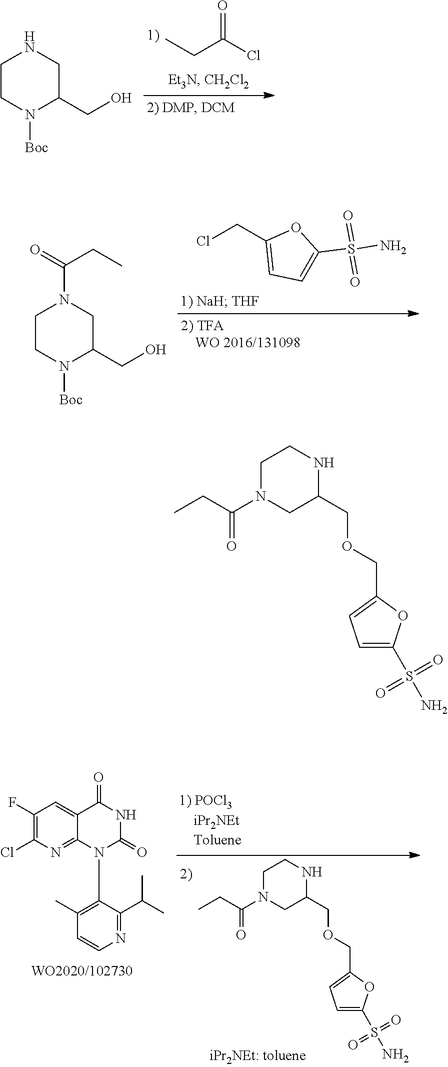

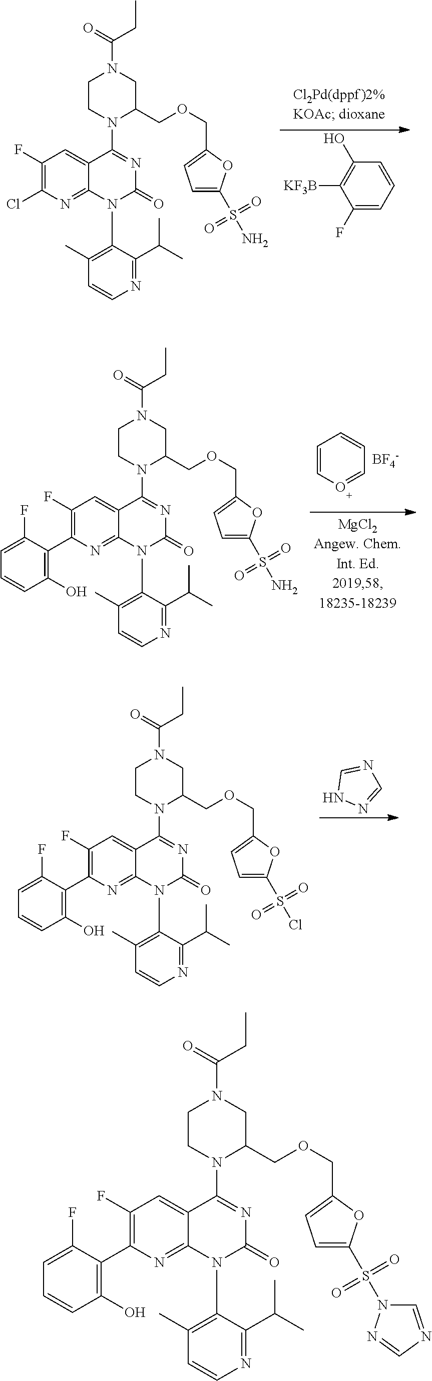

### Synthesis of Compound 128

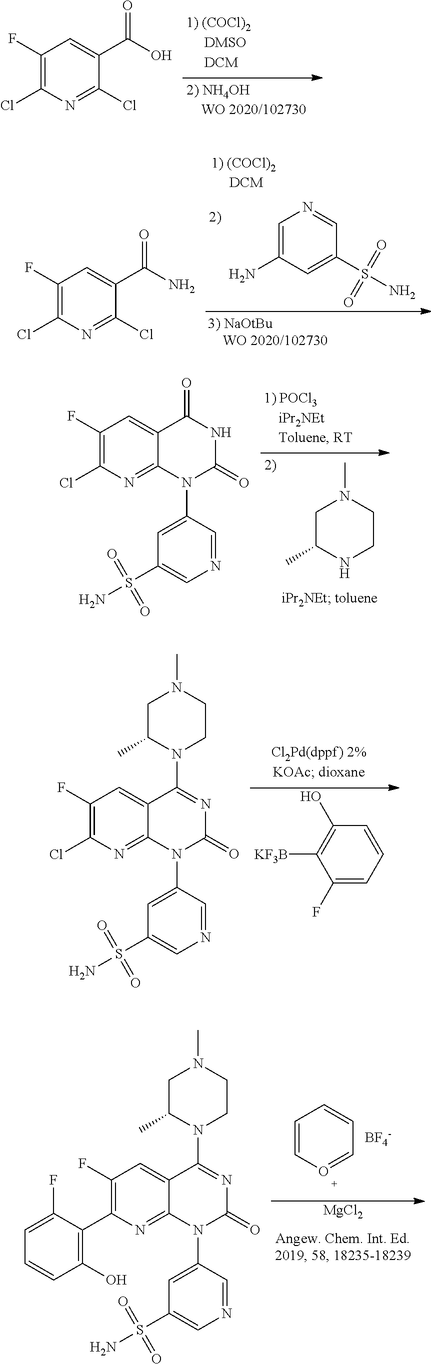

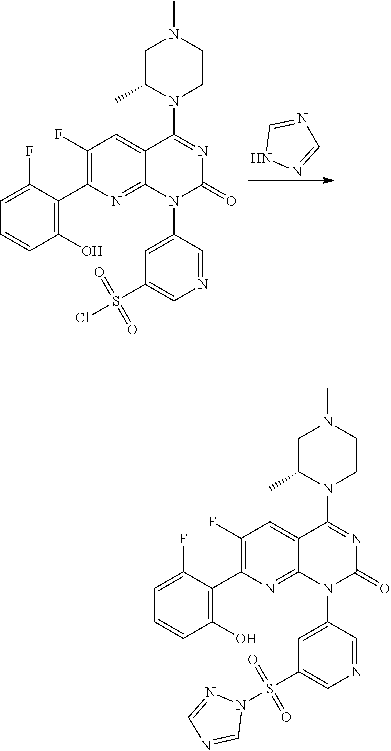 [SMILES: C[C@@H]1CN(C)CCN1c1nc(=O)n(-c2cncc(S(=O)(=O)Cl)c2)c2nc(-c3c(O)cccc3F)c(F)cc12 ; c1nc[nH]n1 ; C[C@@H]1CN(C)CCN1c1nc(=O)n(-c2cncc(S(=O)(=O)n3cncn3)c2)c2nc(-c3c(O)cccc3F)c(F)cc12]

### Synthesis of Compound 129

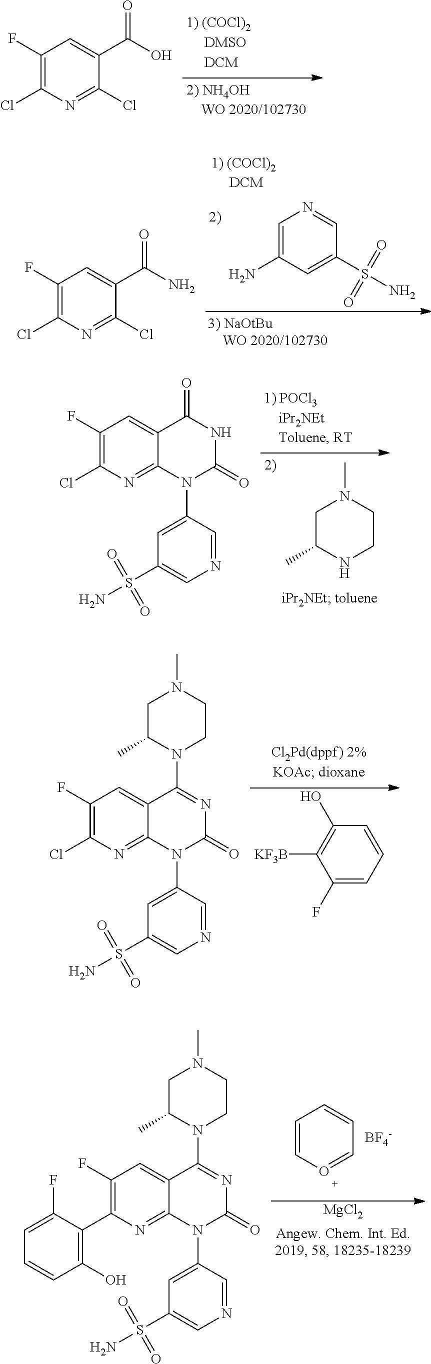

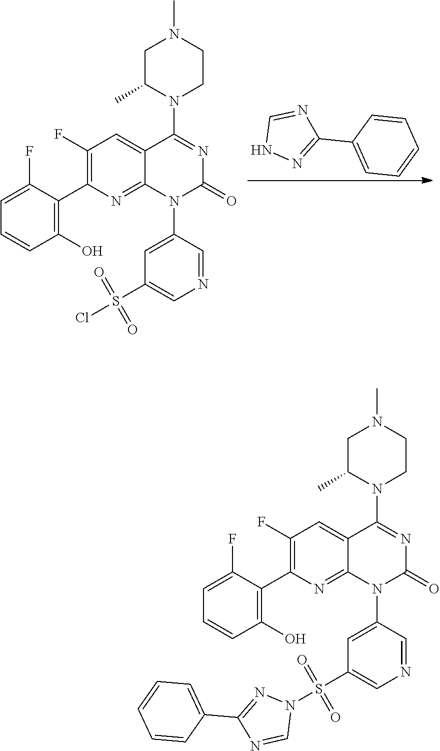 [SMILES: C[C@@H]1CN(C)CCN1c1nc(=O)n(-c2cncc(S(=O)(=O)Cl)c2)c2nc(-c3c(O)cccc3F)c(F)cc12 ; c1ccc(-c2nc[nH]n2)cc1 ; C[C@@H]1CN(C)CCN1c1nc(=O)n(-c2cncc(S(=O)(=O)n3cnc(-c4ccccc4)n3)c2)c2nc(-c3c(O)cccc3F)c(F)cc12]

### Synthesis of Compound 130

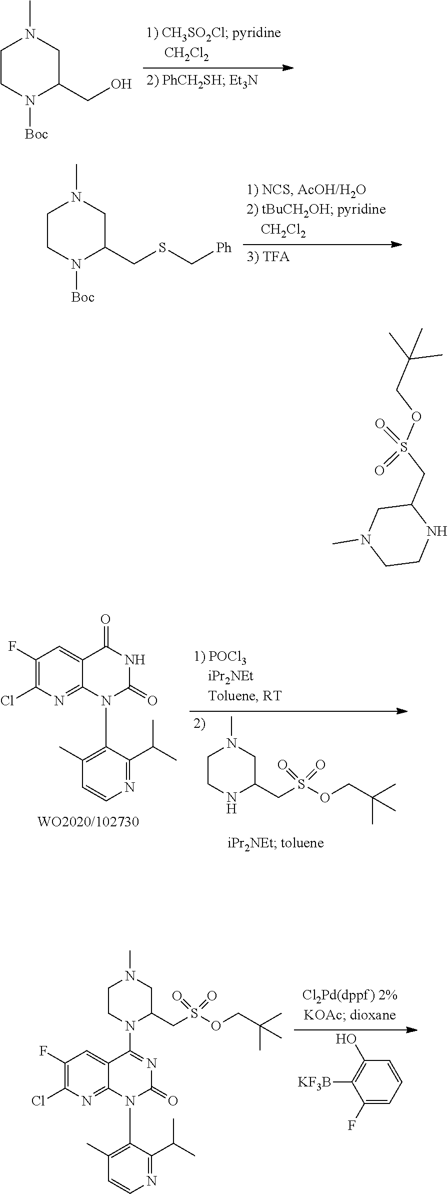

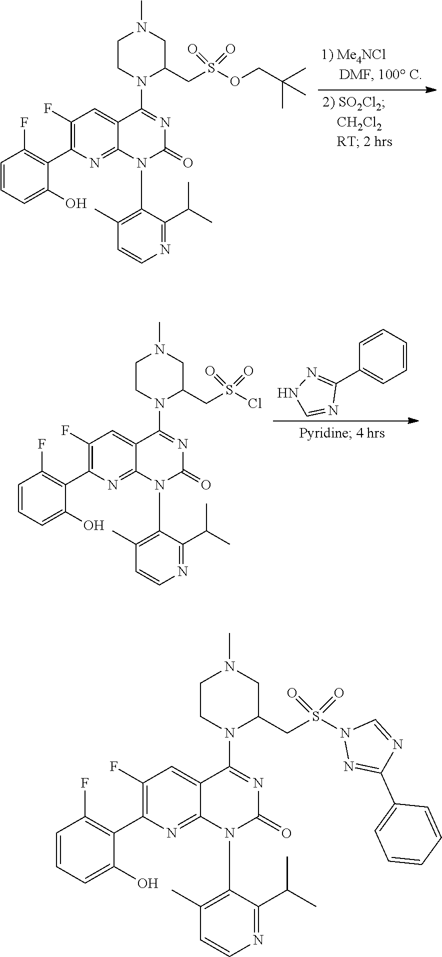 [SMILES: Cc1ccnc(C(C)C)c1-n1c(=O)nc(N2CCN(C)CC2CS(=O)(=O)OCC(C)(C)C)c2cc(F)c(-c3c(O)cccc3F)nc21 ; Cc1ccnc(C(C)C)c1-n1c(=O)nc(N2CCN(C)CC2CS(=O)(=O)Cl)c2cc(F)c(-c3c(O)cccc3F)nc21 ; c1ccc(-c2nc[nH]n2)cc1 ; Cc1ccnc(C(C)C)c1-n1c(=O)nc(N2CCN(C)CC2CS(=O)(=O)n2cnc(-c3ccccc3)n2)c2cc(F)c(-c3c(O)cccc3F)nc21]

### Synthesis of Compound 131

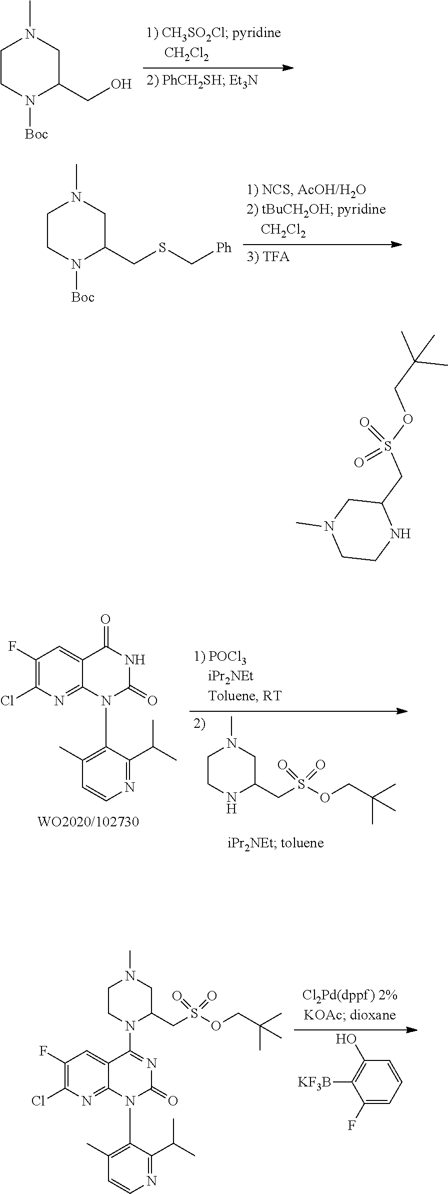

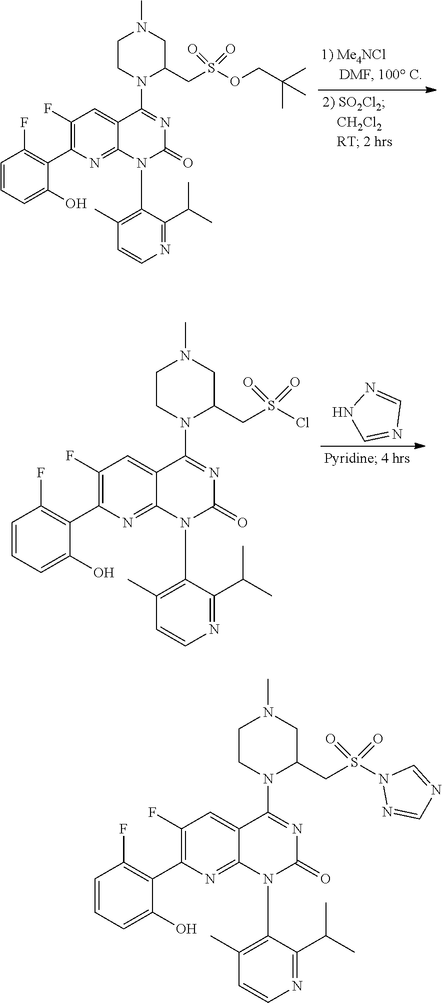 [SMILES: Cc1ccnc(C(C)C)c1-n1c(=O)nc(N2CCN(C)CC2CS(=O)(=O)OCC(C)(C)C)c2cc(F)c(-c3c(O)cccc3F)nc21 ; Cc1ccnc(C(C)C)c1-n1c(=O)nc(N2CCN(C)CC2CS(=O)(=O)Cl)c2cc(F)c(-c3c(O)cccc3F)nc21 ; c1nc[nH]n1 ; Cc1ccnc(C(C)C)c1-n1c(=O)nc(N2CCN(C)CC2CS(=O)(=O)n2cncn2)c2cc(F)c(-c3c(O)cccc3F)nc21]

### Synthesis of Compound 132

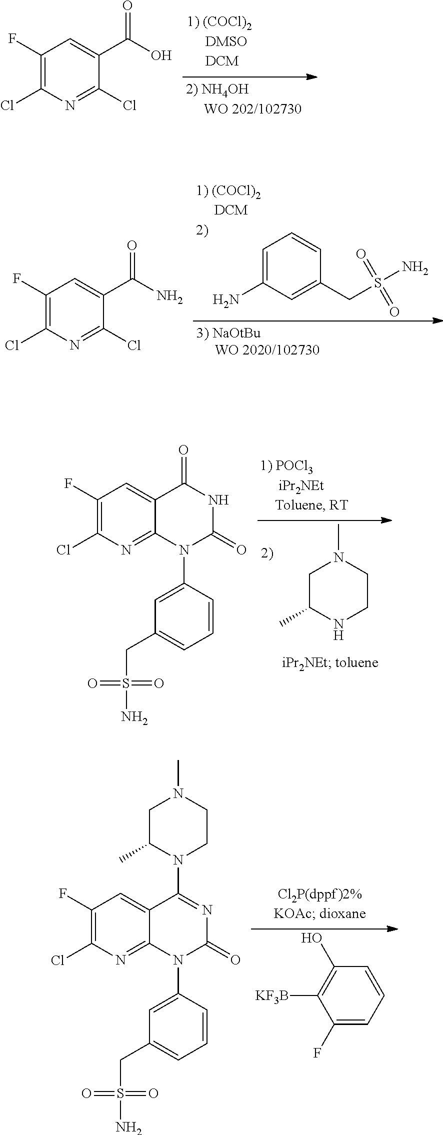

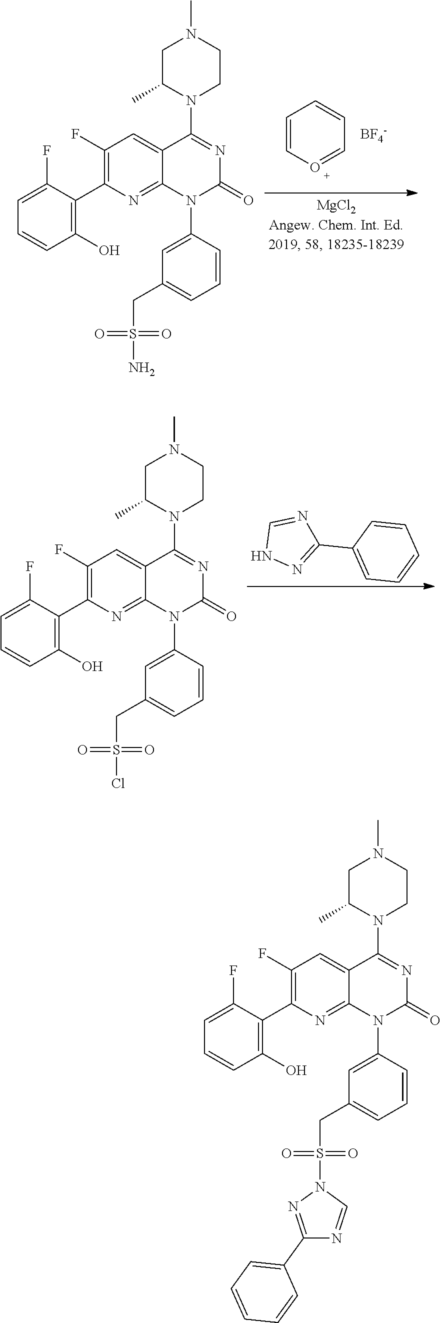

### Synthesis of Compound 133

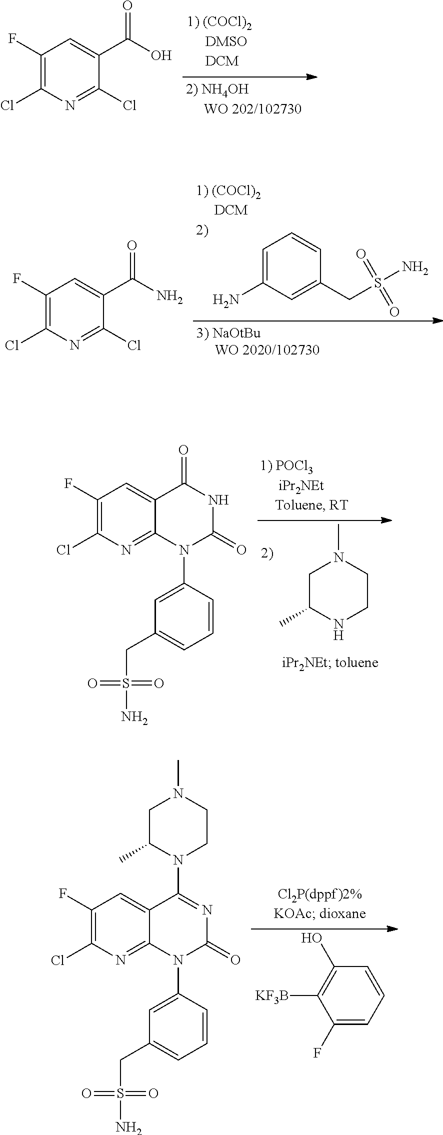

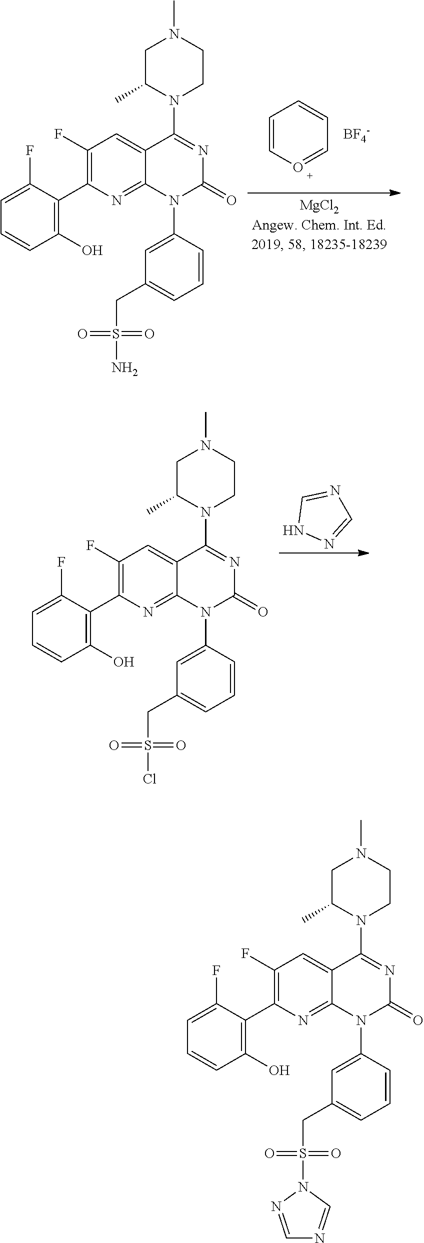

### Synthesis of Compound 134

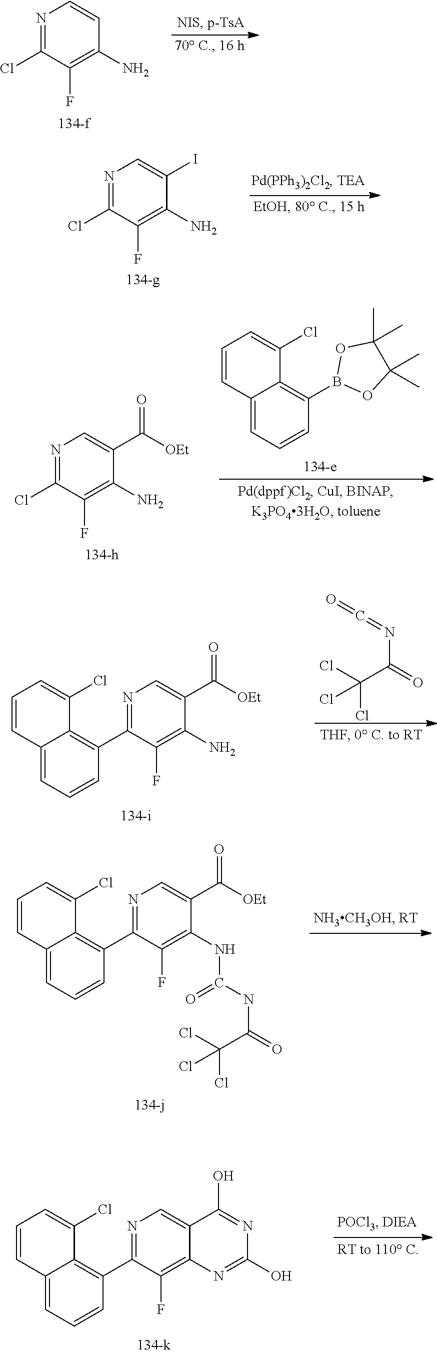

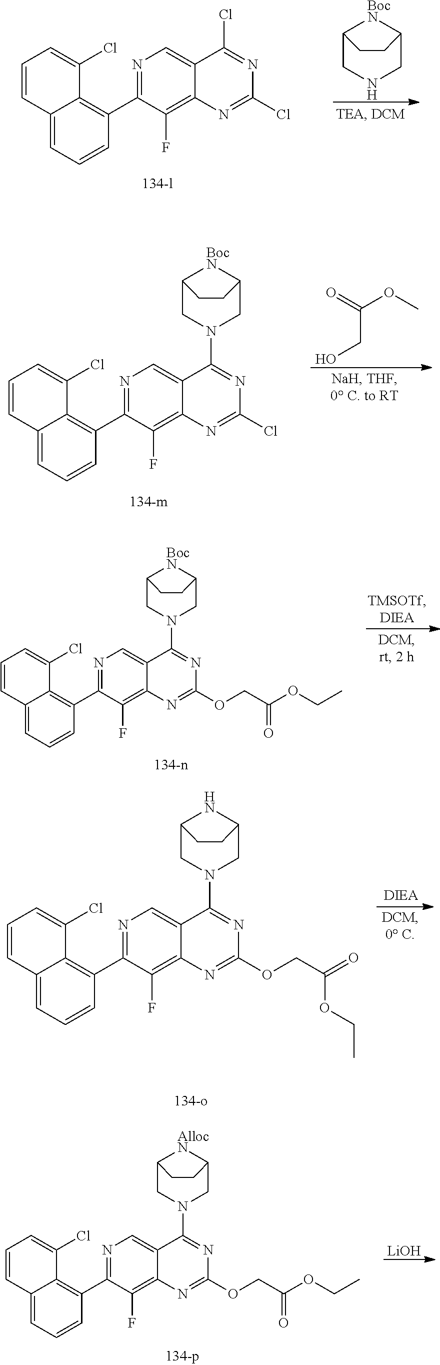

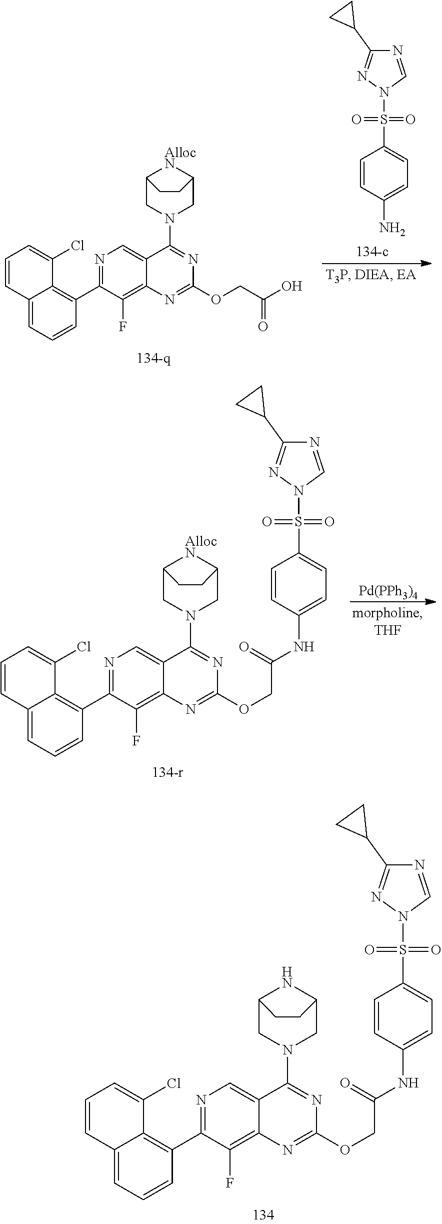

### Intermediate 134-c

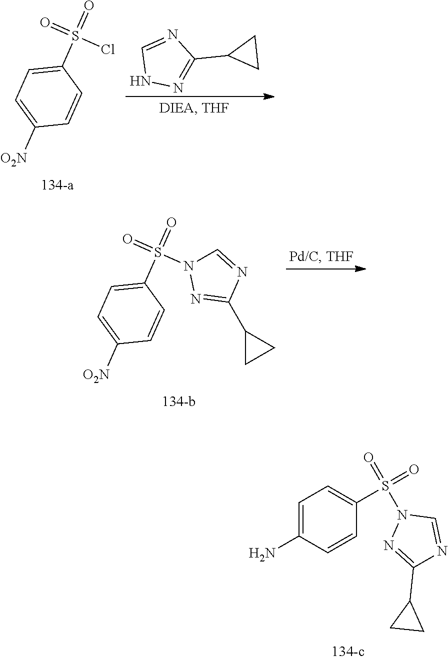

To a solution of 3-cyclopropyl-1H-1,2,4-triazole (164 mg, 1.51 mmol, 1.0 eq.), DIEA (700.5 mg, 5.43 mmol, 3.5 eq.) in THE (3 mL) 4-nitrobenzenesulfonyl chloride (134-a) (400 mg, 1.81 mmol, 1.2 eq.) in THF (2 mL) was added dropwise at 0° C. The residue was stirred at room temperature for 2 h. To the mixture was added Pd/C (200 mg, 10%) and stirred at room temperature for 2 h under H₂. The mixture was filtered, the filtrate was purified by Prep-HPLC (column: Xtimate C18 100*30 mm*3 um, condition: 32-72% B (A=water (0.05% ammonia hydroxide v/v)), B=acetonitrile), flow rate: 25 mL/min, UV Detector 220 nm), and then froze to give 134-c (60 mg, two isomer). The isomers were separated by chiral SFC (Column: CHIRALPAK IF, 3*25 cm, 5 m; Mobile Phase A: CO₂, Mobile Phase B: ACN; Flow rate: 80 mL/min; Gradient: isocratic 50% B; Column Temperature (° C.): 35; Back Pressure (bar): 100; Wave Length: 220 nm; RT₂ (min): 5.2; Sample Solvent: ACN; Injection Volume: 9 mL; Number Of Runs: 3) to give 134-c (23 mg, 20.8%) as a white solid. m/z: [M+H]⁺ Calcd for C₁₁H₁₃N₄O₂S 265.1; Found 295.1. ¹H NMR (400 MHz, DMSO-d6) δ 8.99 (s, 1H), 7.68-7.60 (m, 2H), 6.71-6.62 (m, 4H), 1.99 (tt, J=8.4, 4.8 Hz, 1H), 0.99-0.88 (m, 2H), 0.88-0.76 (m, 2H).

### Intermediate 134-e

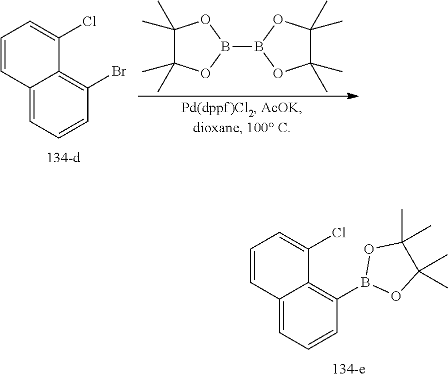

A mixture of 1-bromo-8-chloronaphthalene (40 g, 165 mmol,), potassium acetate (48.76 g, 496 mmol) bis(pinacolato)diboron (84.12 g, 331 mmol), Pd(dppf)Cl₂ (12.12 g, 16 mmol) in dioxane (1000 mL) was stirred at 100° C. for 8 h under N₂ atmosphere. The resulting mixture was filtered, the filter cake was washed with ethyl acetate (3×300 mL) and concentrated under reduced pressure to afford the crude product, which was purified by flash chromatography on silica gel (ethyl acetate/petroleum ether=0/1 to 1/1) to give 134-e (40 g, 83% yield) as a white solid. m/z: [M+H]⁺ Calcd for C₁₆H₁₉BClO₂ 289.1; Found 289.1.

### Step 1

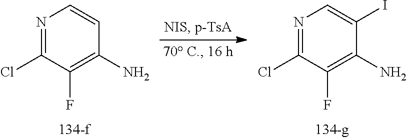 [SMILES: Nc1ccnc(Cl)c1F ; Nc1c(I)cnc(Cl)c1F]

A solution of 2-chloro-3-fluoropyridin-4-amine (42 g, 286.59 mmol, 1 equiv) and NIS (77.37 g, 343.91 mmol, 1.2 equiv) in acetic acid (220 mL) was added p-toluenesulfonic acid (2.47 g, 14.33 mmol, 0.05 equiv) at room temperature and was stirred for 16 h at 70° C. After that, the resulting mixture was diluted with water and ethyl acetate, the organic layer was washed with saturated NaHCO₃ solution, saturated Na₂SO₃ solution and brine, dried over anhydrous Na₂SO₄. After filtration, the filtrate was concentrated under reduced pressure to afford 134-g (90 g, crude) as a yellow solid m/z: [M+H]⁺ Calcd for C₅H₄ClFIN₂ 272.9; Found 272.9.

### Step 2

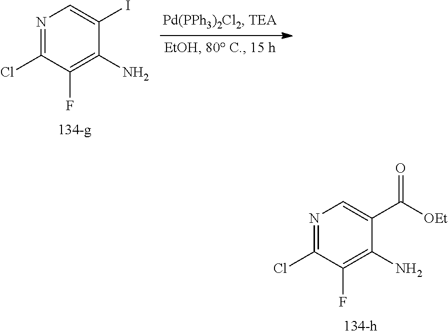 [SMILES: Nc1c(I)cnc(Cl)c1F ; CCOC(=O)c1cnc(Cl)c(F)c1N]

To a solution of 2-chloro-3-fluoro-5-iodopyridin-4-amine (90 g, 330.33 mmol, 1 equiv) in ethanol (1700 mL) was added Pd(PPh₃)₂Cl₂ (23 g, 33.03 mmol, 0.1 equiv) and TEA (120 g, 1189.21 mmol, 3.6 equiv) and was stirred for 15 h at 80° C. under CO (15 Psi) atmosphere. The resulting mixture was filtered, the filtrate was concentrated under reduced pressure to remove 70% of ethanol and the residue was filtered. The filter cake was concentrated under reduced pressure to afford 134-h (68 g, 94.16%) as a yellow solid m/z: [M+H]⁺ Calcd for C₈H₉ClFN₂O₂ 219.0; Found 219.0.

### Step 3

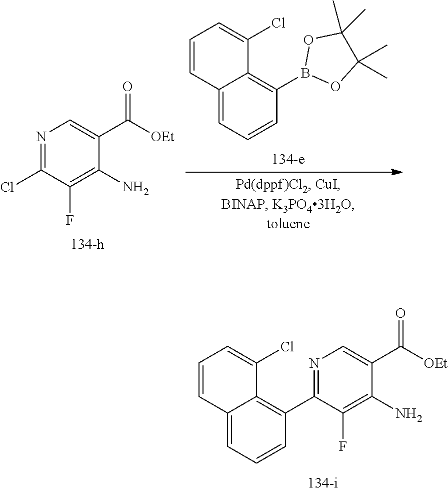 [SMILES: CCOC(=O)c1cnc(Cl)c(F)c1N ; CC1(C)OB(c2cccc3cccc(Cl)c23)OC1(C)C ; CCOC(=O)c1cnc(-c2cccc3cccc(Cl)c23)c(F)c1N]

A mixture of ethyl 4-amino-6-chloro-5-fluoronicotinate (15 g, 68 mmol), 2-(8-chloronaphthalen-1-yl)-4,4,5,5-tetramethyl-1,3,2-dioxaborolane (30 g, 103 mmol), Pd(dppf)Cl₂ (5 g, 6.8 mmol), CuI (2.61 g, 13.7 mmol), BINAP (8.55 g, 13.7 mmol) and K₃PO₄·3H₂O (54 g, 204 mmol) in toluene (300 mL) was stirred at 100° C. for 8 h under N₂ atmosphere. The resulting mixture was filtered, the filter cake was washed with DCM (3×100 ml) and concentrated under reduced pressure to afford the crude product, which was purified by flash chromatography on silica gel (ethyl acetate/petroleum ether=0/1 to 1/1) to give 134-i (16 g, 67% yield) as a white solid. m/z: [M+H]⁺ Calcd for C₁₈H₁₅ClFN₂O₂ 345.1; Found 345.1.

### Step 4

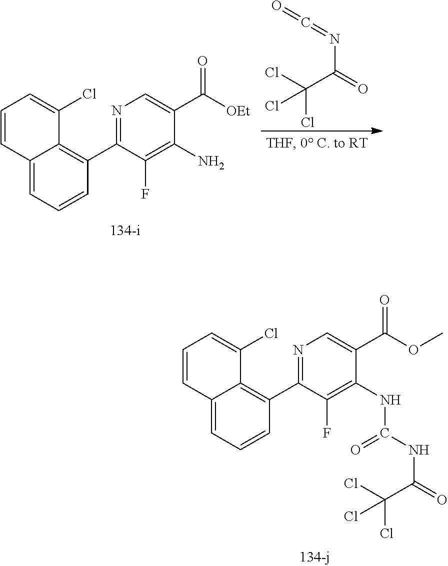

A mixture of 4-amino-6-(8-chloronaphthalen-1-yl)-5-fluoronicotinate (2.5 g, 7.25 mmol, 1 equiv) and trichloroethanecarbonyl isocyanate (1.50 g, 7.976 mmol, 1.1 equiv) in THE (25 mL) was stirred for 2 h at 25° C. under N₂ atmosphere. The residue was purified by trituration with MTBE (30 mL) to afford 134-j (3 g, 79.70%) as a white solid m/z: [M+H]⁺ Calcd for C₂₀H₁₃Cl₄FN₃O₄ 518.0; Found 518.0.

### Step 5

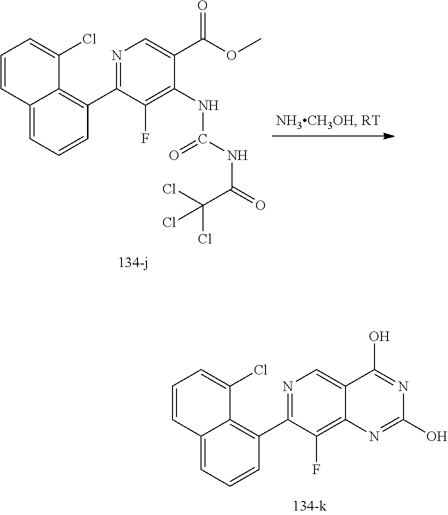 [SMILES: COC(=O)c1cnc(-c2cccc3cccc(Cl)c23)c(F)c1NC(=O)NC(=O)C(Cl)(Cl)Cl ; Oc1nc(O)c2cnc(-c3cccc4cccc(Cl)c34)c(F)c2n1]

A mixture of methyl 6-(8-chloronaphthalen-1-yl)-5-fluoro-4-(3-(2,2,2-trichloroacetyl)ureido)nicotinate (3 g, 5.77 mmol, 1 equiv) in ammonia in methanol (30 mL, 7 M) was stirred for 1 h at 25° C. under N₂ atmosphere. The residue was purified by trituration with MTBE (30 mL) to afford 134-k (1.8 g, 91.15%) as a white solid m/z: [M+H]⁺ Calcd for C₁₇H₁₀ClFN₃O₂ 342.0; Found 342.0.

### Step 6

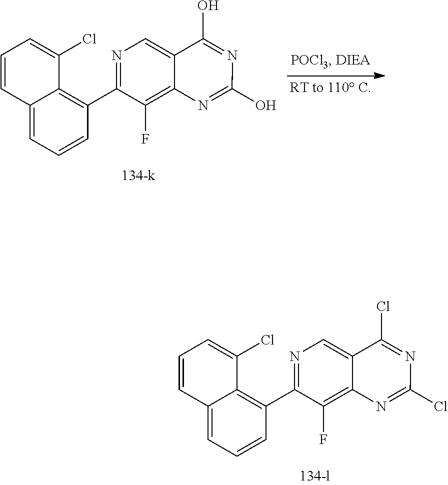 [SMILES: Oc1nc(O)c2cnc(-c3cccc4cccc(Cl)c34)c(F)c2n1 ; Fc1c(-c2cccc3cccc(Cl)c23)ncc2c(Cl)nc(Cl)nc12]

A solution of 7-(8-chloronaphthalen-1-yl)-8-fluoropyrido[4,3-d]pyrimidine-2,4-diol (2 g, 5.85 mmol, 1 equiv) and DIEA (3.78 g, 29.26 mmol, 5 equiv) in POCl₃ (20 mL) was stirred for 16 h at 110° C. under N₂ atmosphere. The resulting mixture was concentrated under reduced pressure. The crude product was used in the next step directly without further purification. m/z: [M+H]⁺ Calcd for C₁₇H₈C₁₃FN₃ 378.0; Found 378.0.

### Step 7

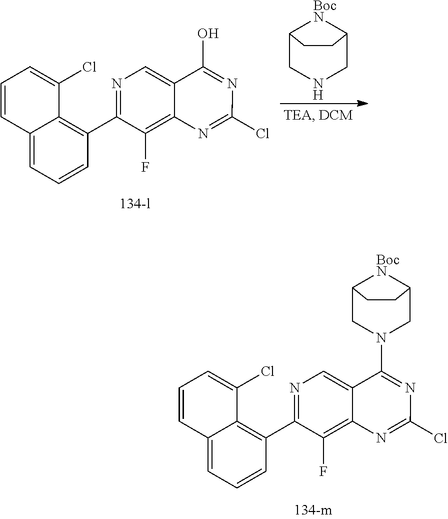 [SMILES: Oc1nc(Cl)nc2c(F)c(-c3cccc4cccc(Cl)c34)ncc12 ; CC(C)(C)OC(=O)N1C2CCC1CNC2 ; CC(C)(C)OC(=O)N1C2CCC1CN(c1nc(Cl)nc3c(F)c(-c4cccc5cccc(Cl)c45)ncc13)C2]

A mixture of 2,4-dichloro-7-(8-chloronaphthalen-1-yl)-8-fluoropyrido[4,3-d]pyrimidine (2 g, 5.28 mmol, 1 equiv) and tert-butyl 3,8-diazabicyclo[3.2.1]octane-8-carboxylate (1.68 g, 7.92 mmol, 1.5 equiv) DIEA (2.05 g, 15.84 mmol, 3 equiv) in DCM (20 mL, 314.61 mmol, 59.56 equiv) was stirred for 1 h at −40° C. under N₂ atmosphere. The resulting mixture was extracted with DCM (20 mL×2). The combined organic layers were washed with brine (10 mL×2) and dried over anhydrous Na₂SO₄. After filtration and concentration, the residue was purified by silica gel column chromatography, eluted with petroleum ether:ethyl acetate (0%˜50%) to afford 134-m (1.4 g, 47.80%) as a white solid m/z: [M+H]⁺ Calcd for C₂₈H₂₇Cl₂FN₅O₂ 554.1; Found 554.1.

### Step 8

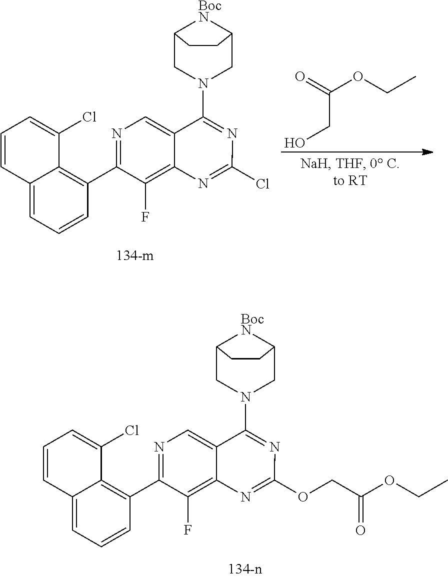

A mixture of tert-butyl (1R,5S)-3-(2-chloro-7-(8-chloronaphthalen-1-yl)-8-fluoropyrido[4,3-d]pyrimidin-4-yl)-3,8-diazabicyclo[3.2.1]octane-8-carboxylate (500 mg, 0.90 mmol, 1 equiv) and ethyl 2-hydroxyacetate (140.82 mg, 1.353 mmol, 1.5 equiv), NaH (32.46 mg, 1.353 mmol, 1.5 equiv) in THE (5 mL) was stirred for 1 h at 0° C. under N₂ atmosphere. The reaction was quenched with NH₄Cl at 0° C. The aqueous layer was extracted with ethyl acetate (10 mL×2). After concentration, the residue was purified by silica gel column chromatography, eluted with petroleum ether:ethyl acetate (0%˜60%) to afford 134-n (460 mg, 82.0%) as a yellow solid m/z: [M+H]⁺ Calcd for C₃₂H₃₄C₁FN₅O₅ 621.1; Found 622.1.

### Step 9

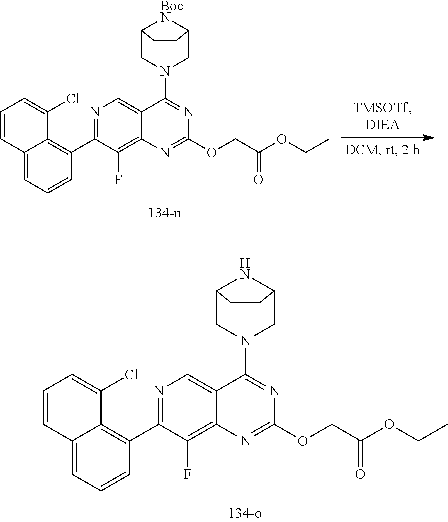 [SMILES: CCOC(=O)COc1nc(N2CC3CCC(C2)N3C(=O)OC(C)(C)C)c2cnc(-c3cccc4cccc(Cl)c34)c(F)c2n1 ; CCOC(=O)COc1nc(N2CC3CCC(C2)N3)c2cnc(-c3cccc4cccc(Cl)c34)c(F)c2n1]

A solution of tert-butyl (1R,5S)-3-(7-(8-chloronaphthalen-1-yl)-2-(2-ethoxy-2-oxoethoxy)-8-fluoropyrido[4,3-d]pyrimidin-4-yl)-3,8-diazabicyclo[3.2.1]octane-8-carboxylate (600 mg, 0.96 mmol, 1 equiv) and TMSOTf (643.07 mg, 2.89 mmol, 3 equiv), DIEA (373.97 mg, 2.89 mmol, 3 equiv) in DCM (6 mL) was stirred for 1 h at 25° C. under N₂ atmosphere. The resulting mixture was extracted with ethyl acetate (10 mL×3). The combined organic layers were washed with brine (5 mL×2), dried over anhydrous Na₂SO₄. After filtration, the filtrate was concentrated under reduced pressure to afford 134-o (800 mg, crude) as a yellow solid, which was used in the next step without any purification. m/z: [M+H]⁺ Calcd for C₂₇H₂₆ClFN₅O₃ 522.2; Found 522.2.

### Step 10

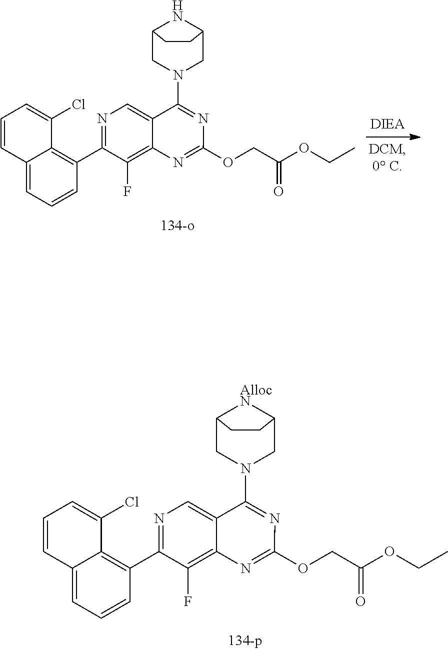

A solution of ethyl ethyl 2-((4-((1R,5S)-3,8-diazabicyclo[3.2.1]octan-3-yl)-7-(8-chloronaphthalen-1-yl)-8-fluoropyrido[4,3-d]pyrimidin-2-yl)oxy)acetate (400 mg, 0.76 mmol, 1 equiv) and allyl chlorocarbonate (138.55 mg, 1.14 mmol, 1.5 equiv), DIEA (297.13 mg, 2.29 mmol, 3 equiv) in DCM (4 mL) was stirred for 0.5 h at 0° C. under N₂ atmosphere. The resulting mixture was extracted with DCM (5 mL×2). The combined organic layers were washed with brine (5 mL×2) and dried over anhydrous Na₂SO₄. After filtration, the filtrate was concentrated under reduced pressure. The residue was purified by flash silica gel column chromatography, eluted with petroleum ether/ethyl acetate (0%˜80%) to afford 134-p (300 mg, 64.60%) as a white solid m/z: [M+H]⁺ Calcd for C₃₁H₃₀ClFN₅O₅ 606.2; Found 606.2.

### Step 11

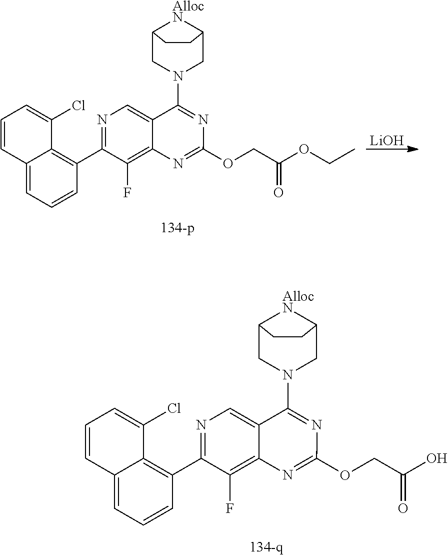

A solution of allyl (1R,5S)-3-(7-(8-chloronaphthalen-1-yl)-2-(2-ethoxy-2-oxoethoxy)-8-fluoropyrido[4,3-d]pyrimidin-4-yl)-3,8-diazabicyclo[3.2.1]octane-8-carboxylate (700 mg, 1.15 mmol, 1 equiv) and LiOH·H₂O (242.32 mg, 5.77 mmol, 5 equiv) in H₂O (4 mL) THF (4 mL) was stirred for 3 h at 25° C. under N₂ atmosphere. The mixture was basified to pH 6 with aqueous HCl (1 M). The resulting mixture was extracted with DCM (10 mL×2). The combined organic layers were washed with brine (10 mL×2) and dried over anhydrous Na₂SO₄. After filtration, the filtrate was concentrated under reduced pressure to afford 134-q (600 mg, 89.87%) as a yellow solid, which was used in the next step without any purification. m/z: [M+H]⁺ Calcd for C₂₉H₂₆ClFN₅O₅ 578.2; Found 578.2

### Step 12

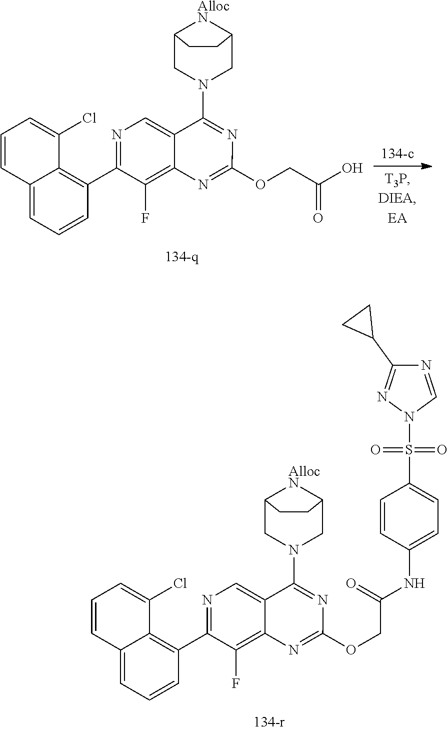

A mixture of 2-((4-((1R,5S)-8-((allyloxy)carbonyl)-3,8-diazabicyclo[3.2.1]octan-3-yl)-7-(8-chloronaphthalen-1-yl)-8-fluoropyrido[4,3-d]pyrimidin-2-yl)oxy)acetic acid (100 mg, 0.173 mmol, 1 equiv) and 134-c (68.59 mg, 0.259 mmol, 1.5 equiv), DIEA (67.08 mg, 0.519 mmol, 3 equiv) in propylphosphonic anhyhdride (2 mL) and ethyl acetate (2 mL) was stirred for 2 h at 50° C. under N₂ atmosphere. The resulting mixture was concentrated under vacuum. The resulting mixture was extracted with DCM (5 mL×2). The combined organic layers were washed with brine (5 mL×2) and dried over anhydrous Na₂SO₄. After filtration, the filtrate was concentrated under reduced pressure. The residue was purified by silica gel column chromatography, eluted with methanol/DCM (0%˜20%) to afford 134-r (80 mg, 56.10%) as a white solid m/z: [M+H]+ Calcd for C₄₀H₃₅ClFN₉O₆S 823.2; Found 824.2

### Step 13

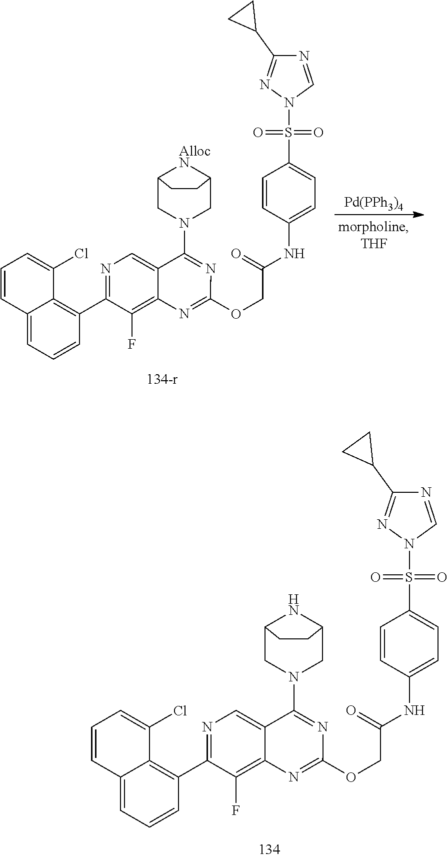

A solution of allyl (1R,5S)-3-(7-(8-chloronaphthalen-1-yl)-2-(2-((4-((3-cyclopropyl-1H-1,2,4-triazol-1-yl)sulfonyl)phenyl)amino)-2-oxoethoxy)-8-fluoropyrido[4,3-d]pyrimidin-4-yl)-3,8-diazabicyclo[3.2.1]octane-8-carboxylate (80 mg, 0.09 mmol, 1 equiv) and tetrakis(triphenylphosphine)palladium(0) (12 mg, 0.01 mmol, 0.1 equiv) morpholine (23 mg, 0.29 mmol, 3 equiv) in THE (3 mL) was stirred for 1 h at 25° C. under N₂ atmosphere. The resulting mixture was filtered. The filtrate was concentrated under reduced pressure to give the crude product which was purified by prep-HPLC (column: Xtimate C18 100*30 mm*3 um, condition: 32-72% B (A=water (0.05% ammonia hydroxide v/v)), B=acetonitrile), flow rate: 25 mL/min, UV Detector 220 nm) to afford 134 (24 mg, 33.41%) m/z: [M+H]⁺ Calcd for C₃₆H₃₂ClFN₉O₄S 740.2; Found 740.3 ¹H NMR (400 MHz, DMSO-d6) δ 10.90 (s, 1H), 9.11 (d, J=17.2 Hz, 2H), 8.20 (dd, J=8.3, 1.4 Hz, 1H), 8.10 (dd, J=8.3, 1.4 Hz, 1H), 8.07-7.98 (m, 2H), 7.96-7.87 (m, 2H), 7.76-7.68 (m, 1H), 7.68-7.51 (m, 3H), 4.98 (s, 2H), 4.45-4.37 (m, 2H), 3.58 (d, J=12.3 Hz, 2H), 3.43 (s, 2H), 2.00 (tt, J=8.6, 4.9 Hz, 1H), 1.46-1.40 (m, 4H), 0.96 (ddt, J=11.9, 8.3, 3.2 Hz, 2H), 0.88-0.76 (m, 2H).

### Synthesis of Compound 135

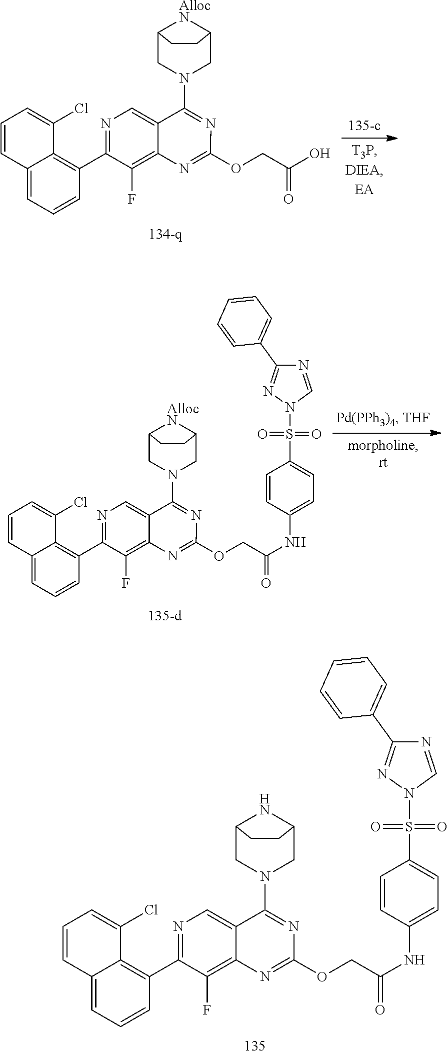

### Intermediate 134-c

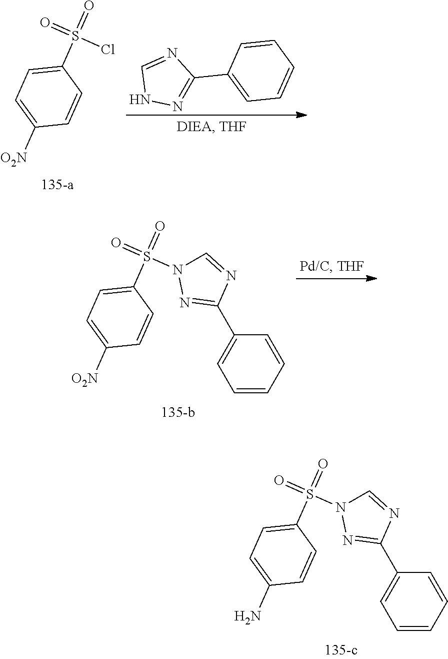

A solution of 4-nitrobenzenesulfonyl chloride (150 mg, 0.67 mmol, 1 equiv) and 3-phenyl-1H-1,2,4-triazole (118 mg, 0.81 mmol, 1.2 equiv) DIEA (263 mg, 2.03 mmol, 3 equiv) in THE (2 mL) was stirred for 1 h at 25° C. under N₂ atmosphere. The resulting solution was used in the next step directly without further purification. To the reaction solution of 1-(4-nitrobenzenesulfonyl)-3-phenyl-1,2,4-triazole in THE was added Pd/C (65 mg, 0.606 mmol, 2 equiv), and diluted with THE (2 mL). The resulting mixture was stirred for 1 h at 25° C. under H₂ atmosphere. The resulting mixture was filtered; the filter cake was washed with THE (2 mL×2). The filtrate was concentrated under reduced pressure. The residue was purified by prep-HPLC (column: Xtimate C18 100*30 mm*3 um, condition: 22-53% B (A=water (0.05% ammonia hydroxide v/v)), B=acetonitrile), flow rate: 40 mL/min, UV Detector 220 nm) to afford 4-(3-phenyl-1,2,4-triazol-1-ylsulfonyl)-aniline (135-c) (57 mg, 62.69%) as a white solid. m/z: [M+H]⁺ Calcd for C₁₄H₁₃N₄O₂S 301.1; Found 301.1. (300 MHz, DMSO-d6) δ 9.27 (s, 1H), 8.05-7.93 (m, 2H), 7.78-7.67 (m, 2H), 7.52-7.47 (m, 3H), 6.74-6.63 (m, 4H).

### Step 1

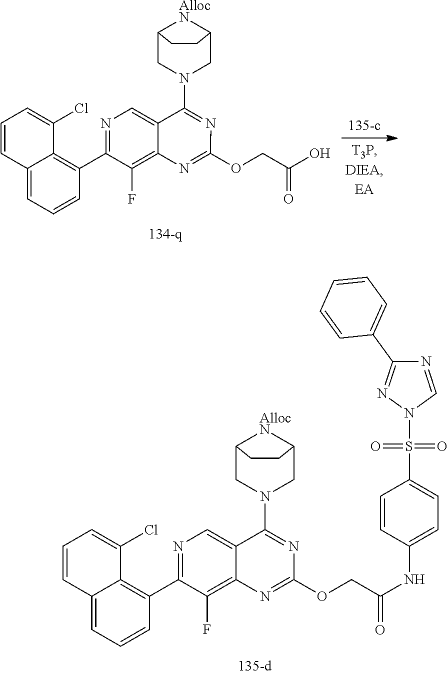

A solution of 2-((4-((1R,5S)-8-((allyloxy)carbonyl)-3,8-diazabicyclo[3.2.1]octan-3-yl)-7-(8-chloronaphthalen-1-yl)-8-fluoropyrido[4,3-d]pyrimidin-2-yl)oxy)acetic acid (100 mg, 0.17 mmol, 1 equiv) and 135-c (78 mg, 0.25 mmol, 1.5 equiv), DIEA (67 mg, 0.51 mmol, 3 equiv) in propylphosphonic anhydride (2 mL, 50% in ethyl acetate) and ethyl acetate (2 mL) was stirred for 1 h at 50° C. under N₂ atmosphere. The resulting mixture was extracted with ethyl acetate (5 mL×2). The combined organic layers were washed with brine (5 mL×2), dried over anhydrous Na₂SO₄. After filtration, the filtrate was concentrated under reduced pressure. The residue was purified by silica gel column chromatography, eluted with methanol/DCM: (0%˜20%) to afford 135-d (80 mg, 53.75%) as a white solid m/z: [M+H]⁺ Calcd for C₄₃H₃₆ClFN₉O₆S 860.2; Found 860.2.

### Step 2

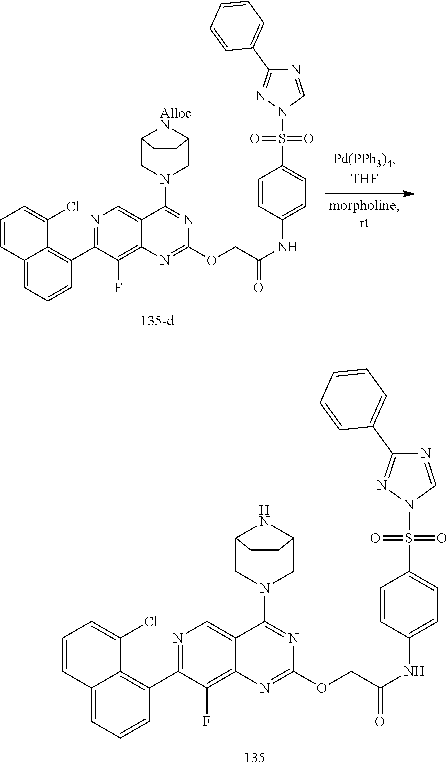

A mixture of allyl (1R,5S)-3-(7-(8-chloronaphthalen-1-yl)-8-fluoro-2-(2-oxo-2-((4-((3-phenyl-1H-1,2,4-triazol-1-yl)sulfonyl)phenyl)amino)ethoxy)pyrido[4,3-d]pyrimidin-4-yl)-3, 8-diazabicyclo[3.2.1]octane-8-carboxylate (80 mg, 0.093 mmol, 1 equiv) and Pd(PPh₃)₄ (4 mg, 0.009 mmol, 0.2 equiv) morpholine (16 mg, 0.18 mmol, 2 equiv) in THF (3 mL) was stirred for 1 h at 25° C. under N₂ atmosphere. The resulting mixture was filtered, the filtrate was concentrated under reduced pressure to give the crude product which was purified by prep-HPLC (column: Xtimate C18 100*30 mm*3 um, condition: 32-72% B (A=water (0.05% ammonia hydroxide v/v)), B=acetonitrile), flow rate: 25 mL/min, UV Detector 220 nm) to afford 135 (10.5 mg, 14.55%) m/z: [M+H]⁺ Calcd for C₃₉H₃₂ClFN₉O₄S 776.2; Found 776.3 ¹H NMR (400 MHz, DMSO-d6) δ 10.91 (s, 1H), 9.42 (s, 1H), 9.06 (s, 1H), 8.18 (dd, J=8.3, 1.4 Hz, 1H), 8.10 (dd, J=10.9, 8.3 Hz, 3H), 7.99 (dd, J=6.6, 2.9 Hz, 2H), 7.96-7.90 (m, 2H), 7.70 (t, J=7.7 Hz, 1H), 7.63 (dd, J=7.5, 1.4 Hz, 1H), 7.62-7.49 (m, 3H), 7.53-7.45 (m, 2H), 4.96 (s, 2H), 4.37 (d, J=11.9 Hz, 2H), 3.54 (d, J=12.2 Hz, 2H), 3.33-3.35 (m, 2H), 1.38-1.33 (m, 4H).

TABLE 1

Non-limiting heteroaryl sulfonyl compounds of the present invention

| Com- | |
|---|---|
| pound # | Structure |
| 101 | 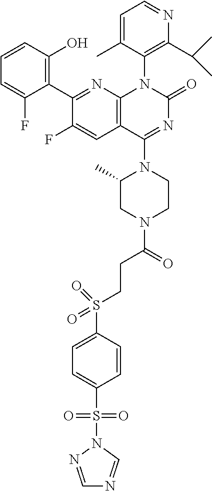 [SMILES: Cc1ccnc(C(C)C)c1-n1c(=O)nc(N2CCN(C(=O)CCS(=O)(=O)c3ccc(S(=O)(=O)n4cncn4)cc3)C[C@@H]2C)c2cc(F)c(-c3c(O)cccc3F)nc21] |
| 102 | 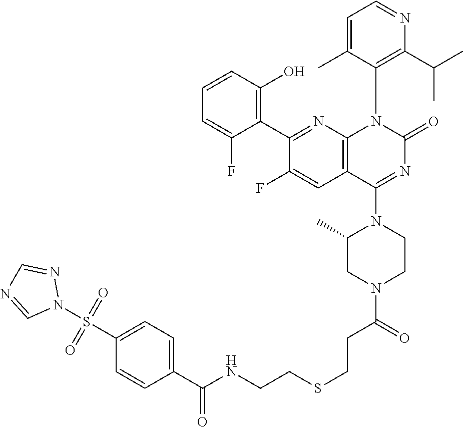 [SMILES: Cc1ccnc(C(C)C)c1-n1c(=O)nc(N2CCN(C(=O)CCSCCNC(=O)c3ccc(S(=O)(=O)n4cncn4)cc3)C[C@@H]2C)c2cc(F)c(-c3c(O)cccc3F)nc21] |
| 103 | 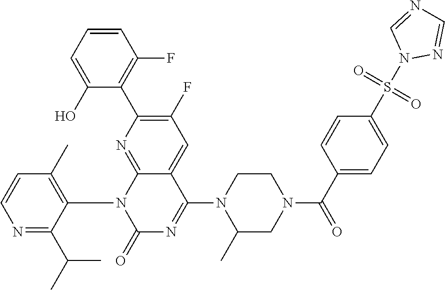 [SMILES: Cc1ccnc(C(C)C)c1-n1c(=O)nc(N2CCN(C(=O)c3ccc(S(=O)(=O)n4cncn4)cc3)CC2C)c2cc(F)c(-c3c(O)cccc3F)nc21] |
| 104 | 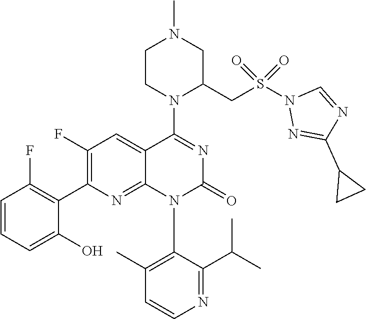 [SMILES: Cc1ccnc(C(C)C)c1-n1c(=O)nc(N2CCN(C)CC2CS(=O)(=O)n2cnc(C3CC3)n2)c2cc(F)c(-c3c(O)cccc3F)nc21] |
| 105 | 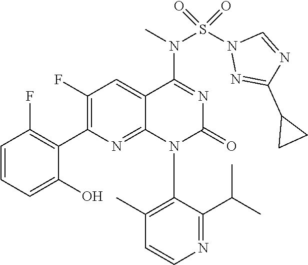 [SMILES: Cc1ccnc(C(C)C)c1-n1c(=O)nc(N(C)S(=O)(=O)n2cnc(C3CC3)n2)c2cc(F)c(-c3c(O)cccc3F)nc21] |
| 106 | 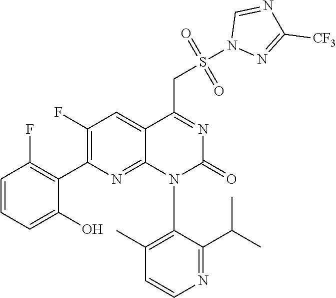 [SMILES: Cc1ccnc(C(C)C)c1-n1c(=O)nc(CS(=O)(=O)n2cnc(C(F)(F)F)n2)c2cc(F)c(-c3c(O)cccc3F)nc21] |
| 107 | 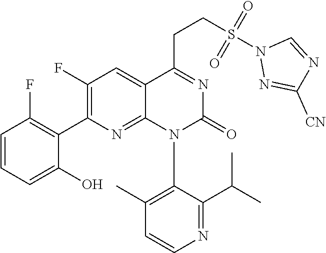 [SMILES: Cc1ccnc(C(C)C)c1-n1c(=O)nc(CCS(=O)(=O)n2cnc(C#N)n2)c2cc(F)c(-c3c(O)cccc3F)nc21] |
| 108 | 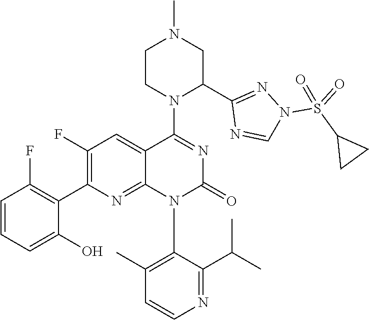 [SMILES: Cc1ccnc(C(C)C)c1-n1c(=O)nc(N2CCN(C)CC2c2ncn(S(=O)(=O)C3CC3)n2)c2cc(F)c(-c3c(O)cccc3F)nc21] |
| 109 | 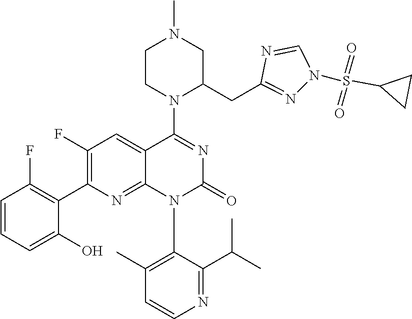 [SMILES: Cc1ccnc(C(C)C)c1-n1c(=O)nc(N2CCN(C)CC2Cc2ncn(S(=O)(=O)C3CC3)n2)c2cc(F)c(-c3c(O)cccc3F)nc21] |
| 110 | 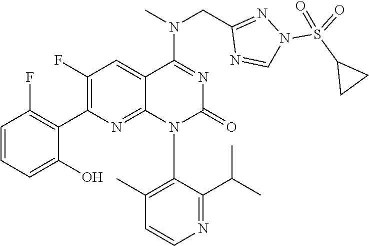 [SMILES: Cc1ccnc(C(C)C)c1-n1c(=O)nc(N(C)Cc2ncn(S(=O)(=O)C3CC3)n2)c2cc(F)c(-c3c(O)cccc3F)nc21] |
| 111 | 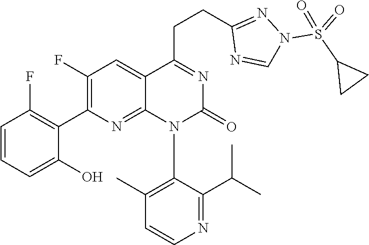 [SMILES: Cc1ccnc(C(C)C)c1-n1c(=O)nc(CCc2ncn(S(=O)(=O)C3CC3)n2)c2cc(F)c(-c3c(O)cccc3F)nc21] |
| 112 | 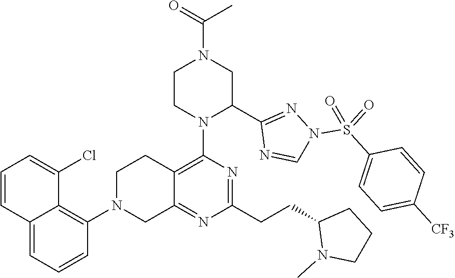 [SMILES: CC(=O)N1CCN(c2nc(CC[C@@H]3CCCN3C)nc3c2CCN(c2cccc4cccc(Cl)c24)C3)C(c2ncn(S(=O)(=O)c3ccc(C(F)(F)F)cc3)n2)C1] |
| 113 | 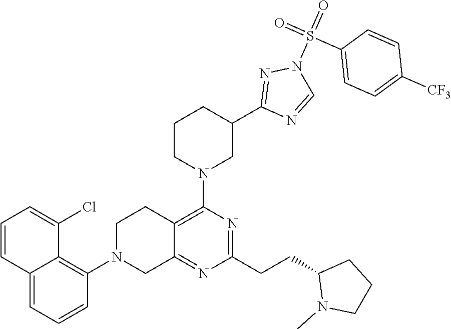 [SMILES: CN1CCC[C@H]1CCc1nc2c(c(N3CCCC(c4ncn(S(=O)(=O)c5ccc(C(F)(F)F)cc5)n4)C3)n1)CCN(c1cccc3cccc(Cl)c13)C2] |
| 114 | 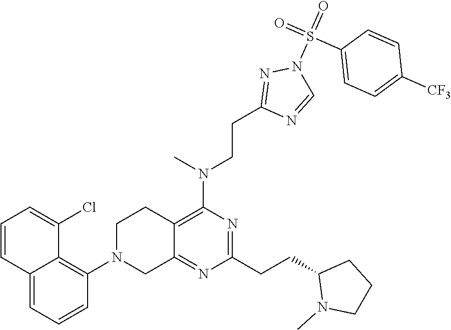 [SMILES: CN(CCc1ncn(S(=O)(=O)c2ccc(C(F)(F)F)cc2)n1)c1nc(CC[C@@H]2CCCN2C)nc2c1CCN(c1cccc3cccc(Cl)c13)C2] |
| 115 | 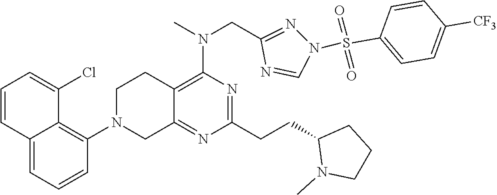 [SMILES: CN(Cc1ncn(S(=O)(=O)c2ccc(C(F)(F)F)cc2)n1)c1nc(CC[C@@H]2CCCN2C)nc2c1CCN(c1cccc3cccc(Cl)c13)C2] |
| 116 | 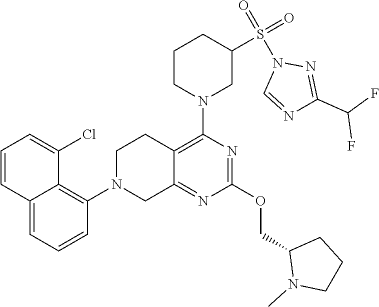 [SMILES: CN1CCC[C@H]1COc1nc2c(c(N3CCCC(S(=O)(=O)n4cnc(C(F)F)n4)C3)n1)CCN(c1cccc3cccc(Cl)c13)C2] |
| 117 | 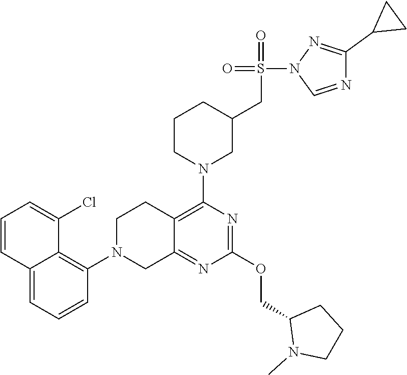 [SMILES: CN1CCC[C@H]1COc1nc2c(c(N3CCCC(CS(=O)(=O)n4cnc(C5CC5)n4)C3)n1)CCN(c1cccc3cccc(Cl)c13)C2] |
| 118 | 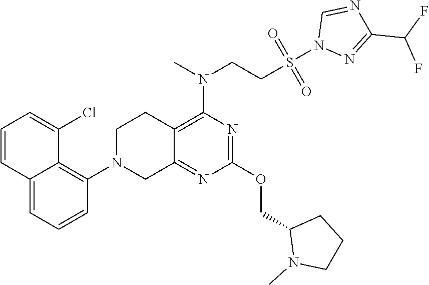 [SMILES: CN(CCS(=O)(=O)n1cnc(C(F)F)n1)c1nc(OC[C@@H]2CCCN2C)nc2c1CCN(c1cccc3cccc(Cl)c13)C2] |
| 119 | 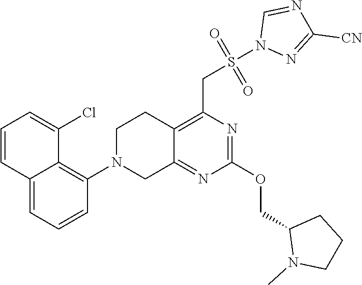 [SMILES: CN1CCC[C@H]1COc1nc2c(c(CS(=O)(=O)n3cnc(C#N)n3)n1)CCN(c1cccc3cccc(Cl)c13)C2] |
| 120 | 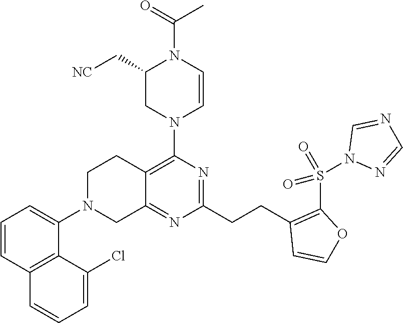 [SMILES: CC(=O)N1C=CN(c2nc(CCc3ccoc3S(=O)(=O)n3cncn3)nc3c2CCN(c2cccc4cccc(Cl)c24)C3)C[C@@H]1CC#N] |
| 121 | 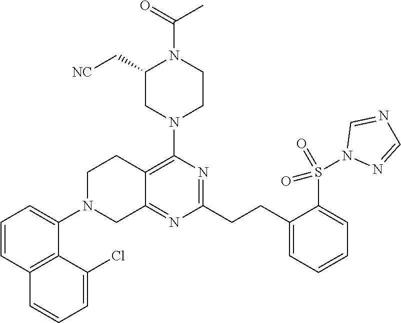 [SMILES: CC(=O)N1CCN(c2nc(CCc3ccccc3S(=O)(=O)n3cncn3)nc3c2CCN(c2cccc4cccc(Cl)c24)C3)C[C@@H]1CC#N] |
| 122 | 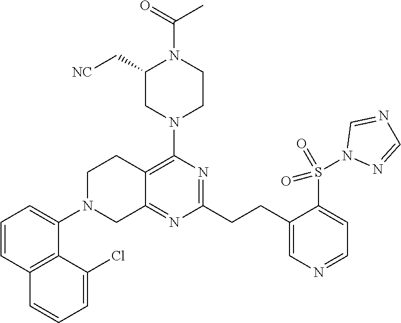 [SMILES: CC(=O)N1CCN(c2nc(CCc3cnccc3S(=O)(=O)n3cncn3)nc3c2CCN(c2cccc4cccc(Cl)c24)C3)C[C@@H]1CC#N] |
| 123 | 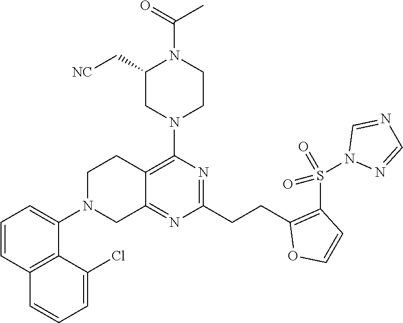 [SMILES: CC(=O)N1CCN(c2nc(CCc3occc3S(=O)(=O)n3cncn3)nc3c2CCN(c2cccc4cccc(Cl)c24)C3)C[C@@H]1CC#N] |
| 124 | 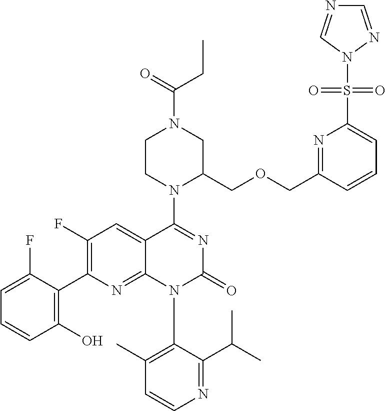 [SMILES: CCC(=O)N1CCN(c2nc(=O)n(-c3c(C)ccnc3C(C)C)c3nc(-c4c(O)cccc4F)c(F)cc23)C(COCc2cccc(S(=O)(=O)n3cncn3)n2)C1] |
| 125 | 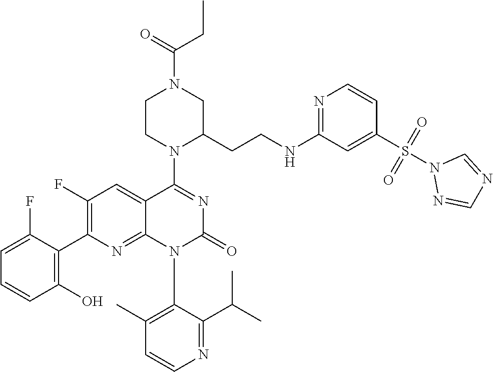 [SMILES: CCC(=O)N1CCN(c2nc(=O)n(-c3c(C)ccnc3C(C)C)c3nc(-c4c(O)cccc4F)c(F)cc23)C(CCNc2cc(S(=O)(=O)n3cncn3)ccn2)C1] |
| 126 | 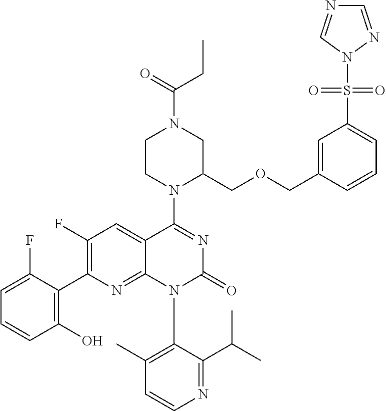 [SMILES: CCC(=O)N1CCN(c2nc(=O)n(-c3c(C)ccnc3C(C)C)c3nc(-c4c(O)cccc4F)c(F)cc23)C(COCc2cccc(S(=O)(=O)n3cncn3)c2)C1] |
| 127 | 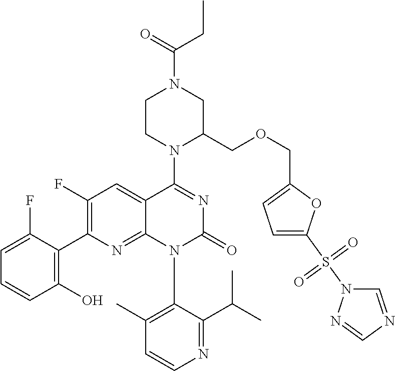 [SMILES: CCC(=O)N1CCN(c2nc(=O)n(-c3c(C)ccnc3C(C)C)c3nc(-c4c(O)cccc4F)c(F)cc23)C(COCc2ccc(S(=O)(=O)n3cncn3)o2)C1] |
| 128 | 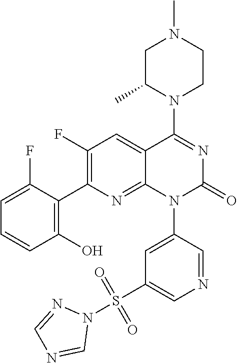 [SMILES: C[C@@H]1CN(C)CCN1c1nc(=O)n(-c2cncc(S(=O)(=O)n3cncn3)c2)c2nc(-c3c(O)cccc3F)c(F)cc12] |
| 129 | 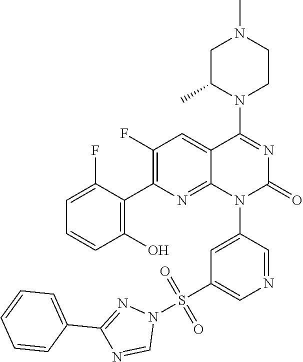 [SMILES: C[C@@H]1CN(C)CCN1c1nc(=O)n(-c2cncc(S(=O)(=O)n3cnc(-c4ccccc4)n3)c2)c2nc(-c3c(O)cccc3F)c(F)cc12] |
| 130 | 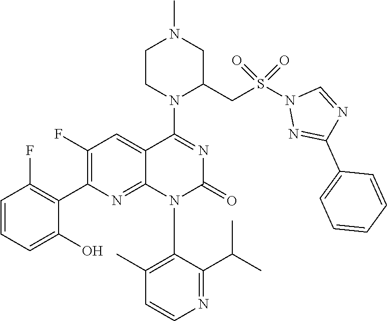 [SMILES: Cc1ccnc(C(C)C)c1-n1c(=O)nc(N2CCN(C)CC2CS(=O)(=O)n2cnc(-c3ccccc3)n2)c2cc(F)c(-c3c(O)cccc3F)nc21] |
| 131 | 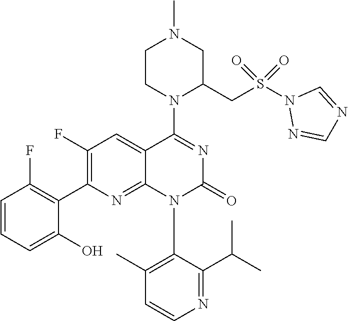 [SMILES: Cc1ccnc(C(C)C)c1-n1c(=O)nc(N2CCN(C)CC2CS(=O)(=O)n2cncn2)c2cc(F)c(-c3c(O)cccc3F)nc21] |
| 132 | 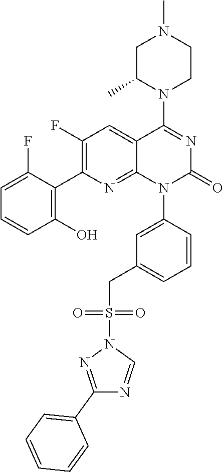 [SMILES: C[C@@H]1CN(C)CCN1c1nc(=O)n(-c2cccc(CS(=O)(=O)n3cnc(-c4ccccc4)n3)c2)c2nc(-c3c(O)cccc3F)c(F)cc12] |
| 133 | 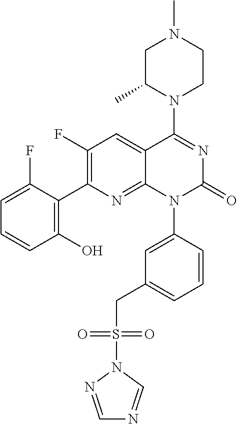 [SMILES: C[C@@H]1CN(C)CCN1c1nc(=O)n(-c2cccc(CS(=O)(=O)n3cncn3)c2)c2nc(-c3c(O)cccc3F)c(F)cc12] |

TABLE 2
Additional non-limiting heteroaryl sulfonyl compounds of the present
invention

| Com- | |
|---|---|
| pound # | Structure |
| 134 | 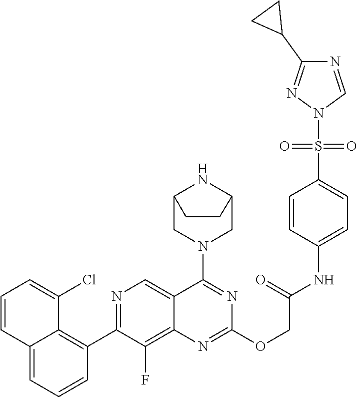 [SMILES: O=C(COc1nc(N2CC3CCC(C2)N3)c2cnc(-c3cccc4cccc(Cl)c34)c(F)c2n1)Nc1ccc(S(=O)(=O)n2cnc(C3CC3)n2)cc1] |
| 135 | 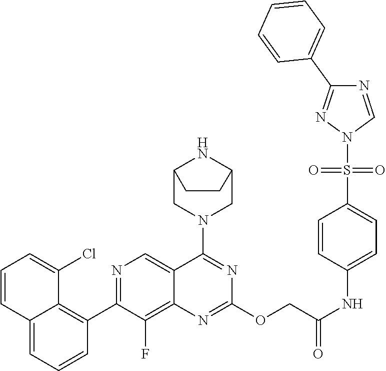 [SMILES: O=C(COc1nc(N2CC3CCC(C2)N3)c2cnc(-c3cccc4cccc(Cl)c34)c(F)c2n1)Nc1ccc(S(=O)(=O)n2cnc(-c3ccccc3)n2)cc1] |

All publications and patent applications cited in this specification are herein incorporated by reference as if each individual publication or patent application were specifically and individually indicated to be incorporated by reference.

Although the foregoing invention has been described in some detail by way of illustration and example for the purposes of clarity of understanding, it will be readily apparent to one of ordinary skill in the art in light of the teaching of this invention that certain changes and modification may be made thereto without departing from the spirit or scope of the invention as defined in the claims.

We claim:

1. A pharmaceutically acceptable compound of Formula: 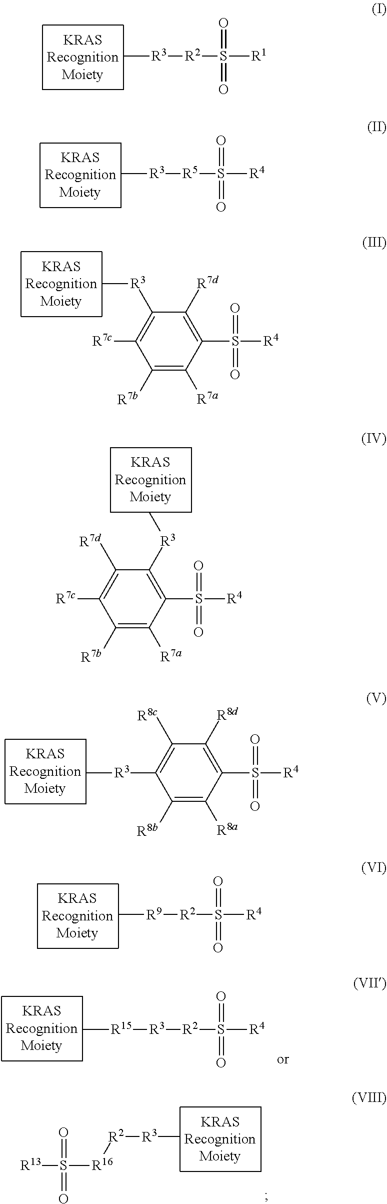
or a pharmaceutically acceptable salt thereof;
wherein:
R¹ is selected from the group consisting of: 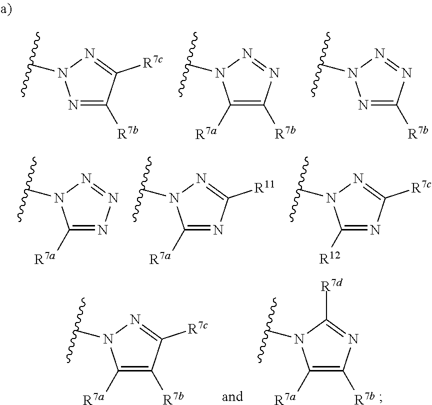
b) a bicyclic heteroaryl which is optionally substituted with 1, 2, or 3 substituents selected from R⁷;
R² is independently selected at each instance from the group consisting of bond, alkyl, cycloalkyl, aryl, heterocycle, —S—, —O—, —NR⁶—, —(CH₂)ₚ—C(O)—NR⁶—, —(CH₂CH₂O)ₚ—, —(OCH₂CH₂)ₚ—, —NR⁶C(O)NR⁶—, —C(O)NR⁶—, —OC(O)NR⁶—, aryl-C(O)—NR⁶—, heteroaryl-C(O)—NR⁶—, bicycle, and heteroaryl, each of which is optionally substituted with 1, 2, or 3 substituents selected from R⁷;
R³ is independently selected at each instance from bond, alkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, and heteroaryl, each of which is optionally substituted with 1, 2, or 3 substituents selected from R⁷;
p is independently selected from 1, 2, 3, 4, 5, and 6;
R⁴ is a heteroaryl group with 1, 2, 3, or 4 nitrogen atoms, where the bond to the sulfur atom is through one of the nitrogen atoms present in the cycle, and each heteroaryl is optionally substituted with 1, 2, or 3 substituents selected from R⁷;
R⁵ is selected from the group consisting of alkyl, cycloalkyl, naphthyl, heterocycle, —S—, —O—, —NR⁶—, —(CH₂)ₚ—C(O)—NR⁶—, —(CH₂CH₂O)ₚ—, —(OCH₂CH₂)ₚ—, —NR⁶C(O)NR⁶—, —C(O)NR⁶—, —OC(O)NR⁶—, heteroaryl-C(O)—NR⁶—, bicycle, and heteroaryl, each of which is optionally substituted with 1, 2, or 3 substituents selected from R⁷;
R⁶ is independently selected at each instance hydrogen or alkyl;
R⁷, R^{7a}, R^{7b}, R^{7c}, and R^{7d} are independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, and —SR⁶, wherein each alkyl, haloalkyl, alkenyl, cycloalkyl, heterocycle, aryl, and heteroaryl is optionally substituted with 1, 2, or 3 substituents selected from R¹⁷;
R¹⁷ is independently selected in each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, —S(O)₂N(R⁶)₂, and —SR⁶;
R^{8a}, R^{8b}, R^{8c}, and R^{8d} are independently selected at each instance from the group consisting of R⁷ and R¹² wherein at least one of R⁸, R^{8b}, R^{8g}, and R^{8d} is R¹²;
R⁹ is alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, or heteroaryl, each of which is optionally substituted with 1, 2, or 3 substituents selected from R⁷;
R¹¹ is hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, or —SR⁶, wherein each alkyl, haloalkyl, alkenyl, cycloalkyl, heterocycle, aryl, and heteroaryl optionally substituted with 1, 2, or 3 substituents selected from R¹⁷;
R¹² is halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, or —SR⁶, wherein each alkyl, haloalkyl, alkenyl, cycloalkyl, heterocycle, aryl, and heteroaryl is optionally substituted with 1, 2, or 3 substituents selected from R¹⁷;
R¹³ is alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, or —NR⁶C(O)N(R⁶)₂, each of which is optionally substituted with 1, 2, or 3 substituents selected from R⁷;
R¹⁵ is alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, or heteroaryl, each of which except bond is optionally substituted with 1, 2, or 3 substituents independently selected from R⁷;
R¹⁶ is a heteroaryl group, where the bond to the sulfur atom is through one of the nitrogen atoms present in the cycle, and R¹⁶ is optionally substituted with 1, 2, or 3 substituents selected from R⁷; and
KRAS Recognition Moiety is a molecule which can bind to or otherwise anchors to KRAS.

2. The pharmaceutically acceptable compound of claim 1, wherein R³ is phenyl, alkyl, or heteroaryl optionally substituted with 1, 2, or 3 substituents selected from R⁷.

3. The pharmaceutically acceptable compound of claim 2, wherein the compound is of Formula: 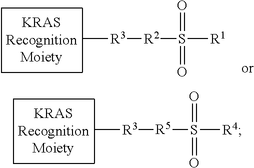
or a pharmaceutically acceptable salt thereof.

4. The pharmaceutically acceptable compound of claim 3, wherein the compound is not substituted.

5. The pharmaceutically acceptable compound of claim 3, wherein R¹ and R⁴ are

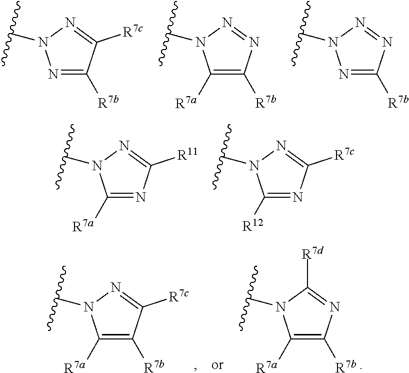

6. The pharmaceutically acceptable compound of claim 5, wherein R^{z} is bond.

7. The pharmaceutically acceptable compound of claim 5, wherein R² is phenyl, alkyl, or heteroaryl optionally substituted with 1, 2, or 3 substituents selected from R⁷.

8. The pharmaceutically acceptable compound of claim 2, wherein the compound is Formula: 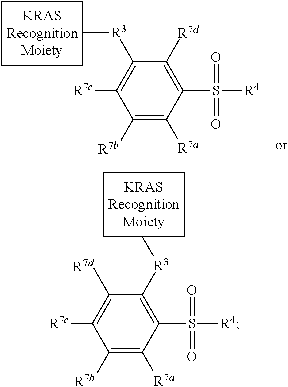
or a pharmaceutically acceptable salt thereof.

9. The pharmaceutically acceptable compound of claim 8, wherein the compound is not substituted.

10. The pharmaceutically acceptable compound of claim 2, wherein the compound is Formula: 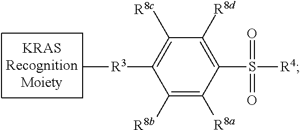
or a pharmaceutically acceptable salt thereof.

11. The pharmaceutically acceptable compound of claim 10, wherein the compound is not substituted.

12. The pharmaceutically acceptable compound of claim 2, wherein the compound is Formula: 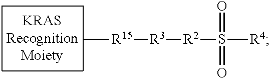
or a pharmaceutically acceptable salt thereof.

13. The pharmaceutically acceptable compound of claim 12, wherein the compound is not substituted.

14. The pharmaceutically acceptable compound of claim 12, wherein R⁴ is

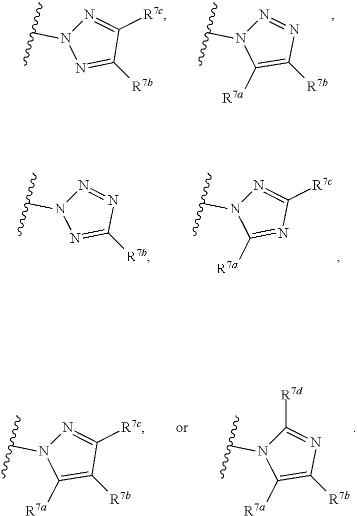

15. The pharmaceutically acceptable compound of claim 14, wherein R² is phenyl, alkyl, or heteroaryl optionally substituted with 1, 2, or 3 substituents selected from R⁷.

16. The pharmaceutically acceptable compound of claim 2, wherein the compound is of Formula: 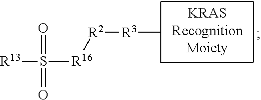
or a pharmaceutically acceptable salt thereof.

17. The pharmaceutically acceptable compound of claim 16, wherein the compound is not substituted.

18. The pharmaceutically acceptable compound of claim 16, wherein R¹³ is phenyl optionally substituted with 1, 2, or 3 substituents selected from R⁷.

19. The pharmaceutically acceptable compound of claim 16, wherein R¹³ is alkyl optionally substituted with 1, 2, or 3 substituents selected from R⁷.

20. The pharmaceutically acceptable compound of claim 16, wherein R¹⁶ is

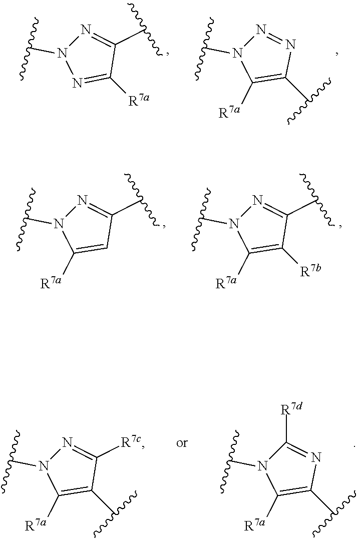

21. The pharmaceutically acceptable compound of claim 2, wherein the compound is of Formula: 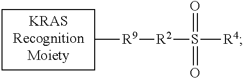
or a pharmaceutically acceptable salt thereof.

22. The pharmaceutically acceptable compound of claim 21, wherein the compound is not substituted.

23. The pharmaceutically acceptable compound of claim 21, wherein R⁴ is

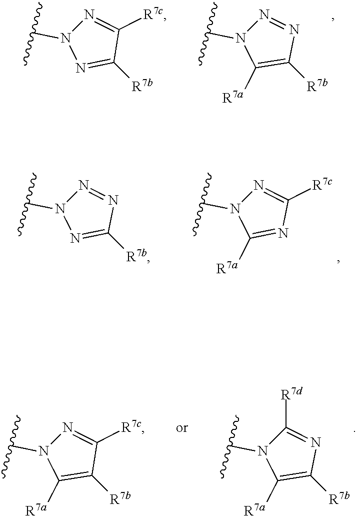

24. The pharmaceutically acceptable compound of claim 23, wherein R⁹ is selected from alkyl, cycloalkyl, and heteroaryl, each of which except bond is optionally substituted with 1, 2, or 3 substituents independently selected from R⁷.

25. The pharmaceutically acceptable compound of claim 2, wherein KRAS Recognition Moiety is 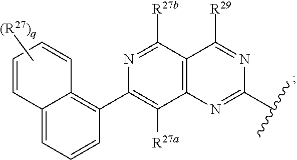
wherein
R²⁷ is independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NRC(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, and —SR⁶;
R^{27a} and R^{27b} are independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, and —SR⁶, wherein each alkyl, haloalkyl, alkenyl, cycloalkyl, heterocycle, aryl, and heteroaryl optionally substituted with 1, 2, 3, or 4 substituents selected from R¹⁸;
R¹⁸ is independently selected at each instance from the group consisting of hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, and —SR¹⁹;
R²⁹ is independently selected from hydrogen, alkyl, haloalkyl, cycloalkyl, aryl, heterocycle, halogen and heteroaryl each of which is optionally substituted with 1 or 2 substituents independently selected from R¹⁷; and
q is 0, 1, 2, or 3.

26. The pharmaceutically acceptable compound of claim 25, wherein q is 0.

27. The pharmaceutically acceptable compound of claim 26, wherein R²⁹ is cycloalkyl, aryl, heterocycle, or heteroaryl each of which is optionally substituted with 1 or 2 substituents independently selected from R¹⁷.

28. The pharmaceutically acceptable compound of claim 2, wherein KRAS Recognition Moiety is 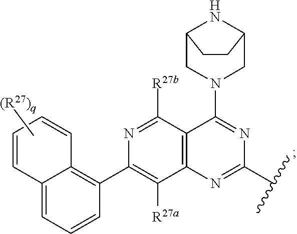
wherein
R²⁷ is independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, and —SR⁶;
R^{27a} and R^{27b} are independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, and —SR⁶, wherein each alkyl, haloalkyl, alkenyl, cycloalkyl, heterocycle, aryl, and heteroaryl optionally substituted with 1, 2, 3, or 4 substituents selected from R¹⁸;
R¹⁸ is independently selected at each instance from the group consisting of hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, and —SR¹⁹; and
q is 0, 1, 2, or 3.

29. The pharmaceutically acceptable compound of claim 28, wherein q is 0.

30. The pharmaceutically acceptable compound of claim 1 of structure: 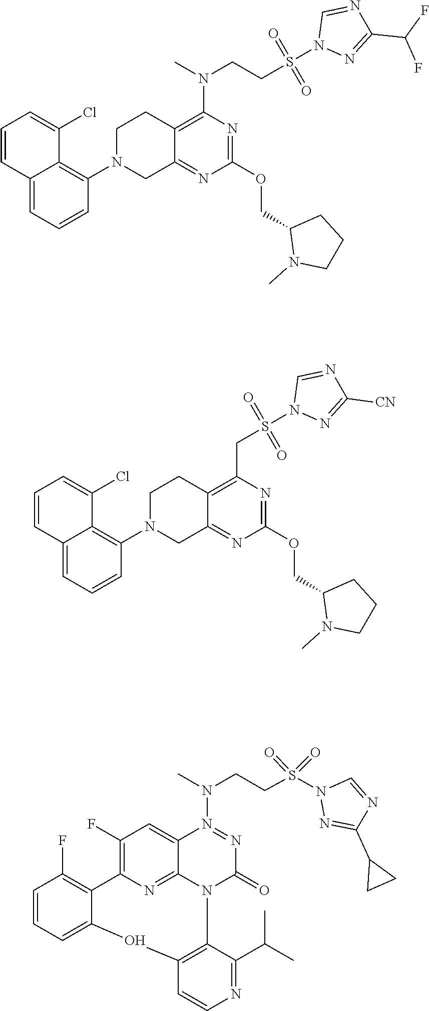 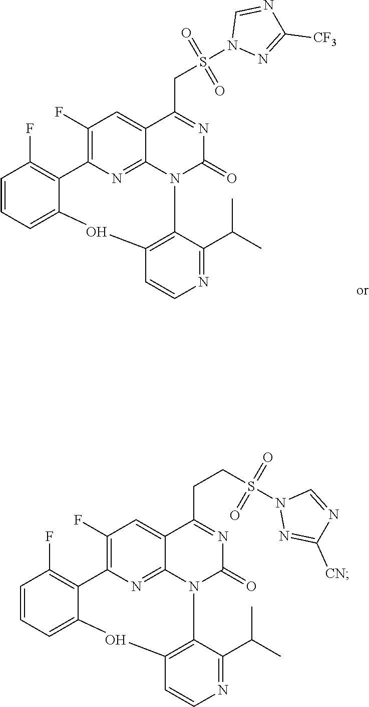 [SMILES: CC(C)c1nccc2c1-n1c(=O)nc(CS(=O)(=O)n3cnc(C(F)(F)F)n3)c3cc(F)c(nc31)-c1c(F)cccc1O2 ; CC(C)c1nccc2c1-n1c(=O)nc(CCS(=O)(=O)n3cnc(C#N)n3)c3cc(F)c(nc31)-c1c(F)cccc1O2]
or a pharmaceutically acceptable salt thereof.

31. The pharmaceutically acceptable compound of claim 1 of structure: 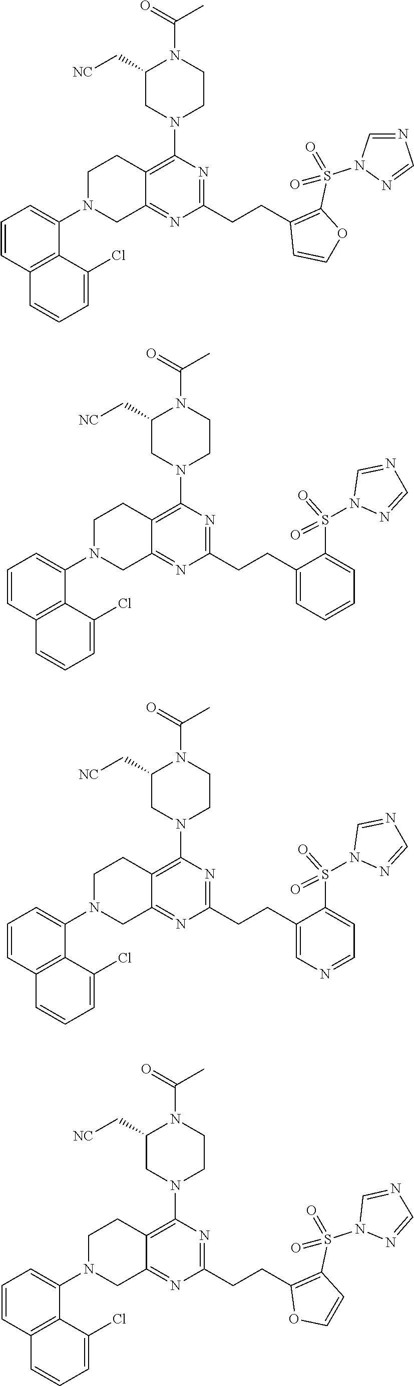 [SMILES: CC(=O)N1CCN(c2nc(CCc3ccoc3S(=O)(=O)n3cncn3)nc3c2CCN(c2cccc4cccc(Cl)c24)C3)C[C@@H]1CC#N ; CC(=O)N1CCN(c2nc(CCc3ccccc3S(=O)(=O)n3cncn3)nc3c2CCN(c2cccc4cccc(Cl)c24)C3)C[C@@H]1CC#N ; CC(=O)N1CCN(c2nc(CCc3cnccc3S(=O)(=O)n3cncn3)nc3c2CCN(c2cccc4cccc(Cl)c24)C3)C[C@@H]1CC#N ; CC(=O)N1CCN(c2nc(CCc3occc3S(=O)(=O)n3cncn3)nc3c2CCN(c2cccc4cccc(Cl)c24)C3)C[C@@H]1CC#N] 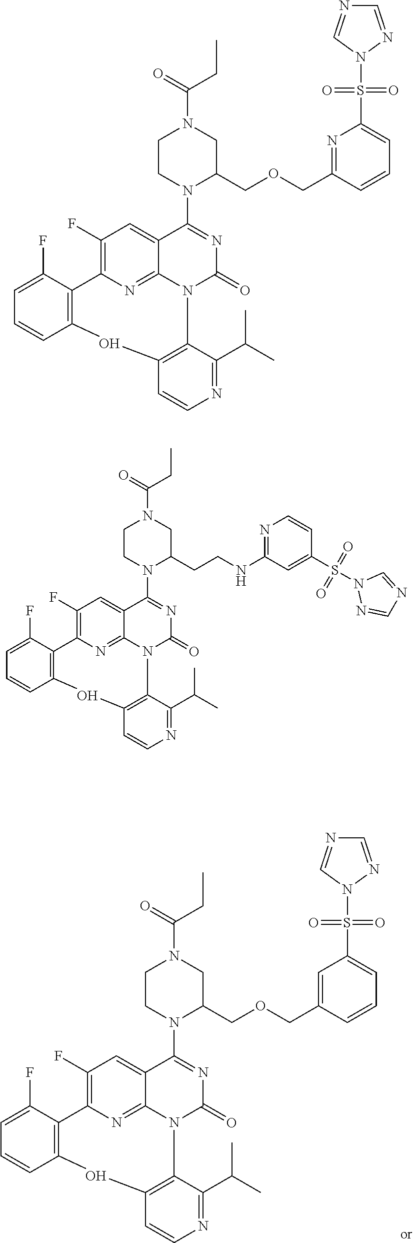 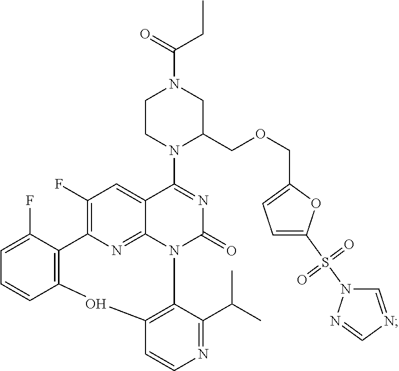 [SMILES: CCC(=O)N1CCN(c2nc(=O)n3c4nc(c(F)cc24)-c2c(F)cccc2Oc2ccnc(C(C)C)c2-3)C(COCc2ccc(S(=O)(=O)n3cncn3)o2)C1]
or a pharmaceutically acceptable salt thereof.

32. The pharmaceutically acceptable compound of claim 1 of structure: 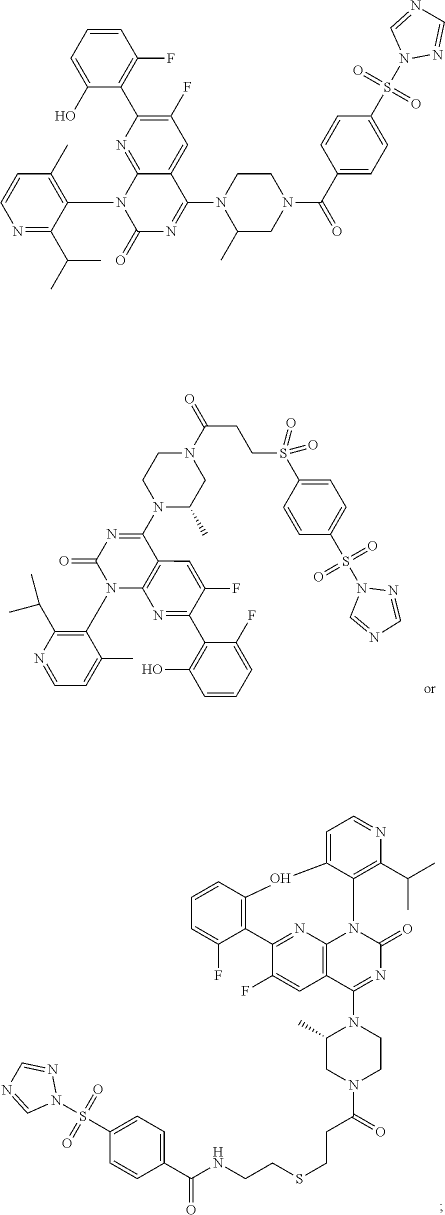 [SMILES: Cc1ccnc(C(C)C)c1-n1c(=O)nc(N2CCN(C(=O)c3ccc(S(=O)(=O)n4cncn4)cc3)CC2C)c2cc(F)c(-c3c(O)cccc3F)nc21 ; Cc1ccnc(C(C)C)c1-n1c(=O)nc(N2CCN(C(=O)CCS(=O)(=O)c3ccc(S(=O)(=O)n4cncn4)cc3)C[C@@H]2C)c2cc(F)c(-c3c(O)cccc3F)nc21 ; CC(C)c1nccc2c1-n1c(=O)nc(N3CCN(C(=O)CCSCCNC(=O)c4ccc(S(=O)(=O)n5cncn5)cc4)C[C@@H]3C)c3cc(F)c(nc31)-c1c(F)cccc1O2]
or a pharmaceutically acceptable salt thereof.

33. The pharmaceutically acceptable compound of claim 1 of structure: 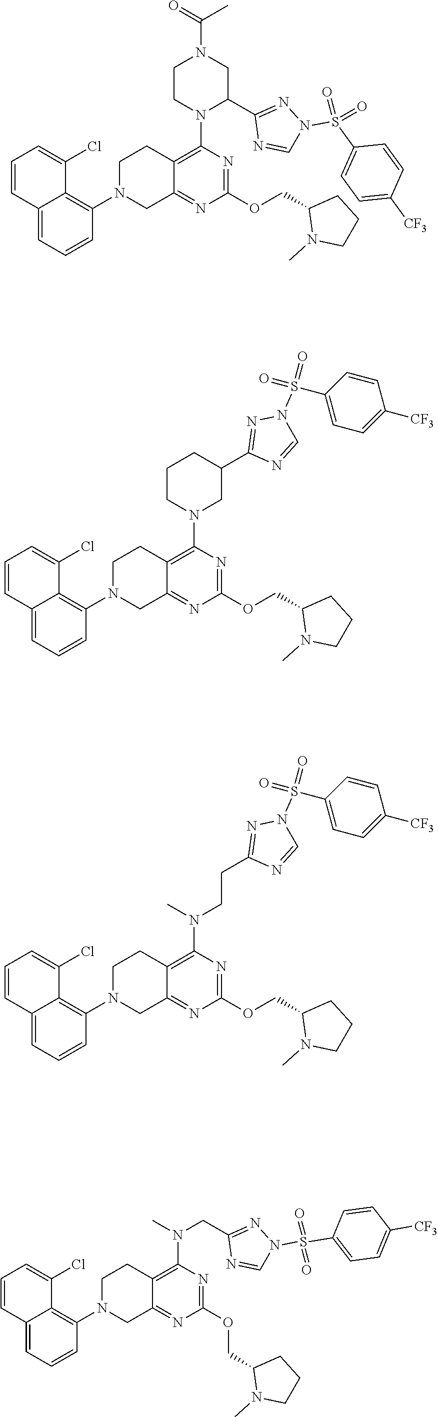 [SMILES: CC(=O)N1CCN(c2nc(OC[C@@H]3CCCN3C)nc3c2CCN(c2cccc4cccc(Cl)c24)C3)C(c2ncn(S(=O)(=O)c3ccc(C(F)(F)F)cc3)n2)C1 ; CN1CCC[C@H]1COc1nc2c(c(N3CCCC(c4ncn(S(=O)(=O)c5ccc(C(F)(F)F)cc5)n4)C3)n1)CCN(c1cccc3cccc(Cl)c13)C2 ; CN(CCc1ncn(S(=O)(=O)c2ccc(C(F)(F)F)cc2)n1)c1nc(OC[C@@H]2CCCN2C)nc2c1CCN(c1cccc3cccc(Cl)c13)C2 ; CN(Cc1ncn(S(=O)(=O)c2ccc(C(F)(F)F)cc2)n1)c1nc(OC[C@@H]2CCCN2C)nc2c1CCN(c1cccc3cccc(Cl)c13)C2] 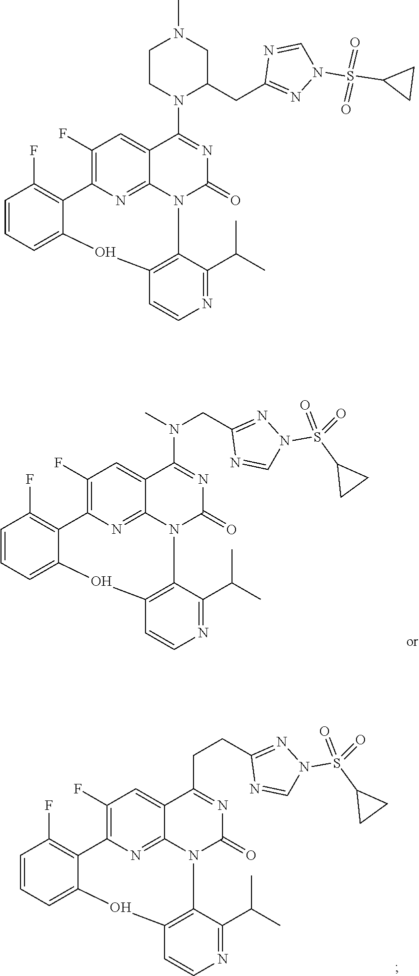 [SMILES: CC(C)c1nccc2c1-n1c(=O)nc(N3CCN(C)CC3Cc3ncn(S(=O)(=O)C4CC4)n3)c3cc(F)c(nc31)-c1c(F)cccc1O2 ; CC(C)c1nccc2c1-n1c(=O)nc(N(C)Cc3ncn(S(=O)(=O)C4CC4)n3)c3cc(F)c(nc31)-c1c(F)cccc1O2 ; CC(C)c1nccc2c1-n1c(=O)nc(CCc3ncn(S(=O)(=O)C4CC4)n3)c3cc(F)c(nc31)-c1c(F)cccc1O2]
or a pharmaceutically acceptable salt thereof.

34. The pharmaceutically acceptable compound of claim 1 of structure: 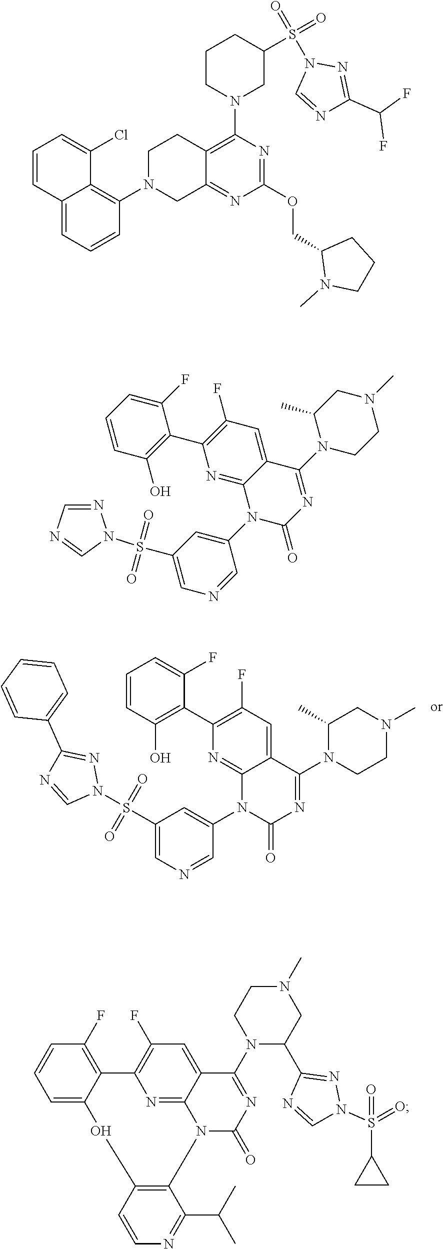 [SMILES: CN1CCC[C@H]1COc1nc2c(c(N3CCCC(S(=O)(=O)n4cnc(C(F)F)n4)C3)n1)CCN(c1cccc3cccc(Cl)c13)C2 ; C[C@@H]1CN(C)CCN1c1nc(=O)n(-c2cncc(S(=O)(=O)n3cncn3)c2)c2nc(-c3c(O)cccc3F)c(F)cc12 ; C[C@@H]1CN(C)CCN1c1nc(=O)n(-c2cncc(S(=O)(=O)n3cnc(-c4ccccc4)n3)c2)c2nc(-c3c(O)cccc3F)c(F)cc12 ; CC(C)c1nccc2c1-n1c(=O)nc(N3CCN(C)CC3c3ncn(S(=O)(=O)C4CC4)n3)c3cc(F)c(nc31)-c1c(F)cccc1O2]
or a pharmaceutically acceptable salt thereof.

35. The pharmaceutically acceptable compound of claim 1 of structure: 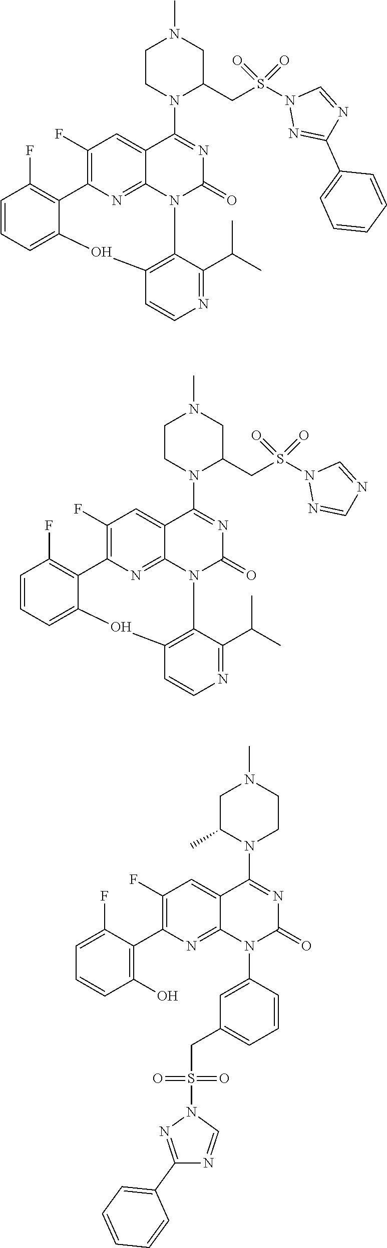 [SMILES: CC(C)c1nccc2c1-n1c(=O)nc(N3CCN(C)CC3CS(=O)(=O)n3cnc(-c4ccccc4)n3)c3cc(F)c(nc31)-c1c(F)cccc1O2 ; CC(C)c1nccc2c1-n1c(=O)nc(N3CCN(C)CC3CS(=O)(=O)n3cncn3)c3cc(F)c(nc31)-c1c(F)cccc1O2 ; C[C@@H]1CN(C)CCN1c1nc(=O)n(-c2cccc(CS(=O)(=O)n3cnc(-c4ccccc4)n3)c2)c2nc(-c3c(O)cccc3F)c(F)cc12] 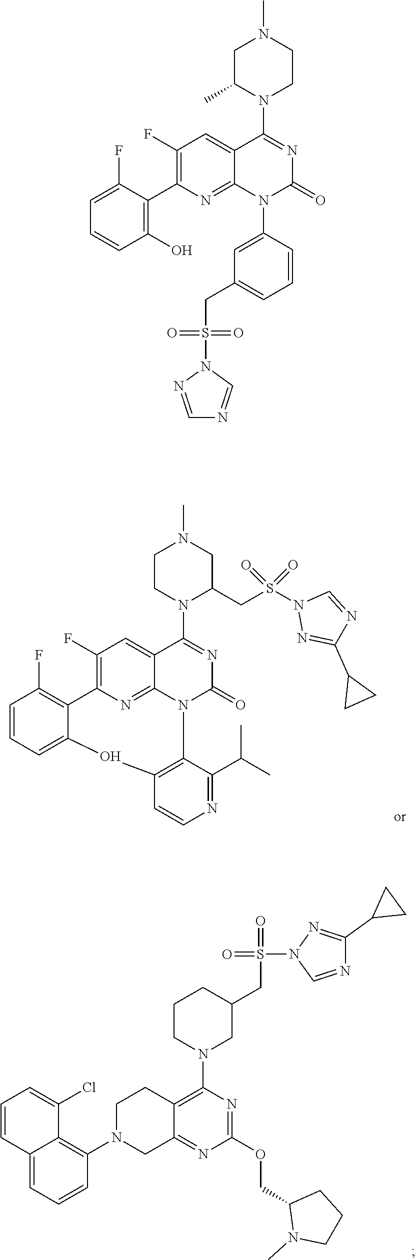 [SMILES: C[C@@H]1CN(C)CCN1c1nc(=O)n(-c2cccc(CS(=O)(=O)n3cncn3)c2)c2nc(-c3c(O)cccc3F)c(F)cc12 ; CC(C)c1nccc2c1-n1c(=O)nc(N3CCN(C)CC3CS(=O)(=O)n3cnc(C4CC4)n3)c3cc(F)c(nc31)-c1c(F)cccc1O2 ; CN1CCC[C@H]1COc1nc2c(c(N3CCCC(CS(=O)(=O)n4cnc(C5CC5)n4)C3)n1)CCN(c1cccc3cccc(Cl)c13)C2]
or a pharmaceutically acceptable salt thereof.

36. A pharmaceutical composition comprising a pharmaceutically acceptable compound of claim 1 or a pharmaceutically acceptable salt thereof and a pharmaceutically acceptable excipient.

37. The pharmaceutical composition of claim 36, wherein the composition is suitable for oral delivery.

38. The pharmaceutical composition of claim 36, wherein the pharmaceutically acceptable compound is of Formula: 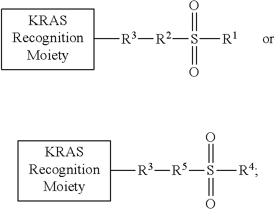
or a pharmaceutically acceptable salt thereof.

39. The pharmaceutical composition of claim 36, wherein the pharmaceutically acceptable compound is Formula: 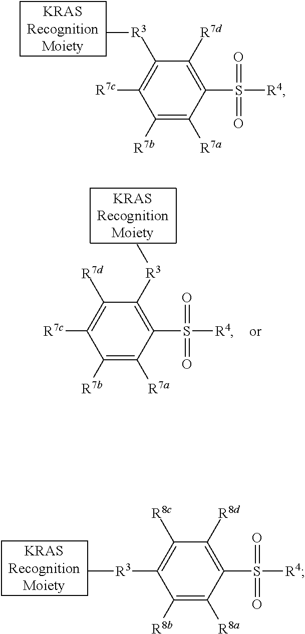
or a pharmaceutically acceptable salt thereof.

40. The pharmaceutical composition of claim 36, wherein the pharmaceutically acceptable compound is of Formula: 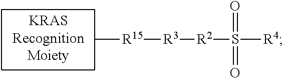
or a pharmaceutically acceptable salt thereof.

41. The pharmaceutical composition of claim 36, wherein the pharmaceutically acceptable compound is of Formula: 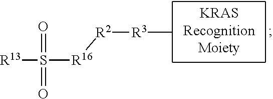
or a pharmaceutically acceptable salt thereof.

42. A method of treating a disorder mediated by KRAS comprising administering an effective amount of a pharmaceutically acceptable compound of claim 1 or a pharmaceutically acceptable salt thereof or a pharmaceutical composition thereof to a human in need thereof.

43. The method of claim 42, wherein the disorder is a cancer.

44. The method of claim 43, wherein the cancer is a solid cancer.

45. The method of claim 43, wherein the cancer is a hematological cancer.

46. The method of claim 43, wherein the cancer is lung cancer.

47. The method of claim 43, wherein the cancer is pancreatic cancer.

48. The method of claim 43, wherein the cancer is colorectal cancer.

49. The method of claim 43, wherein the cancer is ovarian cancer.

50. The method of claim 43, wherein the cancer is metastatic.

51. The method of claim 43, wherein the cancer is relapsed.

52. The method of claim 43, wherein the cancer is refractory.

53. The method of claim 43, wherein the pharmaceutically acceptable compound is of Formula: 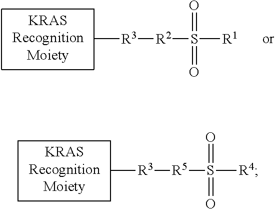
or a pharmaceutically acceptable salt thereof.

54. The method of claim 43, wherein the pharmaceutically acceptable compound is Formula: 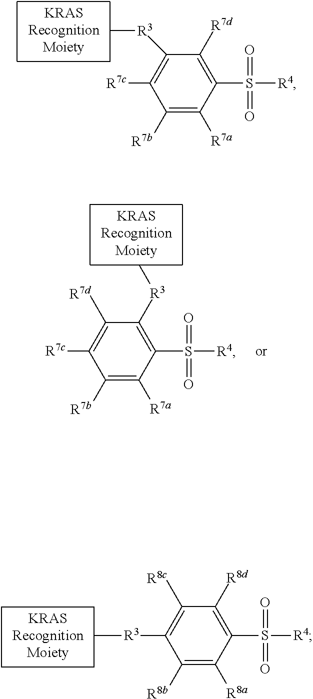
or a pharmaceutically acceptable salt thereof.

55. The method of claim 43, wherein the pharmaceutically acceptable compound is of Formula: 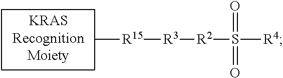
or a pharmaceutically acceptable salt thereof.

56. The method of claim 43, wherein the pharmaceutically acceptable compound is of Formula: 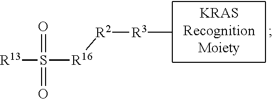
or a pharmaceutically acceptable salt thereof.
